(19) United States
(12) Patent Application Publication
(10) Pub. No.: US 2022/0389465 A1
(43) Pub. Date: Dec. 8, 2022

(54) METHODS OF REDUCING BIURET IN UREA COMPOSITIONS

(71) Applicant: Regents of the University of Minnesota, Minneapolis, MN (US)

(72) Inventors: Lawrence P. Wackett, Minneapolis, MN (US); Lambros J. Tassoulas, Minneapolis, MN (US); Anthony G. Dodge, Minneapolis, MN (US)

(73) Assignee: Regents of the University of Minnesota, Minneapolis, MN (US)

(21) Appl. No.: 17/779,642

(22) PCT Filed: Nov. 25, 2020
(86) PCT No.: PCT/US2020/062367

§ 371 Date: May 25, 2022

Related U.S. Application Data
(60) Provisional application No. 62/941,133, filed on Nov. 27, 2019.

(51) Int. Cl. C12P 13/02; C12N 9/80

(52) U.S. Cl. CPC C12P 13/02; C12N 9/80; C12Y 305/01084

(57) ABSTRACT

Certain embodiments provide a method of reducing biuret in a urea composition, the method comprising contacting the urea composition with an isolated or purified biuret hydrolase enzyme under conditions suitable to reduce the concentration of biuret in the urea composition.

### CROSS-REFERENCE TO RELATED APPLICATION

This application claims priority to U.S. Provisional Application No. 62/941,133 that was filed on Nov. 27, 2019. The entire content of the application referenced above is hereby incorporated by reference herein.

### BACKGROUND OF THE INVENTION

Biuret is a side product present in urea compositions and results from the thermal process that links carbon dioxide and ammonia. For example, typical biuret levels in urea fertilizers are 1-2%. The presence of biuret in fertilizers is undesirable for agriculture because the chemical is toxic to all plants at high levels and to some important crop plants at low levels (e.g., at ˜1%) (see, e.g., Sanford, et al., (1954) Science 120:349-350; Jones, W. W. (1954) Science 1954. 120:499-500; Hasani, et al., (2016) J. Plant Nutrition 39: 749-755; Johnson, et al., (2001) J. Amer. Soc. Hort. Sci. 126:364-370; and Ali, A. G. and C. J. Lova (1994) J. Amer. Soc. Hort. Sci. 119: 1144-1150). In particular, farmers require low-biuret urea (LBU) for major high-value crops, such as oranges, lemons, limes, tree nuts, avocado, cotton and rice. Additionally, LBU can also be used to boost the yield of other crops (e.g., potatoes or sunflowers) (Mikkelson, R. L. (1990) Fertilizer Res. 26: 311-318). Similarly, urea used for diesel exhaust fluids (DEF) must contain low levels of biuret, as the latter interferes with the catalyst in NOx reduction systems for diesel engines that use concentrated urea solutions. DEFs are aqueous urea solutions with a biuret content <0.3%, as mandated by U.S. Environmental Protection Agency, European Union, and other regulators globally. Currently, LBU may be made by thermal chemistry using advanced manufacturing methods with expensive capital equipment. Alternatively, a secondary solvent extraction process might remove biuret. This process does not remove all of the biuret and strips out some urea. Additionally, the solvent biuret-urea mixed extract has an extremely low value and generates large volumes of waste. Other purification methods that have been developed involve adsorption, ion exchange, filtration, or chemical catalysis. These methods are similarly limited. As a consequence, low-biuret urea (LBU) is costly, selling for 2-10 fold more than untreated urea. Thus, new methods for reducing biuret contamination in urea compositions are needed.

### SUMMARY OF THE INVENTION

Accordingly, described herein are methods for the biological remediation of biuret from urea containing compositions (e.g., diesel exhaust fluid (DEF) or fertilizers) using biuret hydrolase.

For example, certain embodiments provide a method of reducing biuret in a urea composition, the method comprising contacting the urea composition with an isolated or purified biuret hydrolase enzyme under conditions suitable to reduce the concentration of biuret in the urea composition.

Certain embodiments provide a composition comprising an isolated or purified biuret hydrolase enzyme and a matrix (e.g., a matrix comprising silica).

Certain embodiments provide a composition comprising a cell (e.g., cross-linked and/or encapsulated) that comprises a biuret hydrolase enzyme.

Certain embodiments provide a device comprising an isolated or purified biuret hydrolase enzyme and a matrix.

Certain embodiments provide a kit comprising an isolated or purified biuret hydrolase enzyme and instructions for contacting a urea composition comprising biuret with the biuret hydrolase enzyme for reducing the concentration of biuret in the composition.

Certain embodiments provide an isolated or purified biuret hydrolase enzyme as described herein.

Certain embodiments provide an isolated or purified triuret hydrolase enzyme comprising an amino acid sequence having an F at position 35, an L at position 39, an N at position 41, an E at position 160, a Y at position 187 and/or and I at position 205, wherein each position is relative to a triuret hydrolase amino acid sequence derived from Herbaspirillum sp. BH-1.

Certain embodiments provide an isolated or purified triuret hydrolase enzyme comprising an amino acid sequence having at least 80% sequence identity to any one of SEQ ID NOs: 169-760.

Certain embodiments provide an isolated or purified triuret hydrolase enzyme as described herein.

Certain embodiments provide an isolated or purified nucleic acid encoding a triuret hydrolase enzyme as described herein.

Certain embodiments provide an expression cassette comprising a nucleic acid as described herein.

Certain embodiments provide a vector comprising an expression cassette as described herein.

Certain embodiments provide a cell comprising an expression cassette as described herein or a vector as described herein.

Certain embodiments provide a composition comprising the isolated or purified triuret hydrolase enzyme as described herein and a matrix (e.g., a matrix comprising silica).

Certain embodiments provide a device comprising a triuret hydrolase enzyme as described herein or a composition as described herein and a matrix.

Certain embodiments provide a method of reducing triuret in a composition, the method comprising contacting the composition with an isolated or purified triuret hydrolase enzyme as described herein, under conditions suitable to reduce the concentration of triuret in the composition.

Certain embodiments provide a kit comprising a triuret hydrolase enzyme as described herein, a cell as described herein, a composition as described herein or a device as described herein and instructions for contacting a first composition comprising triuret with the triuret hydrolase enzyme, cell, composition or device, for reducing the concentration of triuret in the first composition.

### BRIEF DESCRIPTION OF DRAWINGS

FIG. 1. Schematic showing the conversion of carbon dioxide and ammonia to urea; the conversion of urea to biuret; the conversion of biuret to urea using biuret hydrolase.

FIGS. 2A-2B. FIG. 2A. Schematic showing the enzyme biuret hydrolase that converts biuret to allophanic acid which spontaneously undergoes decarboxylation to make urea. FIG. 2B. Urea is shown not to inhibit the Berthelot reaction test for ammonia, allowing the Berthelot reaction to be used to measure the reactivity of biuret hydrolase.

FIGS. 3A-3C. Separation of biuret and urea using High Pressure Liquid Chromatography (HPLC). FIG. 3A. Separation using C₁₈ reverse phase column as the stationary phase and 5% MeOH/95% H₂O isocratic solvent was the mobile phase. FIG. 3B. Separation using anion column and isocratic 5 mM phosphate buffer (pH 8). FIG. 3C. Separation using Thermo Acclaim™ Mixed Mode Wax-1 column using isocratic 25 mM phosphate buffer (pH 6.2).

FIG. 4. Evaluation of urea inhibition of biuret hydrolase and various urea concentrations. Biuret was added at 0.8 mM concentration and 30 microgram enzyme per ml incubated for 30 minutes.

FIGS. 5A-5B. Evaluation of NH₄⁺ released from contaminating biuret in 0.5-8.0 M Fluka urea (>99.5% pure) by biuret hydrolase.

FIG. 6. Schematic showing enzymatic conversion of all fertilizer contaminants (cyanuric acid, triuret, biuret) into the desired product urea.

FIG. 7. Consensus in biuret hydrolase (BiuH) and triuret hydrolase (TrtA) Sequences. Six residue positions shown with arrows were used to separate BiuH and TrtA sequences after SSN clustering. TexShade was used to generate the alignment.

FIG. 8. HPLC evaluation (C18 column) of urea determined by integrating peak area. HPLC was conducted using a C18 reverse phase column as the stationary phase and 5% MeOH/95% H₂O isocratic solvent as the mobile phase.

FIG. 9. Triuret enzymatic hydrolysis by TrtA. HPLC traces of a reaction containing 1 mM triuret (containing 1% wt biuret impurity) in 125 mM sodium phosphate pH 8 before and after 60 minutes of incubation with TrtA enzyme (5 μg).

FIG. 10. Impurities from the manufacturing process for commercial urea.

FIG. 11. Degradation of residual biuret in 3% solution of Loveland urea fertilizer by different amounts of BiuH.

FIGS. 12A-12D. HPLC chromatograms of different commercial urea sources showing the impurities that arise during the pyrolysis manufacturing process that are left, or fail to be eliminated via removal processes (FIG. 12A) Fertilizer grade urea, (FIG. 12B) Low biuret fertilizer (FIG. 12C) Urea sold as diesel emission fluid (DEF), and (FIG. 12D) USP grade urea. The numbers represent the followings: I=urea; II=biuret; III=cyanuric acid; IV=ammelide, V=triuret. Compounds were identified from retention times of authentic standards and absorbance response. The detector was set at 220 nm and elution conditions were as described in the Methods section of Example 6.

FIGS. 13A-13C. Treatment of contaminated urea with an enzyme mixture analyzed by HPLC as described in the Methods section of Example 6. (FIG. 13A) shows the enzymatic reactions including triuret hydrolase, cyanuric acid hydrolase, and biuret hydrolase. (FIG. 13B) Chromatogram at time zero. (FIG. 13C) Chromatogram after 24-hour treatment. The Urea solution contained contaminants and enzyme levels as described in the Methods section of Example 6. Incubations as short as one hour removed contaminants and no further purifications were performed.

FIGS. 14A-14C. Denaturation curves of enzymes in urea solutions. (FIG. 14A) Denaturation curve of biuret hydrolase from Rhizobium leguminosarum by viciae 3841 in urea solutions. (FIG. 14B) Denaturation curve of MtCAH in urea solutions. (FIG. 14C) Denaturation curve of TrtA in urea solutions.

FIG. 15. Rates of residual biuret degradation by BiuH in 3% Loveland fertilizer urea solutions.

FIGS. 16A-16B. Inhibition of BiuH or TrtA in the presence of urea. (FIG. 16A) Limited inhibition of BiuH in the presence of urea. (FIG. 16B) No inhibition of TrtA in the presence of urea.

FIG. 17. Biuret hydrolase, triuret hydrolase, and cyanuric acid hydrolase do not degrade urea.

### DETAILED DESCRIPTION OF THE INVENTION

Described herein are methods for removing contaminants from urea-based compositions, such as urea fertilizers and diesel exhaust fluid (DEF). For example, certain embodiments of the invention provide methods for removing biuret from urea compositions using biuret hydrolase. The feasibility of using a biuret hydrolase in conjunction with a urea composition was completely unexpected based on several factors. In particular, the biuret hydrolase was unexpectedly stable and substrate specific. Urea is commonly used to denature proteins; as described in the Examples (e.g., Examples 1 and 6), the biuret hydrolase was surprisingly stable, even at high urea concentrations (e.g., 4M urea). Additionally, the biuret hydrolase has been shown to be highly stable over a range of temperatures, which is important due to the extreme endothermic reaction that occurs when dissolving urea in water. The activity of the biuret hydrolase was also shown to be exquisitely substrate specific—the enzyme does not accept structurally related compounds as substrates (e.g., urea, cyanuric acid, triuret, and cyanate). As discussed in the Examples, the biuret hydrolase is not inhibited by urea, even at a 10,000 fold higher concentration of urea than biuret. Such specificity is unusual and unexpected, particularly given the fact that 1) urea is structurally similar but generally smaller than biuret; 2) these compounds have the same reactive amide group; and 3) amidases are known for their promiscuity.

The approach of using of biuret hydrolase to remove biuret from urea compositions also provides important and surprising benefits. In particular, this approach achieves lower biuret concentrations than traditional approaches and may be more cost effective and easier to implement. Urea synthesis inherently creates some biuret as a contaminant. Current methods for removing biuret involve a physico-chemical process that has diminishing returns: as the concentration of biuret is lowered, the process begins to extract urea, resulting in negative economic value to the practitioner. Ultra-low-biuret urea-based fertilizers sold commercially still contains biuret (e.g., 0.2%), whereas the enzymatic approach described herein can remove biuret to undetectable levels (e.g., <0.005%). Such purity may improve agricultural practices and enable fewer applications of higher dosed fertilizer since biuret is particularly harmful for certain high value crop plants (citrus, nut, avocado) that must receive numerous foliar applications. It is also noteworthy that biuret hydrolase converts biuret into allophanate, and ultimately urea after spontaneous decarboxylation of allophanate, and urea is the desired compound in urea-based compositions. In other words, the biuret hydrolase enzyme converts a plant toxin into a plant food. This solution has the potential to save famers and consumers money, increase agricultural productivity with less fertilizer application and decrease waste. Similarly, the methods described herein may be advantageously used for other urea-based compositions. For example, urea used for diesel exhaust fluids (DEF) must contain low levels of biuret, as the latter interferes with the catalyst in NOx reduction systems for diesel engines that use concentrated urea solutions.

### Methods of the Invention

Accordingly, certain embodiments provide a method of reducing biuret in a urea composition, the method comprising contacting the urea composition with an isolated or purified biuret hydrolase enzyme under conditions suitable to reduce the concentration of biuret in the urea composition. As used herein, an “isolated” or “purified” enzyme is an enzyme that exists apart from its native environment, and therefore, may be present in a purified form, present in a cell lysate or may be present in a non-native environment such as, for example, in a transgenic host cell. Further, as used herein, the term “enzyme” may be used to refer to an isolated or purified enzyme, an enzyme present in a cell lysate or a cell that expresses the enzyme.

In certain embodiments, the urea composition has a urea concentration between about 0.1M and 8.0M. In certain embodiments, the urea composition has a urea concentration between about 0.1M and 6.0M. In certain embodiments, the urea composition has a urea concentration between about 1M and 6.0M. In certain embodiments, the urea composition has a urea concentration between about 3M and 6.0M. In certain embodiments, the urea composition has a urea concentration between about 5M and 6.0M. In certain embodiments, the urea composition has a urea concentration between about 0.1M and 4.0M. In certain embodiments, the urea composition has a urea concentration between about 0.1M and 2.0M. In certain embodiments, the urea composition has a urea concentration between about 0.5M and 2.0M. In certain embodiments, the urea composition has a urea concentration between about 1.5M and 2.0M. In certain embodiments, the urea composition has a urea concentration between about 1M and 2.0M.

In certain embodiments, the urea composition has a urea concentration of at least about 0.1M, 0.2M, 0.3M, 0.4M, 0.5M, 0.6M, 0.7M, 0.8M, 0.9M, 1M, 1.1M, 1.2M, 1.3M, 1.4M, 1.5M, 1.6M, 1.7M, 1.8M, 1.9M, 2.0M, 2.1M, 2.2M, 2.3M, 2.4M, 2.5M, 2.6M, 2.7M, 2.8M, 2.9M, 3.0M, 3.1M, 3.2M, 3.3M, 3.4M, 3.5M, 3.6M, 3.7M, 3.8M, 3.9M, 4.0M, 4.1M, 4.2M, 4.3M, 4.4M, 4.5M, 4.6M, 4.7M, 4.8M, 4.9M, 5.0M, 5.1M, 5.2M, 5.3M, 5.4M, 5.5M, 5.6M, 5.7M, 5.8M, 5.9M, 6.0M, 6.1M, 6.2M, 6.3M, 6.4M, 6.5M, 6.6M, 6.7M, 6.8M, 6.9M, 7.0M, 7.1M, 7.2M, 7.3M, 7.4M, 7.5M, 7.6M, 7.7M, 7.8M, 7.9M, 8.0M or more. In certain embodiments, the urea composition has a urea concentration of at least about 5.0M, 5.1M, 5.2M, 5.3M, 5.4M, or 5.5M. In certain embodiments, the urea composition has a urea concentration of at least about 5M. In certain embodiments, the urea composition has a urea concentration of at least about 5.4M.

In certain embodiments, the urea composition is in the form of a liquid. In certain embodiments, the liquid urea composition comprises water. In certain embodiments, the liquid urea composition is an aqueous solution of about 32.5% (wt/wt) urea (e.g., undiluted DEF). In certain embodiments, the liquid urea composition comprises at least one organic solvent. In certain embodiments, the liquid urea composition comprises at least one ionic liquid. In certain embodiments the liquid urea composition comprises at least one inorganic or organic buffering component.

In certain embodiments, the urea composition has a pH value from about 3-12, 4-11, or 5-10. In certain embodiments, the urea composition has a pH value of at least about 4.0, 4.5, 5.0, 5.5, 6.0, 6.5, 7.0, 7.5, 8.0, 8.5, 9.0, 9.5, or 10. In certain embodiments, the urea composition has a pH value of at least about 9.0. In certain embodiments, the urea composition has a pH value of at least about 9.1. In certain embodiments, the urea composition has a pH value of at least about 9.2. In certain embodiments, the urea composition has a pH value of at least about 9.3. In certain embodiments, the urea composition has a pH value of at least about 9.4.

In certain embodiments, the urea composition is in the form of a solid (e.g., granule, prill or crystal).

In certain embodiments, the urea composition is a high-biuret urea (e.g., comprises at least about 0.2% biuret). In certain embodiments, the urea composition prior to treatment comprises at least about 5%, 4%, 3%, 2%, 1%, 0.9%, 0.8%, 0.7%, 0.6%, 0.5%, 0.4% or 0.3% biuret.

In certain embodiments, the urea composition prior to treatment comprises at least about 100 fold, 1,000 fold, 10,000, or 100,000 fold more urea than biuret.

In certain embodiments, a method described herein reduces the concentration of biuret in a urea composition by at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300% or more.

In certain embodiments, a method described herein reduces the concentration of biuret in the urea composition to less than about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less. In certain embodiments, a method described herein reduces the concentration of biuret in the urea composition to an undetectable level, e.g., using a method described herein, such as via a Berthelot ammonia assay or HPLC, or using a method known in the art (see, e.g., Murray, et al., 1982: Anal. Chem. 54:1504-1507).

In certain embodiments, a method described herein reduces the concentration of biuret in the urea composition from about 1% or more biuret to about 0.1% or less. In certain embodiments, a method described herein reduces the concentration of biuret in the urea composition from about 1% or more biuret to about 0.01% or less. In certain embodiments, a method described herein reduces the concentration of biuret in the urea composition from about 1% or more biuret to about 0.001% or less. In certain embodiments, a method described herein reduces the concentration of biuret in the urea composition from about 1% or more biuret to undetectable levels (e.g., using a method described herein or known in the art).

In certain embodiments, a method described herein reduces the concentration of biuret in the urea composition from about 0.5% or more biuret to about 0.1% or less. In certain embodiments, a method described herein reduces the concentration of biuret in the urea composition from about 0.5% or more biuret to about 0.01% or less. In certain embodiments, a method described herein reduces the concentration of biuret in the urea composition from about 0.5% or more biuret to about 0.001% or less. In certain embodiments, a method described herein reduces the concentration of biuret in the urea composition from about 0.5% or more biuret to undetectable levels (e.g., using a method described herein or known in the art).

In certain embodiments, a method described herein reduces the concentration of biuret in the urea composition to less than about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less in about 24 hours or less (e.g., less than about 20 hours, about 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, 30 min, 20 min, 10 min, 5 min or 1 min).

Urea compositions described herein are useful for a variety of commercial and industrial applications. For example, in certain embodiments, a urea composition described herein may be used as a raw material in the manufacturing process of chemical(s) or may be incorporated into another composition (e.g., the urea composition may be comprised within another composition). In certain embodiments, a urea composition described herein may be used in the production of certain plastics, polymers, feedstocks (e.g., potassium cyanate), urea nitrates, glues, resins (e.g., urea-formaldehyde resins), adhesives (urea-formaldehyde or urea-melamine-formaldehyde adhesives), fertilizers, toilet bowl cleaners, dish washing machine detergents/dish soaps, hair coloring and conditioning products, pesticides, and fungicides. In certain embodiments, a urea composition described herein may be used to separate chemical mixtures (e.g., racemic mixtures or paraffin), as well as in the production of aviation fuel or lubricating oils. A urea composition described herein may also be used to reduce NOx pollutants in exhaust gases from combustion (e.g., from power plants or diesel engines). Thus, in certain embodiments, a urea composition may be used in a catalytic convertor. In certain embodiments, a urea composition may be used as a laboratory reagent (e.g., for protein denaturing, as a eutectic solvent, or as a hydrogen source). A urea composition described herein, may also be used in a medicinal composition. For example, it may be incorporated in the manufacture of barbiturates, dermatological products (e.g., skin re-hydrating products, facial cleansers, bath oils, skin softeners, lotions, hair removers), tooth whitening products, and diuretics. In may also be used in certain medical tests and procedures, including, e.g., debridement of nails, as an earwax removal aid, in urea injections, urine therapy or in a urea breath test. Certain other uses of urea compositions include, but are not limited to, as a stabilizer in a nitrocellulose explosive; as a de-icer (non-corrosive de-icer); as a flavor-enhancing additive for cigarettes; as a browning agent in factory-produced pretzels; as a reactant in some ready-to-use cold compresses; as a cloud seeding agent; as a flame-proofing agent (e.g., in a urea-potassium bicarbonate mixture); as a yeast nutrient (e.g., in combination with ammonium phosphate); as a nutrient for plankton; as an additive to extend the working temperature and open time of hide glue; or as a solubility-enhancing/moisture-retaining additive to dye baths for textile dyeing or printing.

In certain embodiments, the urea composition is used as a fertilizer. In certain embodiments the urea composition is comprised within a fertilizer composition (e.g., formulated as a fertilizer). In certain embodiments, the fertilizer composition further comprises ammonium nitrate.

In certain embodiments, the urea composition is used as a DEF. In certain embodiments the urea composition is comprised within a DEF composition (e.g., formulated as a DEF).

Depending on the use of the urea composition, there may be differing levels of tolerance for contaminants present in the urea composition. For example, certain crops tolerate only very low levels of biuret or certain medical applications may require high purity urea. Thus, in certain embodiments, a method described herein further comprises contacting the urea composition with one or more additional enzymes. For example, a urea composition may be further contacted with one or more additional enzymes to increase the purity of the urea and to reduce the concentration of other contaminants present in the composition. In certain embodiments, a urea composition may be contacted with a cyanuric acid hydrolase (CAH) enzyme to convert cyanuric acid present in the urea composition into carboxybiuret, which then spontaneously decarboxylates into biuret. Such biuret would then be converted into allophanate by the biuret hydrolase, which is ultimately converted into urea. Similarly, a urea composition may be also contacted with a triuret hydrolase enzyme to convert triuret present in the urea composition into carboxybiuret (see, FIG. 6). A urea composition may also be contacted with an ammelide hydrolase to reduce ammelide in the urea composition.

Thus, in certain embodiments, a method described herein further comprises contacting the urea composition with one or more additional enzymes as described herein (e.g., a CAH enzyme, a triuret hydrolase enzyme and/or an ammelide hydrolase). In certain embodiments, the urea composition is contacted concurrently with the biuret hydrolase enzyme and the one or more additional enzymes. In certain embodiments, the biuret hydrolase enzyme and the one or more additional enzymes are present in a single composition or device. In certain embodiments, the biuret hydrolase enzyme and the one or more additional enzymes are present in different compositions or different devices. In certain embodiments, the urea composition is contacted sequentially with the biuret hydrolase enzyme and the one or more additional enzymes. In certain embodiments, the urea composition is contacted with the biuret hydrolase enzyme first and the one or more additional enzymes second. In certain embodiments, the urea composition is contacted with the biuret hydrolase enzyme second and the one or more additional enzymes first.

In certain embodiments, the one or more additional enzymes are selected from the group consisting of a CAH enzyme, a triuret hydrolase enzyme, and an ammelide hydrolase. Thus, in certain embodiments, a method described herein further comprises contacting the urea composition with a CAH enzyme as described herein. In certain embodiments, a method described herein further comprises contacting the urea composition with a triuret hydrolase enzyme as described herein. In certain embodiments, a method described herein further comprises contacting the urea composition with an ammelide hydrolase enzyme as described herein. In certain embodiments, a method described herein further comprises contacting the urea composition with at least one enzyme selected from the group consisting of a CAH enzyme, a triuret hydrolase enzyme, and an ammelide hydrolase. In certain embodiments, a method described herein further comprises contacting the urea composition with a CAH enzyme, a triuret hydrolase enzyme, and an ammelide hydrolase. In certain embodiments, the one or more additional enzymes are present in a composition or a device, as described herein.

Thus, in certain embodiments, a method described herein reduces the concentration of cyanuric acid, triuret, and/or ammelide in a urea composition by at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300% or more.

In certain embodiments, a method described herein reduces the concentration of cyanuric acid, triuret, and/or ammelide in the urea composition to less than about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less. In certain embodiments, a method described herein reduces the concentration of cyanuric acid, triuret, and/or ammelide in the urea composition to an undetectable level, e.g., using a method described herein or using a method known in the art.

In certain embodiments, a method described herein increases the concentration of urea in the urea composition. In certain embodiments, a method described herein increases the concentration of urea in the urea composition by at least about 1%, 2%, 3%, 4%, 5%, 6%, 7%, 8%, 9%, 10%, or more.

In certain embodiments, the urea composition is treated at a factory prior to being sold. For example, in certain embodiments, the urea composition may be contacted with the enzyme(s) (e.g., the biuret hydrolase, CAH enzyme, triuret hydrolase, and/or ammelide hydrolase enzyme) after the melt manufacturing process (e.g., after the urea composition is cooled down by dissolution in water) (see, e.g., Meessen, J. H. (2012) Ullmann's Encyclopedia of Industrial Chemistry, 6th Edition VCH: Weinheim, Germany). In certain embodiments, the urea composition is contacted with a solution comprising the enzyme(s). In certain embodiments, the urea composition is in the form of a solid (e.g., urea prills, granules or crystals) and is coated with the enzyme solution. In certain embodiments, the enzyme solution is misted/sprayed onto the urea composition. In such an embodiment, the enzyme solution coating the urea composition may be dried; enzyme activation and remediation would occur when the coated urea composition is dissolved in water prior to use.

In certain embodiments, the urea composition is treated by a consumer prior to use (e.g., prior to spraying a field with the urea composition).

In certain embodiments, the urea composition is contacted with the enzyme(s) (e.g., the biuret hydrolase, CAH enzyme, triuret hydrolase enzyme, and/or ammelide hydrolase) in a separate treatment tank.

In certain embodiments, the enzyme (e.g., the biuret hydrolase, CAH enzyme, triuret hydrolase enzyme, and/or ammelide hydrolase) is added directly to a urea composition for remediation.

In certain embodiments, the enzyme(s) is dried. In certain embodiments, the enzyme (e.g., the biuret hydrolase, CAH enzyme, triuret hydrolase enzyme, and/or ammelide hydrolase) is present in pellet form (e.g., a tablet). In certain embodiments, the method further comprises mixing a solid urea composition and the enzyme(s) with water (e.g., the enzyme becomes active upon hydration). In certain embodiments, the method involves adding the enzyme (e.g., the biuret hydrolase, CAH enzyme, triuret hydrolase enzyme, and/or ammelide hydrolase) to a liquid urea composition, wherein the enzyme is in the form of a free enzyme, or wherein the enzyme is part of a device or part of a device through which liquid flows through or over during the process of treating the composition. In certain embodiments, the enzyme is present in a cell or cell lysate (e.g., operably linked to the device or a solid support comprised within the device). In certain embodiments, the enzyme, cell or cell lysate is cross-linked and/or encapsulated (e.g., with glutaraldehyde, and/or beads, such as alginate beads). In certain embodiments, the liquid urea composition is contacted with the device described herein by passing the liquid over or through the device. In certain embodiments, the liquid urea composition flows through the device (e.g., pumped through the device). In certain embodiments, the enzyme is present in a hose and is contacted with the urea composition during discharge. In certain embodiments, the enzyme is comprised within a column and the enzyme is contacted with the urea composition as it passes through the column.

In certain embodiments, the urea treatment is effected during a time period of about 24 hours or less (e.g., less than about 20 hours, less than about 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, 30 min, 20 min, 10 min, 5 min or 1 min).

### Biuret Hydrolase Enzymes

The present invention also provides biuret hydrolase enzymes and compositions and devices comprising such enzymes, e.g., which may be used for reducing the concentration of biuret in a composition, e.g., a urea composition, such as from a urea-based fertilizer.

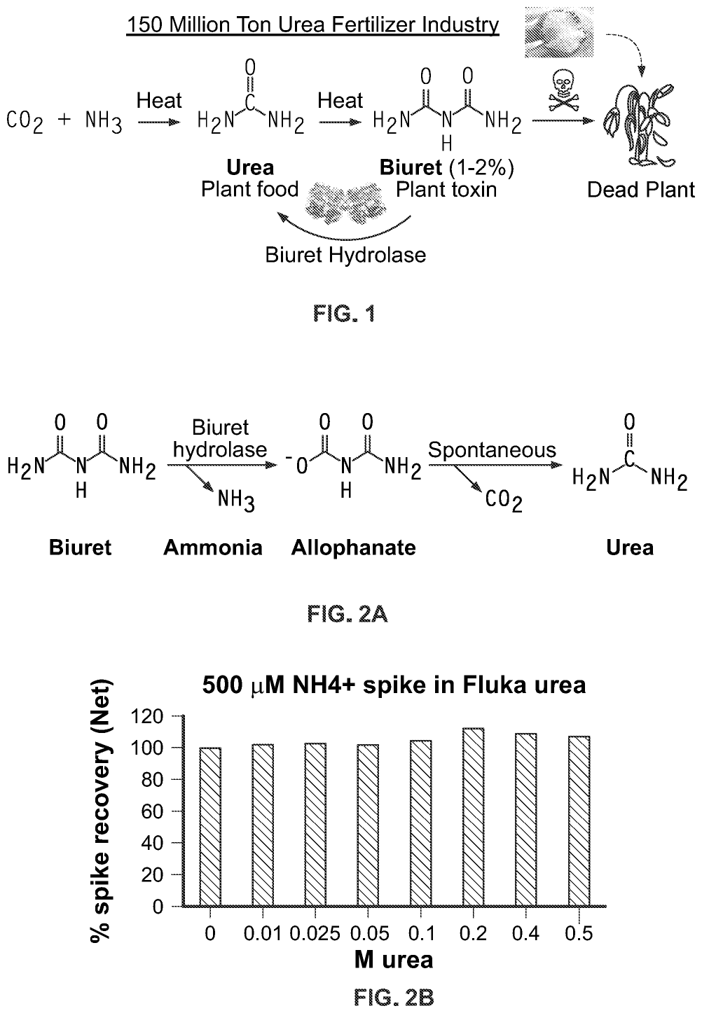

Thus, certain embodiments of the invention provide a biuret hydrolase enzyme (e.g., for use in a method, composition or device described herein). As used herein, the term “biuret hydrolase enzyme” refers to an enzyme that is capable of catalyzing the hydrolysis of biuret to allophanate, which undergoes spontaneous, non-enzymatic decarboxylation to urea (see, FIGS. 1, 2A and 6). As shown in Table 1, biuret hydrolase enzymes are produced by a variety of bacterial species and examples of amino acid sequences encoding biuret hydrolase enzymes are included in Table 1 (see also, Robinson et al., (2018) Environ. Microbiol. 20(6): 2099-2111, Cameron et al. (2011) ACS Catalysis 1:1075-1082; Esquirol et al., (2018) PLoS One 13(2):e0192736; and Nishihara, et al., (1965) Biochem. J. 8: 23-34, which are incorporated by reference herein for all purposes).

Thus, in certain embodiments, the biuret hydrolase is an enzyme derived from a bacterial or eukaryotic species as described in Table 1. In certain embodiments, the biuret hydrolase is derived from a bacterium of Catellatospora citrea, Rhodovulum sp. NI22, Herbaspirillum, Rhizobium or Rhodococcus. In certain embodiments, the biuret hydrolase is derived from a bacterium of Herbaspirillum. In certain embodiments, the biuret hydrolase is derived from a bacterium of Herbaspirillum sp. BH-1. In certain embodiments, the biuret hydrolase is derived from a bacterium of Rhizobium. In certain embodiments, the biuret hydrolase is derived from a bacterium of Rhizobium leguminosarum. In certain embodiments, the biuret hydrolase is derived from a bacterium of Rhodococcus. In certain embodiments, the biuret hydrolase is derived from a bacterium of Rhodococcus sp. Mel. In certain embodiments, the biuret hydrolase is an enzyme derived from a thermophilic bacterial species. In certain embodiments, the biuret hydrolase is derived from a bacterium of Catellatospora citrea. In certain embodiments, the biuret hydrolase is derived from a bacterium of Rhodovulum sp. NI22.

In certain embodiments, the biuret hydrolase enzyme is an enzyme described in Robinson et al., (2018) Environ. Microbiol. 20(6): 2099-2111. In certain embodiments, the biuret hydrolase enzyme comprises a D-K-C catalytic triad amino acid sequence. For example, the D-K-C catalytic triad may be present at positions 30, 139 and 175, respectively, in a biuret hydrolase derived from Herbaspirillum sp. BH-1, or at equivalent residues in a corresponding biuret hydrolase enzyme. In certain embodiments, the biuret hydrolase enzyme comprises a GIT amino acid sequence at residues 166-168 of a biuret hydrolase enzyme derived from Herbaspirillum sp. BH-1, or at equivalent residues in a corresponding biuret hydrolase enzyme. In certain embodiments, the biuret hydrolase enzyme comprises an E at residue 78, a K at residue 142 and/or a Q at residue 212 of a biuret hydrolase enzyme derived from Herbaspirillum sp. BH-1, or at equivalent residues in a corresponding biuret hydrolase enzyme. In certain embodiments, the biuret hydrolase enzyme comprises a R[E/D]AN motif. In certain embodiments, the biuret hydrolase enzyme comprises a R[E/D]ANDRG[F/Y][E/D]C motif.

As described in Example 3, biuret hydrolase enzymes comprise certain amino acids at particular positions that distinguish them from triuret hydrolase enzymes. In particular, the biuret hydrolase enzyme from Herbaspirillum sp. BH-1 comprises Y35, M39, Y41, D160, T187 and V205. Thus, in certain embodiments, the biuret hydrolase enzyme comprises an amino acid sequence having an Y at position 35, an M at position 39, a Y at position 41, a D at position 160, a T at position 187 and/or and V at position 205. As described herein, these amino acid positions are relative to a biuret hydrolase amino acid sequence derived from Herbaspirillum sp. BH-1; however, these amino acids may be located at equivalent positions in corresponding biuret hydrolase enzymes derived from other organisms. Such equivalent positions may be identified by one skilled in the art using methods described herein or known in the art (e.g., BLAST or ALIGN).

In certain embodiments, the biuret hydrolase enzyme comprises an amino acid sequence having at least about 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence described in any one of the following accession numbers: AEX65081.1, NP_791183.1, WP_031595628.1, WP_033263155.1, WP_004883226.1, WP_007177325.1, WP_008346673.1, WP_008877630.1, WP_010106328.1, WP_011427969.1, WP_011828366.1, WP_012427107.1, WP_012489672.1, WP_041935977.1, WP_013107455.1, WP_013233429.1, WP_013652708.1, WP_013673377.1, WP_013963785.1, WP_015795031.1, WP_018333481.1, WP_018449133.1, WP_026179047.1, WP_020563252.1, WP_020617109.1, WP_026468572.1, WP_020923004.1, WP_022713792.1, WP_028614812.1, WP_024315610.1, WP_003421848.1, WP_025398328.1, WP_025418539.1, WP_026784459.1, WP_027195197.1, WP_028228770.1, WP_028739231.1, WP_029007464.1, WP_051392089.1, WP_030472255.1, WP_035078376.1, WP_035256306.1, WP_036050193.1, WP_037459080.1, WP_037484943.1, WP_040114689.1, WP_040119808.1, WP_044431670.1, WP_045672424.1, WP_045774129.1, WP_046572974.1, WP_050475712.1, WP_054985868.1, WP_057403488.1, WP_044530929.1, WP_060717458.1, WP_062363788.1, WP_064243180.1, WP_064823845.1, WP_064837226.1, WP_066257666.1, WP_066811963.1, WP_068803416.1, WP_069307252.1, WP_072378795.1, WP_072642261.1, WP_073055721.1, WP_074637487.1, WP_074830085.1, WP_074987393.1, WP_075290549.1, WP_075633397.1, WP_076625678.1, WP_078814169.1, WP_079177709.1, WP_079417747.1, WP_083726432.1, WP_085560469.1, WP_085780954.1, WP_085861497.1, WP_090877027.1, WP_091276718.1, WP_091583823.1, WP_093084166.1, WP_093153408.1, WP_093620408.1, WP_093645941.1, YP_234257.1, WP_051074034.1, WP_040604119.1, WP_040454192.1, WP_009983899.1, WP_010429021.1, WP_011654379.1, WP_012976323.1, WP_013893344.1, WP_014993113.1, WP_015343698.1, WP_016558329.1, WP_016735441.1, WP_018246800.1, WP_018326144.1, WP_031255153.1, WP_020514528.1, WP_022978704.1, WP_023495169.1, WP_023561466.1, WP_024671285.1, WP_027054243.1, WP_027475322.1, WP_027798423.1, WP_027820346.1, WP_030439668.1, WP_033319363.1, WP_033361216.1, WP_035252302.1, WP_035935333.1, WP_035963207.1, WP_037083615.1, WP_051963325.1, WP_038587753.1, WP_039788660.1, WP_052418263.1, WP_045231530.1, WP_045672421.1, WP_046104327.1, WP_046153182.1, WP_053199920.1, WP_054019041.1, WP_054360926.1, WP_054999487.1, WP_058088296.1, WP_059193874.1, WP_060602508.1, WP_061116979.1, WP_061133981.1, WP_062033021.1, WP_062137725.1, WP_062243904.1, WP_068114315.1, WP_083229793.1, WP_073173303.1, WP_084564509.1, WP_074072734.1, WP_074585157.1, WP_075854492.1, WP_076625677.1, WP_077980810.1, WP_085558546.1, WP_085749770.1, WP_085877124.1, WP_085935041.1, WP_090798859.1, WP_091010500.1, WP_091295461.1, WP_091641346.1, WP_092373934.1, WP_092547462.1, WP_092679559.1, WP_092852955.1, WP_092860340.1, WP_093280567.1, WP_093410371.1, WP_037209122.1, and RKE06538.1. In certain embodiments, the biuret hydrolase enzyme consists of an amino acid sequence having at least about 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence described in any one of the accession numbers listed above.

In certain embodiments, the biuret hydrolase comprises an amino acid sequence having at least about 60% sequence identity to any one of SEQ ID NOs:1-164, 769 and 771. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs:1-164, 769 and 711. In certain embodiments, the amino acid sequence comprises any one of SEQ ID NOs:1-164, 769 and 771. In certain embodiments, biuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs:1-164, 769 and 771.

In certain embodiments, the biuret hydrolase enzyme comprises an amino acid sequence having at least about 60% sequence identity to SEQ ID NO:1. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1. In certain embodiments, the amino acid sequence comprises SEQ ID NO:1. In certain embodiments, the biuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1. In certain embodiments, the biuret hydrolase enzyme consists of SEQ ID NO:1. In certain embodiments, the biuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:761 or SEQ ID NO:762.

In certain embodiments, the biuret hydrolase enzyme comprises an amino acid sequence having at least about 60% sequence identity to SEQ ID NO:2. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:2. In certain embodiments, the amino acid sequence comprises SEQ ID NO:2. In certain embodiments, the biuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:2. In certain embodiments, the biuret hydrolase enzyme consists of SEQ ID NO:2. In certain embodiments, the biuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:763 or SEQ ID NO:764.

In certain embodiments, the biuret hydrolase enzyme comprises an amino acid sequence having at least about 60% sequence identity to SEQ ID NO:95. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:95. In certain embodiments, the amino acid sequence comprises SEQ ID NO:95. In certain embodiments, the biuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:95. In certain embodiments, the biuret hydrolase enzyme consists of SEQ ID NO:95. In certain embodiments, the biuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:765.

In certain embodiments, the biuret hydrolase enzyme comprises an amino acid sequence having at least about 60% sequence identity to SEQ ID NO:769. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:769. In certain embodiments, the amino acid sequence comprises SEQ ID NO:769. In certain embodiments, the biuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:769. In certain embodiments, the biuret hydrolase enzyme consists of SEQ ID NO:769. In certain embodiments, the biuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:761 or SEQ ID NO:770.

In certain embodiments, the biuret hydrolase enzyme comprises an amino acid sequence having at least about 60% sequence identity to SEQ ID NO:771. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:771. In certain embodiments, the amino acid sequence comprises SEQ ID NO:771. In certain embodiments, the biuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:771. In certain embodiments, the biuret hydrolase enzyme consists of SEQ ID NO:771. In certain embodiments, the biuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:778.

In certain embodiments, the biuret hydrolase enzyme is a variant of a biuret hydrolase enzyme as described herein.

In certain embodiments, the biuret hydrolase enzyme is a catalytically active fragment of a biuret hydrolase enzyme as described herein.

In certain embodiments, the biuret hydrolase enzyme is linked to a peptide tag (e.g., a polyhistidine-tag, such as a His tag).

In certain embodiments, the biuret hydrolase enzyme has limited activity with urea. For example, in certain embodiments, the activity of the biuret hydrolase enzyme with urea is at least about 50, 100, 1,000, 10,000, or more times slower than that with biuret. In certain embodiments, the activity of the biuret hydrolase enzyme with urea is undetectable, e.g., using a method described herein, such as via the detection of ammonia formation or using chromatographic quantification of urea (HPLC), or another method known in the art.

In certain embodiments, the biuret hydrolase enzyme is produced by a bacterium (e.g., a naturally occurring bacterium or a recombinant bacterium). In certain embodiments, the biuret hydrolase enzyme is produced by yeast or fungus. In certain embodiments, the biuret hydrolase enzyme is produced recombinantly.

In certain embodiments, the biuret hydrolase enzyme is an isolated or purified biuret hydrolase enzyme. In certain embodiments, the biuret hydrolase enzyme is present in a cell lysate (e.g., a crude protein lysate). In certain embodiments, the enzyme is present in a cell. In certain embodiments, the cell rapidly transports biuret into the cell, facilitating the enzyme reaction inside the cell. In certain embodiments, the cell has been permeabilized to enable biuret to penetrate into the cell. In certain other embodiments, the biuret hydrolase enzyme may be expressed on the surface of a cell (e.g., a bacterial or yeast cell).

In certain embodiments, the biuret hydrolase enzyme is present in a live cell. In certain embodiments, the biuret hydrolase enzyme is present in a dead cell. In certain embodiments, the biuret hydrolase enzyme is present in a fixated or cross-linked cell treated with a cross-linking fixative (e.g., glutaraldehyde or formaldehyde). For example, the biuret hydrolase enzyme can be present in a glutaraldehyde cross-linked cell. In certain embodiments, the cross-linking fixative is glutaraldehyde, formaldehyde, dimethyl suberimidate, disuccinimidyl suberate, m-Maleimidobenzoyl-N-hydroxysuccinimide ester, polyethylenimine, or a photo-activatable cross-linking agent such as N-((2-pyridyldithio)ethyl)-4-azidosalicylamide.

In certain embodiments, the cell is a transgenic cell that recombinantly expresses an exogenously derived biuret hydrolase. In certain embodiments, the cell is an E. coli cell comprising a biuret hydrolase. In certain embodiments, the biuret hydrolase is an enzyme derived from a bacterial or eukaryotic species as described in Table 1. In certain embodiments, the biuret hydrolase is derived from a bacterium of Herbaspirillum, Rhizobium, Rhodococcus, Rhodovulum sp. NI22, or Catellatospora citrea. In certain embodiments, the biuret hydrolase is derived from a bacterium of Catellatospora citrea. In certain embodiments, the biuret hydrolase is derived from a bacterium of Rhodovulum sp. NI22. In certain embodiments, the cell is a native non-recombinant cell comprising an endogenous biuret hydrolase.

In certain embodiments, a cell comprising a biuret hydrolase is immobilized or encapsulated. For example, the cell (e.g., live cell or cross-linked cell) may be immobilized or encapsulated using an encapsulating agent such as hydrogel (e.g., alginate, chitosan, or a polyacrylamide gel). In certain embodiments, the encapsulating agent (e.g., a hydrogel-forming polymer) is selected from the group consisting of polysaccharides, water soluble polyacrylates, polyphosphazenes, poly(acrylic acids), poly(methacrylic acids), copolymers of acrylic acid and methacrylic acid, poly(alkylene oxides), polyacrylamide, poly(vinyl acetate), polyvinyl alcohol, polyvinylpyrrolidones, and combination thereof. In certain embodiments, the encapsulating agent is a polysaccharide selected from the group consisting of alginate, chitosan, agarose, hyaluronan, chondroitin sulfate, and combination thereof. In certain embodiments, the encapsulating agent comprises alginate. In certain embodiments, the encapsulating agent comprises chitosan. In certain embodiments, the cell is encapsulated within a hydrogel bead. In certain embodiments, the bead has a size range of about 1 μm to 10 mm, 2 μm to 5 mm, 3 μm to 3 mm, 5 μm to 1 mm, 6 μm to 500 μm, 7 μm to 300 μm, 8 μm to 200 μm, 10 μm to 100 μm, 20 μm to 80 μm, or 30 μm to 60 μm. In certain embodiments, the cell may be immobilized or encapsulated through entrapment, conjugation or the induction of biofilm formation onto a variety of matrices (e.g., diatomite, celite, diatomaceous earth, silica, plastics, or resins) as described herein. In certain embodiments, the cell is immobilized with a silica matrix. Cellular immobilization or encapsulation methods are described herein and known in the art. For example, methods for cellular immobilization or encapsulation are described in U.S. Pat. Nos. 4,744,933, 5,427,935, 5,635,609, 5,827,707, 6,242,230, 9,034,348, 9,096,845, 10,478,401, 10,548,844, and 10,786,446, which are incorporated by reference for all purposes.

In certain embodiments, a cell comprising a biuret hydrolase as described herein is encapsulated within hydrogel. In certain embodiments, a cell comprising a biuret hydrolase as described herein is encapsulated within alginate or chitosan hydrogel. In certain embodiments, a cross-linked cell (e.g., via glutaraldehyde) comprising a biuret hydrolase is encapsulated within an alginate or chitosan hydrogel.

Without wishing to be bound by theory, the cellular cross-linking and/or encapsulation (e.g., in a hydrogel bead) may provide enhanced cellular structural stability and further protection for the enzyme against chemical denaturation (e.g., high concentration urea or high pH) and/or physical denaturation (e.g., shearing stress) to enhance enzyme stability, longevity, and/or reusability under harsh working conditions (e.g., for contacting DEF or a urea composition wherein the urea concentration is at least about 5M or higher).

Accordingly, in certain embodiments, the methods described herein comprise contacting a urea composition (e.g., fertilizer or DEF) with a biuret hydrolase enzyme under conditions suitable to reduce the concentration of biuret in the urea composition, wherein the biuret hydrolase enzyme is free enzyme, immobilized to a matrix as described herein, or present in a cell as described herein.

Certain embodiments provide a method of reducing biuret in a urea composition, the method comprising contacting the urea composition with a biuret hydrolase enzyme under conditions suitable to reduce the concentration of biuret in the urea composition, wherein the biuret hydrolase is present in a cell as described herein. In certain embodiments, the method comprises contacting the urea composition with a cell comprising a biuret hydrolase enzyme under conditions suitable to reduce the concentration of biuret in the urea composition. In certain embodiments, the method comprises contacting the urea composition with a biuret hydrolase enzyme that is immobilized to a matrix under conditions suitable to reduce the concentration of biuret in the urea composition.

In certain embodiments, a cell as described herein is dispersed in a liquid urea composition (e.g., fertilizer or DEF) for incubation with or without stirring. After biuret reduction, the cell can remain in contact with the liquid urea composition or may be removed from the liquid urea composition by, e.g., via filtration, centrifugation, settlement or any suitable separation technique.

In certain embodiments, the enzyme(s) or cell(s) comprising the enzyme(s) as described herein is encased in a device or immobilized onto a matrix, wherein the liquid urea composition comes into contact with the device or matrix. In certain embodiments, the liquid urea composition flows through a device or matrix continually and can be optionally recirculated through the device or matrix.

The present invention also includes isolated or purified nucleic acids, expression cassettes and vectors that encode the biuret hydrolase enzymes described above (e.g., for use in generating a biuret hydrolase for use in a method described herein).

Accordingly, certain embodiments of the invention provide an isolated or purified nucleic acid encoding a biuret hydrolase enzyme described herein. In certain embodiments, the nucleic acid sequence is codon optimized.

Certain embodiments of the invention also provide an expression cassette comprising the nucleic acid encoding a biuret hydrolase enzyme described herein. In certain embodiments, the expression cassette further comprises a promoter, such as a regulatable promoter or a constitutive promoter. In certain embodiments, the promoter is operably linked to the nucleic acid encoding the biuret hydrolase enzyme. In certain embodiments, the expression cassette further comprises a second nucleic acid encoding a peptide tag. In certain embodiments, the second nucleic acid is operably linked to the nucleic acid encoding the biuret hydrolase enzyme.

Certain embodiments of the invention provide a vector comprising an expression cassette described herein. In certain embodiments, the vector further comprises a nucleic acid sequence encoding a cyanuric acid hydrolase (CAH) enzyme, a triuret hydrolase enzyme, and/or an ammelide hydrolase as described herein.

Certain embodiments of the invention provide a cell comprising an expression cassette or a vector described herein. In certain embodiments, the cell further comprises an expression cassette comprising a nucleic acid sequence encoding a CAH enzyme, a triuret hydrolase enzyme, and/or an ammelide hydrolase as described herein or a vector comprising such an expression cassette.

Certain embodiments of the invention provide a cell lysate derived from a cell described herein.

Certain embodiments also provide a kit comprising a biuret hydrolase enzyme as described herein, packaging material, and instructions for contacting a urea composition comprising biuret with the biuret hydrolase enzyme for reducing the concentration of biuret in the composition. In certain embodiments, the kit further comprises a CAH enzyme, a triuret hydrolase enzyme, and/or an ammelide hydrolase as described herein. In certain embodiments, the enzyme(s) is present in a composition or a device described herein. In certain embodiments, the kit further comprises a urea composition. In certain embodiments, the enzyme is dried. In certain embodiments the urea composition is a solid (e.g., a granule, prill or crystal form). In certain embodiments, the instructions further state the enzyme and urea composition should be mixed with water.

### Additional Enzymes

As described herein, a urea composition may be further contacted with one or more additional enzymes to increase the purity of the urea and to reduce the concentration of other contaminants present in the composition. For example, a urea composition may be contacted with a CAH enzyme to convert cyanuric acid present in the urea composition into carboxybiuret, which then spontaneously decarboxylates into biuret. Such biuret would then be converted into allophanate by the biuret hydrolase, which is ultimately converted into urea. Similarly, a urea composition may be also contacted with a triuret hydrolase enzyme to convert triuret present in the urea composition into carboxybiuret (see, FIG. 6). A urea composition may be also contacted with an ammelide hydrolase to degrade ammelide.

Accordingly, in certain embodiments, a method described herein further comprises contacting a urea composition with a CAH enzyme, a triuret hydrolase enzyme, and/or an ammelide hydrolase.

As used herein, a CAH enzyme refers to an enzyme that hydrolytically catalyzes the ring-opening reaction that converts cyanuric acid to carboxybiuret. Different types of CAH enzymes have been previously reported (Seffernick, J. L. and L. P. Wackett (2016) Appl. Environ. Microbiol. 82: 1638-1645; Seffernick et al., (2012) J. Bacteriol. 194:4579-4588; Aukema, et al., Appl. Environ. Microbiol. 86(2): e01964-19, 2020, which are incorporated by reference in its entirety for all purposes). For example, CAH enzymes are described in U.S. Pat. Nos. 8,367,389 and 10,233,437, which are incorporated by reference in their entirety for all purposes. In certain embodiments, the CAH enzyme is derived from Moorella thermoacetica. In certain embodiments, the CAH enzyme is derived from Pseudomonas sp. ADP. In certain embodiments, the CAH enzyme is derived from Acidovorax citrulli. In certain embodiments, the CAH enzyme is derived from Azorhizobium caulinodans.

The amino acid sequence of an exemplary CAH enzyme is shown in Table 1 as SEQ ID NO:165.

In certain embodiments, SEQ ID NO:165 is mutated and the cysteine at residue 46 is replaced with an alanine (C46A) (see, SEQ ID NO:166).

In certain embodiments, SEQ ID NO:165 is mutated and the cysteine at residue 46 is replaced with a serine (C46S) (see, SEQ ID NO:167).

In certain embodiments, SEQ ID NO:165 is mutated and the cysteine at residue 46 is replaced with a glycine (C46G) (see, SEQ ID NO:168).

The amino acid sequences of additional exemplary CAH enzymes are shown in Table 1 as SEQ ID NOs:772-774.

Thus, in certain embodiments, the CAH enzyme comprises an amino acid sequence having at least about 60% sequence identity to any one of SEQ ID NOs:165-168 and 772-774.

In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs:165-168 and 772-774. In certain embodiments, the amino acid sequence comprises any one of SEQ ID NOs:165-168 and 772-774. In certain embodiments, CAH enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NOs:165-168 and 772-774.

In certain embodiments, the CAH enzyme is linked to a peptide tag (e.g., a polyhistidine-tag, such as a His tag).

In certain embodiments, the CAH enzyme is an isolated or purified CAH enzyme.

The present invention also includes isolated or purified nucleic acids, expression cassettes and vectors that encode the CAH enzymes described above (e.g., for use in a method described herein).

As used herein, an ammelide hydrolase enzyme refers to an enzyme that catalyzes the deamination reaction that converts ammelide to cyanuric acid, which in turn can be degraded by the CAH enzyme. Different types of ammelide hydrolase enzymes are known in the art (Zhou N, et al., 2020. Environ Pollut. 27:115803, doi: 10.1016/j.envpol.2020.115803; Shapir N, et al., 2002. J Bacteriol. 184(19):5376-84, doi: 10.1128/jb.184.19.5376-5384.2002; Eaton R W, et al., 1991. J Bacteriol. 173(3):1363-6, doi: 10.1128/jb.173.3.1363-1366.1991). For example, in certain embodiments, the ammelide hydrolase enzyme is AtzC. In certain embodiments, the ammelide hydrolase enzyme is N-isopropylammelide isopropyl amidohydrolase. In certain embodiments, the ammelide hydrolase enzyme is ammelide aminohydrolase.

In certain embodiments, the ammelide hydrolase enzyme is derived from Pseudomonas sp. (e.g., Pseudomonas sp. ADP). In certain embodiments, the ammelide hydrolase enzyme is derived from Pseudomonas sp. ADP. In certain embodiments, the ammelide hydrolase enzyme is derived from Acidovorax citrulli.

Exemplary ammelide hydrolase enzyme amino acid sequences are shown in Table 1 as SEQ ID NO:775-776).

Thus, in certain embodiments, the ammelide hydrolase enzyme comprises an amino acid sequence having at least about 60% sequence identity to any one of SEQ ID NO:775-776. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:775-776. In certain embodiments, the amino acid sequence comprises any one of SEQ ID NO:775-776. In certain embodiments, ammelide hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:775-776. In certain embodiments, the ammelide hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence described herein (e.g., SEQ ID NO:777).

In certain embodiments, the ammelide hydrolase enzyme is linked to a peptide tag (e.g., a polyhistidine-tag, such as a-His tag).

In certain embodiments, the ammelide hydrolase enzyme is an isolated or purified ammelide hydrolase enzyme.

The present invention also includes isolated or purified nucleic acids, expression cassettes and vectors that encode the ammelide hydrolase enzymes described above (e.g., for use in a method described herein).

In certain embodiments, the triuret hydrolase enzyme is an enzyme as described below.

### Certain Triuret Hydrolase Embodiments

Certain embodiments of the invention also provide triuret hydrolase enzymes and methods of use thereof. As used herein, a triuret hydrolase enzyme refers to an enzyme that converts triuret into carboxybiuret. As described in Example 3, while triuret and biuret hydrolases often comprise similar sequences, at least 6 residues have been shown to be divergent. For example, when comparing the triuret hydrolase and the biuret hydrolase sequences from Herbaspirillum sp. BH-1, residues vary at positions 35, 39, 41, 160, 187 and 205. In particular, triuret hydrolase from Herbaspirillum sp. BH-1 comprises F35, L39, N41, E160, Y187 and 1205, while biuret hydrolase comprises Y35, M39, Y41, D160, T187 and V205. Thus, in certain embodiments, the triuret hydrolase enzyme comprises an amino acid sequence having an F at position 35, an L at position 39, an N at position 41, an E at position 160, a Y at position 187 and/or and I at position 205. As described herein, these amino acid positions are relative to a triuret hydrolase amino acid sequence derived from Herbaspirillum sp. BH-1; however, the amino acids may be located at equivalent positions in corresponding triuret hydrolase enzymes derived from other organisms. Such equivalent positions may be identified by one skilled in the art using methods described herein or known in the art (e.g., BLAST or ALIGN).

Certain triuret hydrolase enzymes are also described in Tassoulas, et al, J Biol Chem. 2020 Nov. 10; jbc.RA120.015631, which incorporated by reference herein.

In certain embodiments, the triuret hydrolase enzyme is derived from Herbaspirillum (e.g., Herbaspirillum sp. BH-1). In certain embodiments, the triuret hydrolase enzyme is derived from Rhzobium. In certain embodiments, the triuret hydrolase enzyme is derived from Actinoplanes. In certain embodiments, the triuret hydrolase enzyme is derived from Rhodobacter.

Exemplary triuret hydrolase enzyme amino acid sequences are shown in Table 1 as SEQ ID NO:169-760).

Thus, in certain embodiments, the triuret hydrolase enzyme comprises an amino acid sequence having at least about 60% sequence identity to any one of SEQ ID NO:169-760. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:169-760. In certain embodiments, the amino acid sequence comprises any one of SEQ ID NO:169-760. In certain embodiments, triuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO:169-760. In certain embodiments, the triuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a sequence described herein.

In certain embodiments, the triuret hydrolase enzyme comprises an amino acid sequence having at least about 60% sequence identity SEQ ID NO:169. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NOs:169. In certain embodiments, the amino acid sequence comprises SEQ ID NO:169. In certain embodiments, triuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:169. In certain embodiments, the triuret hydrolase enzyme is encoded by a nucleic acid sequence comprising/consisting of a nucleic acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:766.

In certain embodiments, the triuret hydrolase enzyme comprises an amino acid sequence having at least about 60% sequence identity SEQ ID NO:170. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NOs:170. In certain embodiments, the amino acid sequence comprises SEQ ID NO:170. In certain embodiments, triuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:170.

In certain embodiments, the triuret hydrolase enzyme comprises an amino acid sequence having at least about 60% sequence identity SEQ ID NO:171. In certain embodiments, the amino acid sequence has at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NOs:171. In certain embodiments, the amino acid sequence comprises SEQ ID NO:171. In certain embodiments, triuret hydrolase enzyme consists of an amino acid sequence having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:171.

In certain embodiments, the triuret hydrolase enzyme is linked to a peptide tag (e.g., a polyhistidine-tag, such as a His tag).

In certain embodiments, the triuret hydrolase enzyme is an isolated or purified triuret hydrolase enzyme.

The present invention also includes isolated or purified nucleic acids, expression cassettes and vectors that encode the triuret hydrolase enzymes described above.

Accordingly, certain embodiments of the invention provide an isolated or purified nucleic acid encoding a triuret hydrolase enzyme described herein. In certain embodiments, the nucleic acid sequence is codon optimized.

Certain embodiments of the invention also provide an expression cassette comprising the nucleic acid encoding a triuret hydrolase enzyme described herein. In certain embodiments, the expression cassette further comprises a promoter, such as a regulatable promoter or a constitutive promoter. In certain embodiments, the promoter is operably linked to the nucleic acid encoding the triuret hydrolase enzyme. In certain embodiments, the expression cassette further comprises a second nucleic acid encoding a peptide tag. In certain embodiments, the second nucleic acid is operably linked to the nucleic acid encoding the triuret hydrolase enzyme.

Certain embodiments of the invention provide a vector comprising an expression cassette described herein. In certain embodiments, the vector further comprises a nucleic acid sequence encoding an additional enzyme described herein (e.g., a biuret hydrolase enzyme or a CAH enzyme).

Certain embodiments of the invention provide a cell comprising an expression cassette or a vector described herein. Certain embodiments of the invention provide a cell lysate derived from a cell described herein.

Certain embodiments also provide a kit comprising a triuret hydrolase enzyme as described herein, packaging material, and instructions for contacting a composition comprising triuret with the triuret hydrolase enzyme to reduce the concentration of triuret in the composition. In certain embodiments, the kit further comprises an additional enzyme described herein (e.g., a biuret hydrolase enzyme or a CAH enzyme).

Certain embodiments also provide a method of reducing triuret in a composition, the method comprising contacting the composition with an isolated or purified triuret hydrolase enzyme under conditions suitable to reduce the concentration of triuret in the composition.

In certain embodiments, the composition is a liquid. In certain embodiments, the composition comprises water. In certain embodiments, the composition comprises urea (e.g., is a urea composition described herein). In certain embodiments, the composition is a composition described herein.

In certain embodiments, the composition prior to treatment comprises at least about 5%, 4%, 3%, 2%, 1%, 0.9%, 0.8%, 0.7%, 0.6%, 0.5%, 0.4%, 0.3%, 0.2% or 0.1% triuret.

In certain embodiments, a method described herein reduces the concentration of triuret in the composition by at least about 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300% or more.

In certain embodiments, a method described herein reduces the concentration of triuret in the composition to less than about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less. In certain embodiments, a method described herein reduces the concentration of triuret in the composition to an undetectable level, e.g., using a method described herein or using a method known in the art.

In certain embodiments, the treatment is effected during a time period of about 24 hours or less (e.g., less than about 20 hours, less than about 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, 30 min, 20 min, 10 min, 5 min or 1 min).

In certain embodiments, a method described herein reduces the concentration of triuret in the composition to less than about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less in about 24 hours or less (e.g., less than about 20 hours, less than about 19 hours, 18 hours, 17 hours, 16 hours, 15 hours, 14 hours, 13 hours, 12 hours, 11 hours, 10 hours, 9 hours, 8 hours, 7 hours, 6 hours, 5 hours, 4 hours, 3 hours, 2 hours, 1 hour, 30 min, 20 min, 10 min, 5 min or 1 min).

In certain embodiments, a method described herein further comprises contacting the composition with one or more additional enzymes as described herein (e.g., a CAH enzyme, an ammelide hydrolase enzyme and/or a biuret hydrolase enzyme). In certain embodiments, the composition is contacted concurrently with the triuret hydrolase enzyme and the one or more additional enzymes. In certain embodiments, the triuret hydrolase enzyme and the one or more additional enzymes are present in a single composition or device. In certain embodiments, the triuret hydrolase enzyme and the one or more additional enzymes are present in different compositions or different devices. In certain embodiments, the composition is contacted sequentially with the triuret hydrolase enzyme and the one or more additional enzymes. In certain embodiments, the composition is contacted with the triuret hydrolase enzyme first and the one or more additional enzymes second. In certain embodiments, the composition is contacted with the triuret hydrolase enzyme second and the one or more additional enzymes first.

In certain embodiments, the method involves adding the enzyme (e.g., the biuret hydrolase, CAH enzyme, triuret hydrolase enzyme, and/or ammelide hydrolase) to a composition, wherein the enzyme is in the form of a free enzyme, or wherein the enzyme is part of a device or part of a device through which the composition flows through or over during the process of treating the composition. In certain embodiments, the composition is contacted with a device described herein by passing the composition over or through the device. In certain embodiments, the composition flows through the device.

Compositions and Devices Certain embodiments of the present invention also provide compositions and devices comprising an enzyme described herein. Such compositions or devices may be used for reducing biuret or triuret in a composition in need of remediation (e.g., a urea composition).

For example, such compositions or devices may be used for reducing biuret in a urea composition, such as a urea fertilizer or DEF. In certain embodiments, the compositions or devices comprise one or more biuret hydrolase enzymes described herein. In certain embodiments, the compositions or devices comprise one or more triuret hydrolase enzymes described herein. As described herein, the term “enzyme” may be used to refer to an isolated or purified enzyme, an enzyme present in a lysate or a cell that expresses the enzyme. Thus, in certain embodiments, the biuret hydrolase enzyme or triuret hydrolase enzyme is isolated or purified. In certain embodiments, the biuret hydrolase is present in a cell or in cell lysate. In certain embodiments, the triuret hydrolase is present in a cell or in cell lysate. In certain embodiments, a cell as described herein may treated with a cross-linking fixative (e.g., glutaraldehyde or formaldehyde). For example, an enzyme as described herein can be present in a glutaraldehyde cross-linked cell. In certain embodiments, a cell described herein may be immobilized or encapsulated, e.g., using a hydrogel (e.g., alginate, or a polyacrylamide gel), or through the induction of biofilm formation onto a variety of matrices (e.g., diatomite, celite, diatomaceous earth, silica, plastics, or resins). Cellular immobilization or encapsulation methods are described herein and known in the art. For example, methods for cellular immobilization or encapsulation are described in U.S. Pat. Nos. 4,744,933, 5,427,935, 5,635,609, 5,827,707, 6,242,230, 9,034,348, 9,096,845, 10,478,401, 10,548,844, and 10,786,446, which are incorporated by reference for all purposes.

In certain embodiments, the composition or device comprises a biuret hydrolase enzyme. In certain embodiments, the composition or device further comprises a CAH enzyme described herein. In certain embodiments, the composition or device further comprises a triuret hydrolase enzyme described herein. In certain embodiments, the composition or device further comprises an ammelide hydrolase enzyme described herein. In certain embodiments, the composition or device further comprises a CAH enzyme, a triuret hydrolase enzyme and/or an ammelide hydrolase enzyme described herein.

In certain embodiments, the composition or device comprises a triuret hydrolase enzyme. In certain embodiments, the composition or device further comprises a CAH enzyme described herein. In certain embodiments, the composition or device further comprises a biuret hydrolase enzyme described herein. In certain embodiments, the composition or device further comprises an ammelide hydrolase enzyme described herein. In certain embodiments, the composition or device further comprises a CAH enzyme and a biuret hydrolase enzyme described herein. In certain embodiments, the composition or device further comprises a CAH enzyme, a biuret hydrolase enzyme described herein and an ammelide hydrolase enzyme described herein.

In certain embodiments, a composition described herein further comprises a carrier.

In certain embodiments, the biuret hydrolase enzyme is incorporated into a carrier. In certain embodiments, the biuret hydrolase enzyme is conjugated to a carrier. In certain embodiments, a CAH enzyme, a triuret hydrolase enzyme and/or an ammelide hydrolase enzyme is incorporated into a carrier or conjugated to a carrier. In certain embodiments, the carrier enables the enzyme to be recycled after its initial use (e.g., isolated from the urea composition and used 2, 3, 4, 5 or more times).

In certain embodiments, the triuret hydrolase enzyme is incorporated into a carrier. In certain embodiments, the triuret hydrolase enzyme is conjugated to a carrier. In certain embodiments, a CAH enzyme, an ammelide hydrolase enzyme and/or a biuret hydrolase enzyme is incorporated into a carrier or conjugated to a carrier. In certain embodiments, the carrier enables the enzyme to be recycled after its initial use (e.g., isolated from the urea composition and used 2, 3, 4, 5 or more times).

In certain embodiments, the enzyme(s) (e.g., biuret hydrolase, triuret hydrolase, CAH and/or an ammelide hydrolase enzyme) is present in a cell(s) as described herein. In certain embodiments, the enzyme(s) is present in a native cell that expresses an endogenous enzyme. In certain embodiments, the enzyme(s) is present in a transgenic host cell that expresses an exogenous enzyme. In certain embodiments, the enzyme(s) is present in a cross-linked and/or encapsulated cell(s) as described herein. In certain embodiments, the composition comprises one or more cell(s) comprising biuret hydrolase, triuret hydrolase and/or CAH enzyme(s) as described herein. In certain embodiments, the composition comprises one or more cell(s) comprising biuret hydrolase, triuret hydrolase, CAH enzyme(s) as described herein and/or an ammelide hydrolase enzyme as described herein.

In certain embodiments, the composition may comprise a cell comprising biuret hydrolase, triuret hydrolase, CAH, and/or an ammelide hydrolase enzyme. In certain embodiments, the composition may comprise a cell comprising biuret hydrolase and CAH. In certain embodiments, the composition may comprise a cell comprising biuret hydrolase and triuret hydrolase. In certain embodiments, the composition may comprise a cell comprising CAH and triuret hydrolase. In certain embodiments, the composition may comprise two cell types, each comprising biuret hydrolase or CAH respectively. In certain embodiments, the composition may comprise two cell types, each comprising biuret hydrolase or triuret hydrolase respectively. In certain embodiments, the composition may comprise two cell types, each comprising CAH or triuret hydrolase respectively. In certain embodiments, the composition may comprise three cell types each comprising biuret hydrolase, triuret hydrolase, or CAH respectively. In certain embodiments, the composition may comprise one or more cell types comprising an ammelide hydrolase enzyme.

In certain embodiments, a composition described herein is formulated in pellet form (e.g., as a tablet).

Certain embodiments of the invention also provide a device comprising a composition as described herein.

In certain embodiments, a composition or a device described herein further comprises a matrix (e.g., a matrix comprising silica). In certain embodiments, the enzyme(s) present in a composition or device described herein are incorporated in, into, or on a matrix. In certain embodiments, the enzyme(s) incorporated in, into, or on a matrix is a biuret hydrolase enzyme, a CAH enzyme, a triuret hydrolase enzyme, and/or an ammelide hydrolase enzyme. In certain embodiments, the enzyme(s) is immobilized to a matrix. For example, in certain embodiments, the enzyme(s) can be adsorbed, complexed or conjugated to a matrix. In certain embodiments, the enzyme(s) has an affinity tag (e.g., a polyhistidine-tag) to facilitate its immobilization within a matrix. In certain embodiments, the matrix has chelated ions (e.g., Fe(III), Co(II), Ni(II),

Cu(II), Zn(II)) for binding with an affinity tag (e.g., a polyhistidine-tag) of the enzyme(s). In certain embodiments, the enzyme(s) is treated with a cross-linking agent as described herein (e.g., glutaraldehyde and/or polyethylenimine (PEI)). The enzyme(s) can be treated with a cross-linking agent before or after the enzyme(s) is immobilized to a matrix (e.g., a glass resin). In certain embodiments, the enzyme(s) is treated with glutaraldehyde. In certain embodiments, the enzyme(s) is treated with polyethylenimine (PEI). In certain embodiments, the enzyme(s) is treated with glutaraldehyde and PEI. In certain embodiments, the matrix is water-insoluble. In certain embodiments, the enzyme(s) are incorporated in or on an insoluble matrix (i.e., insoluble in a liquid urea composition), which serves as a solid support for the enzyme, namely, it provides a stationary object with respect to the composition in need of remediation (e.g., urea composition). The insoluble matrix allows performing a continuous and/or repetitive contact of the treated composition (e.g., urea composition) with the enzyme, as well as maintaining the enzyme affixed, thus eliminating loss of the enzyme due to leaching out. In certain embodiments, the insoluble matrix is granular and/or porous. In certain embodiments, the insoluble matrix is an organic matrix or an inorganic matrix. In certain embodiments, the matrix is an organic matrix and the organic matrix is plastic, nylon, activated carbon, cellulose, agarose, chitin, chitosan, collagen and/or polystyrene. In certain embodiments, the matrix is an inorganic matrix and the inorganic matrix is glass, zeolite, silica, alumina, titania, zirconia, calcium alginate and/or celite. In certain embodiments, the matrix comprises silica. In certain embodiments, the matrix comprises agarose (e.g., cross-linked agarose). For example, the matrix comprises Sepharose. In certain embodiments, the agarose is cyanogen bromide-activated Sepharose, epoxy-activated-Sepharose, N-hydroxysuccinimidyl-Sepharose, or glyoxal-agarose. In certain embodiments, the matrix comprises glass. In certain embodiments, the matrix is a glass resin such as a porous glass particle.

In certain embodiments, the enzyme is encapsulated in a silica-matrix, as described in WO 2012/116013, which is hereby incorporated by reference in its entirety. In certain embodiments, the silica nanoparticles are cross-linked with alkoxysiloxanes (e.g., tetraethoxysiloxane (TEOS)) to encapsulate the enzyme.

Many commercially available solid-phase synthesis columns, purification and ion-exchange columns are packed with granular and/or porous matrices that are suitable for protein immobilization applications, or can readily be modified so as to be suitable for protein immobilization, and therefore are suitable for use as the insoluble matrix according to the present invention. Such granular and/or porous insoluble matrices are well known in the art and are used in various applications such as filtration and chromatography. Representative examples include, without limitation, organic substances such as nylons, polystyrenes, polyurethanes and other synthetic polymers and co-polymers, activated carbon, cellulose, agarose, chitin, chitosan and collagen, and inorganic substances such as beads, filters, cloth, glass, plastic, zeolite, silica, alumina, titania, zirconia, calcium alginate and celite.

Other forms of organic polymers, copolymers and cross-linked derivatives thereof, and inorganic materials such as diatomaceous earths and other types of molecular sieves, typically used in various filtrations, can be used as a granular and/or porous insoluble matrix, according to the present invention, on or in which an enzyme can be incorporated.

The term “incorporated,” as used herein, refers to any mode of contact between the matrix and the enzyme, which achieves immobilization of the enzyme with respect to the matrix, thus rendering a biochemically active enzyme insoluble, or in other words immobilized, and in some cases more protected, than the soluble enzyme.

Incorporation of an enzyme (e.g., a cell expressing the enzyme) into or on the matrix can be effected by attachment via any type of chemical bonding, including covalent bonds, ionic (electrostatic) bonds, hydrogen bonding, hydrophobic interactions, metal-mediated complexation, affinity-pair bonding and the like, and/or by attachment via any type of physical interaction such as magnetic interaction, surface adsorption, encapsulation, entrapment, entanglement and the like. The enzyme(s) can be incorporated in and/or on physical structural elements of an insoluble matrix. In cases where the structural elements of the matrix are granular but not porous, such as, for example, in cases where the matrix is made of solid glass beads or particles, or solid plastic beads or particles, the enzyme(s) is incorporated on the surface of the beads or particles, and the composition (e.g., urea composition) that flows in the channels between the beads or particles comes in contact with the enzyme(s), thus allowing the amide-containing compounds dissolved in the water to be enzymatically degraded.

In cases where the structural element of the matrix is porous but not granular, such as, for example, in cases where the matrix is extruded zeolite blocks, carbonaceous blocks or solid plastic foam blocks, the enzyme(s) is incorporated in the cavities, on the inner surface of the innate inter-connected pores and channels which are characteristic to such matrices, as well as on the outer surface of the block, and the composition (e.g., urea composition) that flows in the inter-connected pores and channels comes in contact with the enzyme(s). In cases where the structural elements of the matrix are granular and porous, such as, for example, in cases where the matrix is zeolite granules or molecular sieves pellets, the enzyme(s) is incorporated on the surface of the granules or pellets and in the inner surface of the pores and channels of these matrices, and the composition (e.g., urea composition) that flows between the granules or pellets as well as through them comes in contact with the enzyme(s), thus allowing the amide-containing compounds dissolved in the composition (urea composition) to be enzymatically degraded.

In certain embodiments, the incorporation of the enzyme to the insoluble matrix is effected by a combination of chemical and physical attachments such as covalent bonding and entanglement.

In certain embodiments of the present invention, the incorporation of the enzyme to the insoluble matrix is effected by covalently attaching the enzyme to the insoluble matrix (the solid support) by conventional methods known in the art for enzyme immobilization.

Exemplary immobilization techniques are described for example in U.S. Pat. Nos. 4,071,409, 4,090,919, 4,258,133, 4,888,285, 5,177,013, 5,310,469, 5,998,183, 6,905,733, and 6,987,079, U.S. Patent Application Publication No. 2003/0096383, and in Yan -A-X. et al, 2002, Applied Biochemistry and Biotechnology, Vol. 101(2), pp. 113-130(18); and Ye, Yun-hua et al, 2004, Peptide Science, Vol. 41, pp 613-616, which are incorporated herein by reference. Briefly, protein immobilization by covalent bonding to a solid matrix, according to certain embodiments of the present invention, is based on coupling two functional groups, as these are defined herein below, one within the matrix (e.g., on its surface) and the other within the enzyme (e.g., on its surface), either directly or via a spacer. The spacer can be, for example, a bifunctional moiety, namely, a compound having at least two functional groups which are capable of forming covalent bonds with functional groups of both the matrix and the enzyme. As used herein, the phrase “functional group” describes a chemical group that has certain functionality and therefore can participate in chemical reactions with other components which lead to chemical interactions as described hereinabove (e.g., a bond formation). The phrase “cross-linking agent,” as used herein, refers to a bifunctional compound that can promote or regulate intermolecular interactions between polymer chains, linking them together to create a more rigid structure. Cross-links are bonds linking functional groups of polymers and/or other substances, so as to form intermolecular interactions there-between and, as a result, a three-dimensional network interconnecting these substances. Cross-linking can be effected via covalent bonds, metal complexation, hydrogen bonding, ionic bonds and the like.

In certain embodiments, a device described herein further comprises at least one casing or housing for the matrix. In certain embodiments, the composition (e.g., urea composition) flows through the at least one casing and contacts the enzyme (e.g., a biuret hydrolase enzyme, a triuret hydrolase enzyme and/or an additional enzyme described herein). For example, in certain embodiments, the device may be a flow through reactor, a tea-bag-type device as described below, a pipe optionally linked to a pump, a skimmer that moves around the top of a liquid/composition (e.g., urea composition), a device that attaches to a sprayer, or a sand bed filter. In certain embodiments, the device further comprises a permeable layer. In certain embodiments, the enzyme(s) is imbedded in or on the permeable layer.

The casing may be used so as to avoid sweeping of the enzyme(s) by the liquid/composition (e.g., urea composition) passing through the device. Another purpose of a casing is to form the desired shape and cross-section of the device, which will optimize its function and maintain a continuous, void-free bed of the enzyme(s) presented herein. The casing material is preferably selected suitable for high-pressure, and is typically insoluble in the composition (e.g., urea composition) and water-tight. Furthermore, the casing material is preferably selected inactive and stable with respect to composition in need of remediation (e.g., urea and other chemicals typically present in fertilizers). Examples for suitable casing materials include, without limitation, plastic (e.g., mesh), galvanized metal and glass.

In certain embodiments, the device for treatment of a composition (e.g., urea composition) includes a casing with two parallel perforated faces, constituting a semi-closed compartment, whereby the composition presented herein fills, or partially fills the compartment. The casing thus has one perforated face for an inlet for the composition in need of remediation (e.g., urea composition), and the other perforated face for an outlet. The composition (e.g., urea composition) to be treated (containing the amide-containing compound(s)) enters the inlet and comes in contact with the permeable and insoluble matrix having the enzyme(s) incorporated therein or thereon.

In certain embodiments, the device for remediation of a composition (e.g., urea composition) comprises a mesh or porous casing, wherein the casing forms a compartment (e.g., a mesh or porous bag, e.g., a mesh or porous bag similar to a tea bag), whereby the enzyme and matrix fills or partially fills the compartment of the mesh/porous casing. The device may be placed in a composition to be treated (e.g., a urea composition) and natural diffusion processes allow the composition to permeate the casing and contact the enzyme (e.g., a biuret hydrolase enzyme, a triuret hydrolase enzyme, an ammelide hydrolase and/or a CAH enzyme), thereby resulting in the degradation of biuret, cyanuric acid, ammelide, and/or triuret.

In certain embodiments, the device may include an immobilizing matrix that has a permeable layer.

Other exemplary devices typically for used for water treatment may be modified for the treatment of a liquid/composition (e.g., urea composition). For example, a device for use in the present invention may be a filter cartridge, similar to that disclosed, for example, in U.S. Pat. No. 6,325,929, and containing, as the composition, an extruded solid, water-permeable carbonaceous material block as a water-insoluble matrix and one or more biuret hydrolase enzyme(s) or one or more triuret hydrolase enzyme(s) incorporated in and on the carbonaceous block.

Other water-treatment devices that are suitable for use in the context of the present invention are also described, for example, in U.S. Pat. Nos. 4,532,040, 4,935,116, 5,055,183, 5,478,467, 5,855,777, 5,980,761, 6,257,242 and 6,325,929, which are incorporated by reference.

Treatment devices utilized in circulating reservoirs typically form a part of a larger system, which is typically referred to as a plant (e.g., a plant at a factory that generates urea fertilizers). Typical treatment devices used in plants of circulating reservoirs exert their designated treatment action when liquid flows there-through, either by means of a pump or by gravity. The liquid flows into the system, enters the device, and passes through a water-permeable and water-insoluble matrix within the device, which effects the designated treatment action, typically filtration of insoluble particulates and objects, chemical exchange of solutes and ions and dissolution and addition of chemicals into the liquid.

The device containing a biuret hydrolase enzyme, a triuret hydrolase enzyme, an ammelide hydrolase enzyme, and/or CAH enzyme described herein, or a composition described herein, can therefore be any device, or part of a device through which liquid flows during the process of treating the liquid. Such a device can be, for example, one or more of a filter, a filter cartridge, an ion-exchanger, an erosion feeder and the likes, as is exemplified hereinbelow. The device may be a removable device such as a removable filter cartridge. Such a removable device can be manufactured and sold separately as a “replacement” cartridge.

Thus, according to certain embodiments, a biuret hydrolase enzyme, a triuret hydrolase enzyme, an ammelide hydrolase enzyme, and/or a CAH enzyme described herein, or composition as described herein, can be added to a liquid-treatment device having a liquid-treatment substance embedded therein which effects the originally designated treatment action of these devices, or replace that substance altogether.

The device, according to the present embodiments, can form a part of a comprehensive liquid treatment system, which exerts other treatment actions, such as filtration of solid particulates and addition of chemicals. Liquid that flows through such a treatment system also flows through the device presented herein. The system can be designed such that all its liquid capacity flows through the device, or such that only a part of its liquid capacity flows through.

Typically, the flow rate can be adjusted per device for the optimal function of the system and every device in it. For an efficient function of the present device, which includes an immobilized active enzyme (e.g., a biuret hydrolase enzyme, a triuret hydrolase enzyme, an ammelide hydrolase enzyme, and/or a CAH enzyme described herein), the amount of enzyme, amount of water-insoluble matrix, overall shape of the device and flow-rate need to be designed to as to suit the system's layout, capacity (power) and the expected rate at which the concentration of an amide-containing compound such as, for example, biuret or triuret, is required to be reduced. The rate of an amide-containing compound reduction depends on the enzymatically catalyzed reaction condition, e.g., temperature, pH, ionic strength and, in relevance to this case, liquid flow. All the above mentioned parameters are considered while designing the device.

The incorporation of enzymes (e.g., a biuret hydrolase enzyme, a triuret hydrolase enzyme, an ammelide hydrolase enzyme, and/or CAH enzyme described herein) to insoluble matrices is typically measured in international units of activity. An international unit (IU) of an enzyme is defined as the amount of enzyme that produces one micromole of a reaction product in one minute under defined reaction conditions. The amount of IU which can be incorporated to a matrix depends on the type of matrix and incorporation technique, surface area of the matrix, the availability and chemical reactivity of functional groups suitable for conjugation in both the enzyme and the matrix, and on the residual enzymatic activity subsequent to the incorporation process. Typical enzyme load ranges from a few IU to hundreds of IU of an enzyme per cm³ of matrix material. An optimal load, namely, the optimal amount of enzyme to be incorporated per a unit volume of insoluble matrix material, is an example of one parameter that is considered while designing the device.

### Certain Definitions

The term “nucleic acid” and “polynucleotide” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“Synthetic” nucleic acids are those prepared by chemical synthesis. The nucleic acids may also be produced by recombinant nucleic acid methods. “Recombinant nucleic acid molecule” is a combination of nucleic acid sequences that are joined together using recombinant nucleic acid technology and procedures used to join together nucleic acid sequences as described, for example, in Sambrook and Russell (2001). As used herein, the term “recombinant nucleic acid,” e.g., “recombinant DNA sequence or segment” refers to a nucleic acid, e.g., to DNA, that has been derived or isolated from any appropriate cellular source, that may be subsequently chemically altered in vitro, so that its sequence is not naturally occurring, or corresponds to naturally occurring sequences that are not positioned as they would be positioned in a genome that has not been transformed with exogenous DNA. An example of preselected DNA “derived” from a source would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “recombinant DNA” includes completely synthetic DNA sequences, semi-synthetic DNA sequences, DNA sequences isolated from biological sources, and DNA sequences derived from RNA, as well as mixtures thereof.

The invention encompasses isolated or substantially purified nucleic acid compositions. In the context of the present invention, an “isolated” or “purified” DNA molecule or an “isolated” or “purified” polypeptide is a DNA molecule that exists apart from its native environment. An isolated DNA molecule may exist in a purified form or may exist in a non-native environment such as, for example, a transgenic host cell or bacteriophage. For example, an “isolated” or “purified” nucleic acid molecule, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. In one embodiment, the RNA or DNA is “isolated” in that it is free from at least one contaminating nucleic acid with which it is normally associated in the natural source of the RNA or DNA and in one embodiment of the invention is substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid. In one embodiment, an “isolated nucleic acid” may be a DNA molecule that is complementary or hybridizes to a sequence in a gene of interest and remains stably bound under stringent conditions (as defined by methods well known in the art). Fragments and variants of the disclosed nucleotide sequences encoded thereby are also encompassed by the present invention. By “fragment” or “portion” is meant a full length or less than full length of the nucleotide sequence encoding the amino acid sequence of a protein.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

A “vector” is defined to include, inter alia, any plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably-linked” nucleic acids refers to the association of nucleic acid sequences on single nucleic acid fragment so that the function of one is affected by the other, e.g., an arrangement of elements wherein the components so described are configured so as to perform their usual function. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. Control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The term “amino acid” includes the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., dehydroalanine, homoserine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein) The term also comprises natural and unnatural amino acids bearing a cyclopropyl side chain or an ethyl side chain.

The invention encompasses isolated or substantially purified protein compositions. In the context of the present invention, an “isolated” or “purified” polypeptide is a polypeptide that exists apart from its native environment. The terms “polypeptide” and “protein” are used interchangeably herein. An isolated protein molecule may exist in a purified form or may exist in a non-native environment such as, for example, a transgenic host cell or bacteriophage. For example, an “isolated” or “purified” protein, or biologically active portion thereof, may be substantially free of other cellular material, or culture medium when produced by recombinant techniques, or substantially free of chemical precursors or other chemicals when chemically synthesized. A protein that is substantially free of cellular material includes preparations of protein or polypeptide having less than about 30%, 20%, 10%, 5%, (by dry weight) of contaminating protein. In certain embodiments, an “isolated” or “purified” protein may include cell lysates. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed proteins or partial-length proteins encoded thereby are also encompassed by the present invention. By “fragment” or “portion” is meant a full length or less than full length of the amino acid sequence of a protein.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “comparison window” makes reference to a contiguous and specified segment of an amino acid or polynucleotide sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous amino acid residues or nucleotides in length, and optionally can be 30, 40, 50, 100, or longer.

As used herein, “percentage of sequence identity” means the value determined by comparing two optimally aligned sequences over a comparison window, wherein the portion of the polypeptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or at least 95%.

The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

The invention will now be illustrated by the following non-limiting Examples.

### Example 1. Degradation of Residual Biuret in Urea Using Biuret Hydrolase

This example describes the bioremediation of a urea composition using a biuret hydrolase (see, FIG. 1).

### Materials and Methods

Chemicals: High purity urea was obtained from Fluka Chemical Corp. (Buchs, Switzerland) with purity listed as ≥99.5% pure and <0.1% biuret. Urea fertilizer (46-0-0) was from Loveland Products (Loveland, Colo.), with composition listed as 46% total nitrogen. Other chemicals were obtained from Sigma-Aldrich (St. Louis, Mo.) unless otherwise noted.

Analytical methods: The colorimetric Berthelot ammonia assay was used to measure residual ammonium (NH₄⁺) present in the urea and to detect NH₄⁺ released from the residual biuret in urea by addition of biuret hydrolase (BiuH) enzyme. The assay was conducted by adding 0.100 ml of sample directly to 0.300 ml of solution A (10 g/L phenol and 0.050 g/L sodium nitroprusside), followed by addition of 0.400 ml of solution B (5 g/L sodium hydroxide and 8.25 ml/L of commercial chlorine bleach or 5.25% sodium hypochlorite). The reactions were pulsed on a vortex mixer, incubated at 37° C. for 60 min, and then absorbance at 630 nm was read with a Beckman-Coulter DU-640 spectrophotometer. Quantification of NH₄⁺ was done via a standard curve prepared from ammonium chloride (NH₄Cl) (Sigma-Aldrich, St. Louis, Mo.) standards at 5-1000 μM in deionized reverse osmosis (DI/RO) water that were analyzed with the Berthelot reaction.

To validate performance of the Berthlelot reaction in the presence of urea, the assay was conducted on 0.1, 0.25, and 0.5 M urea standards spiked with 800 μM NH₄Cl. The urea standards were prepared by diluting an 8 M urea stock solution that was prepared in DURO water. The percentage of the NH₄⁺ spike that was recovered by the assay was determined by subtraction of the residual NH₄⁺ detected in urea standards that had not been spiked with NH₄⁺. Performance of the Berthelot reaction at higher urea concentrations was tested by conducting the assay on standards from 1-8 M urea that were not spiked with NH₄Cl (residual NH₄⁺ in urea detected only). Adherence to Beer's Law was verified by plotting the detected residual NH₄⁺ concentrations vs concentrations of the urea standards.

A Hewlett-Packard (now Agilent Technologies, Santa Clara, Calif.) 1100 series HPLC system was also used to characterize materials and to measure and track enzyme reactions as follows. Samples were injected in 10-100 μl aliquots onto an Agilent Eclipse Plus C18 column (4.6×250 mm, 5 μM particle size) or a Waters (Milford, Mass.) IC-PAK Anion column (4.6×150 mm, 10 μM particle size). The mobile phase was isocratic 5% methanol in water or 5% methanol in 5 mM phosphoric acid (pH 8.0), respectively. Elution of compounds from the column was monitored at 200 nm. Quantitation was done by analyzing standard biuret solutions over a concentration from 0.01-1.0 mM and then plotting a standard curve of concentration vs peak area.

Enzyme purification: A synthetic gene encoding the native biuret hydrolase from Herbaspirillum sp. BH-1 was expressed with a C- or N-terminal six-histidine tag from an isopropyl-β-D-thiogalactoside (IPTG)-inducible promoter T7 promoter on a plasmid in Eschrichia coli BL2(DE3). Cells were harvested by centrifugation and lysed with a French pressure cell (two passages at 124 MPa) in a buffer of 20 mM sodium phosphate (pH 7.4), 500 mM sodium chloride, 10 mM imidazole, and 10 mM 2-mercaptoethanol. The resulting crude lysate was centrifuged at 19,000×g for 60 min and the supernatant was passed through a 0.45 μM filter. An AKTA FPLC system (GE Healthcare, Chicago, Ill.) was used to inject the filtrate (cleared crude lysate) onto a HisTrap affinity column (GE Healthcare) charged with Ni²⁺ ions. Bound BiuH was eluted from the column with a linear gradient of 20-250 mM imidazole in the same buffer. Imidazole was removed from the pooled BiuH fractions and BiuH was concentrated by exchanging the buffer with imidazole-free buffer using spin concentrators (50,000 molecular weight cut-off) (Millipore, Burlington, Mass.). Total protein concentration was determined with the BioRad (Hercules, Calif.) Bradford protein assay reagent and BiuH purity was verified by SDS-PAGE. Aliquots of purified BiuH solution were dispensed into 0.5 ml microcentrifuge tubes and frozen by dropping the tubes into liquid nitrogen. Frozen samples were stored at ⁻80° C. Other enzymes tested were similarly expressed and purified.

BiuH activity in the presence of urea: Biuret degradation reactions were performed by adding 10-20 μg purified BiuH to 0.5 ml of urea standards (0.5-8 M) in DI/RO water in 1.7 ml microcentrifuge tubes. Reactions were pulsed once on a vortex mixer, spun briefly in a microcentrifuge, and then incubated at room temperature or 37° C. for 1-2 h without mixing or agitation. Total ammonium (NH₄⁺) was quantified with the Berthelot assay. Residual biuret present in urea was determined by treating unspiked urea standards with BiuH and subtracting the amount of residual NH₄⁺ detected above. Enzyme efficacy was verified in 0.5 M urea spiked with 800 μM biuret (97% pure, Acros Organics, Geel, Belgium). Net NH₄⁺ released from the spiked biuret by BiuH was calculated by subtracting the amounts of residual biuret and NH₄⁺ detected in the control treatments. Inhibition of BiuH at high urea concentrations was tested by treating unspiked urea standards (0.5-8 M) with BiuH as above and plotting the net NH₄⁺ released from residual biuret by BiuH vs the urea concentrations. The amount of enzyme added, incubation time, and incubation temperature were not optimized in this study.

### Results

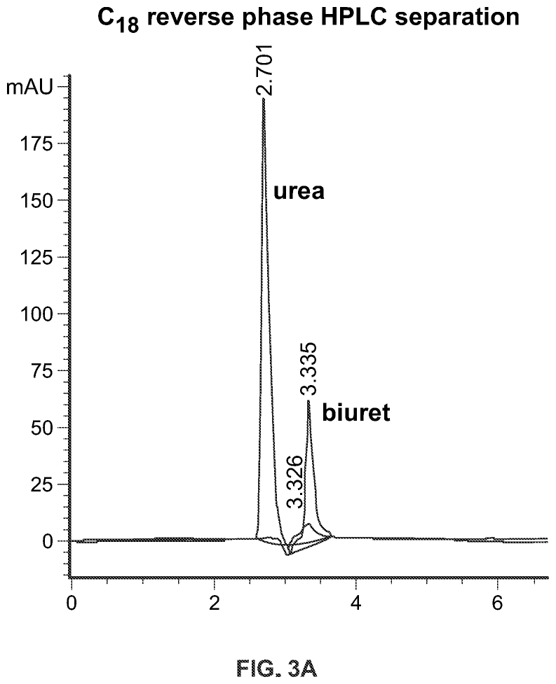

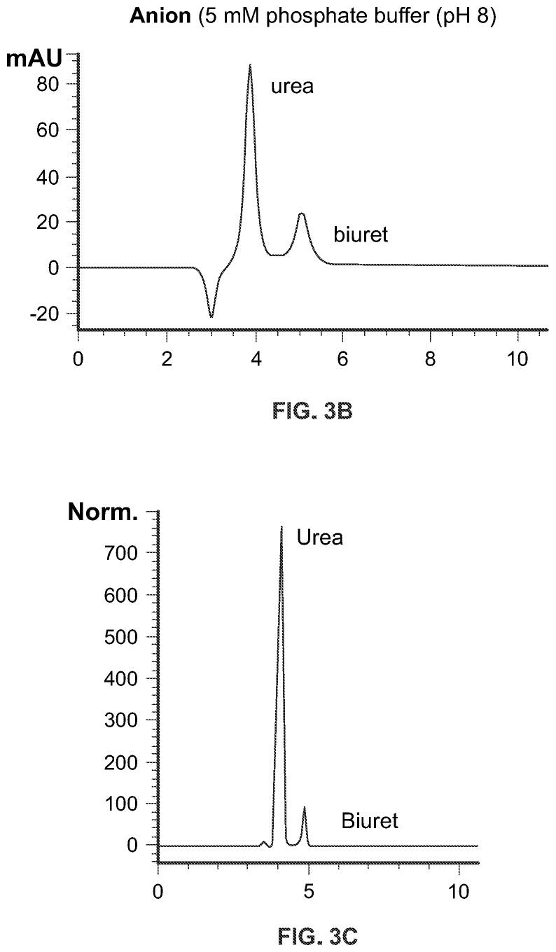

As shown in FIG. 2B, urea does not inhibit the detection of NH₄⁺ via the Berthelot method of determination. Accordingly, this method may be used to monitor the biuret hydrolase reaction (FIG. 2A). Additionally, HPLC may also be used to measure the reaction (FIGS. 3A-3B). Two different columns and conditions gave similar results.

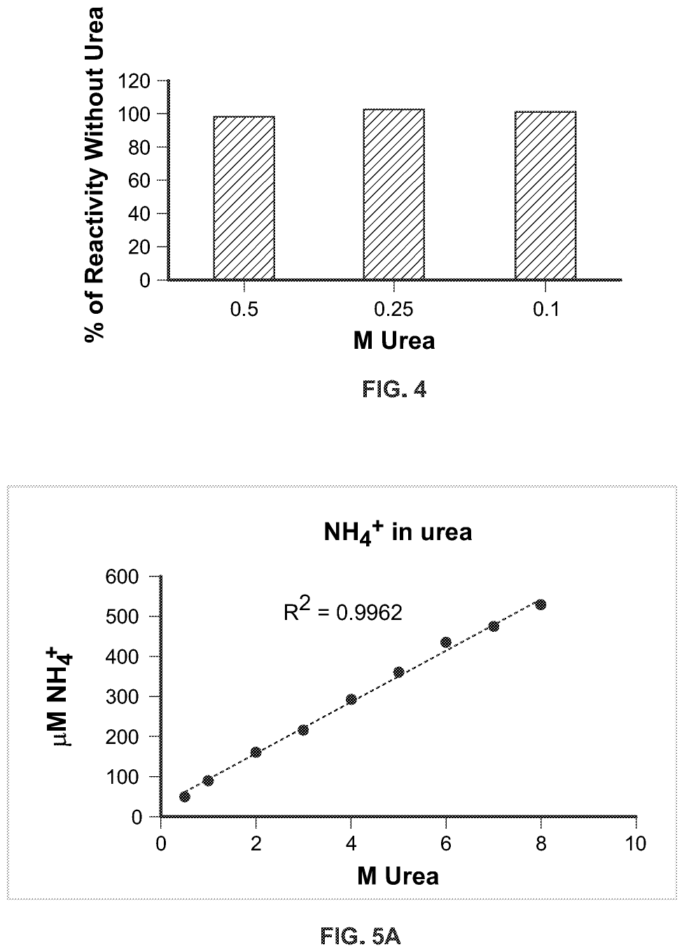

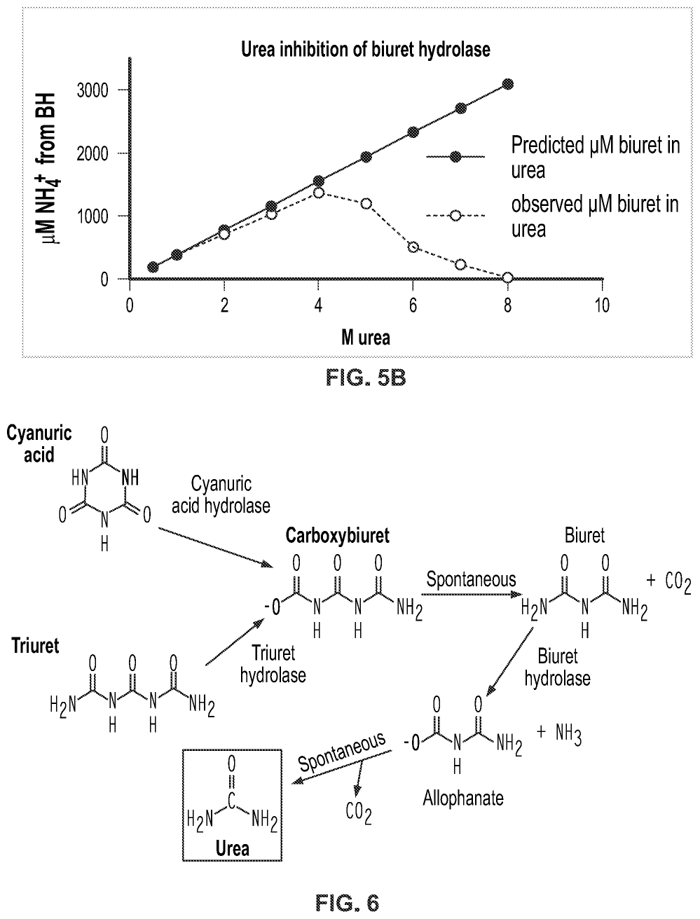

Importantly, it was also shown that biuret hydrolase is not inhibited by urea up to 0.5M (FIG. 4). Therefore, to further evaluate the effects of urea on biuret hydrolase activity, NH₄⁺ in 0.5-8.0 M Fluka urea (>99.5% pure) was analyzed. As shown in FIGS. 5A-5B, a slight inhibition of biuret hydrolase was observed at 4M urea and stronger inhibition was observed at higher concentrations.

### Example 2

This example evaluated the effects of CAH and allophanate hydrolase on 1) biuret hydrolase; 2) urea; and 3) biuret.

### Methods

Enzyme reactions were performed in 0.5 ml aliquots of 30 g/L Loveland urea fertilizer in DI/RO water (approximately 0.5 M urea). Aliquots of enzyme solutions containing 10 μg of individual enzymes were added to the reaction tubes, which were then incubated for 120 min in a water bath set to 37° C. and analyzed for total NH₄⁺ using the Berthelot method described above. Because the reaction of CAH with cyanuric acid yields biuret but no NH₄⁺, CAH and BiuH were added together and the Berthelot assay result was compared with the result using only BiuH to determine if the presence of CAH generated biuret in addition to the amount of residual biuret already present in the Loveland urea fertilizer. Allophanate hydrolase was added alone to the urea solution and net NH₄⁺ released by the enzyme was calculated by subtracting the residual NH₄⁺ detected in the urea solution without enzymes added from the result of the reaction with allophanate hydrolase added.

### Results

Biuret and cyanuric acid are present is urea-based fertilizers as a contaminant. As shown in Table A below, the urea fertilizer evaluated herein had very low levels of cyanuric acid and CAH had no effect on biuret hydrolase. Additionally, the allophanate hydrolase was shown to have no apparent reactivity with biuret or urea. Accordingly, biuret hydrolase may be used in combination with, e.g., CAH, without diminishing the urea content of the composition.

TABLE A

| Enzyme added | μM NH₄⁺ formed |
|---|---|
| Biuret Hydrolase | 538 |
| Biuret Hydrolase + CAH | 541 |
| Allophanate Hydrolase | <10 |

### Example 3. Evaluation of Biuret Hydrolase and Triuret Hydrolase (TrtA) Amino Acid Sequences

This example describes the isolation and evaluation of BiuH and TrtA sequences.

### Methods and Results

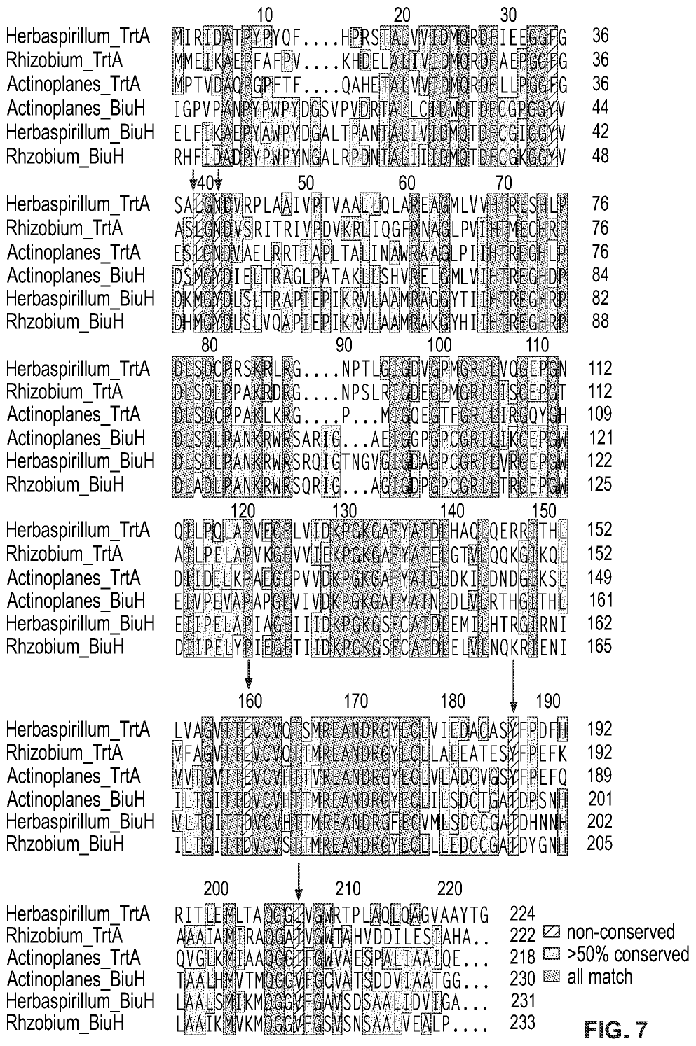

A Sequence Similarity Network was developed, and at an appropriate cutoff value, a cluster was identified to only contain sequences of BiuH and TrtA. By multiple sequence alignment, the close homologous sequences encoding BiuH were separated from TrtA by evaluating six signature residues (F35, L39, N41, E160, Y187, 1205 in TrtA from Herbaspirillum sp. BH-1) near the periphery of the active site of both TrtA and BiuH where there is a strict consensus for each enzyme (see, FIG. 7). In BiuH, the residues are Y35, M39, Y41, D160, T187 and V205.

Creating HMMs and Genome Context Annotation. Once the set of BiuH and TrtA sequences were created, a Hidden Markov Model was then trained for each set for use in annotating genomic contexts. The software HMIMER v3.1b2 (hmmer.org) was used to create these models, and with the tool RODEO (http://rodeo.scs.illinois.edu/), gene contexts were analyzed. Amino acid sequences for various BiuH and TrtA enzymes are shown in Table 1 below.

### Example 4. Reactivity of Biuret Hydrolase with Urea

This example describes a direct evaluation of biuret hydrolase and its potential reactivity with urea. Consistent with the results described in Example 1, BiuH was shown to have zero/undetectable levels of reactivity with urea.

### Methods

To explicitly test for low-level degradation of urea by biuret hydrolase (BiuH), 200 μg of the enzyme was added to 10 ml of 0.1 M Fluka urea in DI/RO water in a 15 ml conical centrifuge tube (Sarstedt, Nümbrecht, Germany). The reaction tube, and a control tube containing 0.1 M urea without BiuH added, were incubated at room temperature on a rocking platform. Aliquots (1 ml) were removed at 6, 24, and 48 h intervals and transferred to 1.7 ml microcentrifuge tubes. All samples were then immersed in a boiling water bath for 2 min to inactivate the enzyme and then were centrifuged at 17,000×g prior to storage at −80° C. The samples were then thawed and analyzed by HPLC as described above. Urea peak areas obtained from the HPLC chromatograms were converted to concentration using a standard curve prepared from urea solutions of 1-100 mM that were analyzed by the same HPLC method.

### Results

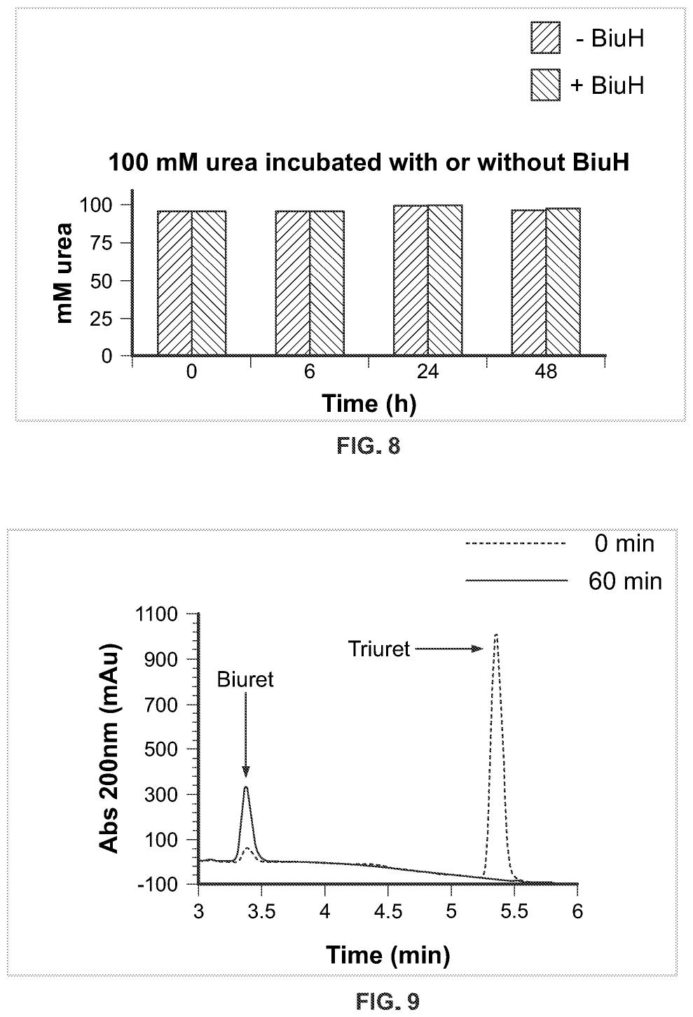

After incubation of 0.1 M urea with 20 μg/ml BiuH for 48 h there was no detectable decline in the urea peak as observed by HPLC with respect to the control treatment without BiuH added (FIG. 8). This demonstrates that BiuH has zero or very low reactivity with urea and therefore no measurable amount of urea would be degraded during treatment with BiuH.

### Example 5. Triuret Enzymatic Hydrolysis by TrtA

This example describes the evaluation of triuret degradation by a triuret hydrolase using HPLC.

### Methods.

A synthetic gene encoding the native triuret hydrolase from Herbapirillum sp. BH-1 was expressed with a N-terminal histidine tag from an isopropyl-β-D-thiogalactoside (IPTG)-inducible promoter T7 promoter on a plasmid in Eschrichia coli BL2(DE3). The enzyme was purified using methods similar to those described in Example 1.

A Hewlett-Packard (now Agilent Technologies, Santa Clara, Calif.) 1100 series HPLC system was also used to characterize materials and to measure and track enzyme reactions as follows. The reaction contained 1 mM triuret (containing 1% wt biuret impurity) in 125 mM sodium phosphate pH 8. The reaction was measured before and after 60 minutes of incubation with TrtA enzyme (5 μg). The separation method of the HPLC was an isocratic 95/5 (v/v) aqueous buffer (50 mM sodium phosphate pH 8)/methanol using a C18 (5 μm Eclipse Plus, 4.6×250 mm) column with a 1 mL/min flow rate and absorbance is measured at 200 nm wavelength.

### Results

As shown in FIG. 9, triuret was completely degraded leading to the formation of biuret.

### Example 6. Enzyme Processed Ultra-High Purity Urea for High-Grade Fertilizer, Diesel Emissions Fluid and Pharmaceuticals

Urea is the largest volume direct-use commercial chemical, providing great benefits to society as a nitrogen fertilizer, catalytic convertor component, industrial, consumer, and medical product additive. The myriad uses require purity greater than 98%, in some cases greater than 99.5%. Purity is achieved via advanced physic-chemical manufacturing methods and additional purification steps via adsorption, solvent extraction, or filtration. This example demonstrates a purity of urea >99.99%, significantly higher than previously described methods, via an inexpensive, efficient enzyme-based process. The enzymatic degradation converts the contaminants into urea, simultaneously increasing yield and purity. The enzymes are highly specific, showing no detectable activity with urea. The enzymes are significantly stable, even in the presence of high concentration urea (e.g., 1-2M). Urea is not a significant competitive inhibitor for the enzymes. Structures of the enzymes, as well as sequence signatures, have been described and may be found in a large number of microbial genomes (see, e.g., Table 1). The properties of the enzymes make them amenable to industrial scale-up. As described herein, one use for enzyme treatment is with respect to urea used for diesel exhaust fluid (DEF). Strict regulations mandate that DEF must contain low levels of biuret, as the latter interferes with the catalyst in urea-based NOx reductions systems used for diesel engines.

### Introduction

Industrial production of urea is enormous. At greater than 100 billion kg annually, it is more than twice the volume of the second leading organic chemical ethylene. The major use of urea is as a nitrogen fertilizer in agriculture. More fertilizer nitrogen is applied as urea than all other forms of nitrogen combined, and urea is projected to have an even higher market share in the next two decades. Similarly, urea is the major component in the diesel catalytic convertor market, where it serves to convert noxious oxides of nitrogen contained within the exhaust into harmless atmospheric dinitrogen. Another use of urea for removing nitrogen oxides is for selective catalytic reduction systems in coal power plants. Medically, urea is used, for example, in dermatological products for skin hydration, diuretics, and to manufacture barbiturates. There is a myriad of other uses for urea in industrial, consumer and medical products including, but not limited to, animal feed, roadside deicers, flame-proof materials, urea-formaldehyde polymers, cigarette additive, hair removers, hair conditioners, facial cleansers, psoriasis treatment, callous abatement, finger and toenail removal, diuresis for ICU patients, and drug delivery.

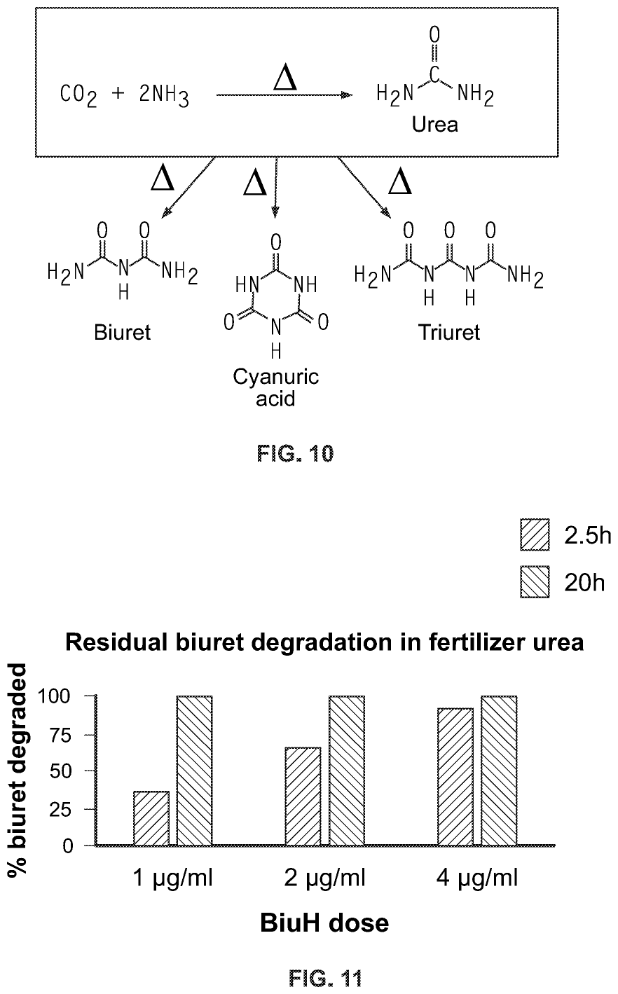

Most urea is made in large manufacturing facilities from NH₃ and CO₂ in a thermal process. Well-controlled manufacturing facilities make high purity urea directly, typically ˜99%. However, even under well-controlled manufacturing conditions, urea further reacts with additional ammonia and reaction intermediates to form biuret, cyanuric acid, and triuret (FIG. 10). Biuret is typically the major contaminant, although cyanuric acid and triuret can also be substantial. All these impurities are found in fertilizers, diesel catalytic converter fluid, and urea used for other purposes.

The impurities can be problematic in different applications, even in agriculture where the urea is designed to break down in soil by plant and microbial urease enzymes, releasing ammonia. Biuret, in particular, is undesirable in urea fertilizers because of its toxicity to plants. The susceptibility of crop plants to biuret toxicity is quite variable. Corn plants are fairly tolerant to low levels (˜5%) of biuret whereas cotton, avocado and fruit trees (e.g., citrus) are much more susceptible. The susceptibility is heightened when foliar application of nitrogen fertilizer is desirable. Foliar fertilizer is often made with “ultra-low biuret” urea, which typically contains 0.1-0.4% biuret.

Urea used for diesel exhaust fluids (DEF) must contain low levels of biuret, as the latter interferes with the catalyst in NOx reduction systems for diesel engines that use concentrated urea solutions. DEFs are aqueous urea solutions with a biuret content <0.3%, as mandated by U.S. Environmental Protection Agency, European Union, and other regulators globally. Other impurities, such as triuret and cyanuric acid, are also considered undesirable for the performance of DEF urea. The impurities decrease the efficiency of the exhaust system in removing nitrogen oxides and clog the catalyst chamber over time, diminishing catalytic converter lifetime. Triuret is particularly problematic because of its poor solubility and caking properties in the convertor system (Brack, et al, Emiss. Control Sci. Technol. 2: 115-123, 2016).

An even higher grade of urea is necessary to attain a grade denoted as US Pharmacopeia (USP) urea. USP urea is utilized in cell culture and protein methodologies, particularly pertaining to human pharmaceuticals. As such, the biuret content is described to be less than 0.1%. Other impurities are also constrained against, such as cyanuric acid and triuret.

Due to the many commercial uses of urea and the large cost differential as purity increases, a large number of processes have been developed for urea purification (e.g., as described in U.S. Pat. No. 4,701,555). Previously developed purification methods involve adsorption, ion exchange, filtration, solvent extraction, and chemical catalysis. Additionally, “ultra-low biuret urea” (≤0.1% biuret) manufacturing may involve pressing crystalline urea directly into pellets without melting and heating, and “reduced biuret urea” (≤0.4%) manufacturing may involve a short melting and prilling process to limit biuret formation. While there are a wide range of options, methods to date generally require extra capital equipment and knowledge, and/or an additional unit operation, have limitations in impurity removal, and can generate a waste that needs to be separated or disposed of The requirement for these additional methods typically increases the cost of urea significantly.

As described herein, specific enzymes that transform urea impurities have been identified and characterized (see, e.g., Table 1). Biuret, triuret and cyanuric acid biosynthetic pathways in living things are not known, unlike urea which is formed via a known biosynthetic pathway that makes a nitrogen excretion product in many animals. Urea metabolism by soil bacteria and fungi is known to occur via two distinct enzymes, urea carboxylase and urease. Plants also make a urease enzyme. Biuret biodegradation is carried out by an enzyme denoted biuret hydrolase (Cameron, et al, ACS Catal. 2011(1):1075-1082.) that is a member of the isochorismatase-like hydrolase (IHL) superfamily (Robinson, et al, Environ. Microbiol. 20(6): 2099-2111, 2018). Biuret hydrolases are small, stable tetrameric proteins and an X-ray structure is now available (Esquirol, et al, PLoS One. 13(2): e0192736, 2018). Certain triuret hydrolases are described herein (Tassoulas L. 2020. Novel discrimination of biuret and triuret degradation by enzymatic deamination: regulation and significance for slow-release nitrogen fertilizers. University of Minnesota, St. Paul, Minn.). It is a homolog of biuret hydrolase and its X-ray structure has recently been determined (Tassoulas, et al, J Biol Chem. 2020 Nov. 10; jbc.RA120.015631, which incorporated by reference herein). Cyanuric acid hydrolase is a member of a protein family found, to our knowledge, only in bacteria and fungi (Seffernick, Appl. Environ. Microbiol. 82: 1638-1645, 2016). It has an unusual fold with a three-fold symmetrical active site binding the three-fold symmetrical substrate at the interface of three domains of a single polypeptide (Shi, et al, PLoS One 14(6): e0216979, 2019). The percentage of bacteria containing each of biuret hydrolase, triuret hydrolase and cyanuric acid hydrolase are known to be much less that the percentage of bacteria containing urease, hence urea in fertilizer is rapidly degraded to ammonia and nitrate in soil and is readily assimilated by plants whereas contaminants like biuret can persist and manifest toxicity. Plants are not indicated to have a biuret hydrolase and so it can accumulate in certain plants and cause foliar damage.

This example investigates the feasibility of using these enzymes, which react with urea impurities, to treat urea and thus make extra-high purity urea. By combining cyanuric acid hydrolase, triuret hydrolase and biuret hydrolase, all major contaminants of urea can be removed. It is shown here that the purity achieved is much greater than obtainable by physicochemical methods. It is especially favorable that the ultimate products of all the reactions combined produce urea. Thus, unlike many other purification methods, the enzymatic process described here is easier, cheaper and increases the urea content while simultaneously removing undesired contaminants.

### Materials and Methods

### HPLC

Contaminants in urea solutions (Fertilizer from Greenway Biotech, Blue DEF from PEAK, USP urea from Research Products International) were analyzed by high-performance liquid chromatography (HPLC) with an established method (Woldemariam et al. PDA J Pharm Sci Technol. 2020; 74(1):2-14) using an Agilent Technologies (Santa Clara, Calif.) 1100 HPLC-UV with a diode array detector (DAD). Samples were injected (10 μl) onto a ThermoFisher Scientific (Waltham, Mass.) Acclaim Mixed-Mode WAX-1 (150 mm×4.6 mm, 5 μM particle size) and separations were achieved in an ioscratic mobile phase of 25 mM phosphate buffer (pH 6.2) at a flow rate of 0.5 ml/min for 35 min at room temperature. The mobile phase was prepared from HPLC grade phosphoric acid (ThermoFisher Scientific) and potassium hydroxide (Sigma-Aldrich, St. Louis, Mo.) and sample matrices were adjusted to the mobile phase composition with a 10× mobile phase buffer concentrate prior to injection. Chromatograms were acquired by monitoring at 200 or 220 nm. Resulting peaks were identified by comparing retention times with those of authentic commercial or synthesized chemical standards and by characteristic UV absorbance maxima when possible (214 nm for cyanuric acid, 221 nm for ammelide).

### Enzyme Purification

Enzymes used and the original source strains were as follows: biuret hydrolase from Rhizobium leguminosarum by viciae 3841, biuret hydrolase and triuret hydrolase from Herbaspirillum sp. BH-1, cyanuric acid hydrolase from Moorella thermoacetica ATCC 39073, and N-Isopropylammelide aminohydrolase (AtzC) from Pseudomonas sp. ADP. All enzymes were produced as previously described (Robinson et al., Environ. Microbiol. 20(6): 2099-211, 2018; Tassoulas, et al., J Biol Chem. 2020 Nov. 10; jbc.RA120.015631; Li et al., Appl Environ Microbiol. 2009; 75(22):6986-6991; Hernandez et al. Nat Chem. 2019; 11(7):605-614) and stored at −80° C. All were expressed heterologously in Escherichia coli BL21(DE3) from synthetic or cloned genes with an N-terminal or C-terminal (biuret hydrolase) six-histidine tag added. Proteins were purified by affinity chromatography in a single step on a GE Healthcare, (Piscataway, N.J.) HisTrap HP 5 ml column charged with NiSO₄ on a GE Äkta Purifier fast liquid protein chromatography (FPLC) system. AtzC was similarly purified on a 5 ml open column with Qiagen (Hilden, Germany) Ni-NTA agarose resin (Hernandez et al., Nat Chem. 2019; 11(7):605-614). Bound proteins were eluted with an imidazole (Sigma-Aldrich) gradient, enzyme fractions were pooled, and imidazole removal/buffer exchange was accomplished as described.

### Enzyme Incubation

All enzyme reactions were incubated at room temperature without mixing. Different concentrations of enzyme (from 1 to 4 ug/ml) were used to treat 3% fertilizer urea solution for removal of biuret impurity (FIG. 11).

Alternatively, 50 ml of 1M Fluka urea solution was treated with 200 ug Herbaspirillum BiuH (enzyme concentration at 4 ug/ml) for two days at room temperature. The reaction was conducted in a 125 ml glass screw-capped bottle.

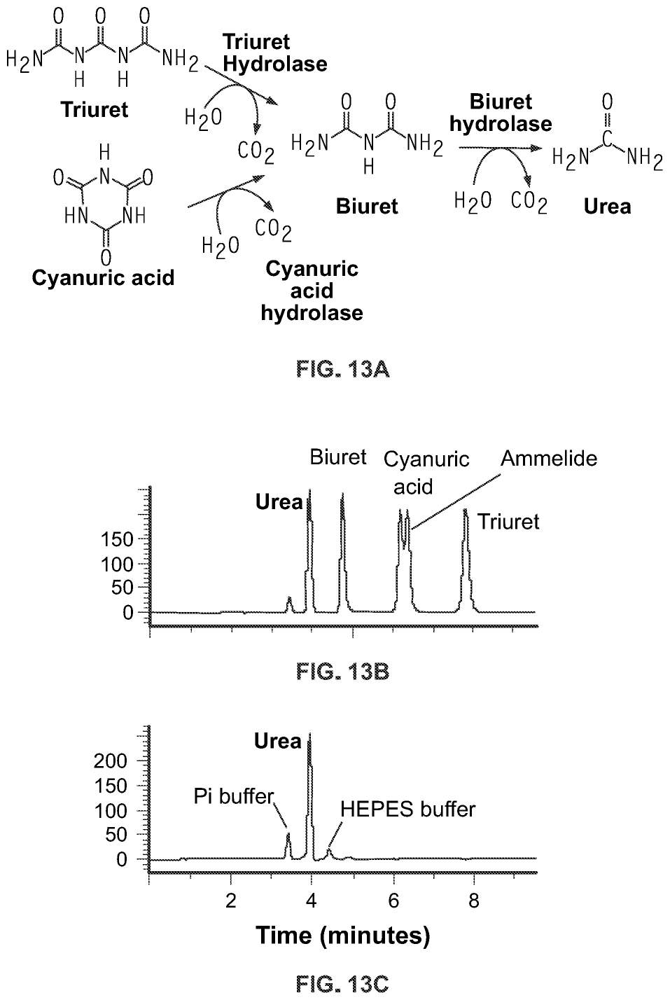

Alternatively, sub-milligram quantities of enzymes (BiuH, AtzD, and TrtA were used at 0.4 ug/ml; AtzC was used at 2 ug/ml) were incubated with a 10 mM solution of urea containing 0.35 mM biuret, 0.65 mM cyanuric acid, 0.13 mM ammelide, and 0.1 mM triuret (FIG. 13).

### Results

### Biuret Hydrolase Mediated Enzymatic Degradation of Biuret Impurity in Urea Solution

Residual biuret in 3% Loveland urea solution could be fully degraded in ≤20 h by 1 μg/ml BiuH (FIG. 11). The 2.5 h time point shows partial degradation with 1, 2, or 4 μg/ml BiuH added, demonstrating dosage control of the biuret degradation rate. (FIG. 11).

### Analysis of Different Urea Sources

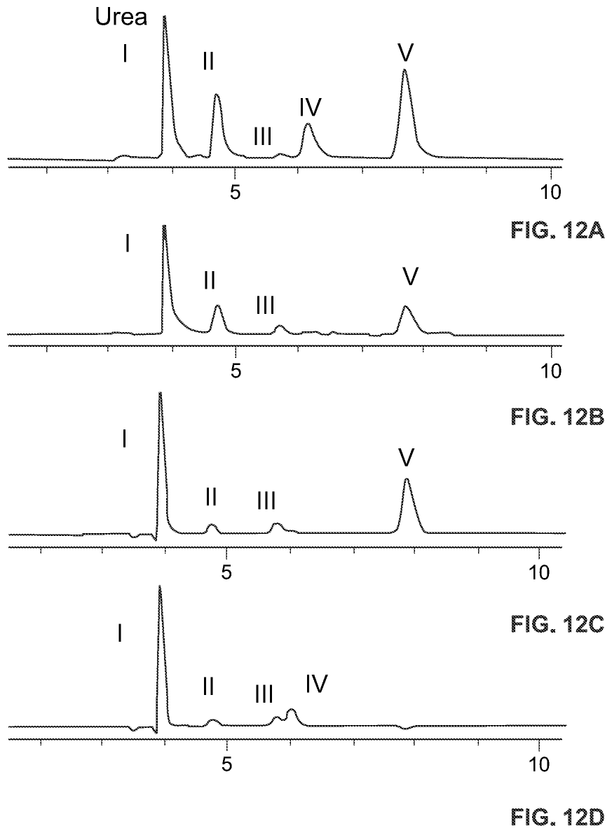

Commercial urea sold for different applications was analyzed by HPLC. The major contaminant seen in all urea sources was biuret. Other contaminants observed were triuret, cyanuric acid, and sometimes ammelide, consistent with known contamination problems derived from the pyrolytic process used in making commercial urea (FIG. 10). The amount of contamination varied. Not surprisingly, the impurities were higher in urea used in greater bulk fertilizer. The purity seen in FIG. 12A-12D increases and that tracks with a very steep increase in cost of goods. HPLC analytical methods allowed the sensitive detection of impurities. 1H-NMR of urea in d6-DMSO was also conducted with results confirmatory to the HPLC results presented herein.

### Enzymatic Removal of Contaminants to Baseline Levels

All urea samples tested contained biuret and cyanuric acid, most contained triuret and some contained ammelide. Enzymes that degrade each were purified and characterized: biuret hydrolase (BiuH) from Herbaspirillum BH1, cyanuric acid hydrolase (AtzD) from Moorella thermoacetica, triuret hydrolase from Herbaspirillum BH1 (TrtA), and N-isopropylammelide hydrolase (AtzC) from Pseudomonas sp ADP. Sub-milligram quantities of each (BiuH, AtzD, and TrtA were used at 0.4 ug/ml; AtzC was used at 2 ug/ml) were incubated with a 10 mM solution of urea containing biuret, cyanuric acid, ammelide, and triuret to give similar peak areas in an HPLC chromatogram (FIG. 13B) at time zero as described in the Methods section. Following an overnight incubation and adjustment of pH to match the HPLC elution phases, all contaminants were removed (FIG. 13C). The small peaks in the chromatograms have been demonstrated to be HEPES and phosphate buffers from the enzyme solutions. The urea peak was increased only marginally because the contaminants that are converted to urea represent at most ˜10% of the total molar mass. The contaminants have greater absorbance than urea and so their peak area is overrepresented in the appearance of the chromatogram. In this Example (e.g., see FIG. 13), the enzymes (BiuH, AtzD, and TrtA were used at 0.4 ug/ml; AtzC was used at 2 ug/ml) degrading the major contaminants are present at a level equivalent to 2.5 g of each enzyme per ton of urea purified.

### Stability of Biuret Hydrolase

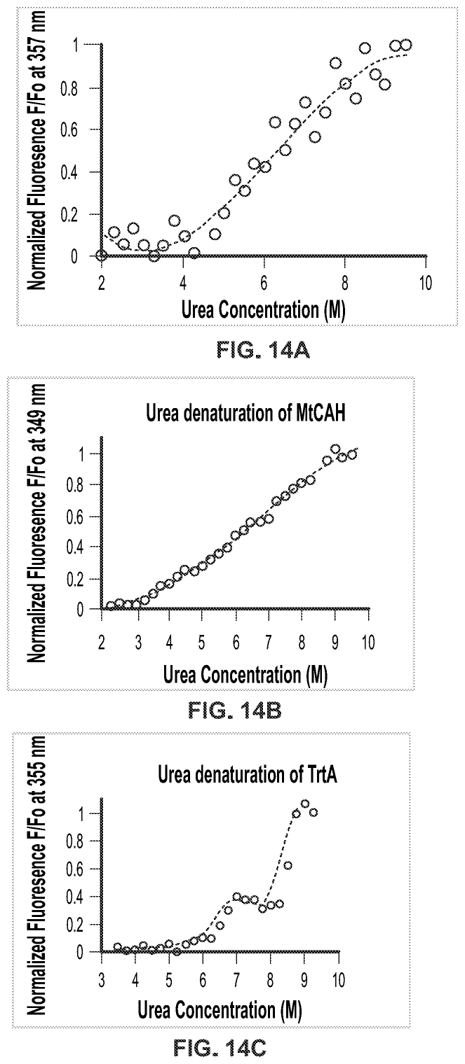

The major contaminant in most urea formulations is biuret. In applications such as the DEF urea, it would be ideal if biuret hydrolase were to be active in the fluid, which is an aqueous solution of 32.5% (wt/wt) urea. That is equivalent to 5.4M, a concentration that will denature most proteins. The biuret hydrolase from Rhizobium leguminosarum by viciae 3841 is a reasonably stable protein with a melting temperature of about 58° C. The denaturation of the protein was tested directly, using the native fluorescence of the protein's aromatic groups, principally tryptophan residues at subunit interfaces as known from the X-ray structure. The midpoint of denaturation was observed at 6.4M (FIG. 14A), fully one-unit molar concentration above the concentration of DEF fluid. Consistent with the denaturation determination, biuret hydrolase enzyme activity was tested by measuring ammonia release from biuret in the presence of 1, 2, 4, and 8 M urea. Ammonia formation was observed at 1, 2, and 4 M urea but not 8 M urea. These results indicate that biuret hydrolase is unusually stable to urea as a denaturant.

Similarly, triuret hydrolase does not show evidence of denaturation until above 5M urea. It shows a bimodal denaturation curve (FIG. 14C). It is a dimeric protein and perhaps subunit separation precedes subunit unraveling. One cyanuric acid hydrolase (CAH) was tested, choosing the most thermostable one presently known, from Moorella thermoaceticum. Unexpectedly, it was the most labile with respect to urea, showing denaturation above 3M (FIG. 14B).

### Inhibition of Biuret Hydrolase by Urea

In addition to potential denaturation, it was also considered that high urea concentrations could effectively inhibit the biuret hydrolase reaction. Urea resembles biuret structurally but is smaller, suggesting that it might compete for binding. Enzymatic urea decontamination will require enzyme to convert millimolar biuret in molar urea concentrations, and that was tested here.

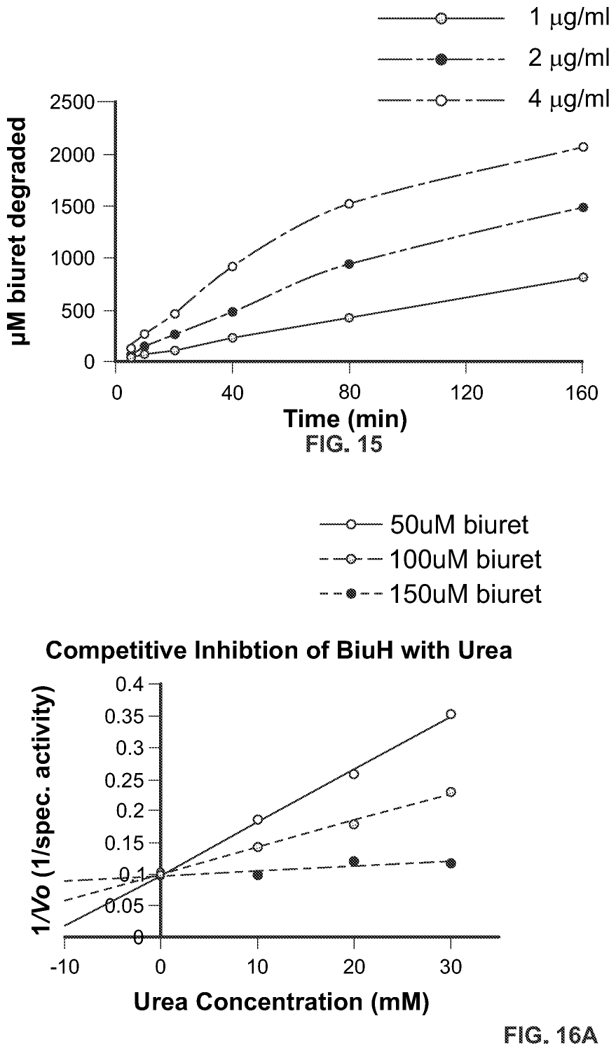

As shown in FIG. 15, a 0.5M urea fertilizer solution containing 2.4 mM biuret was treated with three different concentrations of biuret hydrolase. The curves showing amounts of biuret removed reveal several things. One, the initial rate is proportional to enzyme added, as would be expected if enzyme inhibition and denaturation are not significant. With 1 ug enzyme, the rate was linear over the course of the experiment, during which time 30% of the initial biuret present was degraded, again consistent with low or no inhibition. With 4 ug enzyme, degradation slowed after removal of >50% of the biuret. One would expect inhibition to increase significantly as the urea/biuret ratio increases dramatically from the initial 208:1 to more than 1000:1 after >80% degradation, the point reached with 4 ug enzyme after 160 minutes (FIG. 15).

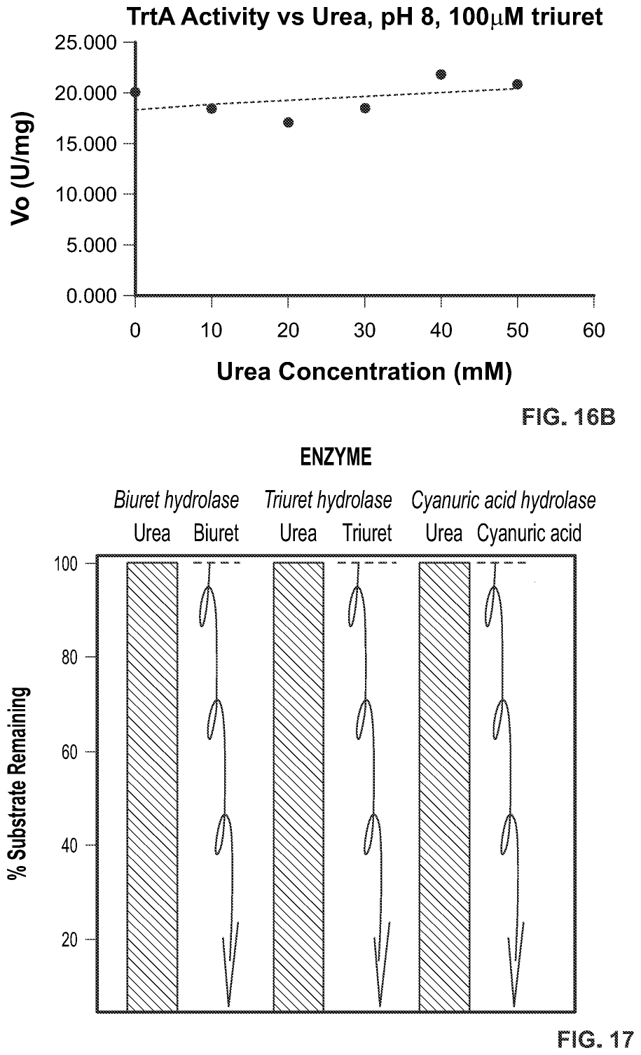

Kinetic constants will allow modeling for applications that use various concentrations of enzyme, urea and contaminants (FIG. 16). Urea is a weak competitive inhibitor of biuret hydrolase. The Ki for urea was determined to be 34 mM, more than one thousand-fold higher than the Km for biuret at 23 uM. With triuret hydrolase, which has a Km for triuret of about 20˜21 uM, no inhibition was measurable at urea concentrations up to 50 mM. These inhibition data are consistent with the enzyme treatment experiment shown in FIG. 13 in which triuret present at 10,000-fold lower than urea is nonetheless completely removed.

### Specificity of Enzymes

If any of the enzymes showed activity in degrading urea, that would require care in the timing of treatments to remove contaminants without removing any desired material. In that context, each enzyme was tested in 24-hour incubations with urea (FIG. 17). Biuret hydrolase, triuret hydrolase, and cyanuric acid hydrolase did not degrade urea. Positive controls in which each enzyme was incubated with its natural substrate showed complete disappearance.

### Discussion

There are both practical and theoretical implications for the observed conversion of linear and cyclic ureides to urea under conditions where there is no demonstrable transformation of urea. With respect to the latter, well-established theory and experiment all point to an explanation. Urease was purified more than one hundred years ago, has been extensively studied, and the urease reaction modeled. It is well accepted that urea is highly resonance-stabilized, such that overall urea hydrolysis has not been demonstrated, either enzymatically or chemically. Instead, urease catalyzes an ammonia elimination reaction, using a binuclear nickel cofactor at the active site. This explains the significant energy expenditure of cells to make the urease subunits and a nickel insertion system that used GTP.

The failure of biuret hydrolase, triuret hydrolase and cyanuric acid hydrolase to hydrolyze urea can be interpreted in light of the energetic and reaction mechanism barriers imposed by the urea molecule. Urea imposes an energy barrier to hydrolysis of at least 30-40 kcal/mol greater than molecules such as formamide. Moreover, the three enzymes used in this study are set up for C—N bond hydrolysis, not elimination. All now have X-ray structures solved, been studied mechanistically, and are not known to use a metal in catalysis, unlike urease. Biuret hydrolase is known to catalyze an overall hydrolysis of the terminal biuret amide bond via an intervening enzyme cysteine nucleophile, characteristic of members of the IHL protein superfamily to which it belongs. Triuret hydrolase is a member of the IHL superfamily catalyzing an analogous reaction. Cyanuric acid hydrolase is proposed to directly activate water for attack on one of the substrate's symmetrical-ring carbonyl carbons.

The greater reactivity of biuret than urea is also represented by the known method of treatment of urea fertilizer to deaminate biuret using sodium hydroxide and heat. The biuret will undergo base catalyzed hydrolysis to allophanate and urea is unreactive under the conditions that hydrolyze biuret. While this method is conceptually parallel to the enzymatic methods described here, significant base is required, and it must be neutralized with a strong mineral acid while salts are generated in the basification/acidification. Cyanuric acid is unreactive with sodium hydroxide and would persist. In general, the base-catalyzed deamination of biuret has not been implemented because of the drawbacks; an enzyme-based treatment can be carried out under mild conditions of temperature and does not produce salt. Low levels of enzyme are sufficient.

It is remarkable that the enzymes used in this study, that all work on ureide substrates, would show such stringent substrate selectivity. Indeed, despite over 15 years of studies, a substrate other than cyanuric acid has never been demonstrated for cyanuric acid hydrolase, with several dozen having been tested. Highly analogous barbituric acid has been shown to be an inhibitor with no turnover observed. Biuret hydrolase and triuret hydrolases show very high stringency with analogous compounds only showing <1% of activity as their ideal substrates. It is most surprising that triuret hydrolase shows virtually no activity with biuret. The structural basis of the exquisite substrate specificity has recently become better understood from solving the structure of triuret hydrolase with biuret bound and showing how it binds in an unfavorable position (Tassoulas, et al, J Biol Chem. 2020 Nov. 10; jbc.RA120.015631). A generalist enzyme with activity against both biuret and hydrolase has been identified but it has sufficiently lower k_{cat}/K_{M} with either substrate as to be less desirable.

Given the activities observed, and expression levels of the enzymes, it is projected that enzymatic treatment as described herein gives the highest purity urea and at a treatment cost lower than other conventional methods. The levels of contaminants in the urea after enzyme treatment are indistinguishable by HPLC or NMR. It was estimated that after enzyme treatment as described herein impurity levels fall below 0.01%. Enhanced stability of the enzymes, for example, from immobilization of the respective enzymes, singly or in combination, may further improve the cost-effectiveness in producing ultra-pure urea products compared to other conventional methods.

### Example 7. Encapsulated and/or Glutaraldehyde Cross-Linked Whole Cells

### Methods

E. coli cells expressing BiuH from C. citrea or Rhodovulum sp. N122 (enzymes were selected in this Example, in part, because of their predicted Tm of 64° C.) were cross-linked with glutaraldehyde by adapting the method of Strong et al., Environ Microbiol. 2000 February; 2(1):91-8. Cells were harvested by centrifugation, the pellets were resuspended at 0.1 g/ml in 5 mM potassium phosphate buffer (pH 7.0) containing 0.3% glutaraldehyde (Sigma), and the reaction was incubated on a rotary shaker at 180 rpm at room temperature. After 60 min, the cells were pelleted by centrifugation, resuspended in 50 mM sodium tetraborate decahydrate (pH 8.8), and incubated on the shaker for 60 min, pelleted and resuspended in 20 mM tris base (Fisher) (pH 8.6), and then incubated overnight on the shaker. The cross-linked cells were washed with three aliquots of 1× phosphate buffered saline and resuspended to 0.1 g/ml. Specific biuret hydrolase activity of free and cross-linked cells was determined by adding 0.1-1.0 mg of wet cells to 5 ml of 1 mM biuret in 50 mM potassium phosphate buffer (pH 7.3) and incubating at room temperature on a rocking platform for 10 min. Aliquots were centrifuged to pellet cells and supernatants were analyzed for NH₄⁺ release via the Berthelot reaction as described above. Biuret degradation activity in DEF was tested by adding 5 mg of cross-linked cells to 5 ml undiluted Audi (Ingolstadt, Germany) or PEAK (Old World Industries, Northbrook, Ill.) brand DEF incubating overnight, and then analyzing supernatants by HPLC using the method in Example 6. All samples were diluted with water and 10× mobile phase buffer to give 0.050 M urea (108× dilution factor) in 1× mobile phase buffer prior to HPLC analysis. The DEF samples used ranged in pH from 9.45 (Audi) to 9.70 (PEAK).

Cross-linked cells containing the expressed C. citrea BiuH were encapsulated in calcium alginate or chitosan beads (˜3 mm diameter) as follows. Cell suspension (0.1 mg/ml) was combined 1:3 with 4% sodium alginate (Sigma) dissolved in water. This mixture was slowly dripped from a syringe through a 22-guage needle into a solution of 0.1 M calcium chloride and 0.1% sodium chloride in water that was gently stirred. The beads were left in the gelling solution for 60 min and were then washed 3× with phosphate buffered saline. Chitosan solution (1%) was prepared by dissolving chitosan (Sigma, medium molecular weight, 75-85% deacetylated) in 1% acetic acid. This solution was used to resuspend cell pellets of cross-linked or fresh (not cross-linked) cells at 25 mg/ml. Beads containing the cross-linked cells were formed by dripping the mixture through a syringe and needle as above into 0.1 M NaOH in water. Beads containing fresh cells were formed by dripping the mixture into 1.0 M NaOH plus 5% glutaraldehyde. Chitosan beads were left in the gelling solution for 60 min and then washed 3× with 0.1M potassium phosphate buffer (pH 7.0). Beads of either type containing 25 mg wet cells were added to 5 ml of Audi DEF and incubated overnight with slow rocking at room temperature. The DEF was removed from the beads after incubation by pipetting and biuret degradation was assessed by HPLC. A fresh DEF aliquot was added to the beads and incubation was repeated.

### Results

E. coli cells expressing either BiuH from C. citrea or Rhodovulum sp. N122 had specific biuret hydrolase activity of ˜0.3 μmol NH₄⁺ min-¹mg⁻¹ wet cells prior to cross-linking. After cross-linking, cells that expressed the C. citrea or Rhodovulum sp. N122 BiuH retained 63% or 10% of specific activity, respectively. In overnight incubations in undiluted DEF, the cross-linked cells containing C. citrea BiuH degraded 80% of biuret in undiluted DEF. No biuret degradation was detected in a parallel treatment with the cross-linked cells containing Rhodovulum sp. N122 BiuH.

Cross-linked cells (25 mg) that expressed C. citrea BiuH and were encapsulated in 3% calcium alginate beads degraded biuret in Audi DEF to below detection (≥95% biuret degraded) within 20 h. However, the beads had reduced structural integrity after the second aliquot of DEF was added. Both previously cross-linked cells and fresh cells encapsulated in 1% chitosan degraded 22% of biuret in Audi DEF; biuret degradation in the second applied DEF aliquot was <10%.

### Discussion

The C. citrea and Rhodovulum sp. N122 BiuHs were selected for their high predicted melting temperatures (Tm) and because purified BiuH from Herbaspirillum sp. BH-1 or Rhizobium leguminosarum bv. viciae 3841 did not show detectable activity in undiluted DEF. Cells that expressed the C. citrea BiuH maintained sufficient activity after glutaraldehyde fixation to be an effective biocatalyst for biuret remediation, but adding whole cells directly to DEF is not practical. Use of a whole cell catalyst requires a design that allows for separation from the DEF after treatment and a means for re-use of the catalyst. Results with calcium alginate beads showed that sufficient BiuH activity was maintained after encapsulation to remediate biuret in DEF, but poor stability of the beads in DEF limited its re-use. In contrast, cells encapsulated in chitosan beads maintained structural integrity in DEF, but reduced BiuH activity limited effectiveness of the catalyst. The reduced BiuH activity could have been due to the harsh conditions used to encapsulate the cells in this Example (chitosan solution at pH 3.0, gelling solutions at pH 12) and/or the flocculation of cells in the chitosan solution. Substrate diffusion also could have limited biuret degradation by cells in the chitosan beads. A practical whole-cell biocatalyst may include modifications of the immobilization method/a formulation that maintains BiuH activity and material structural integrity during repeated use in DEF.

### Example 8. EnginZyme EziG3 (Amber) His-Tag Attachment Resin (“Semi-Hydrophilic Polymer” Support)

### Methods

EziG3 resin (20 mg) was combined with 8 mg purified C. citrea BH (N-terminal six-his tag) in 20 mM sodium phosphate (pH 7.4) plus 0.5 M NaCl and incubated on shaking platform at 4° C. for 30 min. The resin was sedimented by brief centrifugation and the protein content of the supernatant was determined using the BioRad (Hercules, Calif.) Bradford Protein Assay reagent. Results indicated ˜98% loading efficiency, corresponding to ˜0.4 mg protein/mg resin. The resin was then washed with 10×1 ml aliquots of 5 mM potassium phosphate buffer (pH 7.0) and free protein in the supernatant of the tenth wash was measured as <1.2 μg/ml. To cross-link the immobilized enzyme, an aliquot of resin was incubated overnight in 0.5 ml of 25 mg/ml polyethyleneimine (PEI) (25,000 MW) (pH 7) on a shaker at 4° C. The treatment was then washed 10 times as above and an aliquot was removed and added to 0.5 ml of 0.5% glutaraldehyde, incubated for 60 min on a shaker at 4° C., washed as above, and stored overnight in the wash buffer at on a shaker at 4° C. Resin aliquots from each attachment/treatment stage containing ˜0.2 mg enzyme were added to 0.25 ml undiluted Audi or Peak DEF and incubated on a shaker at room temperature. Supernatants were diluted and analyzed by HPLC as above. Stability during repeated use was tested by incubating an enzyme aliquot in DEF overnight, removing and analyzing the treated DEF, and adding a fresh aliquot of DEF to the enzyme and repeating the incubation.

### Results

The untreated attached enzyme degraded 90% of biuret in undiluted PEAK DEF. The PEI and PEI plus glutaraldehyde treated attached enzymes degraded 95-100% of biuret in Peak DEF. Because single usage of this amount of enzyme would not be economically feasible, stability of the attached enzyme treated with PEI and glutaraldehyde was tested by repeated incubations of a single enzyme aliquot with fresh DEF aliquots. Biuret degradation measured after 4 h incubation in PEAK or Audi DEF indicated a specific activity of ˜3 μmol min⁻¹mg⁻¹, which was 46% of the free enzyme activity measured in 1 mM biuret at pH 8.0. Subsequently, the same immobilized enzyme aliquot was able to degrade biuret to below detection (≥95% of initial biuret degraded) in undiluted DEF within 20-24 h in seven sequential aliquots of undiluted DEF, indicating stability of BiuH activity during re-use in multiple DEF aliquots.

### Discussion

As with whole cells, addition of free enzyme directly to DEF is a less practical and economical treatment strategy. Therefore, the enzyme may be immobilized to avoid contamination of the DEF and may be sufficiently stabilized to allow multiple re-use treatment cycles. In the example described here, BiuH with a high predicted Tm from C. citrea was immobilized by attachment to his-tag affinity resin and further stabilized by polymer coating (PEI) and cross-linking (glutaraldehyde). Multiple strategies for immobilizing and stabilizing enzymes are known. The maintenance of C. citrea BiuH activity during repeated use in multiple DEF aliquots provides a model for an immobilized and stable catalyst to degrade biuret in undiluted DEF.

TABLE 1
Informal Sequence Listing

| SEQ | |
|---|---|
| ID | |
| NO. | Embodiments of Biuret Hydrolase Amino Acid Sequences |
| 1 | Herbaspirillum_biuret_hydrolase_bonafide: |
| | MPELFIKAEPYAWPYDGALTPANTALIVIDMQTDFCGIGGYVDKMGYDLSLTRAPIEPIKRVLAAMRAGG |
| | YTIIHTREGHRPDLSDLPANKRWRSRQIGTNGVGIGDAGPCGRILVRGEPGWEIIPELAPIAGEIIIDKP |
| | GKGSFCATDLEMILHTRGIRNIVLTGITTDVCVHTTMREANDRGFECVMLSDCCGATDHNNHLAALSMIK |
| | MQGGVFGAVSDSAALIDVIGA |
| 2 | AEX65081.1 biuret hydrolase (plasmid) [Rhodococcus sp. Mel]_mel: |
| | MIYSTVNANPYAWPYDGSIDPAHTALILIDWQIDFCGPGGYVDSMGYDLSLTRSGLEPTARVLAAARDTG |
| | MTVIHTREGHRPDLADLPPNKRWRSASAGAEIGSVGPCGRILVRGEPGWEIVPEVAPREGEPIIDKPGKG |
| | AFYATDLDLLLRTRGITHLILTGITTDVCVHTTMREANDRGYECLILSDCTGATDRKHHEAALSMVTMQG |
| | GVFGATAHSDDLLAALGTTVPAAAGPRARTE |
| 3 | NP_791183.1 isochorismatase family protein [[Pseudomonas syringae] pv. |
| | tomato str. DC3000]: |
| | MSERHVDSAPYPWPWNGQLHAHNTALIVIDMQTDFCGVGGYVDSMGYDLALTRAPIEPIRALLAVMRPLG |
| | FTIIHTREGHRPDLSDLPANKRWRSQRIGAGIGDPGPCGKILVRGEPGWEIIDELAPLPGEIIIDKPGKG |
| | SFCATDLELILRTRGIDNLILTGITTDVCVHTTMREANDRGFECLLLEDCCGATDPANHAAALSMVKMQG |
| | GVFGAVGHSSLLRTVLEA |
| 4 | WP_031595628.1 MULTISPECIES: cysteine hydrolase [Pseudomonas syringae |
| | group]: |
| | MSERHIASAPYPWPWNGQLHAHNTALIVIDMQTDFCGVGGYVDSMGYDLSLTRAPIEPIKALLAVMRPLG |
| | FTIIHTREGHRPDLSDLPANKRWRSQRIGAGIGDPGPCGKILVRGEPGWEIIEELAPLPGEIIIDKPGKG |
| | SFCATDLELILRTRGIDNLILTGITTDVCVHTTMREANDRGFECLLLEDCCGATDPANHAAALSMVKMQG |
| | GVFGAVGHSSMFRDLFGA |
| 5 | WP_033263155.1 cysteine hydrolase [Amycolatopsis vancoresmycina]: |
| | MTARIGPVQADPYPWPYDSVVPIDRVALLCIDWQTDFCGPGGYVERMGYDLELTRAGLPGTQKLLAHARD |
| | VGMLVIHTREGHLPDLTDLPPNKRWRSARIGAEIGSAGPAGRILVRGEPGWEIVGEVSPAPGEVVIDKPG |
| | KGAFYATNLDLVLRANAISHLILTGITTDVCVHTTMREANDRGLECLILSDCTGATDPGNHAAALKMVTM |
| | QGGVFGAVATSDAVIGATTTDG |
| 6 | WP_004883226.1 MULTISPECIES: cysteine hydrolase [Pseudomonas syringae |
| | group]: |
| | MSERYLASEPYPWPWNGKLNARNTALIVIDMQTDFCGVGGYVDSMGYDLALTRAPIEPIKGLLALMRPLG |
| | FTIIHTREGHRPDLSDLPANKRWRSQRIGAGIGDPGPCGKILVRGEPGWELIDELAPLPGEIVIDKPGKG |
| | SFYATDLELVLRTRGIENLILTGITTDVCVHTTLRDANDRGFECILLEDCCGATDPANHAAALSMIKMQG |
| | GVFGAVGHSSMLRDVLGA |
| 7 | WP_007177325.1 MULTISPECIES: cysteine hydrolase [Burkholderiaceae]: |
| | MTRFIEAKPYPWPYDGNLRPENTALVIIDMQTDFCGHGGYVDKMGYDLSLTRAPIEPIKRVLKAMREQGF |
| | TIIHTREGHRPDLSDLPANKRWRSRRAGTDGVGIGDDGPCGRILVRGEAGWEIIDELKPLAGEIVIDKPG |
| | KGSFCATDLELILRTRGIENLILTGITTDVCVHTTMREANDRGFECTVLADCCGATDKGNHDAALHMITM |
| | QGGVFGTVSDSGALLHTLGG |
| 8 | WP_008346673.1 MULTISPECIES: cysteine hydrolase [Burkholderiaceae]: |
| | MSRFIEARPYPWPYDGNLRPENTALVIIDMQTDFCGIGGYVDKMGYDLSMTRAPIEPIRNVLTLMREQGF |
| | TIIHTREGHRPDLSDLPANKRWRSRRAGTNGIGIGDEGPCGKILVRGEPGWEIIDELKPIEGEIVIDKPG |
| | KGSFCATDLEMVLRTRGIVNLVLTGITTDVCVHTTMREANDRGFECTILADCCGATDQGNHDAALNMVLM |
| | QGGVFGTVSDSKALLATLGR |
| 9 | WP_008877630.1 cysteine hydrolase [Mesorhizobium metallidurans]: |
| | MNARAGQRYIEADPYPWPYNGDLRSDNTALIIIDMQTDFCGPGGYVDHMGYDLSLVRAPIEPIKSVLSAM |
| | RAKGYTIIHTREGHRPDLADLPANKRWRSRRINAGIGDPGPCGRILVRGEPGWDIISDLYPAEGEPIIDK |
| | PGKGSFCATDLELILNQRGIENIVLTGITTDVCVHTTMREANDRGFECVMLEDCCGATDYGNHLAAIKMI |
| | KMQGGVFGAVSNSVALVAQLP |
| 10 | WP_010106328.1 cysteine hydrolase [Verminephrobacter aporrectodeae]: |
| | MHITANPYPWPWNGDLRPDNTALIVIDMQTDFCGVGGYVDKMGYDVAQTRAPIAPLQTLMAALRAAGYAV |
| | MHTREGHRPDLSDLPANKRWRSRQIGAQGVGIGDDGPCGRILVRGEPGWNIIDELAPLPGEVVIDKPGKG |
| | SFYATDLELLLRTRGIVNLLLAGITTDVCVHTTMREANDRGLECLLLSDCTAATDHGNHLAALKMITMQG |
| | GVFGAHAASSAVLQALSVLPCE |
| 11 | WP_011427969.1 cysteine hydrolase [Rhizobium etli]: |
| | MDAMVETKGHYIDADPYPWPYNGALRPDNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGFECLLLDDCCGATDYGNHLAAIK |
| | MVKMQGGVFGSVSNSATLVSQLP |
| 12 | WP_011828366.1 cysteine hydrolase [Methylibium petroleiphilum]: |
| | MPNESFVHAEPYPWPYDGDLRPDNTALIVIDMQTDFCGVGGYVDKMGYDLALTRAPIEPIKRLMARLRTL |
| | GFHIIHTREGHRPDLADLPANKRWRSRRAGTGGVGIGDVGPCGRILVRGEPGWEIIPELAPLPGEPVIDK |
| | PGKGSFYATDLDMLLRVRGIRNLLLAGITTDVCVHTTMRDANDRGYECLLLSDCTAATDHGNHLAALHMV |
| | KMQGGVFGAVASSTAVLEALA |
| 13 | WP_012427107.1 cysteine hydrolase [Paraburkholderia phytofirmans]: |
| | MTRFIEARPYPWPYDGNLRPENTALVIIDMQTDFCGHGGYVDKMGYDLSLTRAPIEPIKRVLKTMREQGF |
| | TIIHTREGHRPDLSDLPANKRWRSRRAGTDGVGIGDDGPCGKILVRGEPGWDIIDELKPVAGEIVIDKPG |
| | KGSFCATDLELILRTRGIENLILTGITTDVCVHTTMREANDRGFECTVLADCCGATDKGNHDAALHMITM |
| | QGGVFGTVSDSGALLLTLGG |
| 14 | WP_012489672.1 MULTISPECIES: cysteine hydrolase [Rhizobium]: |
| | MDAMVETKGHYIDADPYAWPYNGDLRPENTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIANIILTGITTDVCVSTTMREANDRGFECLLLEDCCGATDYGNHLAAIK |
| | MVKMQGGVFGSVSNSQTLVSQLP |
| 15 | WP_041935977.1 cysteine hydrolase [Rhizobium leguminosarum]: |
| | MDAMVETNGHFIDADPYPWPYNGALRPDNTALIIIDMQTDFCGKGGYVDHMGYDLSLVQAPIEPIKRVLA |
| | AMRAKGYHIIHTREGHRPDLADLPANKRWRSQRIGAGIGDAGPCGRILTRGEPGWDIIPELYPIEGETII |
| | DKPGKGSFCATDLELILNQKRIENIILTGITTDVCVSTTMREANDRGYECLLLEDCCGATDYGNHLAAIK |
| | MVKMQGGVFGSVSNSAALVEALP |
| 16 | WP_013107455.1 MULTISPECIES: cysteine hydrolase [Thiomonas]: |
| | MPSIASHPYPWPFDGDLRPGNTALVVIDMQTDFCGVGGYVDAMGYDLSLTRAPIEPIRKVLTAMRAVGCT |
| | IIHTREGHRPDLSDLPANKRWRSQRIGAGIGDPGPCGKILVRGEPGWEIIPELAPLPGEIVIDKPGKGSF |
| | CATDLELILHTRGIRNLVLTGITTDVCVHTTMREANDRGFECLLLTDCCGATDAGNHAAAIKMVTMQGGV |
| | FGAVSDAATLLQVWEKV |
| 17 | WP_013233429.1 cysteine hydrolase [Herbaspirillum seropedicae]: |
| | MSARFIQAEPYPWPYDGALTPANTALIVIDMQTDFCGIGGYVDKMGYDLSLTRAPIEPIRKVLAAMRAGG |
| | YTIIHTREGHRPDLSDLPANKRWRSRQIGANGVGIGDAGPCGRILVRGEPGWEIIPELAPIAGEIIIDKP |
| | GKGSFCATDLEMILHTRGIRNIVLTGITTDVCVHTTMREANDRGFECVMLSDCCGATDYNNHLAALSMIK |
| | MQGGVFGAVADSAALIAVIGA |
| 18 | WP_013652708.1 cysteine hydrolase [Polymorphum gilvum]: |
| | MSVVDIRTGRDVTSRVGPVKADPYPWPFDGDLRPDNTALIVIDMQTDFCGQGGYVDAMGYDLSLTRAPIE |
| | PIGRVLAAMRAQGYHVLHTREGHRPDLADLPANKRWRSRRIGAGIGDPGPCGRILVRGEPGWEIIPELAP |
| | LPGEPVIDKPGKGSFCATDLELILATRGIRNLVLTGITTDVCVHTTMREANDRGFECLLLEDCCGATDHG |
| | NHLAALKMIRMQGGVFGAVATSDALLAALDAGE |
| 19 | WP_013673377.1 cysteine hydrolase [Pseudonocardia dioxanivorans]: |
| | MTVSPHVATDTRTATIGPVAARPYAWPYDGAVPASRTALICIDWQVDFCGPGGYVDRMGYDIALTRRGLG |
| | PTARLLAHARETGMLVIHTREGHAPDLSDLPANKRWRSRQIGAEIGSAGPAGRILVRGEPGWEIVPEVAP |
| | VPGEVLIDKPGKGAFYATQLDLVLRSNGITHILLTGITTDVCVHTTMREANDRGYECLILSDCTGATDPS |
| | NHDAALHMVTMQGGVFGAVGTADAVVEATSWSTT |
| 20 | WP_013963785.1 cysteine hydrolase [Roseobacter litoralis]: |
| | MTIQANPYAWPYNGDLRPENTALMIIDMQTDFCGEGGYVDKMGYDLSMTQAPIEPIKAVLKAMRAGGYHI |
| | IHTREGHRADLTDLPANKRWRSRQIGAGIGDPGPCGKILVRGEAGWDIVPELYPQDGEPIIDKPGKGSFY |
| | ATDLEMILRTRGIENIILTGITTDVCVSTTMREGNDRGFECLVLSDCCGATDQGNHNAALKMVTMQGGVF |
| | GAVSDSRAVLDQLP |
| 21 | WP_015795031.1 cysteine hydrolase [Catenulispora acidiphila]: |
| | MTEAQAAPQTAPRTYGTVAAEPYAWPYDGVLNPAATALVCIDWQTDFCGPGGYVDTMGYDLALTRAPLIP |
| | TARVLAAARALGFTVIHTREGHRADLADCPPNKLWRSRQIGAGIGDSGPCGRILVRGEPGWQIVPEAAPL |
| | EGELIVDKPGKGAFYATDLDLLLRTRGITHIVLTGITTDVCVHTTMREANDRGYECLLLTDCTGATDPAN |
| | HEAAVRMVTMQGGVFGAVASSEALLKTLDMG |
| 22 | WP_018333481.1 cysteine hydrolase [Actinomycetospora chiangmaiensis]: |
| | MTTDSLPTTGTAPPAPRPAVIGPVDAAPYAWPYDGSVPTERVALICIDWQIDFCGPGGYVDRMGYDIALT |
| | REGLAPTARMLALARETGMLVVHTREGHAPDLSDLPANKRWRSAQIGAEIGSEGPTGRILVRGEPGWEIV |
| | PEVAPWPGEVLIDKPGKGAFYATQLDLVLRTHGITHLILTGITTDVCVHTTMREANDRGLECVILSDATG |
| | ATDPANHAAALHMVTMQGGVFGAVATSDAVLAGVRS |
| 23 | WP_018449133.1 cysteine hydrolase [Rhizobium gallicum]: |
| | MDAMVETKGHFIDADPYPWPYNGALRPGNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPVEGETII |
| | DKPGKGSFCATDLELILNQKRIENIILTGITTDVCVSTTMREANDRGYECLLLEDCCGATDHGNHLAAIK |
| | MVKMQGGVFGSVSNSAALIEALP |
| 24 | WP_026179047.1 cysteine hydrolase [Streptomyces hokutonensis]: |
| | MVGPVTAKPYAWPYDTSVPADRVAVLCIDWQTDFCGPGGYVDTMGYDISLTRAGLPATQKLLAHARSTGM |
| | LVVHTREGHAPDLADLPANKRWRSAQIGAEIGAAGPCGRILVRGEPGWEIVPEVAPLPGEVIVDKPGKGA |
| | FYATNLDLVLRTRGITHLVLTGITTDVCVHTTMREANDRGYECLILSDCTGATDPSNHEAALHMVTMQGG |
| | VFGCVSTADDLITATTEATS |
| 25 | WP_020563252.1 cysteine hydrolase [Methylosarcina fibrata]: |
| | MPRFVKSDPYPYPYNGDLRPENTCLIVIDMQTDFCGEGGYVDKMGYDLSLTRAPIEPIRRVLSVCREQGF |
| | HVVHTREGHRPDLSDLPDNKRWRSRQIGAGIGDPGPCGRILVRGEPGWEIIPELAPLDGEPVVDKPGKGS |
| | FYATDLDLLLRRRGIDNLILTGITTDVCVHTTMRDANDRGYECLLLGDCCGATDYGNHLAALKMIRMQGG |
| | VFGAVSTSDCLIEALS |
| 26 | WP_020617109.1 cysteine hydrolase [Paenibacillus daejeonensis]: |
| | MKLSGEVVANPYAWPYDGRLIPSRTALLVLDMQTDFCGKSGYVDRMGYDVFSTARAIEPTRRLLEMVRSI |
| | PEFTVIYTREGHRPDLADLAPNKRWRSRLIGAEIGTEGPAGRILVRGEPGWQIVPQLTPLPGETIVDKPG |
| | KGSFYGTDLDLILRSRGITHLILTGMTTDVGVQSTMREANDRGYECLILEDCTGATDIDNHVAALNMVTM |
| | QGGVFGAVTTSDQLIRVLLRLSLESARLTEGELTI |
| 27 | WP_026468572.1 cysteine hydrolase [Amycolatopsis balhimycina]: |
| | MTARIGPVQADPYPWPYDTVVPIDRVALLCIDWQTDFCGPGGYVERMGYDLELTRAGLPGTQKLLAHARD |
| | VGMLVIHTREGHLPDLADLPPNKRWRSARIGAEIGSAGPAGRILVRGEPGWEIVDEVSPAPGEVVIDKPG |
| | KGAFYATNLDLVLRANAISHLILTGITTDVCVHTTMREANDRGLECLILSDCTGATDPGNHAAALKMVTM |
| | QGGVFGAVATSDAVIGATTTDD |
| 28 | WP_020923004.1 cysteine hydrolase [Rhizobium etli]: |
| | MDATVETKGHYIDADPYPWPYNGALRPDNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGFECLLLDDCCGATDYGNHLAAIE |
| | MVKMQGGVFGSVSNSATLVSQLP |
| 29 | WP_022713792.1 cysteine hydrolase [Rhizobium mongolense]: |
| | MDAIAESKGHFIDADPYPWPYNGALRPDNTALIIIDMQTDFCGKGGYVDHMGYDLSLVQAPIEPIKHVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGYECLLLEDCCGATDYGNHLAAIK |
| | MVKMQGGVFGSVSNSETFVSQLP |
| 30 | WP_028614812.1 cysteine hydrolase [Pseudomonas pelagia]: |
| | MSQRFLSSDPYPWPYNGQLHPANTALIIIDMQTDFCGEGGYVDTMGYDLAAVRAPIEPISRVLNMMREQG |
| | FHIIHTREGHRPDLSDLPANKRWRSQRIGAGIGDAGPCGRILVRGEPGWELIPELQPLDGEVIIDKPGKG |
| | SFCATDLELILRVRGIENLILCGITTDVCVHTTMREANDRGFECLLLEDCCGATDPGNHLAAVHMVKMQG |
| | GVFGAVSDSTSLVELLSER |
| 31 | WP_024315610.1 cysteine hydrolase [Rhizobium favelukesii]: |
| | MDALVETKGHYINADPYAWPYNGALRPDNTALIIIDMQTDFCGPGGYVDHMGYDLSLVRAPIEPIKKVLA |
| | AMRAKGYHVIHTREGHRPDLADLPANKRWRSQRINAGIGDAGPCGRILVRGEPGWDIIDELKPIEGEIII |
| | DKPGKGSFCATDLELILNQKRIENIILTGITTDVCVHTTMREANDRGFECLLVEDCCGATDYGNHLAAIK |
| | MVKMQGGVFGSVSNSAILIEALP |
| 32 | WP_003421848.1 cysteine hydrolase [Pseudomonas syringae]: |
| | MSERHIASAPYPWPWNGQLHAHNTALIVIDMQTDFCGVGGYVDSMGYDLALTRAPIEPIKALLAAMRPLG |
| | FTIIHTREGHRPDLSDLPANKRWRSQRIGAGIGDPGPCGKILVRGEPGWEIIDELAPLPGEIVLDKPGKG |
| | SFCATDLELILRTRGIDNLILTGITTDVCVHTTLREANDRGFECLLLEDCCGATDPGNHAAALSMVKMQG |
| | GVFGAVGHSSMLRDLLGA |
| 33 | WP_025398328.1 cysteine hydrolase [Rhizobium leguminosarum]: |
| | MDAMVKTNGHFINADPYPWPYNGALRHDNTALIIIDMQTDFCGKGGYVDHMGYDLTLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILSQKRIENIILTGITTDVCVSTTMREANDRGYECLLLEDCCGATDYGNHLAAIK |
| | MVKMQGGVFGSVSNSAALVEALP |
| 34 | WP_025418539.1 MULTISPECIES: cysteine hydrolase [Rhizobium]: |
| | MDAMVETKGHYIDADPYPWPYNGALRPDNTALIIIDMQTDFCGKGGYVDHMGYDLSLVQAPIGPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGFECLLLEDCCGATDYGNHLAAIK |
| | MVKMQGGVFGSVSNSETLVSQLR |
| 35 | WP_026784459.1 cysteine hydrolase [Pleomorphomonas koreensis]: |
| | MTATSRTLAADPYPWPYNGDLRPENTALVIIDMQTDFCGKGGYVDAMGYDLSLTRAPIEPISRVLAAFRA |
| | GGYHVLHTREGHRADLSDLPNNKRWRSRRIGAGIGDAGPCGRILVRGEPGWEIIDELAPLPGETIIDKPG |
| | KGSFCATDLELILRQKGIDNLVLTGITTDVCVHTTMREANDRGFECLLLEDCCGATDHGNHLAAIKMVKM |
| | QGGVFGAVADSATLIEALG |
| 36 | WP_027195197.1 cysteine hydrolase [Paraburkholderia sprentiae]: |
| | MTRFIEARPYPWPYDGALRADNTALIIIDMQTDFCGIGGYVDKMGYDLSLTRAPIEPISRVLATMREQDF |
| | TIIHTREGHRPDLSDLPANKRWRSRRAGTEGIGIGDDGPCGKILVRGEPGWDIIDELTPLPGEIVIDKPG |
| | KGSFCATDLELILRTRGIVNLVLTGITTDVCVHTTMREANDRGFECTVLADCCGATDKGNHDAALNMVLM |
| | QGGVFGTVSDSSALIAALGR |
| 37 | WP_028228770.1 cysteine hydrolase [Paraburkholderia ferrariae]: |
| | MTRHIEARPYPWPYDGNLRPDNTALVIIDMQTDFCGYGGYVDKMGYDLSLTRAPIEPIKKVLGLMRELGF |
| | TIIHTREGHRPDLSDLPANKRWRSRRAGTDGIGIGDAGPCGRILVRGEPGWEIIDELAPLPGEIVIDKPG |
| | KGSFCATDLELILRTRGIVNLVLTGITTDVCVHTTMREANDRGFECTVLADCCGATDPGNHDAALNMILM |
| | QGGVFGTVSGSAAMIAALGQ |
| 38 | WP_028739231.1 MULTISPECIES: cysteine hydrolase [Rhizobium]: |
| | MAPAASDLSYIDADPYNWPYNGKLRPDNTALIIIDMQTDFCGPGGYVDHMGYDLALVRAPIEPIKRVLAA |
| | MRAKGYHIIHTREGHRPDLADLPANKRWRSQRIHAGIGDPGPCGRILVRGEPGWDIIEELYPIDGEVIID |
| | KPGKGSFCATDLELILNQKRIENIILTGITTDVCVSTTMREANDRGFECLLLEDCCGATDYGNHLAAIKM |
| | VKMQGGVFGSVSNSETLVRQLP |
| 39 | WP_029007464.1 cysteine hydrolase [Azospirillum halopraeferens]: |
| | MTERFIPADPYPWPYNGDLRPDNTALIVIDMQTDFCGKGGYVDCMGYDLELTRAPIEPIRRVLAAMRAKG |
| | YHVLHTREGHRPDLSDLPANKRWRSRRIGAGIGDPGPCGRILVRGEPGWEIIPELAPLPGEPVIDKPGKG |
| | SFCATDLELILNTRGIRNIVLTGITTDVCVHTTMREANDRGFECLLLEDCCGATDPGNHAAAVAMVKMQG |
| | GVFGSVSDSAAFTGHLP |
| 40 | WP_051392089.1 cysteine hydrolase [Rhodoferax saidenbachensis]: |
| | MHIHANPYAWPWNGDLRPDNTALIVIDMQTDFCGAGGYVDKMGYDISLTRAPIEPLKVLMAALRAAGYPV |
| | MHTREGHRPDLSDLPANKRWRSQQIGTNGVGIGDAGPCGRILVRGEPGWELIPELAPLPGEVVIDKPGKG |
| | SFYATDLELVLRTRGITNLLLAGITTDVCVHTTMRDANDRGFECLLLSDCTAATDHGNHLAALKMVTMQG |
| | GVFGAHAPSSAVLAALSNTTVRPEPVEGILQ |
| 41 | WP_030472255.1 cysteine hydrolase [Lechevalieria aerocolonigenes]: |
| | MTARIGPVQADPYPWPYDTVVPVDRVALLCIDWQTDFCGPGGYVERMGYDLELTRAGLPGTQKLLAHARD |
| | VGMLVIHTREGHLPDLTDLPPNKRWRSARIGAEIGSAGPAGRILVRGEPGWEIVDEVSPAPGEVVIDKPG |
| | KGAFYATNLDLVLRANAISHLILTGITTDVCVHTTMREANDRGLECLILSDCTGATDPGNHAAALKMVTM |
| | QGGVFGAVSTSDAVIGATTTDD |
| 42 | WP_035078376.1 cysteine hydrolase [Devosia riboflavina]: |
| | MVAGGSTIASADPYPWPFDGNWGPHNTALVVIDMQVDFCAPGGYVDTMGYDIGLTRAPIEPIQQVLTAMR |
| | AKGYTIIHTREGHKPDLSDLPANKRWRSQRIGAGIGDQGPCGRILVRGEPGWEIIPELQPLPGEQIIDKP |
| | GKGTFIATDFELVLRMKNIRNIIFTGVTTDVCVHTTMRDANDRGYECLLLEDCCAATKQSNHDAAVDMIK |
| | MQGGVFGAVSTSAALIEVLP |
| 43 | WP_035256306.1 cysteine hydrolase [Actibacterium mucosum]: |
| | MTTIESHPYKWPYNGDLRPENTALIIIDMQTDFCGKGGYVDAMGYDLSLTRAPIAPIKAVLTAMRAKGYH |
| | ILHTREGHRPDLSDLPANKRWRSQQIGAGIGDPGPCGKILIRGEPGWDIIDELYPLPGETIIDKPGKGSF |
| | CATDLEMILRTRGIENLIICGITTDVCVSTTMREANDRGFECVVLEDCCGATDRGNHDAAIKMVTMQGGV |
| | FGAVSDSNALIAGLV |
| 44 | WP_036050193.1 cysteine hydrolase [Burkholderia gladioli]: |
| | MNRHIEAKPYPWPYDGALRPDNTALVVIDMQTDFCGHGGYVDKMGYDLSLTRAPIEPIGRVLAAMRAQGY |
| | TIIHTREGHRPDLSDLPANKRWRSRQAGTDGVGIGDDGPCGRILVRGEPGWEIIEELAPLAGEVVIDKPG |
| | KGSFYATDLELILRTRGIANLILTGITTDVCVHTTMREANDRGFECVILADCCGATDPANHAAALHMVTM |
| | QGGVFGAVSSSAALLATLGAAS |
| 45 | WP_037459080.1 cysteine hydrolase [Skermanella stibiiresistens]: |
| | MTHIDSDPYPWPYDGDLRPANTALVVIDMQTDFCGKGGYVDAMGYDLALTRAPIEPIARLMAAMRQGGFT |
| | ILHTREGHRPDLADLPDNKRWRSRRIGAGIGDPGPCGRVLVRGEPGWEIIPELSPLPGEPIIDKPGKGSF |
| | CATDLDLMLRQRGIRNLVLTGITTDVCVHTTMREANDRGYECVLVEDCCAATDRSNHDAAIRMVKMQGGV |
| | FGAVARSDALLTVL |
| 46 | WP_037484943.1 cysteine hydrolase [Sphaerotilus natans]: |
| | MPVHLNSVPYPWPCDGQFTPANTALVIIDMQTDFCGVGGYVDTMGYDISLTRAPIEPLQRLLAEARRTGL |
| | HVIHTREGHRPDLSDLPANKRWRSQRIGAGIGDPGPCGRILVRGEPGWDIIPELYPIAGEPIIDKPGKGS |
| | FCATDLELILHTRGIRNLILTGITTDVCVHTTMREANDRGFECVMLGDCTGATDRGNHEAALKMIQMQGG |
| | VFGAVADSASVLAVLAGWPDPTG |
| 47 | WP_040114689.1 cysteine hydrolase [Rhizobium gallicum]: |
| | MDAMAESKGHFIDADPYPWPYNGALRPDNTALIIIDMQTDFCGKGGYVDHMGYDLSLVQAPIEPIKQVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGYECLLLDDCCGATDCGNHLAAIK |
| | MVKMQGGVFGSVSSSETFVSQLP |
| 48 | WP_040119808.1 MULTISPECIES: cysteine hydrolase [Enterobacteriaceae]: |
| | MNERYLASDPYPWPYDGQLTTSNTALVIIDMQTDFCGTGGYVDSMGYDLSLTRAPIAPIKRVLARMREKG |
| | FPIIHTREGHRPDLSDLPDNKRWRSQRLGAGIGDVGTCGRILVRGEPGWEIIPELAPLPGEVIIDKPGKG |
| | SFYATDLELILRTRGITHLILTGITTDVCVHTTLREANDRGFECLILEDCCGATDYQNHLAALSMVKMQG |
| | GVFGAVASAAMLLDALGGE |
| 49 | WP_044431670.1 cysteine hydrolase [Skermanella aerolata]: |
| | MTHIDSDPYPWPYDGDFRPANTALIVIDMQTDFCGKGGYVDAMGYDLSLTRAPIEPIARLMAAMRQGGFT |
| | IFHTREGHRPDLADLPANKRWRSQRIGAGIGDPGPCGRILVRGEPGWDIIPELAPLPGEPVIDKPGKGSF |
| | CATDLDLMLRQRGIRNIVLTGITTDVCVHTTMREANDRGYECLLLEDCCGATDRGNHEAAVKMVKMQGGV |
| | FGAVARSTDLLRVLS |
| 50 | WP_045672424.1 cysteine hydrolase [Paenibacillus beijingensis]: |
| | MFIEGNPYPFPYNRDLRAGNSALVIIDMQIDFCGKGGYVDRMGYDISLTRSAIEPIKLLLEAARSIPGFT |
| | IIHTREGHRKDLSDLPANKRWRSKQIGAEIGSDGPAGKILIRGEPGWDIIEELAPQAGEIVIDKPGKGSF |
| | YATDLDLLLRTKGIQNLILTGITTDVCVHTTMREANDRGYECLILEDCTGATDYQNHLAALKMVTMQGGV |
| | FGSVSRSEHVLPVLRTLANEGS |
| 51 | WP_045774129.1 cysteine hydrolase [Elstera litoralis]: |
| | MPMPLIASAPYEWPWNADLRPQNTALIVIDMQTDFCGTGGYVDTMGYDLSLTRAPIEPIKRLLAAMRAKG |
| | YFIIHTREGHRPDLSDLPPNKRWRSQQIGAGIGDAGPCGRILVRGEPGWEIIPDLAPLPGEVIIDKPGKG |
| | SFCATDLDLILRQQGIANIILTGITTDVCVHTTMREANDRGYECLLLEDCCGATDHSNHLAALSMVKMQG |
| | GVFGAVASSEALLAALP |
| 52 | WP_046572974.1 cysteine hydrolase [Paraburkholderia fungorum]: |
| | MNRFIEAKPYPWPYDGNLRADNTALVIIDMQTDFCGHGGYVDKMGYDLSLTRAPIEPIKSVLQLMRQLGF |
| | TIIHTREGHRPDLSDLPANKRWRSRRAGTDGVGIGDDGPCGRILVRGEPGWEIIDELKPLPGEIIIDKPG |
| | KGSFCATDLELILRTRGIVNLVLTGITTDVCVHTTMREANDRGFECTVLADCCGATDKRNHDAALHMITM |
| | QGGVFGTVSDSHALLATLLATTTAPAAALATSGR |
| 53 | WP_050475712.1 cysteine hydrolase [Herbaspirillum rhizosphaerae]: |
| | MSELFIQAEPYMWPYDGALTPGNTALVIIDMQTDFCGIGGYVDKMGYDLSLTRAPIAPIKSVLSAMRAGG |
| | YTIIHTREGHRPDLSDLPANKRWRSRQIGTNGVGIGDDGPCGKILVRGEAGWEIIPELAPLPGEIIIDKP |
| | GKGSFCATDLELVLHTRGIRNLILTGITTDVCVHTTMREANDRGFECVMLADCCGATDHNNHLAALSMIK |
| | MQGGVFGAVSDSSSLLQAIGK |
| 54 | WP_054985868.1 MULTISPECIES: cysteine hydrolase [Pseudomonas syringae |
| | group]: |
| | MSERHIASAPYPWPWNGQLHAHNTALIVIDMQTDFCGVGGYVDSMGYDLALTRAPIEPIKALLAAMRPLG |
| | FTIIHTREGHRPDLSDLPANKRWRSQRIGAGIGDPGPCGKILVRGEPGWEIIDELAPVPGEIVIDKPGKG |
| | SFCATDLELILRTRGIDNLILTGITTDVCVHTTMREANDRGFECLLLEDCCGATDPANHAAALSMVKMQG |
| | GVFGAVGHSSMLRDLLGA |
| 55 | WP_057403488.1 cysteine hydrolase [Pseudomonas amygdali]: |
| | MSERHIASAPYPWPWNGQLHAHNTALIVIDMQTDFCGVGGYVDSMGYDLSLTRAPIEPIKALLAVMRPLG |
| | FTIIHTREGHRPDLSDLPANKRWRSQRIGAGIGDPGPCGKILVRGEPGWEIIDELAPLPGEIVLDKPGKG |
| | SFCATDLELILRTRGIDNLILTGITTDVCVHTTMREANDRGFECVLLEDCCGATDPGNHAAALSMVKMQG |
| | GVFGAVGHSSMFRDLFGA |
| 56 | WP_044530929.1 MULTISPECIES: cysteine hydrolase [Herbaspirillum]: |
| | MSALYIQAEPYPWPYDGALTPANTALIVIDMQTDFCGIGGYVDKMGYDLSLTRAPIEPIKTVLAAMRAGG |
| | YTIIHTREGHRPDLSDLPANKRWRSRQIGTDGVGIGDAGPCGRILVRGEPGWEIIPELAPIAGEIIIDKP |
| | GKGSFCATDLEMILHTRGIRNIVLTGITTDVCVHTTMREANDRGFECVMLSDCCGATDYSNHLAALSMIK |
| | MQGGVFGAVSDSAALIDVIGA |
| 57 | WP_060717458.1 cysteine hydrolase [Agrobacterium vitis]: |
| | MDMSTETKLHTLAADPYPWPYNGEWRPDNTALIIIDMQTDFCGPGGYVDHMGYDLSLVRAPIEPIKAVLA |
| | AMRAKGYHIIHTREGHRPDLADLPANKRWRSQRIKSGIGDPGPCGRILVRGEPGWNIIEELAPLDGETII |
| | DKPGKGSFCATDLELILNQKRIQNIILTGITTDVCVHTTMREANDRGFECLLLEDCCGATDYGNHLAAIK |
| | MVKMQGGVFGAVSHSKDLIEALP |
| 58 | WP_062363788.1 cysteine hydrolase [Variovorax paradoxus]: |
| | MANTASPRHVAAEPYGWPYNGALRPGNTALIVIDMQTDFCGTGGYVDVMGYDLSLVQAPIQPIARTLAAL |
| | RPLGFHIIHTREGHRPDLSDLPANKRWRSRQIGANGVGIGDDGPCGRILVRGEPGWEIIPELAPLPGEVV |
| | IDKPGKGSFYATDLEPILRTRGIENLILAGITTDVCVHTTMRDANDRGFECLLLSDCTAATDHGNHLAAL |
| | KMITMQGGVFGAHATSQALLAALD |
| 59 | WP_064243180.1 cysteine hydrolase [Ensifer glycinis]: |
| | MNSLASLPITSQKLSHIDADPYPWPYNGDLRPENTALIIIDMQADFCGPGGYVDHMGYDLSLVRAPIEPI |
| | RKVLSAMRAKGYHIIHTREGHRPDLADLPANKRWRSQRIKAGIGDPGPCGRILVRGEPGWEIIDELKPIE |
| | RETIIDKPGKGSFCATDLELILNQKRIDNIVLTGITTDVCVHTTMREANDRGFECLLLADCCGATDYGNH |
| | LAAIKMVKMQGGVFGSVSNSATLVGQLP |
| 60 | WP_064823845.1 cysteine hydrolase [Rhizobium phaseoli]: |
| | MDAMVETKGHYIVADPYAWPYNGDLRPENTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIISELYPIEGETII |
| | DKPGKGSFCATDLELILNQKRIANIILTGITTDVCVSTTMREANDRGFECLLLEDCCGATDYGNHLAAIK |
| | MVKMQGGVFGSVSNSQTLVSQLP |
| 61 | WP_064837226.1 cysteine hydrolase [Rhizobium phaseoli]: |
| | MDAMVETKGHYIDADPYAWPYNGDLRPENTALIIIDMQTDFCGKGGYVDNMGYDLSLVQAPIEPIKRVLA |
| | AMRVKGYHIIHTREGHRPDLADLPANKRWRSQRIGAGIGDPGPCGRILTRGEPGWDIISELYPIEGETII |
| | DKPGKGSFCATDLELILNQKRIANIILTGITTDVCVSTTMREANDRGFECLLLEDCCGATDYGNHLAAIK |
| | MVKMQGGVFGSVCNSQTLVSQLP |
| 62 | WP_066257666.1 cysteine hydrolase [Hydrogenophaga flava]: |
| | MERFIDANPYAWPYNGDLRPENTALIVIDMQTDFCGIGGYVDQMGYDISLTRAPIAPLQTLMAAMRGAGY |
| | TVIHTREGHRPDLSDLPANKRWRSRQIGTNGVGIGDDGPCGRILVRGEPGWEIIPELAPLPGEVVIDKPG |
| | KGSFYATDLELLLRTRGISNLLLAGITTDVCVHTTMRDANDRGFECLLLSDCTAATDRGNHEAALKMITM |
| | QGGVFGAHAPSTAVLEALA |
| 63 | WP_066811963.1 cysteine hydrolase [Defluviimonas alba]: |
| | MTTLASTPYAWPWNGDLRPENTALIIIDMQADFCGKGGYVDQMGYDLSLTQAPIRPIGTVLAAMRAKGYP |
| | VIHTREGHRPDLSDLPPNKRWRSRQIGAGIGEDGPCGRILIRGEPGWDIIPELYPIAGETIIDKPGKGSF |
| | CATDLELILRTRGIDNLILTGITTDVCVSTTMREANDRGFECLILSDCCAATDPGNHAAALKMVTMQGGV |
| | FGAVSDSATLIGALP |
| 64 | WP_068803416.1 cysteine hydrolase [Immundisolibacter cernigliae]: |
| | MPERFLDAEPYPWPFDGDLRPANTALIIIDMQTDFCGPGGYVDTMGYDISLTRAPIEPIRAVLAAFRAGG |
| | FHVIHTREGHRPDLADLPANKRWRSRRIGAGIGDPGPCGRILVRGEPGWEIIPELAPLAGEPVIDKPGKG |
| | SFCATDLELLLHVRGIRNLVLTGITTDVCVHTTMREANDRGFECLLVADCCGATDHGNHLAALNMIKMQG |
| | GVFGAVADSAALLAGLRA |
| 65 | WP_069307252.1 cysteine hydrolase [Methylobrevis pamukkalensis]: |
| | MTASLDERIEAVRVPGGRTIDSEPYAWPFDGDLRPENTAIVVIDMQVDFCAPGGYVDSMGYDIALTRAPI |
| | APIARLLEAVREKGFTVIHTREGHKPDLSDLPANKRWRSQRIGAGIGDQGPCGRILVRGEPGWEIIPELQ |
| | PIAGEAIIDKPGKGSFLATDFDLVLQTKRIRNIVLTGVTTDVCVHTTMRDANDRGYECLLLSDCTAATKL |
| | ENHLAALDMVKMQGGVFGAVATSDAFIAGIA |
| 66 | WP_072378795.1 cysteine hydrolase [Rhizobium tibeticum]: |
| | MDALVETKGHYINADPYAWPYNGALRPDNTALIIIDMQTDFCGPGGYVDHMGYDLSLVRAPIEPIKGVLS |
| | AMRAKGYHIIHTREGHRPDLADLPANKRWRSQRINAGIGDAGPCGRILVRGEPGWDIIDELKPIEGETII |
| | DKPGKGSFCATDLELILNQKRIENIILTGITTDVCVHTTMREANDRGFECLLLEDCCGATDYGNHLAAIK |
| | MVKMQGGVFGSVSNSATLIEALP |
| 67 | WP_072642261.1 cysteine hydrolase [Rhizobium leguminosarum]: |
| | MDAMVETEGHFIDADPYPWPYNGALRPDNTALIIIDMQTDFCGKGGYVDHMGYDLTLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGYECLLLEDCCGATDYGNHLAAIK |
| | MVKMQGGVFGSVSNSETFVSQLP |
| 68 | WP_073055721.1 cysteine hydrolase [Kaistia soli]: |
| | MSTDTLDERVAAAVVPGGRTIASDPYPWPFDGNLKPENTALIVIDMQVDFCAPGGYVDSMGYDISLTREP |
| | IEPIRRVLDAMRAKGYHIIHTREGHRPDLADLPANKRWRSQRIGAGIGDQGPCGRILVRGEPGWEIIPEL |
| | QPVEGETIIDKPGKGSFVATDLELVLRQKGIRNIVLAGVTTDVCVHTTMRDANDLGYECVLLSDCTAATK |
| | RENHLAAIDMVKMQGGVFGAVAT SDALIAGLV |
| 69 | WP_074637487.1 cysteine hydrolase [Sulfitobacter pontiacus]: |
| | MQRAGATDVTTVQSHPYAWPYNGDLRPENTALVIVDMQTDFCGVGGYVDHMGYDLSLTQAPIAPIKALLA |
| | DMRAKGYHIIHTREGHRLDMADLPANKRWRSQQIGAGIGDSGPCGKILIRGEAGWDIIPELAPLEGETII |
| | DKPGKGSFYATDLELILRTRQIDNLILTGITTDVCVSTTMREANDRGFECVVVEDCCGATDPANHAAAIK |
| | MVTMQGGVFGAVTTSADLIAGLPS |
| 70 | WP_074830085.1 MULTISPECIES: cysteine hydrolase [Pseudomonas]: |
| | MNLRHLASAPYPWPYNGRLDPANTALVIIDMQTDFCGVGGYVDTMGYDLSLTRAPIEPIKRVLEVMRAQG |
| | FPIIHTREGHRPDLADLPANKRWRSQRIGAGIGDDGPCGRILVRGEPGWEIIPELAPLPGEIIIDKPGKG |
| | SFYATDLELVLRNHGIDNLVLTGITTDVCVHTTMREGNDRGFECILLEDCCGATDHGNHLAALNMIKMQG |
| | GVFGAVGNSSMLLDVLGRA |
| 71 | WP_074987393.1 cysteine hydrolase [Paraburkholderia tropica]: |
| | MTRFIEARPYPWPYDGALRADNTALVIIDMQTDFCGFGGYVDKMGYDLSLTRAPIEPIKHVLATMRAQGF |
| | TIIHTREGHRPDLSDLPANKRWRSRQAGTNGIGIGDDGPCGKILVRGEPGWEIIDELAPLPGEIVIDKPG |
| | KGSFCATDLELVLRTRGIANLVLTGITTDVCVHTTMREANDRGFECTILADCCGATDQGNHDAALKMVLM |
| | QGGVFGTVSDSHALLATLGR |
| 72 | WP_075290549.1 cysteine hydrolase [Rhizobium arenae]: |
| | MDAQATATGQYVKADPYPWPYNGALRPDNTALIIIDMQTDFCGPGGYVDHMGYDLSLVRAPIEPIKAVLA |
| | AMRAKGYHIIHTREGHRPDLGDLPANKRWRSQRIGAGIGDVGPCGRILVRGEPGWDIIEELYPIEGETII |
| | DKPGKGSFCATDLELILNQKRIENIILTGITTDVCVHTTMREANDRGFECLILGDCCGATDYGNHLAALK |
| | MVTMQGGVFGSVSNSKDLIAALP |
| 73 | WP_075633397.1 cysteine hydrolase [Rhizobium rhizosphaerae]: |
| | MTTIHADPYLWPYNGDLRPENTAFIIIDMQTDFCGPGGYVDTMGYDIALTRAPIEPIKTVLAAMREKGYH |
| | VIHTREGHRPDLSDLPPNKRWRSQQIGAGIGDAGPCGRILVRGEPGWEIIDELKPLAGEPIIDKPGKGSF |
| | CATDLELLLRTRGIENIVLSGITTDVCVHTTMREANDRGFECLLLEDCCAATDPGNHAAAIKMVKMQGGV |
| | FGAVSNSAAFVEALP |
| 74 | WP_076625678.1 cysteine hydrolase [Thiobacimonas profunda]: |
| | MTAYPQVKSDPYAWPFDGRFTPADTALIIIDMQRDFCDVDGWVGQHGADPAPMRAVVEPIRAVLGRMREL |
| | GFPIIHTREGHRPDLADLNDNKRWRSAREGAEIGTAGPCGRMLTRGEPGWEIVPELTPAAGEPVIDKPGK |
| | GAFYATDLEQILHARGIRNLIFTGVTTDCCVHTTMRDANDRGFECMLLDDCCAASLAHNHQAILKFTKMG |
| | DGLFGTVGTSAQLFEALA |
| 75 | WP_078814169.1 cysteine hydrolase [Prosthecobacter debontii]: |
| | MPYVEADPYPWPYNGDLRPENTTFLVIDMQTDFCGPGGYVDKMGYDLSLTRAPIEPIKKVFEAVRAKGYH |
| | VMHTREGHRPDLADLPANKKWRSQRIGAGIGDVGPCGRILTRGEPGWDIIPELYPEPGEAIIDKPGKGSF |
| | YGTDLDMLLRQKGIQNIVLAGITTDVCVHTTMREANDRGFECLLLSDCTGATDYGNYLAALKMIKMQGGV |
| | FGAVSDSTAFIQAVMA |
| 76 | WP_079177709.1 cysteine hydrolase [Streptomyces mangrovisoli]: |
| | MPATPTPEPSPPASAPHAAAPEPPAPTAPGTVAADPYTWPYDGPIRPERTALLCIDWQTDFCGPGGYVDA |
| | MGYDLALTRAPLGPTAKVLAAARSVGLTVVHTREGHRPDLSDCPPNKLWRSRRIGAGIGDAGPCGRILVR |
| | GEPGWEIVPEAAPLPGELIIDKPGKGSFYATDLDLLLRTRGITHLILTGITTDVCVHTTMRDANDRGYEC |
| | LLLTDCTGATDPANHAAALHMVTMQGGVFGAIAPSAAVLEALAAL |
| 77 | WP_079417747.1 cysteine hydrolase [Thiomonas intermedia]: |
| | MADFSSDAAAGPVAANPYPWPFDGDLRPANTALIVIDMQTDFCGIGGYVDRMGYDLSLTRAPIEPIGRVL |
| | AAMRAGGYTIFHTREGHRPDLSDLPANKRWRSQRIGAGIGDPGPCGKILVRGEPGWDIIPELAPRPGEII |
| | IDKPGKGSFCATDLELILHQRGIRNLVLTGITTDVCVHTTMREANDRGFECLLLADCCGATDATNHAAAL |
| | KMVTLQGGVFGAVANSAELLRALETQA |
| 78 | WP_083726432.1 cysteine hydrolase [Pseudomonas pachastrellae]: |
| | MTERYLQSAPYPWPYNGNLTPENTALIVIDMQTDFCGKGGYVDSMGYDLSLTRAPIEPISRVLEVLREQG |
| | FWIIHTREGHRPDLSDLPANKRWRSQRIGAGIGDTGPCGRILVRGEPGWEIIPELAPIDGEIIIDKPGKG |
| | SFCATDLELILRTRGIENIILAGITTDVCVHTTMREANDRGFECILLEDCCGATDHANHLAALSMVKMQG |
| | GVFGAIGDSAMLIDCLKQV |
| 79 | WP_085560469.1 cysteine hydrolase [Carnobacterium iners]: |
| | MQLEKDYKLFQEEKIIPYPTWQYGEIKGKIITLEVEDAPDFGETAYVELDSGRTAFISVDMQTDFCGENG |
| | YVDVMGYDLSLTASAIKPIKNVLDAIRGSDIQIIHTREGHSPDLSDAPYLKVLRSKIIGKGIGIGDRPEK |
| | GLGRLLIRGEKNWDIIDELYPLEGEIIIDKAGKGAFASSNIHLILKNLGITHLVLTGITADVCVHTIMRE |
| | ANDYGYGCILLKDATGATDQGNCESAIKSIKMQGGVFGNVSDSEKFIKAFKEAL |
| 80 | WP_085780954.1 cysteine hydrolase [Rhizobium sp. NXC14]: |
| | MDAMVETKGHYIDADPYAWPYNGALRADNTALIIIDMQTDFCGKGGYVDHMGYDLSLVQAPIEPIKKVLA |
| | AMRAKGYHIIHTREGHRPDLADLPANKRWRSQRIGAGIGDPGPCGRILTRGEPGWDIIPELYPIEGEAII |
| | DKPGKGSFCATDLELILNRKRIENIILTGITTDVCVSTTMREANDRGFECLLLEDCCGATDYGNHLAAIK |
| | MVKMQGGVFGSVSNSATLVSQLP |
| 81 | WP_085861497.1 MULTISPECIES: cysteine hydrolase [Rhizobium]: |
| | MDATVETKGHYIDADPYPWPYNGALRPDNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGFECLLLEDCCGATDYGNHLAAIK |
| | MVKMQGGVFGSVSSSATLVSQLP |
| 82 | WP_090877027.1 cysteine hydrolase [Bauldia litoralis]: |
| | MSVTEFPVEHRATGRTVPADPYPWPYDGALRPDNTALIVIDMQTDFCGPGGYVDKMGYDLSLTRAPIEPI |
| | RSVLGAMRAKGYHVIHTREGHRPDLSDLPHNKRWRSRQIGAGIGDAGPCGQILVRGEPGWQIIPELAPAP |
| | GEPVIDKPGKGSFYATDLELLMRTRGIHNLVLTGITTDVCVHTTMREANDRGFECLLLEDCCGATDHDNH |
| | LAAIRMIKMQGGVFGAVATADAFVGALP |
| 83 | WP_091276718.1 cysteine hydrolase [Micromonospora haikouensis]: |
| | MARIGPVTANPYPWPYDGAVDTTRTALLCIDWQTDFCGPGGYVDAMGYDISLTRSGLPATARLLAHARSL |
| | GMLVVHTREGHDPDLADLPPNKRWRSARIGAEIGGAGPCGRILVRGEPGWEIVPEVAPTPGEVVVDKPGK |
| | GAFYATNLDLVLRTRGITHLILTGITTDVCVHTTMREANDRGYECLILADCTGATDKGNHDAALHMVTMQ |
| | GGVFGCVATSDDVIAATAR |
| 84 | WP_091583823.1 cysteine hydrolase [Mesorhizobium qingshengii]: |
| | MNARAEITQRYIDADPYPWPYNGDLRPDNTALIIIDMQTDFCGPGGYVDHMGYDLSLVRAPIEPIKSVLS |
| | AMRAKGYTIIHTREGHRPDLADLPANKRWRSRRINAGIGDAGPCGRILVRGEPGWDIIPDLYPIEGEPII |
| | DKPGKGSFCATDLELILNQRGIQNIVLTGITTDVCVHTTMREANDRGYECMMLEDCCGATDHGNHLAAIK |
| | MIKTQGGVFGTVSNSNALVAQLP |
| 85 | WP_093084166.1 cysteine hydrolase [Pseudonocardia oroxyli]: |
| | MTGSTDLSTSTRTATIGPVAARPYAWPYDGAVPASKTALICIDWQVDFCGPGGYVDRMGYDIGLTRKGLG |
| | PTARLLAHARETGMLVVHTREGHAPDLSDLPANKRWRSKQIGAEIGSPGPTGRILVRGEPGWEIVPEVAP |
| | VPGEVLIDKPGKGAFYATQLDLVLRSNGITHILLTGITTDVCVHTTMREANDRGYECLILSDCTGATDPA |
| | NHDAALHMVTMQGGVFGAVGTADAVVEATTWSDS |
| 86 | WP_093153408.1 cysteine hydrolase [Pseudoruegeria lutimaris]: |
| | MATIPSDPYPWPYNGDLRPENTALIVIDMQTDFCGKGGYVDKMGYDLKMTRAPIEPIKAVLAVMRAKGYH |
| | IIHTREGHRPDLSDLPANKRWRSQQIGAGIGDPGPCGKILVRGEPGWDIIEELFPDPGEIIIDKPGKGSF |
| | CATDLELILRTRGIENLVICGITTDVCVSTTMREANDRGFECLVLEDCCGATDLGNHNAALKMVKMQGGV |
| | FGAVSDSATMIAGLA |
| 87 | WP_093620408.1 cysteine hydrolase [Actinoplanes philippinensis]: |
| | MTARIGPVPANPYPWPYDGSVPVDRTALLCIDWQTDFCGPGGYVDSMGYDIELTRAGLPATAKLLSHVRE |
| | LGMLVIHTREGHDPDLSDLPANKRWRSARIGAEIGGPGPCGRILIKGEPGWEIVPEVAPAPGEVIVDKPG |
| | KGAFYATNLDLVLRTHGITHLILTGITTDVCVHTTMREANDRGYECLILSDCTGATDPSNHTAALHMVTM |
| | QGGVFGCVATSDDVIAATGG |
| 88 | WP_093645941.1 cysteine hydrolase [Paraburkholderia aspalathi]: |
| | MNRFIEAKPYPWPYDGDLRPDNTALVIIDMQTDFCGHGGYVDKMGYDLSLTRAPIEPIKSVLKPMRELGF |
| | TIIHTREGHRPDLSDLPANKRWRSRRAGTDGVGIGDDGPCGKILVRGEPGWEIIDELKPLPGEIIIDKPG |
| | KGSFCATDLELILRTRGIVNLVLTGITTDVCVHTTMREANDRGFECTVLADCCGATDKGNHDAALHMITM |
| | QGGVFGTVSDSHALLATLLAKTAAPAAALATSGR |
| 89 | YP_234257.1 isochorismatase hydrolase [Pseudomonas syringae pv. |
| | syringae B728a]: |
| | MSERHIASAPYPWPWNGQLHAHNTALIVIDMQTDFCGVGGYVDSMGYDLALTRAPIEPIKALLATMRPLG |
| | FTIIHTREGHRPDLSDLPANKRWRSQRIGAGIGDPGPCGKILVRGEPGWEIIDELAPLPGEIVLDKPGKG |
| | SFCATDLELILRTRGIDNLILTGITTDVCVHTTLREANDRGFECLLLEDCCGATDPDNHAAALSMVKMQG |
| | GVFGAVGHSSMLRDLLGA |
| 90 | WP_051074034.1 cysteine hydrolase [Rhizobium freirei]: |
| | MNYPAAASTEQTLAYVDADPYGWPYNGALRPDNTALIIIDMQTDFCGPGGYVDHMGYDLSLVRAPIEPIK |
| | RVLAAMRAKGYHIIHTREGHRPDLADLPANKRWRSQRINAGIGDPGPCGRILVRGEPGWDIIDELKPIDG |
| | ETIIDKPGKGSFCATDLELILNQKRIENIILTGITTDVCVHTTMREANDRGFECLLLEDCCGATDYGNHL |
| | AAIKMVKMQGGVFGSVSNSANLVSQLP |
| 91 | WP_040604119.1 cysteine hydrolase [Sagittula stellata]: |
| | MTETPVGTTIDSAPYAWPWNGDLRPDNTALIIIDMQTDFCGVGGYVDSMGYDISLTRAPIQPIQSVLKAF |
| | RDKGYMVIHTREGHRPDLSDLPDNKRWRSRQIGAGIGDPGPCGRILTRGEPGWEIIGELTPEPGEVIVDK |
| | PGKGCFCATDLEMILRLRGIDNIVLTGITTDVCVHTTMREANDRGFECVMLTDCCAATDPANHAAAIHMI |
| | HMQGGVFGATATSDALLKVLP |
| 92 | WP_040454192.1 cysteine hydrolase [Hydrocarboniphaga effusa]: |
| | MTERTIDAEPYRWPYNGDLRPQNTALVIIDMQTDFCGVGGYVDKMGYDLSLTRAPIEPIKRVLTRFRELG |
| | FHVIHTREGHRPDLSDLPANKRWRSRQIGAGIGDPGPCGRILVRGEPGWDIIEELYPLPGEPIIDKPGKG |
| | SFCATDLELMLRVKGIDNIVLTGITTDVCVHTTMREGNDRGFECVLLADCCGATDYNNHLAAQQMIKMQG |
| | GVFGAVSNSEALLAALA |
| 93 | WP_009983899.1 MULTISPECIES: cysteine hydrolase [Rhizobium]: |
| | MDAMVETKGDYIDADPYAWPYNGDLRPDNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPIVGETII |
| | DKPGKGSFCATDLELILNQKRIANIILTGITTDVCVSTTMREANDRGFECLLLEDCCGATDYGNHLAAIK |
| | MVKMQGGVFGSVSNAQTLVSQLP |
| 94 | WP_010429021.1 MULTISPECIES: cysteine hydrolase [Pseudomonas]: |
| | MSERHIASAPYPWPWNGQLHAHNTALIVIDMQTDFCGVGGYVDSMGYDLALTRAPIEPIKALLAAMRPLG |
| | FTIIHTREGHRPDLSDLPANKRWRSQRIGAGIGDPGPCGKILVRGEPGWEIIDDLAPLPGEIVIDKPGKG |
| | SFCATDLELILRTRGIDNLILTGITTDVCVHTTMREANDRGFECLLLEDCCGATDAGNHAAALSMVKMQG |
| | GVFGAVGHSSMLRDLLGA |
| 95 | WP_011654379.1 cysteine hydrolase [Rhizobium leguminosarum]: |
| | MDAMVETNRHFIDADPYPWPYNGALRPDNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELVLNQKRIENIILTGITTDVCVSTTMREANDRGYECLLLEDCCGATDYGNHLAAIK |
| | MVKMQGGVFGSVSNSAALVEALP |
| 96 | WP_012976323.1 cysteine hydrolase [Azospirillum lipoferum]: |
| | MTERFVPAAPYPWPYNGDLTPANTALIVIDMQTDFCGTGGYVDSMGYDLSLTRAPIEPIRALLAAMRAGG |
| | YHILHTREGHRPDLSDLPANKRWRSRRIGAGIGDPGPCGRILVRGEPGWEIIPDLAPLPGEPVIDKPGKG |
| | SFCATDLELILTTRGIRNLVLTGITTDVCVHTTMREANDRGFECLLLADCCGATDPGNHAAAVRMVTMQG |
| | GVFGAVANSRDLIEALP |
| 97 | WP_013893344.1 cysteine hydrolase [Mesorhizobium opportunistum]: |
| | MNARTGQRYIEADPYPWPYNGDLRPDNTALIIIDMQTDFCGPGGYVDHMGYDLSLVRAPIEPIRSVLSAM |
| | REKGYTIIHTREGHRPDLADLPANKRWRSRRINAGIGDPGPCGRILVRGEPGWDIIPDLYPAEGEPIIDK |
| | PGKGSFCATDLELILNQRGIDNIVLTGITTDVCVHTTMREANDRGFECVMLEDCCGATDYGNHLAAIKMI |
| | KMQGGVFGVVSSAASLVAQLP |
| 98 | WP_014993113.1 cysteine hydrolase [Alcanivorax dieselolei]: |
| | MPPRYLDSEPYPWPYNGELTPENTALIVIDMQTDFCGAGGYVDTMGYDLSLTRAPIEPIKAVLTLMREQG |
| | FCIIHTREGHRPDLSDLPANKRWRSRRIGAGIGDQGPCGRILVRGEPGWEIIPELAPLDGEVIIDKPGKG |
| | SFCATDLELILRTRGIENLILTGITTDVCVHTTMREANDRGFECLLLEDCCGATDTGNHTAAINMVKMQG |
| | GVFGAVSDSEALLRTLDGV |
| 99 | WP_015343698.1 cysteine hydrolase [Rhizobium tropici]: |
| | MNSLAAAAIGQKLSYIDADPYNWPYNGALRPDNTALIIIDMQTDFCGPGGYVDHMGYDLSLVRAPIEPIK |
| | RVLVAMRAKGYHIIHTREGHRPDLADLPANKRWRSQRINAGIGDPGPCGRILVRGEPGWDIIEELKPIDG |
| | ETIIDKPGKGSFCATDLELILNQKRIENIILTGITTDVCVHTTMREANDRGFECLLLEDCCGATDYGNHL |
| | AAIKMVKMQGGVFGSVANSQALIEALP |
| 100 | WP_016558329.1 cysteine hydrolase [Rhizobium grahamii]: |
| | MDTLVETKTQYITADPYPWPYNGALRPDNTALIIIDMQTDFCGKGGYVDHMGYDLALVQAPITPIKAVLS |
| | SMRAKGYHVIHTREGHRPDLADLPANKRWRSQRIGAGIGDPGPCGRILTRGEPGWDIIPELYPIAGETII |
| | DKPGKGSFCATDLELILNQKRIENIILTGITTDVCVSTTMREANDRGFECLLLEDCCGATDFGNHLAAIN |
| | MVKMQGGVFGSVSNSKTLIEALP |
| 101 | WP_016735441.1 MULTISPECIES: cysteine hydrolase [Rhizobium]: |
| | MDAMVETKGHYIDADPYAWPYNGDLRPENTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIISELYPIEGETII |
| | DKPGKGSFCATDLELILNQKRIANIILTGITTDVCVSTTMREANDRGFECLLLEDCCGATDYGNHLAAIK |
| | MVKMQGGVFGSVSNSQTLVSQLP |
| 102 | WP_018246800.1 cysteine hydrolase [Rhizobium leguminosarum]: |
| | MDAMVETKGHFIDADPYPWPYNGALRPDNTALIIIDMQTDFCGKGGYVDHMGYDLSLVQAPIEPIKRVLA |
| | AMRAKGYHIIHTREGHRPDLADLPANKRWRSQRIGAGIGDPGPCGRILTRGEPGWDIIPELYPVEGETII |
| | DKPGKGSFCATDLELILSQKRIENIILTGITTDVCVSTTMREANDRGYECLLLEDCCGATDYGNHLAAIK |
| | MVKMQGGVFGSVSNSAALVEALP |
| 103 | WP_018326144.1 cysteine hydrolase [Rhizobium giardinii]: |
| | MTSLAATAIPTGENLSYIDADPYPWPYNGALRPDNTALIIIDMQTDFCGPGGYVDHMGYSLSLVRAPIEP |
| | IRQVLAAMRAKGYHIIHTREGHRPDLADLPANKRWRSRRINAGIGDFGPCGRILVRGEPGWDIIDELYPI |
| | EGETIIDKPGKGSFCATDLELILSQKRIENIILTGITTDVCVHTTMREANDRGFECLLLEDCCGATDYGN |
| | HLAAIKMVKMQGGVFGSVSNSESLVRQLP |
| 104 | WP_031255153.1 cysteine hydrolase [Curvibacter lanceolatus]: |
| | MSPLLPTPTALHVDAQPYAWPWNGALRADNTALIVIDMQTDFCGPGGYVDVMGYDISLTRAPIQPLRQVL |
| | ARLRALGFLVIHTREGHRPDLSDLPANKRWRSRQIGRDGLGIGDAGPCGRILVRGEPGWEIIPELAPLPG |
| | ELVIDKPGKGSFYATDLDMVLRLAGIENLILGGITTDVCVHTTMRDANDRGFECLLLSDGTAATDPANHQ |
| | AALNMITMQGGVFGAHASSNQLLEALAGLSSI |
| 105 | WP_020514528.1 cysteine hydrolase [Actinoplanes globisporus]: |
| | MTVTIGPVPADPYPWPYDGSVPVTRTALICIDWQTDFCGKGGYVDSMGYDIELTRAGLPATARLLAHARD |
| | IGMLVIHTREGHDPDLSDLPANKRWRSARIGAEIGSDGPQGRILVRGEPGWEIVPEVEPVAGEVVIDKPG |
| | KGAFYATNLDLVLRTHGISHLILTGITTDVCVHTTMREANDRGYECLILSDCTGATDPSNHTAALHMVTM |
| | QGGVFGCVSTSADVINGTEF |
| 106 | WP_022978704.1 cysteine hydrolase [Nevskia ramosa]: |
| | MTFQTVVAEPYAWPWNGDFTPANTALIVIDMQTDFCGIGGYVDSMGYDLSLTRAPIAPIKMLLTRMRALG |
| | FTIIHTREGHRPDLSDLPANKRWRSRQMGAGIGDPGPCGKILVRGEPGWDIIPELYPEPGEIVLDKPGKG |
| | SFCATDLELILRTQGIVNIVLTGITTDVCVHTTMREGNDRGFECILIEDCCGATDHGNHLAALKMVKMQG |
| | GVFGAVATSSAFLAALG |
| 107 | WP_023495169.1 cysteine hydrolase [Methyloglobulus morosus]: |
| | MNKFVKSEPYPFPYNGDLRPENTCLIIIDMQIDFCGEGGYVDKMGYDISLTRVPIEPIRRVLETCRKQGF |
| | HIIHTREGHRPDLSDLPKNKRWRSQQIGAGIGDVGPCGRILVRGEPGWEIIPELAPLAGESIIDKPGKGS |
| | FYATDLDLLLHNRGIDNIVLTGITTDVCVHTTMRDANDRGFECLLLADCCGATDFGNHQAALNMIKMQGG |
| | VFGAVSDSESFIEAIS |
| 108 | WP_023561466.1 cysteine hydrolase [Actinoplanes friuliensis]: |
| | MTASIGPVKATPYLWPYDGSVPVERTALICIDWQTDFCGPGGYVESMGYDIALTRAGLPATAKLLAHVRS |
| | LGMLVIHTREGHDPDLSDLPANKRWRSAQIGAEIGSQGPCGRILTKGEPGWEIVPEVAPVAGEVIVDKPG |
| | KGAFYATNLDLVLRTHGITHLILTGITTDVCVHTTMREANDRGYECLILSDCTGATDPANHTAALHMVTM |
| | QGGVFGCVSTSDDVIAATEV |
| 109 | WP_024671285.1 MULTISPECIES: cysteine hydrolase [Pseudomonas syringae |
| | group]: |
| | MSERHIASAPYPWPWNGQLHAHNTALIVIDMQTDFCGVGGYVDSMGYDLALTRAPIEPIKALLAVMRPLG |
| | FSIIHTREGHRPDLSDLPANKRWRSQRIGAGIGDAGPCGKILVRGEPGWEIIDELAPLPGEIVIDKPGKG |
| | SFCATDLELILRTRGIDNLILTGITTDVCVHTTMREANDRGFECLLLEDCCGATDPANHAAALSMVKMQG |
| | GVFGAVGHSSMLHDLWEA |
| 110 | WP_027054243.1 cysteine hydrolase [Mesorhizobium erdmanii]: |
| | MNARAEITQHTIDAEPYPWPYNGDLRPDNTALIIIDMQTDFCGPGGYVDHMGYDLSLVRAPIEPIKAVLS |
| | AMRAKDYTIIHTREGHRPDLADLPANKRWRSRRINAGIGDAGPCGRILVRGEPGWDIIPDLYPIEGEPII |
| | DKPGKGSFCATDLELILNQRGIENIVLTGITTDVCVHTTMREANDRGYECMMLEDCCGATDHGNHLAAIK |
| | MIKMQGGVFGTVSNSKALVAQLP |
| 111 | WP_027475322.1 cysteine hydrolase [Curvibacter gracilis]: |
| | MSPLLPTPTELHVDAQPYAWPWNGALRADNTALIVIDMQTDFCGPGGYVDVMGYDISLTRAPIQPLRQVL |
| | ARLRALGFLVIHTREGHRPDLSDLPANKRWRSRQIGRDGLGIGDAGPCGRILVRGEPGWEIIPELAPLPG |
| | ELVIDKPGKGSFYATDLDMVLRLAGIENLILGGITTDVCVHTTMRDANDRGFECLLLSDGTAATDPANHQ |
| | AALNMITMQGGVFGAHASSDQLLEALSGLSSI |
| 112 | WP_027798423.1 cysteine hydrolase [Paraburkholderia dilworthii]: |
| | MTCFIEARPYPWPFDGALRADNTALIIIDMQTDFCGIGGYVDKMGYDLSLTRAPIEPISRVLATMREQGF |
| | TVIHTREGHRPDLSDLPANKRWRSRRAGTDGIGIGDDGPCGKILVRGQPGWDIIEELAPLPGEIIIDKPG |
| | KGSFCATDLELILRTRGIVNLVLTGITTDVCVHTTMREANDRGFECTVLADCCGATDKGNHDAALNMVLM |
| | QGGVFGTVSDSSALIAALGR |
| 113 | WP_027820346.1 cysteine hydrolase [Paraburkholderia bannensis]: |
| | MTRFIEARPYPWPYDGALRADNTALVIIDMQTDFCGIGGYVDKMGYDLSLTRAPIEPIQRVLAAMRAQGF |
| | TIIHTREGHRPDLSDLPANKRWRSRQAGTDGIGIGDDGPCGKILVRGQRGWEIIDELAPLPGEIVIDKPG |
| | KGSFCATDLELVLRTRGIANLVLTGITTDVCVHTTMREANDRGFECTILADCCGATDKSNHDAALNMVLM |
| | QGGVFGTVSDSHVLLATLGR |
| 114 | WP_030439668.1 cysteine hydrolase [Actinoplanes subtropicus]: |
| | MTATIGPVQADPYPWPFDGAAPVARTALICIDWQTDFCGKGGYVDSMGYDIELTRAGLPATAKLLAHARD |
| | LGMLVIHTREGHDPDLSDLPANKRWRSARIGAEIGGDGPCGRILVRGEPGWEIVPEVAPVAGEVVIDKPG |
| | KGAFYATNLDLVLRTHGISHLILTGITTDVCVHTTMREANDRGYECLILSDCTGATDPGNHAAALHMVTM |
| | QGGVFGCVATSDDVIAATEA |
| 115 | WP_033319363.1 cysteine hydrolase [Streptomyces yerevanensis]: |
| | MVGPVTAKPYAWPYDTSVPADRVAVLCIDWQTDFCGPGGYVDTMGYDISLTRAGLPATQKLLAHARSTGM |
| | LVVHTREGHAPDLADLPANKRWRSAQIGAEIGAAGPCGRILVRGEPGWEIVPEVAPFPGEVIVDKPGKGA |
| | FYATNLDLVLRTRGITHLVLTGITTDVCVHTTMREANDRGYECLILSDCTGATDPSNHEAALHMVTMQGG |
| | VFGCVSTADDLIAATTEATS |
| 116 | WP_033361216.1 cysteine hydrolase [Dactylosporangium aurantiacum]: |
| | MTATIGPVVGARPYAWPYDGSVPVGRTALLCIDWQTDFCGPGGYVDSMGYDIGLTRAGLPATAKLLDHVR |
| | GLGMLVVHTREGHDPDLSDLPPNKRWRSARIGAEIGSAGPCGRILIKGEPGWQIVPEVAPVPGEVIVDKP |
| | GKGAFYATNLDLVLRTHGITHIILTGITTDVCVHTTMREANDRGYECLILSDCTGATDPSNHAAALHMVT |
| | MQGGVFGCVATSEDVIAATVSD |
| 117 | WP_035252302.1 cysteine hydrolase [Actibacterium atlanticum]: |
| | MSYIDADPYNWPYNGDLRPENTALIIIDMQTDFCGKGGYVDTMGYDLSLTQAPIEPIKALLAKMRAGGYH |
| | IIHTREGHRPDLADLPPNKRWRSQQIGAGIGDPGPCGKILIRGEPGWDIIPELYPAEGEPIIDKPGKGSF |
| | CATDLELLLRTRGIENILLTGITTDVCVHTTMREANDRGFECLLVEDCCGATDKGNHDAAIKMVKMQGGV |
| | FGSVSDSATLIAQLP |
| 118 | WP_035935333.1 cysteine hydrolase [Caballeronia glathei]: |
| | MNRFIEARPYPWPYDGNLRPDNTALVIIDMQTDFCGYGGYVDKMGYDLSLTRAPIEPIRRVLATMREQGF |
| | TIIHTREGHRPDLSDLPANKRWRSRRAGTDGVGIGDAGPCGRILVRGEPGWEIIDELAPLPGEVVIDKPG |
| | KGSFCSTDLELILRTRGIVNLVLTGITTDVCVHTTMREANDRGFECTVLADCCGATDKGNHDAALHMITM |
| | QGGVFGAVSDSRSLLATLEA |
| 119 | WP_035963207.1 cysteine hydrolase [Caballeronia grimmiae]: |
| | MTRFIEARPYPWPYDGNLRPDNTALVIIDMQTDFCGIGGYVDKMGYDLSMTRAPIQPIRNVLTLMREQGF |
| | TIIHTREGHRPDLSDLPANKRWRSRRAGTNGVGIGDDGPCGKILVRGEPGWEIIDELKPVEGEIVIDKPG |
| | KGSFCATDLEMVLRTRGIVNLVLTGITTDVCVHTTMREANDRGFECTILADCCGATDQGNHDAALNMVLM |
| | QGGVFGTVSDSNALLAALGR |
| 120 | WP_037083615.1 cysteine hydrolase [Rhizobium vignae]: |
| | MDAMGETKGHYIDADPYAWPYNGDLRPQNTALIIIDMQTDFCGKGGYVDHMGYDLSLVQAPIEPIKTVLA |
| | AMRAKGYHIIHTREGHRPDLADLPANKRWRSQRIGAGIGDPGPCGRILTRGEPGWDIIPELYPIEGEVII |
| | DKPGKGSFCATDLELILNQKRIENIILTGITTDVCVSTTMREANDRGFECLMLEDCCGATDYGNHLAAIK |
| | MVKMQGGVFGSVSNSATLVSQLP |
| 121 | WP_051963325.1 MULTISPECIES: cysteine hydrolase [Rhizobium]: |
| | MNHPATATADQTLNYIDADPYVWPYNGALRPDNTALIIIDMQTDFCGPGGYVDHMGYDLSLVRAPIEPIK |
| | RVLAAMRAKGYHIIHTREGHRPDLADLPANKRWRSQRINAGIGDPGPCGRILVRGEPGWDIIDELKPIEG |
| | ETIIDKPGKGSFCATDLELILNQKRIENIILTGITTDVCVHTTMREANDRGFECLLLEDCCGATDYGNHL |
| | AAIKMVKMQGGVFGSVSNSTSLVSQLP |
| 122 | WP_038587753.1 cysteine hydrolase [Neorhizobium galegae]: |
| | MNSLSPAVVAGQTLSYIDADPYAWPYNGALRPDNTALIIIDMQTDFCGKGGYVDHMGYDLSLVQAPIEPI |
| | KRVLAAMRAKGYHIIHTREGHRPDLADLPANKRWRSQRIGAGIGDPGPCGRILTRGEPGWDIIEELYPIE |
| | GEVIIDKPGKGSFCATDLELILNQKRIENIVLTGITTDVCVSTTMREANDRGFECLMLEDCCGATDYGNH |
| | LAAIKMVKMQGGVFGAVSNSDALVKALP |
| 123 | WP_039788660.1 cysteine hydrolase [Herbaspirillum huttiense]: |
| | MSERYIQAEPYPWPYDGALTPANTALIVIDMQTDFCGIGGYVDKMGYDLSLTRAPIEPIKKVLAAMRAGG |
| | YTIIHTREGHRPDLSDLPANKRWRSRQIGTNGVGIGDAGPCGRILVRGEPGWEIIPELAPMAGEIIIDKP |
| | GKGSFCATDLEMILHTRGIRNIVLTGITTDVCVHTTMREANDRGFECVMLADCCGATDYNNHLAALSMIK |
| | MQGGVFGAVSDAAALIDVIGA |
| 124 | WP_052418263.1 cysteine hydrolase [Pseudooceanicola atlanticus]: |
| | MNDMSDTPAGTTIASTPYPWPWNGDLRPENTALIIIDMQTDFCGPGGYVDSMGYDISLTRAPIEPIKALM |
| | KAFRDKGYMVIHTREGHRPDLADLPANKQWRSRQIGAGIGDPGPCGKILTRGEPGWEIIDDLAPLPGEVI |
| | IDKPGKGSFCATDLEMILRLKGIDNIVLTGITTDVCVHTTMREANDRGFECVMLTDCCAATDPKNHEAAI |
| | NMIHMQGGVFGATALSTDLLAVLP |
| 125 | WP_045231530.1 cysteine hydrolase [Agrobacterium rubi]: |
| | MDAMVETAGHYIGADPYPWPYNGALRPDNTALVIIDMQTDFCGKGGYVDHMGYDLAMVQAPIQPIKTVLA |
| | AMRAKGYHIIHTREGHRPDLADLPANKRWRSQRIGAGIGDAGPCGRILTRGEPGWDIIPELYPIDGETII |
| | DKPGKGSFCATDLELILHQKRIENLILTGITTDVCVSTTMREANDRGFECLLLEDCCGATDYGNHLAAIK |
| | MVKMQGGVFGAVSNSKTLVEALP |
| 126 | WP_045672421.1 cysteine hydrolase [Paenibacillus beijingensis]: |
| | MNAYVPSMQVENALPYPFGFDPASTAVVVIDMQNDFCAPGGFGQRLGNDIAAVRAIIPTISRVLDAARSA |
| | GLLIIHTREGHLPDLSDCPPSKQERSRRQGAGIGDAGPMGRILIRGEPGHEIIPELTPIPGEPVVDKPGK |
| | GAFYQTNFHDILIEYGIESLILCGVTTHVCVHTTLREANDRGYRCLVLEDATAAFDPDDHAAAIHMVRQQ |
| | GGIFGWTSASISLIHTLRK |
| 127 | WP_046104327.1 cysteine hydrolase [Devosia chinhatensis]: |
| | MSLSDERVEAALVPGGTTIANADPYPWPFDGNWGAHNTALVVIDMQVDFCAPGGYVDTMGYDISLTRAPI |
| | APIQRVLAAMRARGYTIIHTREGHKPDLSDLPANKRWRSQRIGAGIGDQGPCGRILVRGEPGWEIIPELQ |
| | PLPGEQIIDKPGKGTFIATDFELVLRMKHIRNIIFTGVTTDVCVHTTMRDANDRGYECLLLEDCCAATKR |
| | SNHDAAIDMIKMQGGVFGAVSISDALIEVLP |
| 128 | WP_046153182.1 cysteine hydrolase [Robbsia andropogonis]: |
| | MSLFIEAKPYRWPYNGDLRADNTALIIIDMQTDFCGPGGYVDKMGYDLSLTRAPIAPLSAVLDMMRAQGY |
| | TIIHTREGHRADLSDLPANKRWRSRQAGSNGVGIGDDGPCGKILVRGEDGWQIIEELAPQPGEIVIDKPG |
| | KGSFYATDLELILRTRGIRNLVLTGITTDVCVHTTLREANDRGFECTVLADCCGATDVGNHYAALAMIQM |
| | QGGVFGTVSDSTSLLSALRNS |
| 129 | WP_053199920.1 cysteine hydrolase [Herbaspirillum hiltneri]: |
| | MSELFIQSEPYPWPYDGALKPGNTALVVIDMQTDFCGIGGYVDKMGYDLSLTRAPIAPIRNVLSAMRAGG |
| | YTIIHTREGHRPDLSDLPANKRWRSRRIGANGAGIGDEGPCGKILVRGEPGWEIIPELAPLPGEIIIDKP |
| | GKGSFCATDLELVLHTRGIRNLILTGITTDVCVHTTMREANDRGFECVMLADCCGATDHNNHLAALSMIK |
| | MQGGVFGAVSDSSSLLQAIGK |
| 130 | WP_054019041.1 cysteine hydrolase [Ideonella sakaiensis]: |
| | MPRSVLAQPYAWPYDGQWTPADTALVVIDMQTDFCGVGGYVDSMGYDLALTRAPIGPIGRLLERMRALGF |
| | HVIHTREGHRPDLADLPANKRWRSRQMGAGIGDAGPCGRILVRGEPGWEIIPELAPLPGEVVIDKPGKGS |
| | FCATDLELILHTRGIRNLVLTGITTDVCVHTTMREANDRGFECLLVSDGTAATDAGNHAAALKMITMQGG |
| | VFGAHATSAALLEALA |
| 131 | WP_054360926.1 cysteine hydrolase [Prosthecomicrobium hirschii]: |
| | MTLIDERVAAATVPGGRTVASEPYPWPYDGDLRPDNTALIVIDMQTDFCGVGGYVDSMGYDIALTRAPIG |
| | PIAAVLEAMRAKGYTIIHTREGHRPDLSDLPANKRWRSQRIGAGIGDQGPAGRILVRGEPGWEIIPELAP |
| | LPGEVIIDKPGKGSFCATDLEMILRLKGLRNIVLTGITTDVCVHTTMREANDRGFECLLLTDCCAATKYD |
| | NHLAAIDMIKMQGGVFGAVSDSRSFLEAIR |
| 132 | WP_054999487.1 MULTISPECIES: cysteine hydrolase [Pseudomonas syringae |
| | group]: |
| | MSERHVDSAPYPWPWNGQLHAHNTALIVIDMQTDFCGVGGYVDSMGYDLALTRAPIEPIKALLAVMRPLG |
| | FTIIHTREGHRPDLSDLPANKRWRSQRIGAGIGDPGPCGKILVRGEPGWEIIDELAPLPGEIIIDKPGKG |
| | SFCATDLELILRTRGIDNLILTGITTDVCVHTTMREANDRGFECLLLEDCCGATDPGNHAAALSMVKMQG |
| | GVFGAVSHSSLLRDLLEA |
| 133 | WP_058088296.1 cysteine hydrolase [Aquabacterium parvum]: |
| | MSPTTYVDAQPYQWPYNGDLRPANTALIIIDMQTDFCGEGGYVDKMGYDISATRAPIEPLKVLLAEARRV |
| | GMLVIHTREGHRPDLSDLPANKRWRSRQIGTNGVGIGDVGPCGRILVRGEPGWDIIPELYPIDGEPIIDK |
| | PGKGSFYATDLELVLHTKGIQNVVLTGITTDVCVHTTMRDANDRGFECVMLTDCTGATDPGNHAAAFSMI |
| | KMQGGVFGAVSDSKALIRAMQAWPSVAPAAGVARAA |
| 134 | WP_059193874.1 cysteine hydrolase [Streptomyces antibioticus]: |
| | MVGPVTAKPYAWPYDTSVPADRVAVLCIDWQTDFCGPGGYVDTMGYDISLTRAGLPATQKLLAHARSTGM |
| | LVVHTREGHAPDLADLPANKRWRSAQIGAEIGAAGPCGRILVRGEPGWEIVPEVAPVPGEVIVDKPGKGA |
| | FYATNLDLVLRTRGITHLVLTGITTDVCVHTTMREANDRGYECLILSDCTGATDPSNHEAALHMVTMQGG |
| | VFGCVSTADDLITATTEATS |
| 135 | WP_060602508.1 cysteine hydrolase [Aureimonas altamirensis]: |
| | MDTTSSTTAGTIASQPYAWPYDASLRPDNTALIVIDMQTDFCGKGGYVDAMGYDLSLTRAPIEPIARVMA |
| | AMRAGGYHIIHTREGHRPDLADLPANKRWRSRNIGAGIGDPGPCGRILVRGEPGWEIIPELAPLPGEVVI |
| | DKPGKGSFCATDLELILNQRGIRNIVLTGITTDVCVHTTMREANDRGYECVILEDCCGATDRSNHDAAIR |
| | MVTMQGGVFGAVAHSDALLEALR |
| 136 | WP_061116979.1 cysteine hydrolase [Caballeronia turbans]: |
| | MSRFIEARPYPWPYDGNLRPDNTALVIIDMQTDFCGIGGYVDKMGYDLSMTRAPIEPIRNVLTLMREQGF |
| | TIIHTREGHRPDLSDLPANKRWRSRRAGTNGVGIGDDGPCGKILVRGEPGWEIIDELKPIEGEIVIDKPG |
| | KGSFCATDLEMVLRTRGIVNLVLTGITTDVCVHTTMREANDRGFECTILADCCGATDQGNHDAALNMVLM |
| | QGGVFGTVSDSKALLATLGR |
| 137 | WP_061133981.1 cysteine hydrolase [Caballeronia fortuita]: |
| | MSRFIEARPYPWPYDGNLRPDNTALVIIDMQTDFCGIGGYVDKMGYDLSMTRAPIEPIRNVLTLMREQGF |
| | TIIHTREGHRPDLSDLPANKRWRSRRAGTNGVGIGDEGPCGKILVRGEPGWEIIDELKPVEGEIVIDKPG |
| | KGSFCATDLDMILRTRGIVNLVLTGITTDVCVHTTMREANDRGFECTILADCCGATDQGNHDAALNMVLM |
| | QGGVFGTVADSKALLATLGR |
| 138 | WP_062033021.1 cysteine hydrolase [Streptomyces phaeopurpureus]: |
| | MADSAPISPQNPTVVIGPVTAKPYAWPYDTSVPADRVAVLCIDWQTDFCGPGGYVDTMGYDISLTRAGLP |
| | ATQKLLAHARSTGMLVVHTREGHAPDLADLPANKRWRSAQIGAEIGAAGPCGRILVRGEPGWEIVPEVAP |
| | LPGEVIVDKPGKGAFYATNLDLVLRTRGITHLVLTGITTDVCVHTTMREANDRGYECLILSDCTGATDPS |
| | NHEAALHMVTMQGGVFGCVSTADDLITATTEATS |
| 139 | WP_062137725.1 cysteine hydrolase [Paraburkholderia monticola]: |
| | MTRYLEARPYPWPYDGNLRPDNTALIIIDMQTDFCGHGGYVDKMGYDLSLTRAPIEPIKRVLAPMRELGF |
| | TIIHTREGHRPDLSDLPANKRWRSRQAGTNGVGIGDVGPCGRILVRGEPGWEIIDELAPLPGEIIIDKPG |
| | KGSFCATDLELILRTRGIANLVLTGITTDVCVHTTMREANDRGFECTLLADCCGATDRSNHAAALNMVLM |
| | QGGVFGTVSDSAALVAALER |
| 140 | WP_062243904.1 cysteine hydrolase [Streptomyces griseorubiginosus]: |
| | MADSAPISPQNPTVVIGPVTAKPYAWPYDTSVPADRVAVLCIDWQTDFCGPGGYVDTMGYDISLTRAGLP |
| | ATQKLLAHARSTGMLVVHTREGHAPDLADLPANKRWRSAQIGAEIGAAGPCGRILVRGEPGWEIVPEVAP |
| | LPGEVIVDKPGKGAFYATNLDLVLRTRGITHLVLTGITTGVCVHTTMREANDRGYECLILSDCTGATDPS |
| | NHEAALHMVTMQGGVFGCVSTADDLITATTEATS |
| 141 | WP_068114315.1 cysteine hydrolase [Pseudoruegeria marinistellae]: |
| | MTTIESHPYAWPYNGDLRPGNTALIVIDMQTDFCGTGGYVDAMGYDLSLTQAPIGPIKALMTDMRAKGYH |
| | IIHTREGHRPDLADLPANKRWRSQQIGAGIGDPGPCGKILVRGEPGWDIIPELYPLDGEVVIDKPGKGSF |
| | CATDLELILRTRGIENLILTGITTDVCVSTTMREANDRGFECVIAEDCCGATDPGNHAAAIKMVTMQGGV |
| | FGAVSDSASLIAGLPA |
| 142 | WP_083229793.1 cysteine hydrolase [Agrobacterium sp. RAC06]: |
| | METEMTTIQADPYLWPYNGDLRPDNTALIIIDMQTDFCGPGGYVDKMGYDIGLTRAPIEPIKAVLQAMRD |
| | KGYHVIHTREGHRPDLSDLPPNKRWRSQQIGAGIGDAGPCGRILVRGEPGWEIIDELKPLDGEPIIDKPG |
| | KGSFCATDLELLLRTRGIENIVLSGITTDVCVHTTMREANDRGFECLLLEDCCAATDPGNHAAAIKMVKM |
| | QGGVFGAVSDSGKFVEALP |
| 143 | WP_073173303.1 cysteine hydrolase [Pseudomonas asturiensis]: |
| | MSERYLACEPYPWPWNGKLNSNNTALIVIDMQTDFCGVGGYVDSMGYDLALTRAPIEPIKGLLALMRPLG |
| | FTIIHTREGHRPDLSDLPANKRWRSQRIGAGIGDPGPCGKILVRGEPGWELIEELAPLPGEIIIDKPGKG |
| | SFYATDLELVLRTRGIENLILTGITTDVCVHTTMRDANDRGFECILLEDCCGATDPANHAAALSMIKMQG |
| | GVFGAVGHSSMLRDLLEA |
| 144 | WP_084564509.1 cysteine hydrolase [Pseudoxanthobacter soli]: |
| | MSLSLLQAPEAPADAEAGGEGRIHVDAAPYPWPFDGDLRPANTALIIIDMQTDFCGPGGYVDAMGYDLTL |
| | PRATIAPISRVLAAMRAKGFHVFHTREGHKPDLSDLPENKRWRSRRIGAGIGDPGPCGRVLVRGEPGWEI |
| | IPELAPIDGEPIIDKPGKGSFCATDLELILRLKGVRNIVLAGLTTDVCVHTTMREANDRGFECLLLEDCC |
| | AATDPANHAAAISMIQKQGGVFGAVASSSRLLEVLS |
| 145 | WP_074072734.1 cysteine hydrolase [Rhizobium gallicum]: |
| | MDAMAETKGHFIDADPYPWPYNGALRPDNTALIIIDMQTDFCGKGGYVDHMGYDLSLVQAPIEPIKHVLA |
| | AMRAKGYHIIHTREGHRPDLADLPANKRWRSQRIGAGIGDPGPCGRILTRGEPGWDIIPELYPIEGETII |
| | DKPGKGSFCATDLELILNQKRIENIILTGITTDVCVSTTMREANDRGYECLLLEDCCGATDYGNHLAAIK |
| | MVKMQGGVFGSVSNSEMFVSQLP |
| 146 | WP_074585157.1 cysteine hydrolase [Pseudomonas psychrotolerans]: |
| | MSPRLLASAPYPWPYDGRLDPANTALVIIDMQTDFCGVGGYVDAMGYDLSLTRAPIEPIRSVLEVMRAQG |
| | FPIIHTREGHRPDLSDLPANKRWRSRNIGAGIGDDGPCGRILIRGEPGWAIIPELAPLPGEIVIDKPGKG |
| | SFYATDLELILRTRGIANLILTGITTDVCVHTTMREGNDRGFECILLEDCCGATDHGNHLAALNMVKMQG |
| | GVFGAVGDSSMLLAVLAGD |
| 147 | WP_075854492.1 cysteine hydrolase [Rhizobium hainanense]: |
| | MNSLPKAELAGQTLSHIDADPYPWPYNGDLRPDNTALIIIDMQTDFCGKGGYVDHMGYDLSLVQAPIEPI |
| | KSVLSAMRAKGYHIIHTREGHRPDLADLPANKRWRSQRIGAGIGDSGPCGRILVRGEPGWDIIPELYPIK |
| | GEAIIDKPGKGSFCATDLELILNQKRIENIILTGITTDVCVSTTMREANDRGFECLLLEDCCGATDYGNH |
| | LAAIKMVKMQGGVFGSVSNSKALIEALP |
| 148 | WP_076625677.1 cysteine hydrolase [Thiobacimonas profunda]: |
| | MTSHVQGLYPWPFDGDLRPENTALIIIDMQIDFCGEGGWVHSRGSDLRNTRRPIEPLQNLLKVLRPAGYT |
| | IIHTREGHRPDLSDLPANKLWRSQQLNGNGIGAMGPLGRYLIRGEPNWDIIPELAPAEGEVVIDKPGKGA |
| | FMGTDLDTVLRTRGIRNLMIAGVTTDCCVQSTLRDANDRGFECLLLEDCCGAADHSYHEAQVEIFRLSNG |
| | LWGSIATSDDVIATLTGAAA |
| 149 | WP_077980810.1 cysteine hydrolase [Rhizobium laguerreae]: |
| | MDAMVETEGHFIDADPYPWPYNGALRPDNTALIIIDMQTDFCGKGGYVDHMGYDLTLVQAPIEPIKRVLA |
| | AMRAKGYHIIHTREGHRPDLADLPANKRWRSQRIGAGIGDPGPCGRILTRGEPGWDIIPELYPIEGETII |
| | DKPGKGSFCATDLELILSQKRIENIILTGITTDVCVSTTMREANDRGYECLLLEDCCGATDYGNHLAAIK |
| | MVKMQGGVFGSVSNSVALVEALP |
| 150 | WP_085558546.1 cysteine hydrolase [Carnobacterium iners]: |
| | MTKEFTIQAKPYGFELDLETTALIIIDMQRDFLYPGGFGEQLGNDVSTTSSIIPNVKRVLDKAREKGMLV |
| | IHTREGHRPDLTDLPASKAKRGGGIGEEGPMGRILVRGEYGHDIVDELQPIEGEVILDKPGKGAFYQTDL |
| | ETILKNKNIKSLLLAGVTTHVCVQSTIREANDRGYECLMLEDCCAAFDKKDHEDSIRMINQQGGIFGWTT |
| | ESKNLLEAIN |
| 151 | WP_085749770.1 cysteine hydrolase [Rhizobacter gummiphilus]: |
| | MERYIAAEPYRWPFDGRMSPQDTALVIIDMQVDFCGPGGYVDKMGYDISLTRAPIEPLKRLLAAMRAKGY |
| | PVIHTREGHKPDLSDLPANKRWRSRQIGTNGIGIGDVGPCGRILTIGEPGWEIIPELAPLPGEPVIDKPG |
| | KGSFYATNFELVLKTLGIRNLILTGITTDVCVHTTMRDANDRGYECLIVSDCTAATDAGNHAAALKMVTM |
| | QGGVFGAVSDAASIIEGLA |
| 152 | WP_085877124.1 cysteine hydrolase [Roseisalinus antarcticus]: |
| | MTTISSTPYAWPWNGDLRPENTALIVIDMQTDFCGKGGYVDHMGYDLSLTQAPIGPIKALMANMRAKGYH |
| | IIHTREGHRPDLADLPPNKRWRSQQIGAGIGDAGPCGKILIRGEAGWDIIPELYPQEGETIIDKPGKGSF |
| | CATDLELILRMRGIENLILTGITTDVCVSTTMREANDRGFECVIVEDCCGATDAGNHAAAIKMVTMQGGV |
| | FGAVSDSASLIAGLPG |
| 153 | WP_085935041.1 cysteine hydrolase [Enhydrobacter aerosaccus]: |
| | MARKHVNSRPYPWPWNGDLRPENTALIVIDMQTDFCGVGGYVDKMGYDLSLTRAPIEPIRTLLAASRQAG |
| | WHIFHTREGHRPDLSDLPANKRWRSQQIGAGIGEPGPCGRILVRGEPGWEIIPELAPAKGEPVIDKPGKG |
| | SFCATDLELMLRTRGIDNLVLTGITTDVCVHTTMREANDRGFECLILEDCTGATDMGNHLAALKMVQMQG |
| | GVFGAVARSTDVIEAIA |
| 154 | WP_090798859.1 cysteine hydrolase [Asanoa ishikariensis]: |
| | MAHIGPVKAEPYTWPYDGEVPVDRTALLCIDWQTDFCGPGGYVDSMGYDISLTRAGLPATAALLAHVRAL |
| | GMLVVHTREGHDPGLTDLPANKRWRSRQIGAEIGAAGPCGRILVRGEPGWEIVPEVAPVAGEVVVDKPGK |
| | GAFYATNLDLVLRTHGITHLILTGITTDVCVHTTMREANDRGYECLILSDCTGATDAGNHAAALHMVTMQ |
| | GGVFGCVAASTDVIAATLH |
| 155 | WP_091010500.1 cysteine hydrolase [Paraburkholderia megapolitana]: |
| | MNRYIEARPYPWPYNGDLQAANTALVIIDMQTDFCGYGGYVDKMGYDLSLTRAPIEPIRGVLAVMRAQGF |
| | TIIHTREGHRPDLSDLPANKRWRSRRAGTDGVGIGDAGPCGKILVRGEPGWQIIDELAPLPGEIVIDKPG |
| | KGSFCATDLELILRTRGIENLVLTGITTDVCVHTTMREANDRGFECTVLADCCGATDQSNHAAALHMITM |
| | QGGVFGTVSDSQALLAALGG |
| 156 | WP_091295461.1 cysteine hydrolase [Gemmobacter aquatilis]: |
| | MTTVSSTPYAWPWNGDLRPENTALIIIDMQTDFCGTGGYVDMMGYDLSMTQAPIEPIKAVLAAMRAKGYT |
| | IIHTREGHRPDLSDLPPNKRWRSRQIGAGIGDAGPCGKILIRGEPGWDIIPELYPLPGEAIIDKPGKGSF |
| | CATDLELMLRVQGIENIILTGITTDVCVSTTMREANDRGFECLILSDCCGATDPGNHEAALKMVTMQGGV |
| | FGAVSDSASLIAVLP |
| 157 | WP_091641346.1 cysteine hydrolase [Aquisalimonas asiatica]: |
| | MASCYIDATPYRWPFDGLLTPDNTALMIIDMQTDFCGKGGYVDRMGYDLSLTRAPLKPIQRTLEHMRQGG |
| | FTVIHTREGHRRDLSDLPENKRWRSRQIGAGIGDPGPAGRILVRGEEGWEIVPELTPLEGEPVIDKPGKG |
| | SFYATDLDLILRTQGIRNLILTGITTDVCVHTTMREANDRGYECLLLEDCCGATDRSNHLAAIEMIKMQG |
| | GVFGSVSDSEALVAGC |
| 158 | WP_092373934.1 cysteine hydrolase [Xiangella phaseoli]: |
| | MGRIGPVTANPYPWPYDGAADTARTALLCIDWQTDFCGPGGYVDAMGYDIGLTRAGLPATARLLEHARSL |
| | GMLVVHTREGHDPDLSDLPSNKRWRSAQIGAEIGAAGPCGRILVKGEPGWEIVPEVAPAPGEVVVDKPGK |
| | GAFYATNLDLVLRTRGITHLILTGITTDVCVHTTMREANDRGYECLILSDCTGATDKGNHDAALHMVTMQ |
| | GGVFGCVATSDDVIAATTK |
| 159 | WP_092547462.1 cysteine hydrolase [Actinoplanes derwentensis]: |
| | MTARIGPVQADPYHWPYDGSVPVDRTALLCIDWQTDFCGPGGYVDSMGYDIGLTRAGLPATAKILSHVRE |
| | LGMLVIHTREGHDPDLSDLPANKRWRSARIGAEIGGQGPCGRILIKGEPGWEIVPEVAPAPGEVVIDKPG |
| | KGAFYATSLDLVLRTHGITHLILTGITTDVCVHTTMREANDRGYECLILSDCTGATDPSNHTAALHMVTM |
| | QGGVFGCVATSDDVIAATQSSHPPKAD |
| 160 | WP_092679559.1 cysteine hydrolase [Albimonas donghaensis]: |
| | MNAMDEIRTGPLAADPYAWPWNGDLRPENTALIIIDMQIDFCGPGGYVDKMGYDLSNTRAPIAPIRTVLA |
| | AMRGWGGLVIHTREGHRPDLSDLPANKRWRSRQMGAGIGDMGPCGRILTRGEPGWEIIDELAPAEGEPII |
| | DKPGKGSFYATDLDLILRTRGIRNLVLTGITTDVCVHTTMRDANDRGYECLLLEDCCGATDMGNHHAAIK |
| | MIKMQGGVFGAVSNAADFVEVLA |
| 161 | WP_092852955.1 cysteine hydrolase [Rhizobium miluonense]: |
| | MNYPAIAPASQTLAYIDADPYVWPYNGALRPGNTALIIIDMQTDFCGPGGYVDHMGYDLSLVRAPIEPIK |
| | RVLAAMRAKGYHIIHTREGHRPDLADLPANKRWRSKRINAGIGDAGPCGRILVRGEPGWDIIDELKPMDG |
| | ETIIDKPGKGSFCATDLELILNQKRIENIILTGITTDVCVHTTMREANDRGFECLLLEDCCGATDYGNHL |
| | AAIKMVKMQGGVFGSVSNSANLVSQLP |
| 162 | WP_092860340.1 cysteine hydrolase [Albimonas pacifica]: |
| | MDGTLETAGPLAADPYPWPYNGDLRPENTALIVIDMQTDFCGVGGYVDKMGYDLSNTRAPIEPIKSVLAA |
| | MRAWGGLVIHTREGHRPDLGDLPPNKRWRSRRIGAGIGDEGPCGRILTRGEPGWEIIEELAPIEGEPIID |
| | KPGKGSFYATDLELLLRTKGIQNFVLTGITTDVCVHTTMRDANDRGFECLLLEDCCGATDMGNHHAAIKM |
| | IKMQGGVFGAVSNSKDFTACLKAVGK |
| 163 | WP_093280567.1 cysteine hydrolase [Solimonas aquatica]: |
| | MSSRHLVSEPYPWPYNGDLRAENTALIIIDMQTDFCGPGGYVDKMGYDLSLTRAPIEPIGRVLARFRKLG |
| | FHVFHTREGHRPDLADLPANKRWRSQRIGAGIGDPGPCGRILVRGEPGWDIIPELAPLPGEPIIDKPGKG |
| | SFCATDLELIMRVRGIDNLILTGITTDVCVHTTMREANDRGFECLMLEDCCGATDYQNHLHAIKMIKMQG |
| | GVFGAVATSEQLLQALS |
| 164 | WP_093410371.1 cysteine hydrolase [Verrucosispora sediminis]: |
| | MGRIGPVTANPYPWPYDGTADTARTALLCIDWQTDFCGPGGYVDAMGYDIGLTRAGLPATARLLDHVRSL |
| | GMLVVHTREGHDPDLSDLPANKRWRSAQIGAEIGAAGPCGRILVKGEPGWEIVPEVAPAPGEVVVDKPGK |
| | GAFYATNLDLVLRTRGITHLILTGITTDVCVHTTMREANDRGYECLILADCTGATDKDNHDAALHMVTMQ |
| | GGVFGCVATSDAVIAATTR |
| SEQ | |
| ID | |
| NO. | Embodiments of Cyanuric Acid Hydrolase Amino Acid Sequences |
| 165 | WP_011393610.1 ring-opening amidohydrolase [Moorella thermoacetica] |
| | MQKVEVFRIPTASPDDISGLATLIDSGKINPAEIVAILGKTEGNGCVNDFTRGFATQSLAMYLAEKLGIS |
| | REEVVKKVAFIMSGGTEGVMTPHITVFVRKDVQEPAKPGKRLAVGVAFTRDFLPEELGRMEQVNEVARAV |
| | KEAMKDAQIDDPRDVHFVQIKCPLLTAERIEDAKRRGKDVVVNDTYKSMAYSRGASALGVALALGEISAD |
| | KISNEAICHDWNLYSSVASTSAGVELLNDEIIVVGNSTNSASDLVIGHSVMKDAIDADAVRAALKDAGLK |
| | FDCCPPAEELAKIVNVLAKAEAASSGTVRGRRNTMLDDSDINHTRSARAVVNAVIASVVGDPMVYVSGGA |
| | EHQGPDGGGPIAVIARV |
| 166 | MQKVEVFRIPTASPDDISGLATLIDSGKINPAEIVAILGKTEGNGAVNDFTRGFATQSLAMYLAEKLGIS |
| | REEVVKKVAFIMSGGTEGVMTPHITVFVRKDVQEPAKPGKRLAVGVAFTRDFLPEELGRMEQVNEVARAV |
| | KEAMKDAQIDDPRDVHFVQIKCPLLTAERIEDAKRRGKDVVVNDTYKSMAYSRGASALGVALALGEISAD |
| | KISNEAICHDWNLYSSVASTSAGVELLNDEIIVVGNSTNSASDLVIGHSVMKDAIDADAVRAALKDAGLK |
| | FDCCPPAEELAKIVNVLAKAEAASSGTVRGRRNTMLDDSDINHTRSARAVVNAVIASVVGDPMVYVSGGA |
| | EHQGPDGGGPIAVIARV |
| 167 | MQKVEVFRIPTASPDDISGLATLIDSGKINPAEIVAILGKTEGNGSVNDFTRGFATQSLAMYLAEKLGIS |
| | REEVVKKVAFIMSGGTEGVMTPHITVFVRKDVQEPAKPGKRLAVGVAFTRDFLPEELGRMEQVNEVARAV |
| | KEAMKDAQIDDPRDVHFVQIKCPLLTAERIEDAKRRGKDVVVNDTYKSMAYSRGASALGVALALGEISAD |
| | KISNEAICHDWNLYSSVASTSAGVELLNDEIIVVGNSTNSASDLVIGHSVMKDAIDADAVRAALKDAGLK |
| | FDCCPPAEELAKIVNVLAKAEAASSGTVRGRRNTMLDDSDINHTRSARAVVNAVIASVVGDPMVYVSGGA |
| | EHQGPDGGGPIAVIARV |
| 168 | MQKVEVFRIPTASPDDISGLATLIDSGKINPAEIVAILGKTEGNGGVNDFTRGFATQSLAMYLAEKLGIS |
| | REEVVKKVAFIMSGGTEGVMTPHITVFVRKDVQEPAKPGKRLAVGVAFTRDFLPEELGRMEQVNEVARAV |
| | KEAMKDAQIDDPRDVHFVQIKCPLLTAERIEDAKRRGKDVVVNDTYKSMAYSRGASALGVALALGEISAD |
| | KISNEAICHDWNLYSSVASTSAGVELLNDEIIVVGNSTNSASDLVIGHSVMKDAIDADAVRAALKDAGLK |
| | FDCCPPAEELAKIVNVLAKAEAASSGTVRGRRNTMLDDSDINHTRSARAVVNAVIASVVGDPMVYVSGGA |
| | EHQGPDGGGPIAVIARV |
| SEQ | |
| ID | |
| NO. | Embodiments of Triuret Hydrolase Amino Acid Sequences |
| 169 | >Herbaspirillum_TrtA: |
| | MIRIDATPYPYQFHPRSTALVVIDMQRDFIEEGGFGSALGNDVRPLAAIVPTVAALLQLAREAGMLVVHT |
| | RESHLPDLSDCPRSKRLRGNPTLGIGDVGPMGRILVQGEPGNQILPQLAPVEGELVIDKPGKGAFYATDL |
| | HAQLQERRITHLLVAGVTTEVCVQTSMREANDRGYECLVIEDACASYFPDFHRITLEMLTAQGGIVGWRT |
| | PLAQLQAGVAAYTGENP |
| 170 | >Rhizobium_TrtA: |
| | MMEIKAEPFAFPVKHDELALIVIDMQRDFAEPGGFGASLGNDVSRITRIVPDVKRLIQGFRNAGLPVIHT |
| | MECHRPDLSDLPPAKRDRGNPSLRIGDEGPMGRILISGEPGTAILPELAPVKGEVVIEKPGKGAFYATEL |
| | GTVLQQKGIKQLVFAGVTTEVCVQTTMREANDRGYECLLAEEATESYFPEFKAAAIAMIRAQGAIVGWTA |
| | HVDDILESIAHA |
| 171 | >Actinoplanes_TrtA: |
| | MPTVDAQPGPFTFQAHETALVVIDMQRDFLLPGGFGESLGNDVAELRRTIAPLTALINAWRAAGLPIIHT |
| | REGHLPDLSDCPPAKLKRGPMIGQEGTFGRILIRGQYGHDIIDELKPAEGEPVVDKPGKGAFYATDLDKI |
| | LDNDGIKSLVVTGVTTEVCVHTTVREANDRGYECLVLADCVGSYFPEFQQVGLKMIAAQGGIFGWVAESP |
| | ALIAAIQE |
| 172 | NP_769365.1 hypothetical protein blr2725 [Bradyrhizobium |
| | diazoefficiens USDA 110]: |
| | MLNSTKPTLGVISAEPEPIRLDWPATALLIIDMQRDFMEPGGFGETLGNDVSQLARAVKPIGAVLRAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKISTSA |
| 173 | NP_791181.1 isochorismatase family protein [[Pseudomonas syringae] pv. |
| | tomato str. DC3000]: |
| | MISISARPDTFTFEPSRTAVVIIDMQRDFLEPGGFGAALGNDVAPLQAIVPTVQQLLALAREQGLVVIHT |
| | RESHLPDLSDCPQAKLDHGLPGLRIGDPGPMGRILVRGEPGNQIIDALTPLASEWVIDKPGKGMFFATDL |
| | QQRLTVAGITHLIFAGVTTEVCVQTSMREACDLGYRCLLIEDATESYFAAFKQATLDMITAQGAIVGRVA |
| | SLANLQHALHTRSTQ |
| 174 | YP_234255.1 isochorismatase hydrolase [Pseudomonas syringae pv. |
| | syringae B728a]: |
| | MNKVNARPDRFAFDTSRTAVVIIDMQLDFLEPGGFGAALGNDVAPLQAIVPSVQRLLTLARDEGMTVIHT |
| | RESHRPDLADCPQAKRDHGSPGLRIGDPGPMGRILIRGEPGNQIIDALAPLADEWVIDKPGKGMFFATDL |
| | QQRLSQAGITHLIFAGVTTEVCVQTSMREANDRGYRCLLIEDATESYFPAFKKATLEMITAQGGIVGRVA |
| | SLTDLEQALQTRSTH |
| 175 | XP_001798644.1 hypothetical protein SNOG 08324 [Parastagonospora |
| | nodorum SN15]: |
| | MSSPVLSFEAKPYAFTFPLEHTALLIIDMQRDFLLAKGFGEIQGGNLEAVQASIAPTKKLLEACRAGGLT |
| | IVHTREGHNPDLADCPSAKLVRQSAAPNNTQHNLVIGDKGELGRLLTRGEYGHDIVDELQPLPGEVVIDK |
| | PGKGSFWNTNILHDLKARSITHLIVSGVTTECCFATTIREANDRGFECCGIEEATSGYNDACFKQSTLDM |
| | IHWSQGLFGFIGNLQPLLDVLAPLSTPSVAAGSTPPRTPPTFNGDLTITSLQQAYKNGMSPVSLIEAIYD |
| | KIEAYKAIDPAVWIHLVPRAQALEAANKIAARFPNRNALPPLFGIPFSVKDSIDVQGIPTTTGCEILSHV |
| | PAVSAVVYKKLIAEGALFIGKVNLDQLATGLVGCRSPFGIPHSVYHKDYISGGSSSGSAVSVGANLVSFS |
| | LATDTAGSGRVPAGFNGIVGFKPTRGTISFRGITPACLSLDCIAISAKTITDARTVWHTLEGHDPLDPYA |
| | KPTLSFERHINSIGPQSQTFKFGIPPPSALAICSRPTRRMFNETVEQLQKIGGILKPIDWTPFQKAGELL |
| | YDGTFVSERLASLPDDFLEKNRSALHPVIAQLMGRCRRAEKYGCRRVPRLASQSTLHASSRASLCIRRFG |
| | R |
| 176 | XP_001905267.1 uncharacterized protein PODANS_5_7430 [Podospora |
| | anserina S mat+]: |
| | MAPALKTILALQDAKPYAFECPTATTALIIIDIQRDFVDPGGFGSIQCGNDAVFSRARAIVPVVKKLLDA |
| | FRSFGGHVIHTREGHEPGLADLPAAKRLRQISNPVGHHSLGIGDQGPMGKLLVRGEYGHDIVDELTPWPD |
| | ETVIDKPGKGSFWGTNIHRILLARGITHLVFAGVTTEYLTHSHSAVEKPSLTFATTGAVSVQHCANVLTE |
| | AINALCWRTARRGSMLSSPDFFQAIDRASAKALTQGLALTPPAKPMGNKDSKPRFGFLPDESVPSVDQLL |
| | TDYRQSIRCPVEVIKSLYKRINQYKDVDPAVWIHLEPEANVLHAATKLVNKYKGKPLPSLYGIPFSVKDT |
| | IDVAGVPTTAACPSYAYTPQVSATAVRRVLDAGALFIGKVNLDQLATGLSGCRSPYGTPHSVFSDKHIPG |
| | GSSSGSCVSVGERLVSFGLATDTAGSGRVPAAFNGIVGFKPTKGTVSARGLVPACRTLDTITVVAPSITE |
| | ARKVWQVIAHHDPEDPYSKLPHTLPTWHIDYRGPRVGGFTFAVPPPTILKVCKKEYRELFSSAVSALQSC |
| | GGTLKEVEYTPFSAAGDLLYDGSLLHERIHCIGHRFLQSNLPDMHPVIRELFDKAMSNPPLVYDAFRDQA |
| | LQARLTREVQGVFDVLNGGVDVLVVPTTTQHPTIKEMEADPLKLNSELGTFTHCANVVDLCGVSVPAGTW |
| | LWGQEGDERKMPFGITILSGSGYDAKVLDIAGVFEEEMMQRETFRL |
| 177 | XP_001941969.1 glutamyl-tRNA(Gln) amidotransferase subunit A |
| | [Pyrenophora tritici-repentis Pt-1C-BFP]: |
| | MAKMASDSTVLSFDAKPYAFSFPLAHTALLIIDMQRDFLLAKGFGEIQGGNLKAVQASIAPTKRLLEACR |
| | GAGMAIFHTREGHKPDLSDCPSAKLIRQEAAPGNTQHKLVIGDKGELGRLLTRGEHGHDIIDELFLEYDH |
| | LHQLKARAITHLIVSGVTTECCFATTIREANDRGFECCGIEEATSGYNDACFKETTLDMIHWSQGLFGFI |
| | GCLEPLLGALAHVSTKKIQVASTPPQTPRTFDGDLTIPALQQAYKNGLSPVTVSEAIYDKIKEYQKIDSA |
| | VWIHLQPREAILEAARRLELEYPDRSALPPLFGVPFSAKDSIDVAGIPTTTACPPLTHVPSVSAPVYEKV |
| | MAEGALFVGKTNLDQLATGLVGCRSPYGIPHSVYHKDYISGGSSSGSTVSVGASLVSFSLATDTAGSGRV |
| | PAGFNGIVGYKPTRGTISFRGITPACLSLDCIALSTKTVSDARTVWQILEGHDPLDPYAKPQIAFERHIN |
| | SIGPQSRTFKFGVPPPEALAICSTPARRMFNETILRLQKMGGVLTQIDWSPFQKAGQLLYDGTFVSERLA |
| | SLPDDFLGKNRSALHPVTVQLMDAVTDRKSSAVDAYRDLQAKALYTRQAEQVFAYSASGVDVIVVPTAPT |
| | HWKIKEVLADPIRKNSTLGEFTHCGNVLDLCGVAVPAGTYPVAELSGQETDEGVLPFSITLLSGSRLDAE |
| | LLEIARRFEKSFTQ |
| 178 | XP_002143704.1 glutamyl-tRNA(gln) amidotransferase subunit A, putative |
| | [Talaromyces marneffei ATCC 18224]: |
| | MVAAQSIKRIASGVEDNGVVSFEAQPYAFRFNPSTTALLIIDMQRDFLLKDGFGYIQAGDAGVEKVQATI |
| | KPTLAVLRMFRECGIHVIHTREGHRPDLRDLPTPKLLRQAHAPESRHSMVIGDVGPMGRLLTRGEYGHDI |
| | IDELQPVTGEYVVDKPGKGSFFSTTLHEHLVDRGITHLIVAGVTVECCVTTTVREGNDRGFDACILSDCT |
| | DGFVPTFKSASLDMIHFSEGLFGFVSESQPLLAALSSLPADSSKSARDWDGSMSIESLKSAYSGGLSPVT |
| | VVKYVLETISADKSNHSAVWLNLSSTKDLLHRAESLEQLGDRNLPLFGVPFAVKDNIDVAGLPTTAACPE |
| | FEYVPEKSAFVIRKLEAAGAIVIGKTNLDQFATGLVGTRSPYGACHCALDPTRVSGGSSSGSAVAVALGQ |
| | VAFALGTDTAGSGRIPASFNNIIGLKPTKGTISTTGVIPACRTLDCVSFFANTISDARTVWLAAKEHDPE |
| | DPYSRSSPSLASLNSRSILHEESTYTVSFPPIGILESALSPAYNKQFVKVASLVRSLDNVEEINFDWSSY |
| | LSASDLLYKSAFVAERTAALQEVLNSKAKKITLHPVTQQVLDLAGSKSATDAFRDIYEAQRLLKAIEAGF |
| | DKCDILVVPTAPNHPTIAEVEQDPIGPNLKLGYFASAVNVLDLAAVAIPAGHIEGLPFGISIIAPAFKEG |
| | VILQVAQRIQARLGHFVL |
| 179 | XP_002180928.1 predicted protein [ Phaeodactylum tricornutum CCAP |
| | 1055/1]: |
| | MVQIALSMQPHVGEIELDSAALIIIDMQRDFLEPQGFGELLGNDVSKLQRAIDPCQKVLQAARKANLTVL |
| | HTREGHRADMLDVHGHKLQRLGCASQVIGTQGPNGRILIRGEMGHDIIPALYPVDGEAVIDKPGKGSFYG |
| | TDLEVILAARNIRTLFVCGVTTEVCVHTTVREANDRGIHCVVVSDACASFFDDFHRVALEMVVAQGGIFG |
| | STVESKELVGAFERLTK |
| 180 | XP_002291891.1 predicted protein, partial [Thalassiosira pseudonana |
| | CCMP1335]: |
| | LLLIDFQNDFMSPGGFGEQLGNDVSKLRRIIEPTKSVLACARLAGLTVIHTREGHRSNLSDLTSLKASGC |
| | TSIGKEGSSNGRSLIRGQWGNEIISELKPLDDSETIINKPGKGAFYQTDLELVLKNANIDTLIVCGVTTE |
| | VCVHSTVREANDRGIQCIVLEDCTASYIDSFHKVGIEMISAQGGILGKVSDSKSIIEALVR |
| 181 | YP_002822610.1 cysteine hydrolase (plasmid) [Sinorhizobium fredii |
| | NGR234]: |
| | MAEIKAEPFPFRLDRDAVALIVIDMQRDFTEEGGFGESLGNDVARVAKIVPDVKRLIEGFRAAGLPVIHT |
| | MECHRPDLSDLPRAKRERGRPHFRIGDEGPMGRILIAGEPGTAILPELAPAVGETVIEKPGKGAFYATPL |
| | DYILKERDIGQLVFAGVTTEVCVQTTMREANDRGYECLLVEEATESYFPEFKSATLAMIRAQGAIVGWTA |
| | HLADVLRGIAHA |
| 182 | XP_002480025.1 amidase, putative [Talaromyces stipitatus ATCC 10500]: |
| | MAATLSIKRIPSGAEDSEVVSFEAQPYAFQFDPSAAALVIIDMQRDFLLKDGFGYIQAGDAGVEKVQATI |
| | KPTLEVLRKFRERGIHVIHTREGHRPDLRDLPTPKLLRQARAPNTRHSMVIGDLGPMGRLLTRGEYGHDI |
| | IDELQPVAGEFVIDKPGKGSFFSTTLHEHLVDRGITHLIVAGVTVECCVTTTVREANDRGFDACILRDCT |
| | DGFVSTFKSASLDMIHFSEGLFGFVSESQPLLAALSSFPPHPKNLALDWDGSMSIEALKNAYSSGLSPVT |
| | VVRHVLKKISADKPQHSAVWLSLASSKNLLGRAEDLERSGDCNLPLFGVPFAVKDNIDVSGLPTTAACPG |
| | FEYVPEKSAFVVTKLEAAGAIVIGKTNLDQFATGLVGTRSPYGACYCTFSPRHISGGSSSGSAVAVALGH |
| | VSFALGTDTAGSGRIPASFNNIVGLKPTKGTVSTAGVLPACRTLDCVSFFASTISDARIAWLAAKAHDPE |
| | DPYARSSPSLASLNSRSVLHGDALSPAYQEQFAKVLSLVRGLANIEEVNFDWSAYLAASDLLYKSALVTE |
| | RTAAVQELLGSKAKRISLHPVTQKVLDSASSKTATDVFRDVHKAQRLLKVIEAEFDKCDILVVPTAPNHP |
| | TVAEVEQDPIVPNLKLGIFASAVNILDLAAVAIPAEHIDGLPFGISIIAPAFREGFILKVAERIRKRMEH |
| | FVL |
| 183 | XP_002840409.1 hypothetical protein [Tuber melanosporum Mel28]: |
| | MEALSKNITIEAKPYPYTFPLSSTALLLIDLQRDFILPSGFGDIQSGTNLTAVTAVVPNCVRILQAFREL |
| | ELPIFHTREGHLPDLSDCPSSKLGRQASAPGTSHSKVIGDPGELGRLLVRGEHGHDIVDECRPKLGEVVV |
| | DKPGKGAFWNTNLLEELVGWGITHLIVGGVTTECCVTTTVREANDRGFECCIIEECTAGYNDNFKAPSLN |
| | MIHWSQGLFGFVSSLPNFLKALTPALPSPGAPGPDLGLESPPSTPPVWDGCLTLDSLRKSYKSGLSPVTV |
| | ITSLYARIEEYSQTKSVFIRLVDRPISISYAESLQKLFPDLTNLPPLYGVPFTLKDSINVAGIPTTLACP |
| | PLAHIPSRSSKIYSRLISLGAVYIGKTNLDQFATGLTGCRSPYGTPASIYNPDYVSGGSSSGSAVSVGAE |
| | LSSFAIATDTAGSGRVPAGFNGVVGWKPTKGTVSFSGVMKACESLDCLSFMVTPDGGVKDVRKLWNLVRG |
| | YDPDDPYSKTPGSLPLPMVNALGEKKWKFALPDRKAVAECSPEYRKLFYQAIGSLQEIGGEVKEGDWGLF |
| | EEAGKLLYDGALVNERLAALPDNGWVGREKDELHPVIREILQNVLETGASAAEALTRKVNATLFNPSHPS |
| | YIDVLIVPTAPFHPRISAVLKDPIAINTRLGTFTHFGNVLDLCAIAVPAGHYMEDEKKMPFSITFLGRGG |
| | SDARVLEIASLFEGLVGVGAKDSA |
| 184 | XP_002840690.1 hypothetical protein [Tuber melanosporum Mel28]: |
| | MEALPKNITIEAKPYPYTFPLSSTALLLIDLQRDFILPSGFGDIQSGMNLAAVTAVVPNCVRILQAFREL |
| | ELPIFHTREGHLPDLSDCPSSKLNRQASAPGTNHSKVIGDPGKLGRLLVRGEHGHDIVDECQPKLGEVVV |
| | DKPGKGAFWNTNLSEELVGWGITHLIVGGVTTECCVTTTVREANDRGFECCIIEECAAGYNDSFQAPSLN |
| | MIHWSQGLFGFVSSLPNLLKELASVPSPGAPVPDLGLESPPSTPPIWDGRLTLAALRKSYRSGLPPVTVI |
| | TGLYARIEDYSQTKSTFIHLVDRSALISYAESLQKRFPDLANLPPLYGVPFTLKDSINVAGIPTTLACPP |
| | LAHIPSRSSKVYSRLISLGAVYIGKTNLDQFATGLTGCRSPYGVPASVYNPDHVSGGSSSGSAVSVGAEL |
| | SSFAVATDTAGSGRVPAGFNGVVGWKPTKGTVSFSGVMNACESLDCLSFMVTSAGGVKDVRKLWNLVRGY |
| | DPDDPYSKMPGSLPLPMVDALGGKRWKFAIPDRKAVAECSPEYRKLFYQAIGGLHEIGGEVKEGDWGLFE |
| | EAGRLLYDGALVNERLAALPDNKWVERERDELHPMTGLTRKVNATLFNPSHSSYIDVLIVPTAPFHPRIS |
| | AVLKDPIAINSRLGTFTHFGNVLDLCAIAVPAGHYMEDEKKMPFSITFLGRGGFDARVLEIASLFEGLVG |
| | AGARDSV |
| 185 | XP_002999762.1 glutamyl-tRNA(Gln) amidotransferase subunit A |
| | [Verticillium alfalfae VaMs.102]: |
| | MSSASRPSLSLPNARPYPFDFPLATTALVIIDIQRDFVDPGGFGSVQCGNDEIFSKARSIVPAVQRVLEI |
| | FRSTRGHVIHTREGHQPDLADLPAAKKLRQINNPNGHHFMGIGDQGPMGRLLVRGEYGHDIIDELQPWPT |
| | EVVIDKPGKGSFWGTDIHRVLLARGITHLLFAGVTTECCVTTTLRECNDRGYQCCVLEDCTQGFDAQQVT |
| | TSLDTICAQDGLFGFVGNSADFLTATKDVSTAPVSQLGVSGPFPSIDDLQALYKDGQTTPTDVVNAAFDR |
| | IEAYQNEDPAVWTSLAKRADVLVAAKALAEKYKEKPLPPLFGVPFGVKDSIDVEGIETTAACPSYAYVPK |
| | ATATCVQHILDAGGIYVGKTNLDQLATGLSGCRSPYGVPHSIFSKDLIAGGSSSGGCVAVAARLVPFTVA |
| | TDTAGSGRVPAAFNGVVGFKPTKGTISARGLIPACKTLDSIAIVATSVADARAVWRVIAKHDKADPYSKL |
| | PHTLPTWKTDFRGLKDGGFDFAVPPPAALEACTPEYRRLFAEAVKKLQSAGGRLRNTDWEAFERAGELLY |
| | EGALLHERITCIGREFLRSSIQDGGLHPVIQKLFSDALNKAPDAYDVFRDQATQAELSRRTHMAFDTLSG |
| | GVDVLVVPTTVCHPTFEEIAADPIRLNARLGTFTHFANIVDLCGLSVPAGTYLDEKETELPFGVTILAGS |
| | GFDAKALDVARVLEEVIKAK |
| 186 | XP_003297511.1 hypothetical protein PTT 07937 [Pyrenophora teres f. |
| | teres 0-1]: |
| | MAKMASDSMILSFDAKPYAFSFPLAHTALLIIDMQRDFLLAKGFGEIQGGNLEAVQASIAPTKRLLEACR |
| | GVGMTVFHTREGHKPDLSDCPSAKLIRQEAAPGNTQHKLVIGDKGELGRLLTRGEYGHDIIDELKPIPGE |
| | VVIDKPGKGSFWNTTIFHQLKARAITHLIVSGVTTECCFATTIREANDRGFECCGIEEATSGYNDVCFKK |
| | TTLDMIHWSQGLFGFIGCLEPLLEALAPVSTKKIQVAPTPLQTPPTFDGDLTISALQRAYKNGLSPITVA |
| | DKVYDKIEAYQKIDPAVWIHLQPREAILEAARQLASRYPDRSALPPLFGVPFSAKDSIDVSGLPTTTACP |
| | PLAHVPSVSAPVYDKVIAEGALFFGKTNLDQLATGLVGCRSPYGIPHSVYHKDYISGGSSSGSTVSVGAN |
| | LVSFSLATDTAGSGRVPAGFNGIVGYKPTRGTISFRGITPACLSLDCIALSTKTVSDARTVWQILEGHDP |
| | LDPYAKPEIAFERHINSIGPQSRTFKFGVPPPEAMEICSTPARRMFNETILKLQKIGGVLTQIDWSPFQK |
| | AGQLLYDGTFVSERLASLPDDFLEKNRSALHPVTVQLMDTVTNRKSSAVDAYRDLQAKAIYIRQAEQVFA |
| | YSASGVDVIAVPTTPTHWKIEEVLADPIKKNSILGEFTHCGNVLDLCGIAVPAGTYPVAELSGQETDEGV |
| | LPFSVTLLSGSRLDAELLEIARRFEENFA |
| 187 | YP_006122159.1 hypothetical protein NRG857 19090 [Escherichia coli |
| | O83:H1 str. NRG 857C]: |
| | MTQSIFQAQPFELPFDPRTTALVMIDMQRDFVEAGGFGEALGNDVSLVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 188 | XP_003849806.1 hypothetical protein MYCGRDRAFT 75341 [Zymoseptoria |
| | tritici IPO323]: |
| | MELPSARPYSYKFNPESTALIIIDMQRDFVDLNGFGMIQCGNDELFKKVRDIVPKTQRALAAARKLGLHL |
| | VHTREGHRPDLSDLPPSKRLRQISSPSGKHTMTIGDQGPMGRLLVRGEYGHDIIDELKPYPGEVVIDKPG |
| | KGSFWDTTLHRALLARGITHLLFAGVTTECCVNTTVREAADRGFETCVLADCTDGFDASFYSSTLDMLCS |
| | YDGLFGFVGSSEELLKLVPVQSEEVEKDSASFDGDISLEGLRKQYSSGQARPTDVIKEIISRIEEYKIKD |
| | PAVWISLRSPEQLLESARAVEEKFAGRPLPELYGVPFGVKDTIDVAGIPTTAACEAYAYIPEQHATVVKA |
| | LLDAGGIFVGKTNLDQLATGLSGCRSPYGTPRSVYGKDRISGGSSSGSAVAVAAGLVSFALGTDTAGSGR |
| | VPASFNGIVGFKPTKGTLSAHGLVPACASLDCITVLSRTVEESREVWLVLDKGQDPADPHAKTQQSLALW |
| | HADFRGVKTGGFTFGVPPPATLEKCTQVYRALFAAAVERLKRAGGSAKEIPWTPFESATNLLYDASLVHE |
| | RIACIGHEFLTENLDSLHPVTKTLYSTALNSTLKPWDVFRDLQLRAEFTRDAAAVFRDTIDVLLVPTTTS |
| | HPTVQEMEADPLALNAKLGYFTHFGNVLDLCGVALPAGEYESGDGEGERLPFGVTILGAAGMDGKVFDIA |
| | REFERTA |
| 189 | WP_000155780.1 MULTISPECIES: cysteine hydrolase [Enterobacteriaceae]: |
| | MTISIFQAQPFELPFDPCTTALIMIDMQRDFVEAGGFGEALGNDVSLVRTAIAPCKEVLAAARQKGIMVI |
| | HTREGHREDLSDCPSAKLTRGGKTFIGEPGPMGRILVRGEAGHDIIPELYPVTGEPVIDKPGKGAFYQTD |
| | LHLILQKRGIKTLIVCGVTTEVCVTTTVREANDRGYECIIPEDCVGSYFPEFQKYALEMIKAQGAIFGWV |
| | TDSKAIIAGLEG |
| 190 | WP_000194413.1 cysteine hydrolase [Escherichia coli]: |
| | MTQSIFQAQPFELPFDPRTTALVMIDMQRDFVEAGGFGEALGNDVSLVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWI |
| | TDSKAIIAGLEG |
| 191 | WP_000194414.1 MULTISPECIES: cysteine hydrolase [Enterobacteriaceae]: |
| | MTQSIFQAQPFELPFDPRTTALVMIDMQRDFVEAGGFGEALGNDVSLVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 192 | WP_000194416.1 cysteine hydrolase [Escherichia coli]: |
| | MTQSIFQAQPFELPFDPRTTALVMIDMQRDFVEAGGFGEALGNDVSRVRTAIAPCTEVLAAARQKGIMVI |
| | HIREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 193 | WP_000194417.1 cysteine hydrolase [Escherichia coli]: |
| | MTQSIFQAQPFELPFDPRTTALVMIDMQRDFVEAGGFGEALGNDVSRVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLECQRRMKSDPLISPPTAQY |
| 194 | WP_000194418.1 MULTISPECIES: cysteine hydrolase [Enterobacteriaceae]: |
| | MTQSIFQAQPFELPFDPRTTALVMIDMQRDFVEAGGFGEALGNDVSRVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 195 | WP_000194419.1 cysteine hydrolase [Escherichia coli]: |
| | MTQSIFQAQPFELPFDPRTTALVMIDMQRDFVEAGSFGEALGNDVSLVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVYGVTTEVCVTTTVREANDRGYECIIPEDCVGSYFPEFQKYALEMIKAQGAIFGWV |
| | TDSKAIIAGLEG |
| 196 | WP_001517370.1 MULTISPECIES: cysteine hydrolase [Escherichia]: |
| | MTQSIFQAQPFELPFDPCTTALVMIDMQRDFVEAGGFGEALGNDVSLVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 197 | WP_001540240.1 MULTISPECIES: cysteine hydrolase [Escherichia]: |
| | MTQSIFQAQPFELPFDPRTTALVMIDMQRDFVEAGGFGEALGNDVSLVRTAIAPCTEVLAAARQKGTMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 198 | WP_002435279.1 cysteine hydrolase [Atlantibacter hermannii]: |
| | MKTLEAQPFAYSFDPATTALVMIDMQRDFVEPHGFGEALGNDVSLLRRAIEPCTRLLEAARQAGLLIVHT |
| | REGHRADLSNCPAAKLTRGGKTFIGQQGSMGRILIQGEPGHDIIPELYPLSGEPIIDKPGKGAFYATDLH |
| | LILQARGIKSLIICGVTTEVCVQTTAREANDRGYEVLIPEDCCASYFPEFHRAALEMIKAQGAIVGWVSD |
| | ADAVINALR |
| 199 | WP_002552366.1 MULTISPECIES: cysteine hydrolase [Pseudomonas]: |
| | MISVNARPDCFTFAPSCAAVVIIDMQRDFLEPGGFGAALGNDVAPLQAIVPSVQQLLALARDQGIVVIHT |
| | RESHSADLADCPPAKLAHGSPGLRIGDSGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTP |
| 200 | WP_002714198.1 cysteine hydrolase [Afipia clevelandensis]: |
| | MPPTKNLLAAEPAPLELVWAKTALVIIDMQRDFLEPGGFGETLGNDVSQLTRAVKPCGDVLAAFRKAGLL |
| | VVHTREGHLPDLSDAPPAKIERGAPSLRIGDPGPMGRILIRGEAGHDIVPPLYPIKGEIVIDKPGKGAFY |
| | ATGLGDILKARGIENLLVCGVTTEVCVNTTVREANDRGYRCVVLADCCASYFPEFHEMGLRMIKAQGGIF |
| | GWVSSSAEVLAALNSEPSKMIA |
| 201 | WP_003065372.1 cysteine hydrolase [Amycolatopsis vancoresmycina]: |
| | MTTPLAVGADPTSFRFEPATTALLVIDMQRDFVEPGGFGETLGNDVSRLRGVIAPLRRTLAATRAAGVRV |
| | IHTREGHLPDLSDCPPAKLERGRPSMRIGDPGPNGRILVRGEHGHGIIDELAPVDGETVIDKPGKGAFYR |
| | TGLGEVLSAAGITSLVVTGVTTEVCVHTTVREANDRGYECLVLSDCVGSYFPEFQAAGLAMISAQGGIFG |
| | WVAPSAAYVAALSVLATPAR |
| 202 | WP_003291941.1 cysteine hydrolase [Pseudomonas stutzeri]: |
| | MISVPGKPAAFNFDPTRTALVVIDMQRDFLEPGGFGAALGNDVSLLQAIVPAVESLLALAREKGMLVIHT |
| | RESHLPDLSDCPAAKREGGAEGLRIGDPGPMGRILVRGEPGNQIIPSLAPIAGEWVIDKPGKGMFYATGL |
| | GDRLAAQGIECLIFAGVTTEVCVQTSMREANDRGYRCLLIEDATDSYFPAFKQETLEMIVAQGGIVGHTA |
| | TLAALDAAMNEE |
| 203 | WP_003349923.1 MULTISPECIES: cysteine hydrolase [Pseudomonas]: |
| | MIKVNARPDSFAFDTSRTAVVIIDMQRDFLEPGGFGAALGNDVAPLQAIVPSVQRLLTLARDEGIAVIHT |
| | RESHRPDLADCPQAKRDHGSPGLRIGDPGPMGRILIRGEPGNQIIDTLAPLAGEWVIDKPGKGMFFATDL |
| | QPRLTDAGITHLIFAGVTTEVCVQTSMREANDRGYRCLLIEEATESYFPAFKRATLEMITAQGGIVGRVA |
| | SLTDLEQALQTRSTH |
| 204 | WP_003375792.1 cysteine hydrolase [Pseudomonas syringae]: |
| | MISISARPDTFTFEPSRTAVVIIDMQRDFLEPGGFGAALGNDVAPLQAIVPTVQQLLALAREQGLVVIHT |
| | RESHRPDLSDCPQAKLDHGLPGLRIGDPGPMGRILIRGEPGNQIIDALTPLASEWVIDKPGKGMFFATDL |
| | HQRLTAAGITHLIFAGVTTEVCVQTSLREACDRGYRCLLIEDATESYFPAFKQATLDMITAQGAIVGRVT |
| | SLANLQHGLHTRSTP |
| 205 | WP_003421845.1 cysteine hydrolase [Pseudomonas syringae]: |
| | MIKVNARPDRFAFDTSRTAVVIIDMQHDFLEPGGFGAALGNDVAPLQAIVPSVQRLLALARDEGMAVIHT |
| | RESHRPDLADCPQAKRDHGSPGLRIGDPGPMGRILIRGEPGNQIIDTLAPLAGEWIIDKPGKGMFFATDL |
| | QQRLSEAGITHLIFAGVTTEVCVQTSMREANDRGYRCLLIEDATESYFPAFKQATLEMITAQGGIVGRVA |
| | SLTDLEQALLTRSTL |
| 206 | WP_003459764.1 cysteine hydrolase [Pseudomonas oleovorans]: |
| | MIRLPARPATFSFEPTRTALVVIDMQRDFLEPGGFGAALGNDVTLLQAIVPAVASLMALARAQGMLVIHT |
| | RESHLADLSDCPAAKREGGAVGLRIGDAGPMGRILVRGEPGNQIIPALAPMAGEWVIDKPGKGMFYATGL |
| | GDRLVAQGIESLIFAGVTTEVCVQTSMREANDRGYRCLLIEEATESYFPAFKQATLEMIVAQGGIVGHTA |
| | NLSALSAAMTEDRA |
| 207 | WP_003538533.1 cysteine hydrolase [Rhizobium leguminosarum]: |
| | MMGIKAEPFAFPVKHDELALIVIDMQRDFAEPGGFGASLGNDVSRITRIVPDVKRLIQGFRNAGLPVIHT |
| | MECHRPDLSDLPPAKRDRGNPALRIGDEGPMGRILISGEPGTAILPELAPVKGEVVIEKPGKGAFYATDL |
| | GAVLQEKGIRQLVFAGVTTEVCVQTTMREANDRGYECLLAEEATESYFPEFKAAAIAMIRAQGAIVGWTA |
| | HVDDILESIAHA |
| 208 | WP_003568577.1 cysteine hydrolase [Rhizobium leguminosarum]: |
| | MAEIKAEPFAFPVKHDQLALIVIDMQRDFAEPGGFGASLGNDVSRITRIVPDVKRLIQGFRYAGLPVIHT |
| | MECHRPDLSDLPPAKRDRGNPTLRIGDEGPMGRILIAGEPGTAILPELAPVKGEVVIEKPGKGAFYATQL |
| | GEVLQQKRIKQLVFAGVTTEVCVQTTMREANDRGYECLLAVEATESYFPEFKAAAIAMIRAQGAIVGWTA |
| | HVDDILESIAHA |
| 209 | WP_003591773.1 cysteine hydrolase [Rhizobium leguminosarum]: |
| | MAGIKAEPFAFPVKYDELALIVIDMQRDFAEPGGFGASLGNDVSRITRIVPDVKRLIQGFRNAGLPVIHT |
| | MECHRPDLSDLPPAKRDRGNPSLRIGDEGPMGRILISGEPGTAILPELAPVKGEVIIEKPGKGAFYATEL |
| | GAILQQKGISQLVFAGVTTEVCVQTTMREANDRGYECLLAEEATESYFPEFKAAAIAMIRAQGALVGWTA |
| | HVDDILESIANA |
| 210 | WP_003607796.1 cysteine hydrolase, partial [Methylorubrum extorquens]: |
| | AARAAGLLVVHTREGHAPDLSDAPPAKLERGAPTARIGEPGPMGRILIRGEPGHDIIPELAPLDGEPVID |
| | KPGKGAFYATGLAALLEARGIETLIVCGVTTEVCVHTTVREANDRGYRCVVVADACGSYIPAFHEAGLAM |
| | IKAQGGIFGWVSRSAAVIAALGQA |
| 211 | WP_004108650.1 cysteine hydrolase [Rhizobium freirei]: |
| | MADIKAQPFAFPLQRDAVALIVIDMQRDFAEPGGFGASLGNDVSRIMKIVPEVRRLIAGFRDAGLPVIHT |
| | MECHRPDLSDLPAAKRDRGNPSLRIGDVGPMGRILIAGEPGTAILAELAPIDGEIVIEKPGKGAFYATGL |
| | GDILKRKGIKQLVFAGVTTEVCVQTTMREANDRGYESLLAEEATESYFPEFKAAAIAMIRAQGAIVGWTA |
| | HIDDILEAINHA |
| 212 | WP_004126364.1 MULTISPECIES: cysteine hydrolase [Klebsiella]: |
| | MRTIQAQPFDFTFDPASTALVIIDMQRDFVEPAGFGEVLGNDVSHLRRTIAPCRQLLEQARASGLFIIHT |
| | REGHRADMADCPPAKKTRGGKTFIGESGPMGRILIRGEQGHDIIPELTPLPGEPIIDKPGKGAFYATDLG |
| | LILQTRGIKSLIICGVTTEVCVQTTAREANDRGYELVIPEDCCASYFPEFHRAALDMMKAQGAIVGWVSD |
| | SASIIGALQN |
| 213 | WP_004402990.1 cysteine hydrolase [Pseudomonas syringae]: |
| | MIKVNARPDRFAFDTSRTAVVIIDMQRDFLEPGGFGAALGNDVAPLQAIVPSVQRLLALARDEGMAVIHT |
| | RESHRPDLADCPQAKRDHGSPGLRIGDPGPMGRILIRGEPGNQIIDTLAPRAGEWIIDKPGKGMFFATDL |
| | QQRLTDAGIIHLIFAGVTTEVCVQTSMREANDRGYRCLLIEDATESYFPAFKQATLEMITAQGGIVGRVA |
| | SLTDLEQALLTRSTL |
| 214 | WP_004406595.1 MULTISPECIES: cysteine hydrolase [Pseudomonas]: |
| | MNKVNARPDRFAFDTSRTAVVIIDMQRDFLEPGGFGAALGNDVAPLQAIVPSVQRLLTLARDEGMTVIHT |
| | RESHRPDLADCPQAKRDHGSPGLRIGDPGPMGRILIRGEPGNQIIDALAPLADEWVIDKPGKGMFFATDL |
| | QQRLSQAGITHLIFAGVTTEVCVQTSMREANDRGYRCLLIEDATESYFPAFKKATLEMITAQGGIVGRVA |
| | SLTDLEQALQTRSTH |
| 215 | WP_004418899.1 cysteine hydrolase [Pseudomonas syringae]: |
| | MIKVNARPDRFAFDTSRTAVVIIDMQRDFLEPGGFGAALGNDVAPLQAIVTSVQRLLTLARNEGMAVIHT |
| | RESHRPDLADCPQAKRDHGSPGLRIGDPGPMGRILISGEPGNQIIDTLAPRAGEWVIDKPGKGMFFATDL |
| | QQRLSEAGITHLIFAGVTTEVCVQTSMREANDRGYRCLLIEDATESYFPAFKQATLEMITAQGGIVGRVA |
| | SLTDLEQALLTRSTL |
| 216 | WP_004666111.1 MULTISPECIES: cysteine hydrolase [Pseudomonas syringae |
| | group]: |
| | MISVNARPDCFTFAPSCAAVVIIDMQRDFLEPGGFGAALGNDVVPLQAIVPSVQQLLALARDQGITVIHT |
| | RESHSADLANCPPAKLAHGSPGLRIGDSGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTH |
| 217 | WP_004667415.1 cysteine hydrolase [Pseudomonas savastanoi]: |
| | MISVNARPDCFTFAPSCAAVVIIDMQRDFLEPGGFGAALGNDVVPLQAIVPSVQQLLALARDQGITVIHT |
| | RESHSADLANCPPAKLAHGSLGLRIGDSGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTH |
| 218 | WP_004883221.1 MULTISPECIES: cysteine hydrolase [Pseudomonas syringae |
| | group]: |
| | MIDVNAHPARFAFDPASTALVIIDMQRDFLEPGGFGAALGNDVLPLQAIIPTVQQLLALARDQNLTVIHT |
| | RESHAEDLADCPPAKLEHGLPGLRIGDAGPMGRILVRGEPGNQIIDALAPIAGEWVIDKPGKGMFFGTGL |
| | HGRLSTAGITHLIFAGVTTEVCVQSSMREANDRGYRCLLIEDATESYFPAFKQATLDMITAQGGIVGRVT |
| | SLSALEQALQTRSTH |
| 219 | WP_005143268.1 cysteine hydrolase [Mycolicibacterium rhodesiae]: |
| | MPTIENAKPFPFEFGIDHVALVCIDMQRDFCLPGGFADSLGNNLDNIAPCIPVIAKLQAAFRKAGLPIIH |
| | TKECHKPDLSDVPTAKRNRGNPSIKIGDPGPMGRILIDGEEGSDFIPQNAPAEHELVISKPGKDAFYRTI |
| | FYEYLTTRLITHLFITGVTTEVCVQTTMRCANDRGFDCVLVEDGTDSYFPEFKDMTLRAVVAQGGIVGWT |
| | CTSDQIVDALATL |
| 220 | WP_005145287.1 cysteine hydrolase [Mycolicibacterium rhodesiae]: |
| | MATISAEPFPLDFDVASTALVIIDMQRDFVLPGGFGEALGNDTSLLLAAVEPIDRVLTKAREIGMLVIHT |
| | REGHRPDLTDCPPAKLNRGGKTFIGEPGPMGRILVRGEQGHDIIHQLYPIDGEPVIDKPGKGSFHATDLG |
| | QILSDRGIKTLVVCGVTTEVCVHTTVREANDRGYECLVLSDCCASYFPEFHRVALEMVKAQGAIFGWVAD |
| | ADAFIAATS |
| 221 | WP_005355369.1 cysteine hydrolase [Aeromonas diversa]: |
| | MNKRISAQPFDFTFDPATTALLVIDMQRDFVEPNGFGHALGNDVSLVRRAIEPCRKVLDAARAKGMLVIH |
| | TREGHRPDLTDCLPAKLIRGGKRFIGEQGAMGRILVQGEAGHDIIPELYPIAGEPVIDKPGKGAFYSTDL |
| | HLILQARGIRSLIICGVTTEVCVQTTAREANDRGYELVIPADCCASYFPEFHRVTLEMIQAQGAIVGWVS |
| | DAEQLVAALKD |
| 222 | WP_005615644.1 cysteine hydrolase [Pseudomonas avellanae]: |
| | MISISARPDTFTFEPSRTAVVIIDMQRDFLEPGGFGAALGNDVAPLQAIVPTVQQLLALAREQGLVVIHT |
| | RESHRPDLSDCPQAKLDHGLPGLRIGDPGPMGRILVRGEYGNQIIDALTPLASEWVIDKPGKGMFFATDL |
| | HHRLTAAGITHLIFAGVTTEVCVQTSLREACDRGYRCLLIEDATESYFPAFKQATLDMITAQGAIVGRVA |
| | SLANLQHALHTRSTP |
| 223 | WP_005735190.1 cysteine hydrolase [Pseudomonas amygdali]: |
| | MISVNARPDCFTFAPSCAAVVIIDMQRDFLEPGGFGAALGNDVAPLQAIVPSVQQLLALARDQGITVIHT |
| | RESHSADLADCPPAKLAHGSPGLRIGDPGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTP |
| 224 | WP_005736371.1 MULTISPECIES: cysteine hydrolase [Pseudomonas syringae |
| | group]: |
| | MISISARPDTFTFEPSRTAVVIIDMQRDFLEPGGFGAALGNDVAPLQAIVPTVQQLLALAREQGLVVIHT |
| | RESHRPDLSDCPQAKLDHGLPGLRIGDPGPMGRILVRGEPGNQIIDALTPLASEWVIDKPGKGMFFATDL |
| | HQRLTAAGITHLIFAGVTTEVCVQTSLREACDRGYRCLLIEDATESYFPAFKQATLDMITAQGAIVGRVT |
| | SLANLQHGLHTRSTP |
| 225 | WP_005745867.1 cysteine hydrolase [Pseudomonas amygdali]: |
| | MISVNARPDCFTFAPSCAAVVIIDMQRDFLEPGGFGAALGNDVVPLQAIVPSVQQLLALARDQGIVVIHT |
| | RESHSADLADCPPAKLAHGSPGLRIGDPGPMGRILIRGEPGNQIIDSLTPLACEWIIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTS |
| 226 | WP_005763961.1 MULTISPECIES: cysteine hydrolase [Pseudomonas syringae |
| | group]: |
| | MISISARPDTFTFEPSRTAVVIIDMQRDFLEPGGFGAALGNDVAPLQAIVPTVQQLLALAREQGLVVIHT |
| | RESHLPDLSDCPQAKLDHGLPGLRIGDPGPMGRILVRGEPGNQIIDALTPLASEWVIDKPGKGMFFATDL |
| | QQRLTVAGITHLIFAGVTTEVCVQTSMREACDLGYRCLLIEDATESYFAAFKQATLDMITAQGAIVGRVA |
| | SLANLQHALHTRSTQ |
| 227 | WP_005778199.1 cysteine hydrolase [Pseudomonas amygdali]: |
| | MISVNARPDCFTFAPSCAAVVIIDMQRDFLEPGGFGAALGNDVVPLQAIVPSVQQLLALARDQGITVIHT |
| | RESHSADLADCPPAKLAHGPPGLRIGDPGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTS |
| 228 | WP_005855152.1 cysteine hydrolase [Sagittula stellata]: |
| | MRIAAQPFPLDLDPATAALIVIDMQRDFIEPGGFGASLGNDVTRLQAIVPATARLIDGCRKAGIPVIHTR |
| | ECHKPDLSDCPPAKRLRGAPSLRIGDAGPMGRVLIAGEPGAEIVPDLAPIPGEKVIDKPGKGAFYATDLG |
| | PYLACLGTKTLIFAGVTTEVCVQTTMREANDRGFDGLLAEDATESYFPEFKQAALQMIRAQGAIVGWTAP |
| | VATILTALDMADA |
| 229 | WP_005891502.1 cysteine hydrolase [Pseudomonas coronafaciens]: |
| | MIRINARPDSFSCELSQTALVIIDMQRDFLEPGGFGAALGNDVTLLQAIVPSVQRLLALAREQDLIVIHT |
| | RESHPADLSDCPQAKIDHGLPGLRIGDPGPMGRILIQGEPGNQIIEALTPVAGEWVIDKPGKGMFFATDL |
| | HLRLTEAGITHLIFAGVTTEVCVQTSMREACDRGYHCLLIEDATDSYFPAFKQATLDMITAQNAIVGRVA |
| | SLADVQQALPARSTQ |
| 230 | WP_006023006.1 cysteine hydrolase [Afipia broomeae]: |
| | MPPTKNLLAAEPAPVELAWAKTALVIIDMQRDFLEPGGFGETLGNDVSQLTRAVKPCSDVLAAFRGAGLL |
| | VIHTREGHLPDLSDAPPAKIERGAPSLRIGDPGPMGRILIRGEAGHDIVPPLYPVKGEIVIDKPGKGAFY |
| | ATELGEILKQHGIENLLVCGVTTEVCVNTTVREANDRGYRCVVLADCCASYFPEFHEMGLRMIKAQGGIF |
| | GWVSSSSEVLKALNSEPSKMIA |
| 231 | WP_006203441.1 cysteine hydrolase [Mesorhizobium amorphae]: |
| | MAEIAAQPFAFGFKPETTALIVIDMQRDFAEPGGFGASLGNDVSRVVAIVPTVKRLIDGFRAAGLPVIHT |
| | MECHRPDLSDLPPAKRDRGNPSIRIGDMGPMGRLLIAGEPGTAILDELAPLPGEIVIEKPGKGAFYATSL |
| | SNDLKWIGARQLVFAGVTTEVCVQTTMREANDRGYECLVAEDATESYFPEFKAAALAMIRAQGAIVGWTA |
| | TTDQVLEGIANA |
| 232 | WP_006229928.1 cysteine hydrolase [Photobacterium profundum]: |
| | MTRTFNAEPFALEFSPVNTALVIIDMQRDFVEPGGFGEALGNDVSLVRSAIEPCGKVLKAARDAGIMVIH |
| | TREGHRADLSDCPPAKLTRGGQTFIGEESPKGRILIRGEEGHDIIPELYPIAGEPIIDKPGKGAFYQTDL |
| | HLILQNRNIKTLIVCGVTTEVCVTTTVREANDRGYECIIPEDCVGSYFPKFQKYSLEMIKAQGAIFGWVS |
| | NSENIIDGIK |
| 233 | WP_006296381.1 cysteine hydrolase [Hylemonella gracilis]: |
| | MHINAQPFAYECDPRATALVLIDMQRDFIEPGGFGETLGNDVSLLAAIVPACRSVLAAWRRAGGLVLHTR |
| | EAHQPDLSDCPPAKRLRGNPSLRIGDAGPMGRILVAGEPGNQIIDALAPAPGELVIDKPGKGMFWATGLH |
| | EKLQARGVTHLIFMGVTTEVCVQTSMREANDRGYDSLVLEDCTESYFPAFKAATLEMIRAQGAIVGWTAR |
| | SEALLAALK |
| 234 | WP_006332673.1 cysteine hydrolase [Gordonia rhizosphera]: |
| | MTDPVSIPALPEPIALDLARTALVIIDMQRDFLLPGGFGETLGNDVSQLQRVVKPLAGLLAAARDAGMLV |
| | IHTREGHLPDLSDCPPAKLNRGAPSKRIGDPGAFGRILIRGEYGHDIIDELAPADGEIVIDKPGKGAFYA |
| | TDLAKVLADNEITQLLVTGVTTEVCVHTTTREANDRGYECVVVSDCVGSYFPEFQRVGLEMIAAQGGIFG |
| | WTAPSGEVIAAISRHAAASPSSALS |
| 235 | WP_006338345.1 cysteine hydrolase [Mesorhizobium sp. STM 4661]: |
| | MAEIAAQPFPFAFKPRTMALVVIDMQRDFAEPGGFGASLGNDVSRVIAIVPTVKRLIEGFRAAGLPVIHT |
| | MECHRPDLSDLPPAKRNRGNPSIRIGDAGPMGRVLIAGEPGTAILDELAPLPGEIVIEKPGKGAFYATSF |
| | GDDLRKLGAEQLVFAGVTTEVCVQTTMREANDRGYDCLLAEDATESYFPEFKAAALAMIRAQGAIVGWTA |
| | TTDQVLEGIANA |
| 236 | WP_006356764.1 cysteine hydrolase [Gordonia alkanivorans]: |
| | MSETVTLEALPGPIELDLDRTALIIIDMQRDFLLPGGFGETLGNDVAQLQRVVEPLAALLDAARAAGMLV |
| | IHTREGHLPDLSDCPPAKLNRGAPSKRIGDPGAFGRILIRGEYGHDIVDELAPLDTEVVIDKPGKGAFYA |
| | TELSKVLADNQIAQLLVTGVTTEVCVHTTTREANDRGFECVVVSDCVGSYFPEFQRVGLEMIAAQGGIFG |
| | WTAPGAAIIPLLKERAPAEPAV |
| 237 | WP_006434890.1 MULTISPECIES: cysteine hydrolase [Gordonia]: |
| | MPTTVILDALPGPIELDLDQTALIIIDMQRDFLLPGGFGETLGNDVSQLQRVVEPLAALLDAARAVGMLV |
| | IHTREGHLPDLSDCPPAKLNRGQPSKRIGDPGAFGRILIRGEYGHDIIDELAPLDTEVVIDKPGKGAFYA |
| | TELSKVLADNEITQLLVTGVTTEVCVHTTTREANDRGFECVVVSDCVGSYFPDFQRVGLEMIAAQGGIFG |
| | WTAPGTAIIPLLNGRAPVEPAV |
| 238 | WP_006454721.1 isochorismatase family protein [Synechococcus sp. PCC |
| | 7335]: |
| | MLPSHITIPARPYPIALSLEHTALLVIDMQNDFCTPGGWADLKGFDVRETQQPIRPLKALLAALRQTPIT |
| | IIHTREGHRPDLSDCPPHKLDRSKRQKAEIGSEGMMGRLLTRGSKSHDFVDELQPLPDEIVLDKPGKGAF |
| | VATDLDLILRQRNIRQLVLTGVTTECCVHTTLRTANDLGYECLLLEDCCASLNPEFHRISVEMTQTIFGW |
| | VSVSTKLLQAIDF |
| 239 | WP_006455781.1 cysteine hydrolase [Synechococcus sp. PCC 7335]: |
| | MNTIPALPYDYPLPDSLDHLALVIIDMQRDFLEPGGFGDALGNDVTQLQAIVPQLKTLLHTFRDLDLLVI |
| | HTQECHAPNLSDCPTSKLTRGDAKLRIGDRSAMGRILVRGEPGNAIIPALAPRPNEVVIRKPGKGAFYNT |
| | PLSSILQKYSITHLLITGVTTEVCVQSTMREANDRGYECLLVEDCTASYFPEFKEATIQMLRAQGGIIGW |
| | TSIADKVCQSLLKTAQGE |
| 240 | WP_006611979.1 cysteine hydrolase [Bradyrhizobium sp. ORS 285]: |
| | MANPAASATATIIAEPEPIALDLSRTALVIIDMQRDFMEPGGFGETLGNDVSQLARAVQPIANVLAAARK |
| | TGMLVIHTREGHKPDLSDAPPAKVERGAPSLRIGDPGPMGRILIRGEAGHDIIAALYPTDGEVVIDKPGK |
| | GAFYATELGDVLKQHGIENLLVCGVTTEVCVNTTVREANDRGYRCVVISDGCASYFPEFHEMGLKMIKAQ |
| | GGIFGWVADSAAVLKAMT |
| 241 | WP_006725484.1 cysteine hydrolase [Agrobacterium albertimagni]: |
| | MAVIKARPFDITITPEKIALVVIDMQRDFIEPGGFGATLGNDVTLLQAIIPATARLIDGFRRAGLPVIHT |
| | RECHAPDLLDCPPAKRARGKPSLRIGDPGPMGRILIAGEDGADIVAALAPLPGETVIDKPGKGAFYATPL |
| | GDILQEKGISQLVFAGVTTEVCVQTTMREANDRGYECLLATDATESYFPEFKKAAIDMMTAQGAIVGWAA |
| | TVDQIVEALDA |
| 242 | WP_007162804.1 MULTISPECIES: cysteine hydrolase [Pseudomonas]: |
| | MIQVSARPDTFAFEPASTALVVIDMQRDFIEPGGFGAALGNDVTPLKAIIPAVQRLLALARQHRVLVVHT |
| | RESHLPDLSDCPPAKHAHGLPGLRIGDPGPMGRILVRGEPGNQLLAEVAPIEGEWVIDKPGKGMFHATGL |
| | HERLQAEGVSHLVFAGVTTEVCVQTSMREANDRGYRCLLIEDATESYFPAFKRATLDMIVAQGGIVGRTA |
| | SLAALEAALHKDLP |
| 243 | WP_007177323.1 cysteine hydrolase [Burkholderia sp. Ch1-1]: |
| | MTSTHLLSIAAQPGPFSFDPAKTALVVIDMQRDFIEPGGFGESLGNDVSLLAEIVPTVAALLAFARQHHW |
| | LVVHTRESHAADLSDCPPAKRLRGAPNARIGDAGPMGRILVRGEPGNAIIEPLVPLAGELVIDKPGKGAF |
| | YATRLGEELAMRGITHLVFAGVTTEVCVQTSMREANDRGYDSLLIEDATASYFPAFKQATLEMVRSQGGI |
| | VGWTAPLSSLLKIDGTIPAWK |
| 244 | WP_007186015.1 cysteine hydrolase [Hydrocarboniphaga effusa]: |
| | MSTAPIQSKVRVIQAQPYELAFEPASTALLIIDMQRDFIEPGGFGAMLGNDVSLLRRAIEPIGALLSAFR |
| | EAGLLVLHTREGHRPDLSDAPPSKLARGRGETKIGDVGPMGRILIRGEAGHDIIPELYPLAGEPVIDKPG |
| | KGSFCQTDLELILKNRGIKTLIVCGVTTEVCVHTTVREANDRGFECLVPADCAASYFPDFHETALRMIAA |
| | QGGIFGWVSDSASVIAALS |
| 245 | WP_007244854.1 MULTISPECIES: cysteine hydrolase [Pseudomonas syringae |
| | group]: |
| | MISISARPDTFTFEPSRTAVVIIDMQRDFLEPGGFGAALGNDVAPLQAIVPTVQQLLALAREQGLVVIHT |
| | RESHLPDLSDCPQAKLDHGLPGLRIGDPGPMGRILVRGEPGNQIIDALTPLASEWVIDKPGKGMFFATDL |
| | HQRLTVAGITHLIFAGVTTEVCVQTSLREACDRGYRCLLIEDATESYFAAFKQATLDMITAQGAIVGRVA |
| | SLANLQHALHTRSTQ |
| 246 | WP_007248437.1 MULTISPECIES: cysteine hydrolase [Pseudomonas syringae |
| | group]: |
| | MISLPARPSPFLFEPSRTAVVIIDMQRDFLEPGGFGAALGNDVAPLQAIVPVVHRLLTLVRDQGITVIHT |
| | RESHHPDLSDCPQAKLEHGSPGLRIGDPGPMGRILVRGEPGNQIIDALTPIAGEWIIDKPGKGMFFATDL |
| | HALLAEAGIIHLIFAGVTTEVCVQTSMREANDRGYRCLLLEDATESYFPAFKQATLDMITAQGAIVGRVA |
| | ALADLEQALQTRSTH |
| 247 | WP_007261229.1 cysteine hydrolase [Natronolimnobius innermongolicus]: |
| | MVEFDSGRTAFLSIDMQRDFCGENGYVDAMGYDLSRTQRAVQPISNVLEAVRRTDIDVVHTREGHKQDLS |
| | DAPFNKLLRSKMAGDGDGIGETPAGGVGPLLTREHENWDIIDELAPEPGEPVIDKPTKGAFANTNIGLVL |
| | ERLGTTHLVIAGITTDVCVHTIMREANDRGYWCLLLKDATGATDDGNREAAIKQIKMQGGVFGWVSDSER |
| | FIEAVESGVA |
| 248 | WP_007356072.1 MULTISPECIES: cysteine hydrolase [Kamptonema]: |
| | MISIPAKPYDYELPNLNSVALIVIDMQRDFLEPGGFGEILGNDVSLLQSIVPTVKQLLEEFRKFNLPIFH |
| | TIEGHNSDLSDCPISKIKRGKGKLTIGDVGPMGRILVLGEAGNGIIPELAPLPGEIALSKPGKGAFSRTK |
| | LESMLQEKGITHLIFAGVTTEVCVQTTMREANDRGYECLLIEDATASYFPEFKQATLEMIRAQGGIIGWT |
| | TTATQLFKALNH |
| 249 | WP_007511329.1 MULTISPECIES: cysteine hydrolase [Frankia]: |
| | MTSAPLTVSARPYDFTFDPATTALVVIDMQRDFMEPGGFGESLGNDVSQLRSTIEPLTAVFAAARAAGLT |
| | VIHTREGHKPDLSDLPPAKLNRGNAALKIGDVGPKGRILIRGEYGQDIIDELAPIEGEIVIDKPGKGAFY |
| | ATSFGEILAEKGIKSLVVTGVTTEVCVHTTVREANDRGYECLVLSDCVGSYFPEFQRVALEMIAAQGGIF |
| | GWVAPSAAFIDALAPLSAASAAQ |
| 250 | WP_007537281.1 cysteine hydrolase [Rhizobium mesoamericanum]: |
| | MDKIKAEPFSFPVKHDQLALVVIDMQRDFAEPGGFGASLGNDVSRITRIVPDVKRLIEGFRNAGLPIIHT |
| | MECHRPDLSDLPPAKRDRGNPTLRIGDIGPMGRVLISGEPGTAILPELAPVEGEVVIEKPGKGAFYATKL |
| | GEVLQQRGIKQLVFAGVTTEVCVQTTMREANDRGYECLLAEEATESYFAEFKAAAIAMIRAQGAIVGWTA |
| | HVDDILESISHA |
| 251 | WP_007594065.1 cysteine hydrolase [Bradyrhizobium sp. WSM1253]: |
| | MLNSTKPTLGVISAEPEPVKLDWPATALLIIDMQRDFMEPGGFGETLGNDVSQLARAVKPIGAVLKAARD |
| | TGMLVIHTREGHLPDLSDAPPAKVERGAPSLRIGDPGPMGRILIRGEAGHDIIPELYPLDSEIVIDKPGK |
| | GAFYATELTDVLEKYGIENLLVCGVTTEVCVNTTVREANDRGYRCVVISDGCASYFPEFHEMGLKMIKAQ |
| | GGIFGWVASSAAVLEAMKISTSA |
| 252 | WP_007603722.1 cysteine hydrolase [Rhizobium sp. PDO1-076]: |
| | MTDIKALPFAFPLRRDAVALIVIDMQRDFAEPGGFGETLGNDVSHVSVIVPDVKRLIDGFRHAGLPVIHT |
| | QECHRPDLSDLPPAKRNRGNPTLRIGDQGPMGRILIAGEPGTAILPELEPIGGELVIEKPGKGAFYATSL |
| | GEELQNRGITQLVFAGVTTEVCVQTTMREANDRGYECLIVEEATASYFPHFKQAALDMIRAQGGIVGWTA |
| | HLDDLLKGLMHA |
| 253 | WP_007607621.1 MULTISPECIES: cysteine hydrolase [unclassified |
| | Bradyrhizobium]: |
| | MLNSSKPTLGVISAEPGPIELDWPATALLIIDMQRDFMEPGGFGETLGNDVSQLARAVKPIGAVLKAARD |
| | TGMLVIHTREGHLPDLSDAPPAKIERGAPSLRIGDPGPMGRILIRGEAGHDIIPELYPLDSEIVVDKPGK |
| | GAFYATELTDVLEKYGIENLLVCGVTTEVCVNTTVREANDRGYRCVVISDGCASYFPEFHEMGLKMIKAQ |
| | GGIFGWVASSAAVLEAMKTSNIPA |
| 254 | WP_007634561.1 cysteine hydrolase [Rhizobium sp. CCGE 510]: |
| | MAQIKAEPFAFPVKHDELALIVIDMQRDFAEPGGFGASLGNDVSRITRIVPDVKRLIQGFRNAGLPVIHT |
| | MECHRPDLSDLPPAKRNRGNPSLRIGDEGPMGRVLISGEPGTAILPELSPVKGEVVIEKPGKGAFYATEL |
| | GTVLQEKGIRQLVFAGVTTEVCVQTTMREANDRGYECLLAEEATESYFPEFKAAAIAMIRAQGAIVGWTA |
| | HVDDILESIAHA |
| 255 | WP_007766673.1 cysteine hydrolase [Rhizobium sp. CF080]: |
| | MGEIKAEPFAFPAKPDALALIVIDMQRDFAEPGGFGASLGNDVGRITRIVPDVKRLIQGFRDAGLPVIHT |
| | MECHKPDLSDLPPAKRDRGNPTLRIGDVGPMGRVLISGEPGTAIISELAPVDGEVVIEKPGKGAFYATEL |
| | GEVLKDKGIKQLVFAGVTTEVCVQTTMREANDRGYECLLAEEATESYFPEFKAAAIAMIRAQGAIVGWTA |
| | HVDDILEGIAPKGMTNA |
| 256 | WP_007793509.1 cysteine hydrolase [Rhizobium sp. CF122]: |
| | MADIKAQPFAFPTKSDQLALIVIDMQRDFAEPGGFGASLGNDVSRITNIVPDVKRLIQGFRDAGLPVIHT |
| | MECHKPDLSDLSPAKRNRGKPTLRIGDDGPMGRILIAGEAGTAILPELAPIDGEIVIEKPGKGAFYATEL |
| | GDVLKARGISQLVFAGVTTEVCVQTTMREANDRGYECLLAEEATESYFPEFKEAAIAMIRAQGAIVGWTA |
| | HVDDILEWGHA |
| 257 | WP_007826659.1 cysteine hydrolase [Rhizobium sp. CF142]: |
| | MAEIKAEPFDFPAKHDQLALIVIDMQRDFAEPGGFGASLGNDVSRITRIVPDVKRLIQGFRNAGLPVIHT |
| | MECHRSDLSDLPPAKRDRGNPTLRIGDEGPMGRILISGEPGTAILPELAPLKGEVVIEKPGKGAFYATEL |
| | GDVLQRRGIKQLVFAGVTTEVCVQTTMREANDRGYECLLAEEATESYFPEFKAAAIAMIRAQGAIVGWTA |
| | HVDDILESIAHA |
| 258 | WP_007880244.1 cysteine hydrolase [Herbaspirillum sp. CF444]: |
| | MITVDAIPYPYQFDSRHTALVVIDMQRDFVEEGGFGSVLGNDVRPLATIVPAVAKLLTLARAHGMLVVHT |
| | RESHLPDLSDCPPAKLKRGNPTLGIGDEGPMGRILVRGEPGNQILPLLAPLDGELVIDKPGKGAFYATDL |
| | HAQLQARGITHLLFAGVTTEVCVQTSMREANDRGYECLIVEDACASYFPVFHQATLAMLTAQGGIVGWQA |
| | PLSTLQTAFKETAGESVS |
| 259 | WP_008140138.1 cysteine hydrolase [Bradyrhizobium sp. YR681]: |
| | MLNSTKPTPGVISAEPEPIKLDWPSTALLIIDMQRDFMEPGGFGETLGNDVSQLARAVKPIGAVLKAARD |
| | TGMLVVHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVASSAAVLEAMKVSTKQG |
| 260 | WP_008326894.1 cysteine hydrolase [Herbaspirillum sp. GW103]: |
| | MIRIDAFPYPYQFHPRSTALVVIDMQRDFVEEGGFGSALGNDVRPLGAIVPTVAALLALARQTQMLVVHT |
| | RESHLPDLSDCPRAKRLRGNPTLGIGDVGPMGRILVRGEPGNQILPQLAPMAGEIVIDKPGKGAFYATDL |
| | HTQLQERGITHLLVAGVTTEVCVQTSMREANDRGYECLVVEDACASYFPEFHRATLEMLTAQGGIVGWRA |
| | PLAQLQGAVAAYAGENP |
| 261 | WP_008354087.1 cysteine hydrolase [Caballeronia zhejiangensis]: |
| | MTQKHFRAEPFDLAFEPKHTALVMIDMQRDFVEPGGFGEALGNDVSFVRTAIEPCKRVLAAARDAGMLVI |
| | HTREGHRADLTDCPPAKLTRGGKTFIGSDGPMGRILVRGEKGHDLIPELYPVAGEPVIDKPGKGAFYETD |
| | LHLILKNHDTRTLIVCGVTTEVCVTTTVREANDRGFECIVPQDCVGSYFPEFQKYALEMIKAQGAIFGWV |
| | SDAGAVIEALRG |
| 262 | WP_008494190.1 cysteine hydrolase [Acidocella sp. MX-AZ02]: |
| | MSREIAARPAPFVLDIGRVALVIIDMQRDFLEPGGFGAALGNDVTKLRAAIGPIVTVLAAARAAGILVVH |
| | TREGHRPDLADLHPAKHRRAAGIGRAGPMGRILVRGEAGHGIIDDLAPADGEPVVDKPGKGAFYATDLET |
| | ILHKRSITQLILAGVTTEVCVHTTLREANDRGFECLVLEDGTASYFPEFHRAALEMVAAQGGIFGWVAAS |
| | ADVAASLAGA |
| 263 | WP_008524119.1 cysteine hydrolase [Rhizobium sp. Pop5]: |
| | MAEINAEPFAFPVKHDQLALIVIDMQRDFAEPGGFGASLGNDVSRITRIVPDVKRLIHGFRYAGLPVIHT |
| | MECHKPDLSDLPPAKRNRGNPSLRIGDEGPMGRILVAGEPGTAILPELAPVRGEVVIEKPGKGAFYATEL |
| | GEVLQQKGIKQLVFAGVTTEVCVQTTMREANDRGYECLLAEEATESYFPEFKAAAIAMIRAQGAIVGWTA |
| | HVDDILESIAHA |
| 264 | WP_008813988.1 cysteine hydrolase [Hafnia paralvei]: |
| | MTQHQFHAEPFALPFDPTTTALVMIDMQRDFVEPSGFGEALGNDVSRVRTAIEPCKRVLDAARTHGLLVI |
| | HTREGHRSDLTDCPPAKLTRGGKTFIGTEGPMGRILVRGETGHDIIPELYPISGEPVIDKPGKGAFYQTD |
| | LHLVLQNRGIKTLIVCGVTTEVCVTTTVREANDRGYECIIPQDCVGSYFPEFQKYALEMIKAQGAIFGWV |
| | SDADSIIHGLQG |
| 265 | WP_008877628.1 cysteine hydrolase [Mesorhizobium metallidurans]: |
| | MAEIAAQPFPFAFKPRTMALIVIDMQRDFAEPGGFGASLGNDVSRVVAIVPTVKRLIEGFRAAGLPVIHT |
| | MECHRPDLSDLPSAKRDRGNPSIRIGDAGPMGRVLIAGEPGTAILDELAPLPGEIVIEKPGKGAFYATSF |
| | GEDLRKLGAEQLVFAGVTTEVCVQTTMREANDRGYDCLLAEDATESYFPEFKAAAIAMIRAQGAIVGWTA |
| | TTDQVLEGIANA |
| 266 | WP_008962583.1 cysteine hydrolase [Bradyrhizobium sp. STM 3809]: |
| | MANSSAAATATIAAEPEPIALDLSKTALVIIDMQRDFMEPGGFGETLGNDVSQLARAVAPIAAVLAAARK |
| | TGMLVIHTREGHEPDLSDAPPAKVERGAPSLRIGDPGPMGRILIRGEAGHDIIPALYPVDGEVVIDKPGK |
| | GAFYATEMGEVLKHHGIENLLVCGVTTEVCVNTTVREANDRGYRCVVISDGCASYFPEFHEMGLKMIKAQ |
| | GGIFGWVADSAAVLKAMS |
| 267 | WP_008967232.1 cysteine hydrolase [Bradyrhizobium sp. STM 3843]: |
| | MTSSLLATTGTVMAEPEPISLDWSKTALLIIDMQRDFMEPGGFGETLGNDVSQLARAVQPIANLLTAARN |
| | AGMLVIHTREGHKPDLSDAPPAKVERGAPSLRIGDPGPMGRILIRGEPGHDIIPELYPVAGEVVIDKPGK |
| | GAFYATELGDVLKQHAIANLLVCGVTTEVCVNTTVREANDRGYRCVVISDGCASYFPEFHEMGLRMIKAQ |
| | GGIFGWVADSTAVLAAMSLETLNA |
| 268 | WP_009027226.1 cysteine hydrolase [Bradyrhizobium sp. ORS 375]: |
| | MANSSASATATIIAEPEPIALDLSRTALVIIDMQRDFMEPGGFGETLGNDVSQLARAVQPIANVLAAARK |
| | SGMLVIHTREGHEPDLSDAPPAKVERGAPSLRIGDPGPMGRILIRGEAGHDIIPALYPTDGEVVIDKPGK |
| | GAFYATEMGDVLTHHGIDNLLVCGVTTEVCVNTTVREANDRGYRCVVISDGCASYFPEFHEMGLKMIKAQ |
| | GGIFGWVAESAAVLKAIS |
| 269 | WP_009143993.1 cysteine hydrolase [Succinatimonas hippei]: |
| | MIKQIKARPFDFTFDPEKTALMVIDMQRDFVQKGGFGEALGNDVSPMQKAIEPISKVLECCRSQHMLIIH |
| | TREGHRPDLTDCPKAKLTRGGKTFIGTDGPMGRILVRGEYGHDIIPELYPKEGEVVIDKPGKDAFFATDL |
| | YQILLNRGIKSLIICGVTTEVCVQTTSRAANDRGFELVIPEDCCASYFPEFHEAALNMIAAQGAIVGWVS |
| | DSKSVISALEE |
| 270 | WP_009462637.1 cysteine hydrolase [Ahrensia sp. R2A130]: |
| | MVSVPALPFPFPLRVDEAALLVIDMQRDFVEPGGFGESLGNDVRPLQAIIPVIADLLALFRQQGLPVIHT |
| | RECHRPDLTDCPKAKRNRGDPPLRIGDDGPMGRLLIAGEHGAGIVDALAPIAGETMIDKPGKGAFHATPL |
| | GDELVAHRITQLVVAGVTTEVCVQSTIREANDRGFECLLVTDATESYFPAFKQATIDMLTAQGAIGGWAT |
| | SSGELMEVIR |
| 271 | WP_009518696.1 MULTISPECIES: cysteine hydrolase [Hydrogenophaga]: |
| | MTIAAQPFAFELDLAHAALVIIDMQRDFVEPGGFGETLGNDVSLLQAIVPACQAVLHAWRRAGGLVVHTR |
| | EAHRPDLSDCPPAKRLRGQPSLRIGDEGPMGRILIAGEPGNQIIDALAPRPGEIVLDKPGKGAFYATPLH |
| | NLLQQAGVVQLVFMGVTTEVCVQTSMREANDRGYDALLLEDCTESYFPQFKRATLEMVRAQGAIVGWTAS |
| | SAALLTALPPR |
| 272 | WP_009556372.1 cysteine hydrolase [Oscillatoriales cyanobacterium JSC- |
| | 121: |
| | MVYISALPYEYELPESSQVALVVIDMQRDFLEPGGFGDALGNNVARLQAIVPTLKRLIAGFRELGLPIIH |
| | TLECHLPDLSDCPPSKIRRGKGELTIGSEGPMGRILVKGEPGNGIIPELAPLPGEFVIHKPGKGAFYATE |
| | METILQKQGITHLLITGVTTEVCVQTTMREANDRGYECLMVEDCTESYFPEFKQATLDMVRAQGGIVGWT |
| | ATAEEVLAGLRQWQPSKVFV |
| 273 | WP_009626489.1 cysteine hydrolase [Pseudanabaena biceps]: |
| | MSTIAANPYEYELPSEGKIALVIIDMQRDFLEHGGFGDALGNDVMQLQSIVPTVKKLLETFRSLNFPVIH |
| | TIEAHAPDLSDCPPSKLNRGRGNLKIGDQGSMGRILIVGEDGNNIIPELTPLANEIVIVKPGKGAFCRTN |
| | LEEILQKENITHLLFTGVTTEVCVQTTMREANDRGYECLLIEDGTASYFPEFKTSTIEMLRAQGGIIGWT |
| | ANSEAVIAALLPQAMAIA |
| 274 | WP_009734949.1 cysteine hydrolase [Bradyrhizobiaceae bacterium SG-6C]: |
| | MPPTKNLLAAEPAPLELAWPKTALVIIDMQRDFLEPGGFGETLGNDVSQLTRAVKPCGDVLAAFRKAGLL |
| | VVHTREGHLPDLSDAPPAKIERGAPSLRIGDPGPMGRILIRGEAGHDIVPSLYPIKGEIVIDKPGKGAFY |
| | ATGLGDILKTRGIENLLVCGVTTEVCVNTTVREANDRGYRCVVLADCCASYFPEFHEMGLRMIKAQGGIF |
| | GWVSSSAEVLAALNSEPSKMIA |
| 275 | WP_010025761.1 MULTISPECIES: cysteine hydrolase [Rhizobium]: |
| | MAEIKAEPFAFPVKHDQLALIVIDMQRDFAEPGGFGASLGNDVSRIGRIVPDVKRLIQGFRYAGLPVIHT |
| | MECHRPDLSDLPPSKRDRGNPMLRIGDEGPMGRILIAGEPGTAILPELAPIDGEVVIEKPGKGAFYATGL |
| | AEALQRKGIRQLVFAGVTTEVCVQTTMREANDRGYECLLAEEATESYFPEFKAAAIAMIRAQGAIVGWTA |
| | HVDDILESIAHA |
| 276 | WP_010429025.1 MULTISPECIES: cysteine hydrolase [Pseudomonas]: |
| | MNKVNARPDSFAFDTSRTAVVIIDMQRDFLEPGGFGAALGNDVAPLQAIVPSVQRLLALARDEGIAVIHT |
| | RESHRADLADCPQAKRDHGSPGLRIGDPGPMGRILIRGEPGNQIIDALKPLAGEWVIDKPGKGMFFATEL |
| | QQRLSEAGITHLIFAGVTTEVCVQTSMREANDRGYRCLLIEDATESYFPAFKRATLEMITAQGGIVGRVA |
| | SLMDLEQALQTRSTH |
| 277 | WP_010799275.1 cysteine hydrolase [Pseudomonas sp. HPB0071]: |
| | MIRVTANPNDFSFEPASTALVIIDMQRDFIEQGGFGAALGNDVTPLKAIVPAVRRLLELARQQGMLAIHT |
| | RESHLPDLSDCPDAKYAHGLPGLRIGDPGPMGRILVRGEPGNQIVADVAPAEGEWVIDKPGKGMFYATGL |
| | HERLQARGISHLLFAGVTTEVCVQTSMREANDRGYRCLLIEEATESYFPAFKRSTLEMIVAQGGIVGRTA |
| | HLTALEAALQEDRP |
| 278 | WP_010841208.1 cysteine hydrolase [Gordonia terrae]: |
| | MSDTVTLDAQPGPIELDLAHTALIIIDMQRDFLLPGGFGETLGNDVSQLQRVVAPLAGLLDAARAAGMRI |
| | VHTREGHLPDLSDCPPAKLNRGLPSKRIGDPGAFGRILIRGEYGHDIVDELAPLDTEVVIDKPGKGAFYA |
| | TELAAILADNDITQLLVTGVTTEVCVHTTTREANDRGFECVVVSDCVGSYFPEFQRVGLEMIAAQGGIFG |
| | WTAPSEDVIALLTSATAEDWGSA |
| 279 | WP_010914550.1 MULTISPECIES: cysteine hydrolase [Mesorhizobium]: |
| | MAEIAAQPFPFAFKPRTMALVVIDMQRDFAEPGGFGASLGNDVSRVAAIVPTVKRLIEGFRAAGLPVIHT |
| | MECHKPDLSDLPPAKRNRGNPSIRIGDAGPMGRVLIAGEPGTAILDELVPLPGEIVIEKPGKGAFYATSF |
| | GDELKRLGAEQLVFAGVTTEVCVQTTMREANDRGYECLLAEDATESYFPEFKTAAIAMIRAQGAIVGWTA |
| | TTDQVLEGIANA |
| 280 | WP_011085510.1 cysteine hydrolase [Bradyrhizobium diazoefficiens]: |
| | MLNSTKPTLGVISAEPEPIRLDWPATALLIIDMQRDFMEPGGFGETLGNDVSQLARAVKPIGAVLRAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKISTSA |
| 281 | WP_011167969.1 MULTISPECIES: cysteine hydrolase [Pseudomonas]: |
| | MISVNARPDCFTFAPSCAAVVIIDMQRDFLEPGGFGAALGNDVVPLQAIVPSVQQLLALARDQSITVIHT |
| | RESHSADLANCPHAKLAHGSPGLRIGDSGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTH |
| 282 | WP_011266862.1 cysteine hydrolase [Pseudomonas syringae]: |
| | MNKVNARPDRFAFDTSRTAVVIIDMQLDFLEPGGFGAALGNDVAPLQAIVPSVQRLLTLARDEGMTVIHT |
| | RESHRPDLADCPQAKRDHGSPGLRIGDPGPMGRILIRGEPGNQIIDALAPLADEWVIDKPGKGMFFATDL |
| | QQRLSQAGITHLIFAGVTTEVCVQTSMREANDRGYRCLLIEDATESYFPAFKKATLEMITAQGGIVGRVA |
| | SLTDLEQALQTRSTH |
| 283 | WP_011427967.1 cysteine hydrolase [Rhizobium etli]: |
| | MAEIKAEPFAFPVKHDQLALIVIDMQRDFAEPGGFGASLGNDVSRITRIVPDVKRLIHGFRYAGLPVIHT |
| | MECHRPDLSDLPPAKRDRGNPTLRIGDVGPMGRILISGEPGTAILPELAPVKGEVVIEKPGKGAFFATEL |
| | DEVLQQKGIKQLVFAGVTTEVCVQTTMREANDRGYECLLAEEATESYFPEFKAAAIAMIRAQGAIVGWTA |
| | HVNDILESIAHA |
| 284 | WP_011432436.1 cysteine hydrolase [Synechococcus sp. JA-2-3B′a(2-13)]: |
| | MFRIPALPYEYEVPSLSQLALVIIDMQRDFLEPGGFGEMLGNDVTQLGSIVPTLKGLLDFFRQKGLTVIH |
| | TLEGHQPDLSDCPPSKRKRGKGSLTIGDEGPMGRILIRGEPGNTIIPELAPLAGEIVIPKPGKGAFYATE |
| | LQAILQKRGITHLLFTGVTTEVCVQTTMREANDRGYECLLVEDCTASYFPEFKQATLEMIRAQGGIVGWT |
| | SSAQNIQKALSGLQ |
| 285 | WP_011439916.1 cysteine hydrolase [Rhodopseudomonas palustris]: |
| | MAATTFSASGTIDAEPAPIALDWVATALLIIDMQRDFLEPGGFGETLGNDVSRLGRAVGPIAAVLAAARA |
| | MGLLVVHTREGHLPDLTDAPPAKLARGAPSLRIGDPGPMGRILIRGEPGHDIIPELYPRDDEIVIDKPGK |
| | GAFFATELDDVLQKYGIENLLVCGVTTEVCVNTTVREANDRGYRCVVIGDGCASYFPDFHDAGLAMIKAQ |
| | GGIFGWVADSAAVLAAMAPIVDD |
| 286 | WP_011491914.1 cysteine hydrolase [Paraburkholderia xenovorans]: |
| | MTSTHLLTIDAQPGPFSFDPAKTALVVIDMQRDFIEPGGFGESLGNDVSLLAEIVPTVAALLAFARQHQW |
| | LVVHTRESHAADLSDCPPAKRLRGAPNARIGDAGPMGRILVRGEPGNAIIEPLAPLAGELVIDKPGKGAF |
| | YATRLGEELAMRGITHLVFAGVTTEVCVQTSMREANDRGYDSLLIEDATASYFPAFKQATLDMVRSQGGI |
| | VGWTAPLSSLLKIDGTIPAWK |
| 287 | WP_011560158.1 cysteine hydrolase [Mycobacterium sp. KMS]: |
| | MSETIDVPAEPTPFRLVAGKTALIVIDMQRDFLLPGGFGESLGNDVGQLLKVVPPLAALVAAARAAGVTV |
| | IHTREGHRSDLSDCPPAKLSRGAPSKRIGDQGRYGRILIRGEYGHDIVDELSPLPGEVVIDKPGKGAFYA |
| | TGLQEILTAAGITQLLVTGVTTEVCVHTTTREANDRGYECLVVSDCVGSYFPEFQRIGLEMIKAQGGIFG |
| | WVAGSAAVIPALNAMTPTAA |
| 288 | WP_011654382.1 MULTISPECIES: cysteine hydrolase [Rhizobium]: |
| | MAKIKAEPFAFPVKHDELALIVIDMQRDFAEPGGFGASLGNDVSRITRIVPDVKRLIQGFRNAGLPVIHT |
| | MECHRPDLSDLPPAKRDRGNPALRIGDEGPMGRILISGEPGTAILPELAPVKGEVVIEKPGKGAFYATDL |
| | GTVLQQKGIKQLVFAGVTTEVCVQTTMREANDRGYECLLAEEATESYFPEFKAAAIAMIRAQGAIVGWTA |
| | HVDDILESIAHA |
| 289 | WP_011780260.1 cysteine hydrolase [Mycolicibacterium vanbaalenii]: |
| | MNHTVDVPAEPSSFPLVAGRTALIVIDMQRDFLLPGGFGESLGNDVGRLLKVVPPLASLIAAARAAGVMV |
| | VHTREGHQPDLSDCPPAKLNRGTPSKRIGDPGRYGRILIRGEYGHDIIDELAPIDGEIVIDKPGKGAFYA |
| | TSLSDVLTEAGITQLLITGVTTEVCVHTTTREANDRGYQCLVVSDCVGSYFPEFQRVGLEMIKAQGGIFG |
| | WVADTSSVIPALARLSSIPA |
| 290 | WP_011809413.1 cysteine hydrolase [Verminephrobacter eiseniae]: |
| | MITVDAVPYPYQFDRHHTALMVIDMQRDFIEAGGFGSMLGNDVRPLARIVPTVAQLLTLARAQRMWVVHT |
| | RESHLPDLSDCPPAKRRRGNPALAIGDAGPMGRILVRGAPGNQILPLLAPLDGELVSRHGSDVVGCAQPS |
| | ERSARRIAQPIPSVLASDATPRCASMASADRQMIGDATLVIDKPGKGAFHATDLHAQLQARGITHLLFAG |
| | VTTEVCVQTSMREANDRGYESLIVEDACASYFRAFHLATLAMLTAQGGIIGWKAPLALLQAAFKETAGES |
| | A |
| 291 | WP_011925441.1 cysteine hydrolase [Bradyrhizobium sp. ORS 278]: |
| | MANLAASATATIMAEPEPIALDLSKTALVIIDMQRDFMEPGGFGETLGNDVSQLARAVQPIANVLAAARK |
| | AGMLVIHTREGHKPDLSDAPPAKVERGAPSLRIGDPGPMGRILIRGEAGHDIIPALYPTDGEVVIDKPGK |
| | GAFYATEMGDVLKQHGIENLLVCGVTTEVCVNTTVREANDRGYRCVVVSDGCASYFPEFHEMGLKMIKAQ |
| | GGIFGWVAESAAVLKAMV |
| 292 | WP_012042914.1 cysteine hydrolase [Bradyrhizobium sp. BTAi1]: |
| | MANSSAAATATVTAEPEPITLDLSRTALVIIDMQRDFMEPGGFGETLGNDVSQLARAVAPIANVLAAARK |
| | TGMLVIHTREGHEPDLSDAPPAKIERGAPSLRIGDPGPMGRILIRGEAGHDIIPALYPIDGEVVIDKPGK |
| | GAFYATEMGDVLQHHGIENLLVCGVTTEVCVNTTVREANDRGYRCVVISDGCASYFPEFHEMGLKMIKAQ |
| | GGIFGWVAESAAVLQAIG |
| 293 | WP_012237048.1 cysteine hydrolase [Sorangium cellulosum]: |
| | MTARPLEIAARPYPFRVADRDAVALLVIDMQRDFLEPGGFGAALGNDVKRLQRIVPTVRRVLDAFRDHGL |
| | PVIHTKEGHRQDLSDCPPAKRSRGAPGMRIGDVGPMGRILVLGEPGNDFVPELAPAPGELVVPKPGKGAF |
| | YRTGLDARLAALGVSHLLIAGVTTEVCVQTTMREANDRGYECLLIEDATESYFPEFKVATLEMVRAQGAI |
| | IGWTAPAAAVLEAL |
| 294 | WP_012253274.1 cysteine hydrolase [Methylorubrum extorquens]: |
| | MPAPQPLLDAEPAPLPFDPARAALVVIDMQRDFLEPGGFGESLGNDVSLLAAAVPPARALLAAARAAGLL |
| | VVHTREGHAPDLSDAPPAKRERGAPTARIGEPGPMGRILIRGEPGHDIIPELAPLDGEPVIDKPGKGAFY |
| | ATGLAALLEARGIETLIVCGVTTEVCVHTTVREANDRGYRCVVVADACGSYVPAFHEAGLAMIKAQGGIF |
| | GWVSRSAAVIAALGQA |
| 295 | WP_012332503.1 cysteine hydrolase [Methylobacterium sp. 4-46]: |
| | MSVLLDAEPAPLAVDRATTALVIIDMQRDFLEPGGFGETLGNDVGLLGTAIGPCRAVLAAAREVGLLVVH |
| | TREGHAPDLSDAPPAKVARGAPSARIGAPGPMGRILIRGEPGHAIIPELAPAPGEVVIDKPGKGAFYATA |
| | LGEILAARRIATLLVCGVTTEVCVHTTVREGNDRGYRCVVLADACGSYVPHFHEVGLAMIKAQGGIFGWV |
| | SNTAAAIAAIRAA |
| 296 | WP_012348032.1 cysteine hydrolase [Leptothrix cholodnii]: |
| | MKPIVDARPYPYAFDPAHTAVVLIDMQRDFLEPGGFGAMLGNDVTTLQRIVPACQALLALARAHEMRVIH |
| | TQEAHDAQLLDCPPSKRARGGLSCGIGDVGPLGRVLVAGEPGAGFVAELQPLPGETVLRKPGKGAFHATG |
| | LDAMLRASGITHLLIGGVTTEVCVQTTMREANDRGYECLLVEDCAASYFPHFHAAVVEMVVAQGGIVGWA |
| | APLAAVQDALRGAMQEARPAAA |
| 297 | WP_012427105.1 cysteine hydrolase [Paraburkholderia phytofirmans]: |
| | MTSTNTLTIDAQPGPFSFDSAKTALVVIDMQRDFIEPGGFGESLGNDVSLLADIVPTVAALLAFARKHHW |
| | FVVHTRESHAPDLSDCPPAKRLRGAPNARIGDAGPMGRILIRGEPGNAIVEPLAPLAGELVIDKPGKGAF |
| | HATRLGEELAMRGVTHLVFAGVTTEVCVQTSMREANDRGYDSLLIEDATASYFPAFKQATLEMVRSQGGI |
| | VGWTAPFASLIKTDETTQAWK |
| 298 | WP_012453046.1 cysteine hydrolase [Methylorubrum populi]: |
| | MPAPQPLLDAEPAPLPFDPARTALVVIDMQRDFLEPGGFGESLGNDVSLLAAAVPPARALLAAARTAGLL |
| | VVHTREGHAPDLSDAPPAKLERGAPTARIGAPGPMGRILIRGEPGHDIVPELAPLDGEPVIDKPGKGAFY |
| | ATGLAALLEARAIETLIVCGVTTEVCVHTTVREANDRGYRCVVVADACGSYIPAFHEAGLAMIKAQGGIF |
| | GWVSQSTAVIAALGER |
| 299 | WP_012555554.1 cysteine hydrolase [Rhizobium leguminosarum]: |
| | MAGIKAEPFAFPVKHDELALIVIDMQRDFAEPGGFGASLGNDVSRITRIVPDVKRLIQGFRNAGLPVIHT |
| | MECHRPDLSDLPPAKRDRGNPSLRIGDEGPMGRILISGEPGTAILPELAPVKGEIIIEKPGKGAFYATEL |
| | GAMLKQKGISQLVFAGVTTEVCVQTTMREANDRGYECLLAEEATESYFPEFKAAAIAMIRAQGAIVGWTA |
| | HVDDILESIANA |
| 300 | WP_012696785.1 cysteine hydrolase [Laribacter hongkongensis]: |
| | MSLKTLKAEPYELEFDPGTTALIMIDMQRDFVEPGGFGEMLGNDVSLLRSAIEPCRRLLEAARKAGVFVV |
| | HTREGHRADLTDCPLAKRLRGGLECGIGDKGPMGRILVRGEYGHDIIPELYPVAGEPVVDKPGKGAFFAT |
| | DLDLLLRNRNIRTLIVCGVTVEVCVHTTVREANDRGYECVVPSDCVASYFPEFYRVALEMIKAQGGIFGW |
| | VSDADRIVEALDD |
| 301 | WP_012706456.1 cysteine hydrolase [Sinorhizobium fredii]: |
| | MAEIKAEPFPFRLDRDAVALIVIDMQRDFTEEGGFGESLGNDVARVAKIVPDVKRLIEGFRAAGLPVIHT |
| | MECHRPDLSDLPRAKRERGRPHFRIGDEGPMGRILIAGEPGTAILPELAPAVGETVIEKPGKGAFYATPL |
| | DYILKERDIGQLVFAGVTTEVCVQTTMREANDRGYECLLVEEATESYFPEFKSATLAMIRAQGAIVGWTA |
| | HLADVLRGIAHA |
| 302 | WP_012745439.1 cysteine hydrolase [Variovorax paradoxus]: |
| | MRIETANPFPYDFELKNTALVLIDMQRDFIEPGGFGETLGNDVSLLEAIVPATKAALQAWREAGGLVVHT |
| | REAHKADLSDCPPAKRNRGNPSLRIGDQGPMGRILVAGEPGNQIIDALAPVDGEMVIDKPGKGAFHATGL |
| | HELLQARGITHLLFGGVTTEVCVQTSMREANDRGYDSLLLEDCTESYFPAFKAATLDMVRAQGAIVGWTA |
| | PSSALRAVLGQGQ |
| 303 | WP_012760695.1 cysteine hydrolase [Rhizobium leguminosarum]: |
| | MMGIKAEPFAFPVKHDELALIVIDMQRDFAEPGGFGASLGNDVSRITRIVPDVKRLIQGFRNAGLPVIHT |
| | MECHRPDLSDLPPAKRDRGNPALRIGDEGPMGRILISGEPGTAILPELAPVKGEVVIEKPGKGAFYATDL |
| | GTVLQQKGIRQLVFAGVTTEVCVQTTMREANDRGYECLLAEEATESYFPEFKAAAIAMIRAQGAIVGWTA |
| | HIDDILESIAHA |
| 304 | WP_012823729.1 cysteine hydrolase [Halothiobacillus neapolitanus]: |
| | MNMDTLRVDAQPFAWRFPPARTAVIMIDMQRDFILPGGFGDTLGNDVARLKPAVTAALELLDWCRARNML |
| | VVHTKEAHAADLSDCPLAKRLRGDPTLRIGDPGSMGRILIDGEFGADFVEELTPLPGEVIITKPGKGAFY |
| | ATELGEILKAHGITHLLFGGVTTEVCVQTTMREANDRGYECLLVEEATESYFPEFKQATLAMIRAQGGIV |
| | GWTATLEALTHATPQTESQEVSADES |
| 305 | WP_013167236.1 cysteine hydrolase [Starkeya novella]: |
| | MTESLLAAEPAPLAFHPARTALVIIDMQRDFLEPGGFGETLGNDVSLLQAAVGPCKAALKAARAAGMLVI |
| | HTREGHRPDMADAPPAKVERGAPTARIGCAGPMGRILIRGEPGHDIIAELYPAPGEPVLDKPGKGAFYQT |
| | DLELMLKNRGVDTLLVAGVTTEVCVHTTIREGNDRGYRCVALADCCASYFPEFHRVGLEMVKAQGGIFGW |
| | VSDSAALKVALGNKALAASAA |
| 306 | WP_013171136.1 cysteine hydrolase [[Bacillus] selenitireducens]: |
| | MTHVQAQAKPYEFTFDPKTTALVLIDMQRDFVAPGGFGEKLGNDISATRAIIPATQEMLEAAREAGMMVI |
| | HTREGHRTDLSDCPPSKLNRGKKQGAGIGDVGPMGRILVRGEYGHDLVDELKPVEGEVVIDKPGKGAFYM |
| | TDLESVLLNRGITHLLVGGVTTHVCVQSTIREANDRGFDCLLLEDCSAAFDPKDHEDSIRMIHQQGGIFG |
| | WTSTSDEVKKALASRN |
| 307 | WP_013233427.1 cysteine hydrolase [Herbaspirillum seropedicae]: |
| | MIRIDAFPYPYQFHPRSTALVVIDMQRDFVEEGGFGSALGNDVRPLGAIVPTVAALLALARQTQMLVVHT |
| | RESHLPDLSDCPRTKRLRGNPTLGIGDVGPMGRILVRGEPGNQILPQLTPMAGEIVIDKPGKGAFYATDL |
| | HAQLQERGITHLLVAGVTTEVCVQTSMREANDRGYECLVVEDACASYFPAFHRATLDMLTAQGGIVGWRA |
| | PLAQLQSAVTAYAGEHP |
| 308 | WP_013424639.1 cysteine hydrolase [Frankia inefficax]: |
| | MTSAPLTVPARPYEFTFDPATTALVVIDMQRDFMEPGGFGESLGNDVSQLRSTIEPLKAVFAAARAAGLT |
| | VIHTREGHLPDLSDLPPAKLNRGNASLKIGDLGPKGRILIRGEYGQDIIDELAPIEGEFVVDKPGKGAFY |
| | ATSFGDILTEKGITSLVVTGVTTEVCVHTTVREANDRGYECLVLSDCVGSYFPEFQRVALEMIAAQGGIF |
| | GWVAPSAAFIEALAPLPAASAAQ |
| 309 | WP_013538750.1 cysteine hydrolase [Variovorax paradoxus]: |
| | MRIEQANPFAYEFEIRNTALVLIDMQRDFIEPGGFGETLGNDVSLLEAIVPATQAALAAWRKAGGLVVHT |
| | REAHKPDLSDCPPVKRNRGNPSLRIGDEGPMGRILVAGEPGNQIIDGLAPIDGELVIDKPGKGMFYATGL |
| | HKVLQQRGITHLLFGGVTTEVCVQTSMREANDRGYDGLLLEDCTESYFPAFKAATLDMIRAQGAIVGWTA |
| | PSAALLAALSHAT |
| 310 | WP_013652706.1 cysteine hydrolase [Polymorphum gilvum]: |
| | MITVKAQPFDFAFDPASVALIVIDMQRDFIEPGGFGETLGNDVSHLQRAVQPTADLLALFRTRGWPVIHT |
| | REDHLPDLSDCPPSKRNRGAPSLRIGDNGPMGRILVRGEPGAEIVPACAPCAGEIVVDKPGKGAFHATDL |
| | GAILAGLGTRSLVFAGVTTEVCVQTTMREANDRGFDCLLIEDATESYFPAFKAATLDMIRAQGGIVGWTT |
| | PLARLVQALEGEPT |
| 311 | WP_013673376.1 cysteine hydrolase [Pseudonocardia dioxanivorans]: |
| | MRVPVQVDASPAPFTFDPATTALVVIDMQRDFCEPGGFGETLGNDVSLLRSVIPPLQQVLATARELGMTV |
| | IHTREGHVPDLSDCPPAKLNRGNPSLRIGDPGPKGRILVRGEYGHDIVDELAPARGELVIDKPGKGSFHG |
| | TTFGAELEARNIRSLVVTGVTTEVCVHTTVREANDRGYECLVLSDCTGSYFPEFHRVALEMVAAQGGIFG |
| | WVAPSTALFDALVKESAA |
| 312 | WP_013698611.1 cysteine hydrolase [Burkholderia gladioli]: |
| | MHFEVPARPAPYRYDPAHTALIVIDMQRDFIEPGGFGAALGNDVAPLAAIVPSVAALLAFARARGWSVVH |
| | TRESHAPDLSDCPPAKRLRGAPDLRIGDSGPMGRILVRGEPGNQIVEALAPLAGETVIDKPGKGAFHATA |
| | LDALLRERGITHLVFAGVTTEVCVQTSMREANDRGYDCLLVEDATASYFPAFKAACLEMISSQGGIVGWT |
| | ASLRALLEAAPLPAAPSASPRP |
| 313 | WP_013893346.1 cysteine hydrolase [Mesorhizobium opportunistum]: |
| | MAEIAARPFPFAFKPRTMALVVIDMQRDFAEPGGFGASLGNDVSRVVAIVPTVKRLIEGFRAAGLPVIHT |
| | MECHRPDLSDLPPAKRNRGNPSIRIGDAGPMGRVLIAGEPGTAILDALAPLPGEIVIEKPGKGAFYATSF |
| | GDDLKRLGAQHLVFAGVTTEVCVQTTMREANDRGYECLLAEDATESYFPEFKAAALAMIRAQGAIVGWTA |
| | TTDQVLEGIANA |
| 314 | WP_013963783.1 cysteine hydrolase [Roseobacter litoralis]: |
| | MVKIDAALPFVFTFDPATTALVVIDMQRDFIERGGFGETLGNDVSLLQGIVPTASALLAFCRGKGIEVIH |
| | TRECHKPDLSDLPMSKRDRGAPSLRIGDPGPMGRILVAGEDGADIIPELYPIAGELVIDKPGKGAFFATP |
| | LGDHLRQRRITSLIFAGVTTEVCVQSTMREANDRGYDCLLIEDATESYFPSFKTATLEMIRAQGAIVGWT |
| | AQFAQLEAAWHD |
| 315 | WP_014744708.1 cysteine hydrolase [Tistrella mobilis]: |
| | MPVTITIDAAPYAFTASADRLALIVIDMQRDFLEPGGFGASLGNDVARVRPSIAPTRRLLDGFRAAGLPV |
| | FHTRECHLPDLSDCPPAKHGRGPGPLRIGDPGPMGRILIRGEPGADIIPELAPLPGEVVIDKPGKGAFHA |
| | TSLGDELARRGISHLVFAGVTTEVCVQTTMREANDRGFDCLLATDATDSYFPEFKAATVAMITAQAGIVG |
| | WAAPVDAVLAGLRADT |
| 316 | WP_014763478.1 cysteine hydrolase [Sinorhizobium fredii]: |
| | MAQIKAEPFPFRLRRDAVALIVIDMQRDFTEEGGFGASLGNDVSRLMKIVPDVKRLIERFRAAGLPVIHT |
| | MECHRPDLSDLPRAKRERGNPRLRIGDEGPMGRILIAGEPGTAILPELAPVVGETVIEKPGKGAFHATPL |
| | DDILKERRIAQLIFAGVTTEVCVQTTMREANDRGYECLLVEEATESYFPEFKAATLAMIRAQGAIVGWTA |
| | HLADVIKEIAHA |
| 317 | WP_014927395.1 MULTISPECIES: cysteine hydrolase [Gordonia]: |
| | MSDTVTLDAQPGPIELDLAHTALIIIDMQRDFLLPGGFGETLGNDVSQLQRVVAPLAGLLDAARAAGMLI |
| | VHTREGHLPDLSDCPPAKLNRGLPSKRIGDPGAFGRILIRGEYGHDIVDELAPLDTEVVIDKPGKGAFYA |
| | TELAAILADNDITQLLVTGVTTEVCVHTTTREANDRGFECVVVSDCVGSYFPEFQRVGLEMIAAQGGIFG |
| | WTAPSEDVITLLTPATAEDWGSA |
| 318 | WP_014993115.1 cysteine hydrolase [Alcanivorax dieselolei]: |
| | MLSVTAKPDAFPLDPGHCALVVIDMQRDFIEPGGFGAALGNDVSRLAPVVPRVAALLALAREQRLTVIHT |
| | RESHLPDLSDCPPLKRNKLPAGRRIGDDGPMGRILVRGEPGNRILDAVAPEPGEWQVDKPGKGMFHATGL |
| | DQRLRDAGITQLIFAGVTTEVCVQTSMREACDRGYDCLVIEDATESYFPEFKAATLAMIVAQGGIVGRCA |
| | SLDALRRAFQQGVSA |
| 319 | WP_015168474.1 cysteine hydrolase [Synechococcus sp. PCC 7502]: |
| | MTAIAAQPYEYELPTEGKIALVIIDMQRDFLEPGGFGDALGNNVQLLQAIVPTVKALLETWRSLSLPIIH |
| | TIECHKPDLSDCPTSKLNRGKGGLKIGDLGPMGRILVYGEEGNNIIPELAPKDGEIVILKPGKGAFTRTD |
| | LEAILQKEGITHLVITGVTTEVCVQTTMREANDRGYECLLVEDATESYFPEFKEATIKMLRAQGGIIGWT |
| | TDAKSVISALSAHS |
| 320 | WP_015183226.1 cysteine hydrolase [Microcoleus sp. PCC 7113]: |
| | MVSISAQPYDYELPAHGGLALLI1DMQRDFLEEGGFGDALGNDVTRLRAIVPTLKELLAAFRAYKLPIFH |
| | TIEGHQPDLSDCPPSKRHRGRGELKIGDVGPMGRILVLGESGNGIIPELQPLPGETVITKPGKGAFYNTH |
| | LESLLHEQGITHLLITGVTTEVCVQTTMREANDRGFECLLVEDATESYFPAFKQSTLDMIVAQGGIVGWT |
| | ASAANVLQSLAKWKS |
| 321 | WP_015186981.1 cysteine hydrolase [Gloeocapsa sp. PCC 7428]: |
| | MVLIAAQPYDYELPSELQKVALLIIDMQRDFLEPGGFGEALGNDVRHLSAIIPTLKSLLEIFRKRQLPVF |
| | HTVEGHQPDLSDCPPSKLRRGNGQLKIGDPGPMGRILILGELGNAIIPELQPMTGEIVISKPGKGAFYQT |
| | SLESYLHKQGITHLIITGVTTEVCVQTTMREANDRGFECLLVEDATASYFPEFKESTLEMIRAQGGIVGW |
| | TATAANVMQAFGSI |
| 322 | WP_015307204.1 cysteine hydrolase [Mycobacterium sp. JS623]: |
| | MSPIEVLAEPSPFRLVAGQTALIVIDMQRDFLLPGGFGESLGNDVNQLLKVVPPLAELIAAARTAGVLVI |
| | HTREGHEPDLSDCPPAKLNRGAPSKRIGDDGKYGRILIRGEYGHDIIDELAPIDGEVVIDKPGKGAFHAT |
| | DLQDILSDAGITQLLVTGVTTEVCVHTTTREANDRGYECLVVSDCVGSYFPEFQRVGLDMIKAQGGIFGW |
| | VADTAAVIPALRTLTSSAA |
| 323 | WP_015343701.1 MULTISPECIES: cysteine hydrolase [Rhizobium]: |
| | MTDIKAQPFPFPLQREAAALIVIDMQRDFAEPGGFGASLGNDVSRITRIIPDVKRLIAGFRSAGLPVIHT |
| | MECHRPDLSDLPPAKRDRGNPSLRIGDKGPMGRILIAGEPGTAILPELAPIDGEIVIEKPGKGAFYATGL |
| | GDILMRKGIKQLVFAGVTTEVCVQTTMREANDRGYECLLVEEATESYFPEFKSAAIAMIQAQGAIVGWTA |
| | HIDDILEAINHA |
| 324 | WP_015668267.1 cysteine hydrolase [Bradyrhizobium oligotrophicum]: |
| | MANPSANPSVSASATATITAEPEPITLDLAATALVIIDMQRDFMEPGGFGETLGNDVSQLARAVAPIANV |
| | LAAARDMGMLVVHTREGHKPDLSDAPPAKVERGAPSLRIGDPGPMGRILIRGEPGHDIIPALYPAEGEVV |
| | IDKPGKGAFYATELGNILKQHGIANLLVCGVTTEVCVNTTVREANDRGYRCVVISDGCASYFPEFHEMGL |
| | RMIKAQGGIFGWVAPSAAVLEAMSS |
| 325 | WP_015687739.1 cysteine hydrolase [Bradyrhizobium sp. S23321]: |
| | MLNSTKPTLGVISAEPDPIKLDWASTALIIIDMQRDFMEPGGFGETLGNDVSQLARAVKPIGALLNAARD |
| | SGMLVVHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKTSTT |
| 326 | WP_015747308.1 cysteine hydrolase [Nakamurella multipartita]: |
| | MSDPITIPAQPGPFPFDVASTALVVIDMQRDFLLPGGFGETLGNDVALLRQVVPPLVELLAAARAAGMLV |
| | IHTREGHEPDLSDCPPAKLRRGKPSARIGDPGALGRILIRGAYGHDIIDELAPIAGEIVIDKPGKGAFYA |
| | TSFGDVLVEHGITHLIVTGVTTEVCVHTTVREANDRGYDALVVSDCVGSYFPEFQQIGLQMIAAQGGIFG |
| | WVADSAAVIAGLSAGTALETAGAPHTALAG |
| 327 | WP_015795027.1 cysteine hydrolase [Catenulispora acidiphila]: |
| | MTARTVSVPASPAPFALDPGSAALILIDMQRDFLEPGGFGESLGNDVSLLRKTIAPLQAVLAAARASGMP |
| | VIHTREGHLPDLSDCPPSKLNRGAPSMRIGDQGPKGRILIRGEYGHDIVDELAPAPGEPVVDKPGKGAFY |
| | ATAFGEILGGLGVTQLIVTGVTTEVCVHTTVREANDRGFECLVLSDCVGSYFADFQEAALAMIAAQGGIF |
| | GWTATSADYLAALESAAGADAITTAS |
| 328 | WP_015822266.1 cysteine hydrolase [Methylorubrum extorquens]: |
| | MPAPQPLLDAEPAPLPFDPARAALVVIDMQRDFLEPGGFGESLGNDVSLLAAAVPPARALLAAARAAGLL |
| | VVHTREGHAPDLSDAPPAKLERGAPTARIGEPGPMGRILIRGEPGHDIIPELAPLDGEPVIDKPGKGAFY |
| | ATGLAALLEARGIETLIVCGVTTEVCVHTTVREANDRGYRCVVVADACGSYIPAFHEAGLAMIKAQGGIF |
| | GWVSRSAAVIAALGQA |
| 329 | WP_016558332.1 cysteine hydrolase [Rhizobium grahamii]: |
| | MVDIAAEPFDFTAKRDELALVVIDMQRDFAEPGGFGASLGNDVSRIARIVPDVKRLIEGFRRAGLPVIHT |
| | MECHKPDLSDLPPAKRNRGRPSLKIGDEGPMGRILISGEPGTAILPELAPVDGEIVIEKPGKGAFYATPL |
| | GGILQEMGISQLVFAGVTTEVCVQTTMREANDRGFECLLAEEATESYFPEFKRAAIEMIRAQGAIVGWTA |
| | RVDDILKGITDA |
| 330 | WP_016567343.1 cysteine hydrolase [Pseudomonas syringae]: |
| | MIKVNARPDRFAFDTSRTAVVIIDMQRDFLEPGGFGAALGNDVAPLQAIVPSVQRLLTLARNEGMAVIHT |
| | RESHRPDLADCPQAKRDHGSPGLRIGDPGPMGRILIRGEPGNQIIDTLAPLAGEWIIDKPGKGMFFATDL |
| | QQRLSEAGITHLIFAGVTTEVCVQTSMREANDRGYRCLLIEDATESYFPAFKQATLEMITAQGGIVGRVA |
| | SLMDLEQALQTRSTH |
| 331 | WP_016980454.1 MULTISPECIES: cysteine hydrolase [Pseudomonas syringae |
| | group]: |
| | MISVNARPDCFTFAPSCAAVVIIDMQRDFLEPGGFGAALGNDVVPLQAIVPSVQQLLALARDQGITVIHT |
| | RESHSADLADCPPAKLAHGSPGLRIGDPGPMGRILIRGEPGNQIIDSLTPLACEWVIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTS |
| 332 | WP_017288003.1 cysteine hydrolase [Leptolyngbya boryana]: |
| | MPSIAAQPYEYELPSDSQVALIVIDMQRDFLELGGFGEALGNDVKLAQAIVPTVKQLLEGCRSLHLPIFH |
| | TQEGHLPDLSDCPQSKLKRGKGNLTIGDQGKLGRILILGEPGNAIIPELAPLPGEVLIPKPGKGAFYNTD |
| | LEMQLINRNVTHLLIAGVTTEVCVQTTMREANDRGYECLLVEDATESYFPQFKQATLEMVRAQGGIVGWT |
| | ANTEAVLQGLRSWKAGE |
| 333 | WP_017682528.1 cysteine hydrolase [Pseudomonas syringae]: |
| | MISISARPDTFTFEPSRTAVVIIDMQRDFLEPGGFGAALGNDVAPLQAIVPTVQQLLALAREQGLVVIHT |
| | RESHRPDLSDCPQAKLDHGLPGLRIGDPGPMGRILVRGEPGNQIIDALTPLASEWVIDKPGKGMFFATDL |
| | HQRLTAAGITHLIFAGVTTEVCVQTSLREACDRGYRCLLIEDATESYFPAFKQATLDMITAQGAIVGRVT |
| | SLANLQHGLHTRSTQ |
| 334 | WP_017708689.1 cysteine hydrolase [Pseudomonas syringae]: |
| | MISISARPDTFTFKPSRTAVVIIDMQRDFLEPGGFGAALGNDVAPLQAIVPTVQQLLALAREQGLVVIHT |
| | RESHRPDLSDCPQAKLDHGLPGLRIGDPGPMGRILVRGEPGNQIIDALTPLASEWVIDKPGKGMFFATDL |
| | HQRLTAAGITHLIFAGVTTEVCVQTSLREACDRGYRCLLIEDATESYFPAFKQATLDMITAQGAIVGRVT |
| | SLANLQHGLHTRSTQ |
| 335 | WP_017805457.1 cysteine hydrolase [Avibacterium paragallinarum]: |
| | MLKQFQAEPFPLSFNPQTTALLMIDMQRDFVEPGGFGEALGNDVNLVRSAIQPCKRMLSAARQAGIFILH |
| | TREGHRADLSDCPPAKLTRGGKTFIGECGPKGRILIRGEEGHDIIPELYPIAGEPIIDKLGKGAFYQTDL |
| | HLILQNRGIKTLIVCGVTTEVCVNTTVREANDRGYECIIPEDCVGSYFPEFQEYALKMIKAQGAIFGWVS |
| | TSTEIINALMS |
| 336 | WP_018070733.1 cysteine hydrolase [Rhizobium leguminosarum]: |
| | MMGIKAEPFAFPVKHDELALIVIDMQRDFAEPGGFGASLGNDVSRITRIVPDVKRLIQGFRNAGLPVIHT |
| | MECHRPDLSDLPPAKRDRGNPALRIGDEGPMGRILISGEPGTAILPELAPVKGEVVIEKPGKGAFYATDL |
| | GTVLQQKGIRQLVFAGVTTEVCVQTTMREANDRGYECLLAEEATESYFPEFKAAAIAMIRAQGAIVGWTA |
| | HVDDILESIAHA |
| 337 | WP_018246803.1 cysteine hydrolase [Rhizobium leguminosarum]: |
| | MAKIKAEPFAFPVKHDELALIVIDMQRDFAEPGGFGASLGNDVSRITRIVPDVKRLILGFRNAGLPVIHT |
| | MECHRPDLSDLPPAKRDRGNPSLRIGDEGPMGRILISGEPGTAILPELAPVKGEVVIEKPGKGAFYATEL |
| | GTVLQEKGIKQLVFAGVTTEVCVQTTMREANDRGYECLLAEEATESYFPEFKAAAIAMIRAQGAIVGWTA |
| | HVDDILESIAHA |
| 338 | WP_019332186.1 cysteine hydrolase [Pseudomonas syringae]: |
| | MISISARPDTFTFEPSHTAVVIIDMQRDFLEPGGFGAALGNDVAPLQAIVPTVQQLLALAREQGLVVIHT |
| | RESHRPDLSDCPQAKLDQGLPGLRIGDPGPMGRILVRGEPGNQIIDALTPLASEWVIDKPGKGMFFATDL |
| | HQRLTAAGITHLIFAGVTTEVCVQTSLREACDRGYRCLLIEDATESYFPAFKLATLDMITAQGAILGRVA |
| | SLANLQHALHTRSTP |
| 339 | WP_019510032.1 MULTISPECIES: cysteine hydrolase [Mycobacteriaceae]: |
| | MSQHVAIPATPEPFTLVAGRTALVIIDMQRDFLLAGGFGESLGNDVGQLLKVVPPLAALLTAAREAGVMV |
| | IHTREGHRPDLSDCPPAKLQRGVPSKRIGDKGRFGRILIRGEYGHDIIDELRPLDGEVVIDKPGKGAFYD |
| | TELAEVLAGAGITQLLITGVTTEVCVHTTTREANDRGYECLVVSDCVGSYFPEFHRIGLEMIAAQGGIFG |
| | WVADSAAVLTALHTLTIDAA |
| 340 | WP_020103397.1 MULTISPECIES: cysteine hydrolase [unclassified |
| | Mycobacterium]: |
| | MTSVAVPAAPTPFTLTAGQTALIVIDMQRDFLLPGGFGESLGNDVDQLLKVVPPLAALLAAARAAGVMVI |
| | HTREGHEPDLSDCPPAKLNRGAPSKRIGDPGKYGRILIRGEYGHDIVDELAPIDGELVIDKPGKGAFYAT |
| | GLQDALTGAGITQLLVTGVTTEVCVHTTTREANDRGYECLVVSDCVGSYFPEFQRVGLEMIAAQGGIFGW |
| | VADTAAVIPALQQLAAPSPSAV |
| 341 | WP_020304635.1 MULTISPECIES: cysteine hydrolase [Pseudomonas syringae |
| | group]: |
| | MISISARPDTFTFEPSRTAVVIIDMQRDFLEPGGFGAALGNDVAPLQAIVPTVQQLLALAREQGLVVIHT |
| | RESHRPDLSDCPQAKLDQGLPGLRIGDPGPMGRILVRGEPGNQIIDALTPLASEWVIDKPGKGMFFATDL |
| | HQRLTAAGITHLIFAGVTTEVCVQTSLREACDRGYRCLLIEDATESYFPAFKQATLDMITAQGAIVGRVT |
| | SLANLQHGLHTRSTQ |
| 342 | WP_020458383.1 cysteine hydrolase [Frankia sp. EAN1pec]: |
| | MTPTAPLTVSARPYEYTFDPATTALVLIDMQRDFMEPGGFGESLGNDVSQLRSTIEPLAAVLAAARAVGL |
| | TVIHTREGHLPDLSDLPPAKLNRGGAALKIGDVGPKGRILIRGEYGQDIIDELAPAEGEPVIDKPGKGAF |
| | YATEFGDVLKARGITSLVVTGVTTEVCVHTTVREANDRGYECLVLSDCVGSYFSEFQRVALEMIAAQGGI |
| | FGWVASSEQFLDALAVLGASAVAS SAVAAS |
| 343 | WP_020727561.1 cysteine hydrolase [Mycobacterium marinum]: |
| | MPIIDARPFPYQFDINHAALICIDMQRDFVMSGGFAESLGNDVKKVAPCIPVIRELQDACRRIGVPVIHT |
| | KECHKPDLSDLPTAKLNRGNPKMKIGSVGPLGRILIDGEGGSDFIAENYPAPGELAISKPGKDAFYRTNL |
| | HEYLIGRNISNLVITGITTEVCVQTTMRCANDRGYDCLLVEDGTDSYFPEFKEMTLRALVAQGGIVGWTC |
| | TSDKILEALES |
| 344 | WP_020737315.1 cysteine hydrolase [Sorangium cellulosum]: |
| | MTARAPEIAAKPYPFRLAEPDAVALLVIDMQRDFLEPGGFGAALGNDVRRLQRIVPTVRSLLDAFRERGL |
| | TVLHTKEGHRPDLSDCPPAKRSRGAPGMRIGDVGPMGRILVLGEPGNDFVPELAPAPGELVIPKPGKGAF |
| | YRTGLDARLAALGVSQLLIAGVTTEVCVQTTMREANDRGYECLLIEDATESYFPEFKAATLEMVRAQGAI |
| | VGWTAPAAAVLKAL |
| 345 | WP_020923002.1 cysteine hydrolase [Rhizobium etli]: |
| | MAEIKAEPFAFQVKHDQLALIVIDMQRDFAEPGGFGASLGNDVSRITRIVPDVKRLIQGFRYAGLPVIHT |
| | MECHRPDLSDLPPAKRDRGNPTLRIGDVGPMGRILISGEPGTAILPELAPVKGEVVIEKPGKGAFFATEL |
| | DEVLQQKGIKQLVFAGVTTEVCVQTTMREANDRGYECLLAEEATESYFPEFKAAAIAMIRAQGAIVGWTA |
| | HVNDILESIAHA |
| 346 | WP_021005030.1 cysteine hydrolase [Variovorax paradoxus]: |
| | MRIETANPFPYDFELKNTALVLIDMQRDFIEPGGFGETLGNDVSLLEAIVPATQAALEAWRKAGGLVVHT |
| | REAHKADLSDCPPAKRNRGSPSLRIGDEGPMGRILVAGEPGNQIIDALAPVDGEIVIDKPGKGAFYATGL |
| | HELLQRRGITHLLFGGVTTEVCVQTSMREANDRGYDSLLLEDCTESYFPAFKAATLDMVRAQGAIVGWTA |
| | PSTALMAALRQGQ |
| 347 | WP_021523614.1 cysteine hydrolase [Escherichia coli]: |
| | MTQSIFQAQPFELPFDPRTTALVMIDMQRDFVEAGGFGEALGNDVSLVRTAIAPCAEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWI |
| | TDSKAIIAGLEG |
| 348 | XP_005772344.1 hypothetical protein EMIHUDRAFT 65528, partial |
| | [Emiliania huxleyi CCMP1516]: |
| | MPSTGRVALLMIDWQRDFLDEGGFGHCLGNDVAPLRTALKPAAAVLAAARAAGVTVVHTLEAHTADLADC |
| | PPAKLTRCPAIGTVLDAARGRVLVAGEPGNAIVDEVAPVAGELIVHKPGKGAFFNTRLHDELQRLGVTHL |
| | LLTGVTTEVCVQTSMREANDRGYECLVVADATESYFPQLKRAALEMIVAQGGIVGWAAPSADVIAAL |
| 349 | XP_005780363.1 hypothetical protein EMIHUDRAFT 468804 [Emiliania |
| | huxleyi CCMP1516]: |
| | MLRSQPYSWPCGGPLDASTTALVLIDMQHDFCGKGGYVDRMGYDISATRAPIKPLQRVLAAARAAGVRVI |
| | HTREGHRPSLADLPQNKRLRSTAIGTEIGQPGPCGRVLVRGEPGWELIDELRPLPSEDIIDKPGKGSFFA |
| | TDLEHLLRTTGAGPRGHCVKTAASGCCSAASRRTCNHAAAHKMVTMQGGVFGAIASSDAVLQVLDAMPRA |
| | RDSAPAAAPPPWLLPPPPPRAISGMEAAGLVLLAFAAGAALGARLR |
| 350 | XP_005790342.1 hypothetical protein EMIHUDRAFT 70288, partial |
| | [Emiliania huxleyi CCMP1516]: |
| | MPSTGRVALLMIDWQRDFLDEGGFGHCLGNDVAPLRTALKPAAAVLAAARAAGVTVVHTLEAHTADLADC |
| | PPAKLTRCPAIGTVLDAARGRVLVAGEPGNAIVDEVAPVAGELIVHKPGKGAFFNTRLHDELQRLGVTHL |
| | LLTGVTTEVCVQTSMREANDRGYECLVVADATESYFPQLKRAALEMIVAQGGIVGWAAPSADVIAAL |
| 351 | XP_005847593.1 hypothetical protein CHLNCDRAFT 23353, partial |
| | [Chlorella variabilis]: |
| | VAASPYPYPLPVEHTALVMIDFQRDFMEAGGFGETLGNNVALLRTSLEPGARLLAAARQAGMLVVHTLEA |
| | HKADLSDLPPAKQLRGNLPPELRIGAEGDMGRILIRGEPGNGIVPEVAPIDGEWQVHKPGKGAFWATGLH |
| | EKLQARGITHLLFAGVTTEVCVQTSMREANDRGYECLLVTDATDSYFPKFKEAAIEMIRAQGGIVGWTAD |
| | TAAVEAALAAA |
| 352 | XP_005847594.1 hypothetical protein CHLNCDRAFT 133869 [Chlorella |
| | variabilis]: |
| | MSTIPVCAVPEGAVVEVAASPYPYPLPVEHTALVMIDFQRDFMEAGGFGETLGNNVALLRASLEPGARLL |
| | AAARKAGMLVVHTLEAHKPDLSDLPRSKQLRGNLPPELRIGAEGAMGRILVAGEPGNGIVPEVAPIEGEW |
| | QVHKPGKGAFWATGLHEKLQARGITHLLFAGVTTEVCVQTSMREANDRGYECLLVTDATGKPSAAYFPEF |
| | KEAAIKMIEAQGGIVGWTADTAAVEAALTAMPNP |
| 353 | WP_023070916.1 cysteine hydrolase [Leptolyngbya sp. Heron Island J]: |
| | MNVHQITVPARPYSLKLDLAHTALLVIDMQNDFCTLGGWADCKGFDVSQTQKPIKPLQTLLQSLRQTPVT |
| | IIHTREGHRPDLSDCPPHKLARSKKQNAEIGSEGVMGRLLTRGSKSHDFVDELQPISGEIVLDKPGKGAF |
| | VATDLDLILRQRGIQQLLLTGVTTECCVHTTLRTANDLGYECLLLEDCCASLKPEFHRVSVEMTQTIFGW |
| | VTTSKQLLTAVNLQAA |
| 354 | WP_023145545.1 cysteine hydrolase, partial [Escherichia coli]: |
| | MTQSIFQAQPFELPFDPRTTALVMIDMQRDFVEAGGFGEALGNDVSLVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQK |
| 355 | WP_023492001.1 cysteine hydrolase [Serratia sp. DD3]: |
| | MTQQVFHAEPFDLPFDPASTALVMIDMQRDFVEPGGFGEALGNDVSFVRSAIEPCKRVLDAARSQGLLVI |
| | HTREGHRADLSDCPPAKLTRGGQTFIGTHGPMGRILVRGEAGHDIIPELYPQAGEPVIDKPGKGAFYQTD |
| | LHLILQNHGIKTLIVCGVTTEVCVNTTVREANDRGYECIIPQDCVGSYFPEFQKYALEMIKAQGAIFGWV |
| | SDANAIVAGLQK |
| 356 | WP_023495172.1 cysteine hydrolase [Methyloglobulus morosus]: |
| | MSTIEINAEPEAIAIEIAKTAVVMIDMQRDFLEPGGFGESLGNDVSLLSAAIEPCKALLDAARQHEMLVI |
| | HTREGHLPDLSDAHKAKVERGDPSLRIGQLGPMGRILIRGEPGQDIIPELYPQLGEPVIDKPGKGAFYAT |
| | DLQSILETNGIENLIVCGVTTEVCVHTTVREANDRGYRCIVPGDCCGSYIPEFHEVGLRMIKAQSGIFGW |
| | VTDSHTILTAMNI |
| 357 | WP_023561467.1 cysteine hydrolase [Actinoplanes friuliensis]: |
| | MPTVEAQPGPFTFDPATTALLVIDMQRDFLEPGGFGESLGNDVSQLRRTIAPLAAFMTTWRAAGLPVIHT |
| | REGHLPDLSDCPPAKLERGAPSKRIGDPGAFGRILIRGEYGHDIIDELQPAPGEAVVDKPGKGAFYATEL |
| | QELLDKGGIRSLLVAGVTTEVCVHTTVREANDRGYECLVLADCVGSYFPEFQRVGLEMIAAQGGIFGWVA |
| | DSTSVPLQELS |
| 358 | WP_023677214.1 cysteine hydrolase [Mesorhizobium sp. LSJC280B00]: |
| | MAEIEALPFPFAFKPEAMALVVIDMQRDFAEPGGFGASLGNDVSRVVAIVPTVKRLIEGFRTAGLPVIHT |
| | MECHKADLSDLPPAKRNRGNPSIRIGDVGPMGRVLIVGEPGTAILDELAPLPGEIVIEKPGKGAFYATSF |
| | GDDLKRLGAQQLVFAGVTTEVCVQTTMREANDRGYECLLAEDATESYFAEFKAAALAMIRAQGAIVGWTA |
| | TTDQVLEGIANA |
| 359 | WP_023720641.1 cysteine hydrolase [Mesorhizobium sp. LSHC420B00]: |
| | MAEIEALPFPFAFKPEAMALVVIDMQRDFAEPGGFGASLGNDVSRITAIVPTVKRLLEGFRAAGLPVIHT |
| | MECHKADFSDLPPAKRNRGNPSIRIGDIGPMGRVLIVGEPGTAILDELAPLPGEIVIEKPGKGAFYATSF |
| | GDDLKRLGAQQLVFAGVTTEVCVQTTMREANDRGYECLLAEDATESYFPEFKTAALAMIRAQGAIVGWTA |
| | TTDQVLEGISNA |
| 360 | WP_023759783.1 cysteine hydrolase [Mesorhizobium sp. LNHC252B00]: |
| | MAEIAAQPFPFAFKRESMVLVVIDMQRDFAEPGGFGASLGNDVSRVVAIVPTVKRLIEGFRAAGLPVIHT |
| | MECHRPDLSDLPPAKRNRGNPTIRIGDVGPMGRVLIVGEPGTAILDELAPLPGEIVIKKPGKGAFYATSF |
| | NEDLKRLGAGQLVFAGVTTEVCVQTTMREANDRGYECLLAEDATESYFPEFKAAAIAMIRAQGAIVGWTA |
| | TTDQVLKGIANA |
| 361 | WP_023765233.1 MULTISPECIES: cysteine hydrolase [unclassified |
| | Mesorhizobium]: |
| | MAEIAAQPFPFAFKPRTMALVVIDMQRDFAEPGGFGASLGNDVSRVVAIVPTVKRLIEGFRAAGLPVIHT |
| | MECHRPDLSDLPPAKRNRGNPSIRIGDAGPMGRVLIAGEPGTAILDALAPLPGEIVIEKPGKGAFYATSF |
| | GDDLKRLGAQHLVFAGVTTEVCVQTTMREANDRGYECLLAEDATESYFPEFKAAALAMIRAQGAIVGWTA |
| | TTDQVLEGIANA |
| 362 | WP_023781542.1 cysteine hydrolase [Mesorhizobium sp. LNHC220B00]: |
| | MAEIDALPFAFAFKPGTMALVVIDMQRDFAEPGGFGASLGNDVSRITAIVPTVKKLIEGFRAARLPVIHT |
| | MECHRSDLSDLPPAKRNRGNPSIRIGDIGPMGRVLISGEPGTAILDELAPLPGEIVIEKPGKGAFHATSF |
| | GEDLKRLGVEQLVFAGVTTEVCVQTTMREANDRGYECLLAEDATESYFAEFKTAALAMIRAQGAIVGWTA |
| | TTDQVLEGIANAQS |
| 363 | WP_023794306.1 MULTISPECIES: cysteine hydrolase [unclassified |
| | Mesorhizobium]: |
| | MAEIDALPFAFAFKPGTMALVVIDMQRDFAEPGGFGASLGNDVSRITAIVPTVKKLIEGFRAARLPVIHT |
| | MECHRSDLSDLPPAKRNRGNPSIRIGDIGPMGRVLISGEPGTAILDELAPLPGEIVIEKPGKGAFHATSF |
| | GEDLKRLGVEQLVFAGVTTEVCVQTTMREANDRGYECLLAEDATESYFAEFKMAALAMIRAQGAIVGWTA |
| | TTDQVLEGIANAQS |
| 364 | WP_023800140.1 cysteine hydrolase [Mesorhizobium sp. L48C026A00]: |
| | MAEIDALPFPFAFKPEAVALVVIDMQRDFAEPGGFGASLGNDVGRVVAIVPTVKRLIQGFRAAGLPVIHT |
| | MECHRSDLSDLPPAKRNRGNPSIRIGDVGPMGRVLVVGELGTAILDEVAPLPGEIVIEKPGKGAFYATSF |
| | GEDLKRLGVQQLVFAGVTTEVCVQTTMREANDRGYECLLAEDATESYFPEFKAAALAMIRAQGAIVGWTA |
| | MTDQVLKGIADA |
| 365 | WP_023954467.1 cysteine hydrolase [Williamsia sp. D3]: |
| | MTVVTVDTAVPAPFPLELGRTALLVIDMQRDFVLPGGFGESLGNDVSLLLDVVPPLAALIDAARSAGIMI |
| | IHTREGHKPDLSDCPPSKLRRGAASKRIGDPGRYGRILIQGEYGHDIVDELAPIAGEVVIDKPGKGAFYA |
| | TDLQQILTDAGITQLLVTGVTTEVCVHTTTREANDRGYECLVVSDCVGSYFPDFQRVGLEMISAQGGIFG |
| | WVADSTAAIAALSLIPDPSQHS |
| 366 | WP_023959513.1 cysteine hydrolase [Paenibacillus sp. JCM 10914]: |
| | MSEVVHVNVGEARPYSFSFELHHTALIIIDMQNDFCSPGGFGELLGNDIEPARAIIPAVSSILGAARDSG |
| | MLVLHTREGHLPDLSDCPPAKLERSKKQGAGIGDPGPMGRLLIRGEPGQDIVPELYPAEGEVVIDKPGKG |
| | AFYATELEAILQLNGIESLILCGVTTHVCVHTTLREANDRGYRCLVVEDATAAFDERDHEAALHMVRQQG |
| | GIFGWTVPSESLLSSIADSLKNNAG |
| 367 | YP_008998670.1 isochorismatase hydrolase (plasmid) [Escherichia coli |
| | ACN001]: |
| | MTISIFQAQPFELPFDPCTTALIMIDMQRDFVEAGGFGEALGNDVSLVRTAIAPCKEVLAAARQKGIMVI |
| | HTREGHREDLSDCPSAKLTRGGKTFIGEPGPMGRILVRGEAGHDIIPELYPVTGEPVIDKPGKGAFYQTD |
| | LHLILQKRGIKTLIVCGVTTEVCVTTTVREANDRGYECIIPEDCVGSYFPEFQKYALEMIKAQGAIFGWV |
| | TDSKAIIAGLEG |
| 368 | XP_006967923.1 predicted protein [Trichoderma reesei QM6a]: |
| | MASESKLTLSTAFPYAFTFSPSTTAFVVIDMQRDFLDSNGFGSNIDENPAIFPPVCRIVPPLPHFLRVAR |
| | RLGLHIIYTREGYLPNLADLPAARRLRETKAPTGGQSVNISDERPTGRFSVKGEASHDIIDELKPWPTEL |
| | IVDKPGKGSFWGTRLHRLLLARGITHLILTGVNTECCVTSMLHDGNDRGYECCILSGCTTGFDENMVASS |
| | LDLVWGRDGLLGYVSHGSEFYEYGRQINRTKPTLSDAKRVLEVLPSNAELKDLYRAGLIDPEILMLNVLD |
| | RITRYDTINPAVWISRENPEEAIANARKQSTGATFPDESMPPLFGIPFAVKDNIDVKGVVTTAACDRFAY |
| | TATATAPAIQHLLDAGAIYVGKLNLDQLATGLTGCRSPYGIPHSYHSEKHASGGSSSGCAVAVAAGLVSF |
| | AIGTDTAGSVRIPAAFNGIVGFKPTKGTISARGVVPACQSLDTMGIFARSVEEARQVWYVMDQYDALDPY |
| | AKPPESLPTWMVDYRGPGEGGFTFGIPPDSAIELCSAKYQELFRVAVERLQSCGATLVDIDYTPFAQAGD |
| | LIYGASLVHERLASIGHDFITENIETLHPTTKTVFQGLLSAKLSAWEVFRDQATQMQCIAAVRKTFDKLD |
| | GGIDVLVVPTAPFHPTIQEVLDDPLAVNSKLGLFTHPANVVDLCGVSVNAGWVEEGEARLPFGITFLAGS |
| | GCDGKLLDIAAVFERASG |
| 369 | XP_007514569.1 isochorismatase family protein [Bathycoccus prasinos]: |
| | MDAIPNSRPYAWPFPDPSEIYFDAFEHTAFILIDMQLDFCGKNGYVSKMGYDVSLTRKPIERVEKVLRKC |
| | RENGVLVLHTREGHRKSLRDLPENKRWRSAQAGAEIGKDGPLGKILTREANGWNLIEELKPLETEDVIDK |
| | PGKGSFMGTDLDLILRLNKIRRIIFGGITTDVCVHTTMREANDLGYECLLLEDGTGATDEGNHASAIKMV |
| | HMQNGVFGATAKCEDVCTFLDANRFDGAENRDAIIPNAKPFPFTIRAKKTAIVMVDWQLDFTSPKGFGAA |
| | LGNDCEVLREEALPNAVKILEAGREAKCAIVHTLEAHKADLSDCPPSKIRRCDKIGQTVDAKMGRILVRN |
| | EPGNSIEPLVAPIEGELIVHKPGKGAFYNTNLEFQLKRRGIETLIFTGVTTEVCVQTSMREANDRGFECI |
| | VADDATESYFPEFKKACLEMISSQNGIVGWRCLTEDVVNALKI |
| 370 | XP_007581703.1 putative isochorismatase hydrolase protein |
| | [Neofusicoccum parvum UCRNP2]: |
| | MASSSAGPSHLAVDAKPFPFSFPSRHTALLVIDMQRDFLLETGFSHSVGANLSAVQQCVRPVMRLLDACR |
| | DARLPIFHTRVGFEPDLSDCPSITLARQAPAHGNAGLTVGDRGSMGRYLVRGEYGHDIIDELRPLPGEIV |
| | VDKPGKGAFWNTELLHKLKARAITHLLVAGVSTECCLSSTIREASDRGLECLMSSLYRKIEVYKGNDPAV |
| | WIHLESREAVLEAAKAVEEKWPNPRERPPLFGIPFSIKDSIDIAGYQTTTACPPLTRMASTSAPVYEKII |
| | ANGGIFIGKTNMDQLGTGMTGCRSPEGTPHSTYHKDYIAGGSSSGSSVSVIEGYDAADRYSKPPIAFERH |
| | INAVGPQRQTFRFGIPPPEALDACHPLYRKIFNHVIRKLVSIGGVLKPLDWTPFEKAGKLLYDGTFVTER |
| | LANHPDDWLEKNRKYLHPTIVQVMDEVVARQSTAIQAYRDQQAKALYTRRAEEIFSAGANGVDVIVTPTA |
| | PAHWTIEEVLADPIKKNSALGEYTHAANVLDLCAVSVPAGLYPLDELLGTEGNEGRLPFGVQFMGGSRMD |
| | AELLEIARRFEQSLDPTAPDEKNGNGSGNTSRKRSRDETLVEEMNTE |
| 371 | XP_007589450.1 putative allophanate hydrolase protein [Neofusicoccum |
| | parvum UCRNP2]: |
| | MAPSLETPPHVVADHVDGRCAAPAKTAADDARCAAADTATPTATAGPAAADPPRPAVAFAAEPYAFSFAP |
| | RKAALLLVDMQRDFLLRDGFGHIQAGDGGVDAVQRTIAPALGVLRAFRALGLAVLHTREGHRADLRDCPT |
| | TKLVRQARAPHSRHAAVIGDAAPMGRLLTRGHHGHDFVDDLQPRPGEIVVDKPGKGSFFSTLLHEHLVDR |
| | GITHLVVAGVTVECCVTTTVREANDRGFDCCILRDCTDGFVPAFKDASLDMIHFSEGLFGFVADSGPLLD |
| | ALAAYQETQTPLSSSAWDGAFDVQSLRRAYAAGLSPVTVVKTVLERIEHGRHDNPYIWINVAAEADLIAR |
| | AEHLDVHRNLDLPLFGIPFAAKDNIDVAGLPTTAACPAFSYTPSQNATVIEKLLAAGAILIGKTNMDQFA |
| | TGLVGVRSPYGACHSVYSGDHVSGGSSSGSAVAVALEQVTFALGTDTAGSGRVPAALNGIVGLKPSKGTV |
| | STHGVVPACKTLDCVSMFAKSIDDAETAWLVAKGFDAADPYARSSRPVTALTNRALLQPEGTYTYALPSE |
| | DLLLTHLSPEYLKAFKRVQDAVRRLHGAHEVPFDFDSYLSASDLVYKGAHVAERASALRPFVQQPEKRAA |
| | LLPITRQIFDQAFTMNAADAFTDLRRAREHTRIMETEFDKCDVVIMPTAPRHPTFLEVEKDPYGPNLEMG |
| | VFASAVNVLDLSAVAIPAGLTDGMPFGVSLVGPAFREGMLLEVARRLTALLA |
| 372 | XP_007683944.1 hypothetical protein COCMIDRAFT 1760 [Bipolaris oryzae |
| | ATCC 44560]: |
| | MAKPTAKPDMVSFDAKPYAFSFPLNHTALLIIDMQRDFLLPKGFGEIQGGNLEAVQASIAPTKRLLDACR |
| | SAGMTIVHTREGHKPDLSDCPSSKLTRQEAAPGNTQHKLVIGDKGELGRLLTRGEYGHDIIDELKPLPGE |
| | VVIDKPGKGSFWNTPILHQLKARAITHLIVSGVTTECCFATTIREANDRGFECCGIEEATSGYNDACFKS |
| | TLDMIHWSQGLFGFVGSLEPLVEALKPFSTNQIQLGYTPPQTPPEFDGDLTISNLQRAYKNGLSPLTVVE |
| | AVYRKLGAYKKIDPAVWIHLRPLESVLEAARELTTKFPDRTALPPLFGIPFSVKDSIDIAGLPTTTACPP |
| | LAHIPSTSAVVYEKVISQGALFIGKTNLDQLATGLVGCRSPYGTPHSVYHPSYISGGSSSGSAVSVAANL |
| | VSFSLATDTAGSGRVPAGLNGIVGFKPTRGTISFRGITPACLSLDCIALATKTITDARTLWQVLESYDPL |
| | DPYSKPTLAFERHINSIGPRSSTFKFGIPPPEALAICSAPTRRLFNDTVSQLQSLGGVLTPINWTPFQKA |
| | GELLYEGTFVSERLASLPDDFLEKNRAGLHPVTAQLMDAVVQRKSSAVDAYRDLQAKTLYTRQAEEVFAY |
| | AAHGIDVLVVPTTPTHWRIDEVLEDPIRKNSVLGEFTHCGNVLDLCGVAVPAGEYPVKELSGKREDGGVL |
| | PFSVTFLSGSRLDAEMLEIARRFEESVCG |
| 373 | XP_007697715.1 hypothetical protein COCSADRAFT 353196 [Bipolaris |
| | sorokiniana ND90Pr]: |
| | MTKTTAKPNMISFDAKPYAFSIPLDHTALLIIDMQRDFLLPQGFGEIQGGNLEAVQASIAPTKQLLDACR |
| | STGMAIVHTREGHKPDLSDCPSSKFTRQEAAPGNTQHKLVIGDKGGLGRLLTRGEYGHDIIDELKPLHGE |
| | VVIDKPSKGSFWNTPILHQLKARAITYLIVSGVTTECCFATTIREANDRGFECCGIEEATSGYNDACFKK |
| | PTLDMIHWNGLSPLTVVEAVYGKIEAYKKIDPAVWIHLQPFESALEAARDLITKFPNRTALPPLFGIPFS |
| | VKDSIDIAGLPTTTACPPLAHIPSTSAVVYEKVISQSAPFIGKTNLDQLATGLVGCRSPYGTPHSVYHPF |
| | YISGGSSSGSAVSVAANLVSFSLATDTAGSGRVPAGLNGIVGFKPTRGTISFRGIAPACLSLDCIALAIK |
| | TVPDARILWQILESYAALDPYSKPALAFERHINSIGPQSSTFKFGIPPQEALAVCSAPTRRLFNDTVSKL |
| | RALGGVLTPINWSPFQKAGELLYEGTFVSQRLASLPDDFLEKNRAGLHPVTAQLMDAVTQRKSSAVDAYR |
| | DLQAKALYTRQVEDVFAYSAQGIDMLVVPTTPTHWRIDEVLEDPIGKNSVLGEFTHCGNVLDLRGVAVPA |
| | GEHLIRELNGREEDEGVLPFSVTFLSGSRLDAEMLEIARRFEESVCG |
| 374 | XP_007706527.1 hypothetical protein COCCADRAFT_31864 [Bipolaris |
| | zeicola 26-R-13]: |
| | MAKTNTKPNMISFEAKPYAFSFPLDHTALLIIDMQRDFLLPQGFGEIQGGNLEAVQVSIEPTKRLLDACR |
| | SAGMAVFHTREGHKPDLSDCPSSKLIRQEAAPGNTQHKLVIGDKGELGRLLTRGEYGHDIIDELKPLPGE |
| | VIIDKPGKGSFWNTPILHQLKARAITHLIVSGVTTECCFATTIREANDRGFECCGIEEATSGYNDACFKK |
| | PTLDMIHWSQGLFGFIGSLQPLVEALEPFSTKQIQLGSTPPQTPPEFDGDLTISSLQRAYRNGLSPLTVV |
| | EAVYRKIEAYKKIDPAVWIHIQPLESALEAARDLITKFPDRTALPPLFGIPFSVKDSIDIAGLPTTTACP |
| | PLAHIPSTSAVVYEKVISQGALFIGKTNLDQLATGLVGCRSPYGIPHSIYHPSYISGGSSSGSAVSVAAN |
| | LVSFSLATDTAGSGRVPAGLNGIVGFKPTRGTISFRGITPACLSLDCIALATKTIPDARTLWQVLESYDP |
| | LDPYSKPALLAFERHINSTGPQSSTFKFGIPPQEALAVCSAPTRRLFNATVSKLQSLGGILTPIPWSPFQ |
| | KAGNLLYEGTFVSERLASLPNDFLEKNRERLHPVTAQLMDAVTQRKSTAVDAYRDLQAKTLYTRQAEDVF |
| | AYAAHGIDVLVVPTTPTHWRIDEVLEDPIAKNSVLGEFTHCGNVLDLCGVAVPAGTYPVGELSGKEEDEG |
| | VLPFSVTFLSGSRLDAEMLEIARRFEESMCG |
| 375 | XP_007724866.1 hypothetical protein A1O1_05792 [Capronia coronata CBS |
| | 617.96]: |
| | MGESKYEPGVLTVEAKPYSFTFPMKTTALLVIDMQRDFICSGGFGEIQGGNLKAVQDSVAPTKALLNACR |
| | NAGLQIFHTREGQVPSLADCPSSKLIRQSASPANTQHLKVIGDKGEMGRLLVRGEFGHDIVDELQPLASE |
| | VVIDKPGKGSFWNTPILHRLKAQGITHLLVAGVTTECCFTTTIREANDRGFECCGILQCTAGYNAALATA |
| | SLGMIYWSQGLFGFVAELQPVLDALSPWQKLSNDTSTPPQTPPVWDGNLGIADLQTSYKNGLSPAELANV |
| | LYDRIEKYDLVNPAVWIKRQSRDDVLASARRLMELYPDRNSLPPLFGVPFTVKDSIDVQGIETTTACPPL |
| | AFMATKSAACYQKVIGQGGLYLGKVNLDQLATGLSGCRSPYGITHSVFSDTHISGGSSSGSCVSVGADLA |
| | TFSLATDTAGSGRVPAGFNGVVGFKPTRGLVSFAGVTPACLSLDCIALIARTVEDARTLWQVCEGYDEND |
| | RYSRDTFPAERHVNALGSQRDTFQFGIPPPEVLEICSPTYRKLFTEAVQRLQSMGGRLVPVDWTPFQAAG |
| | DLLYGGTFVSERLASLPEDFLEKNRTRLHPVIVELFDQVVARQSTAVQLFRELQTKARCTQQATAQFASA |
| | DKLGIDVLVVPTTPEHPTIEAMLGDPIKLNAKMGTFTHFGNVLDLCAVASPAGSYLESEAGPQLPFGITE |
| | LGSRCTDSEVLRIAGRFQEMMTREAS |
| 376 | XP_007753091.1 hypothetical protein A1O7_00860 [Cladophialophora |
| | yegresii CBS 114405]: |
| | MGVSKDKQGFLTIEAKPYPFSFPLKHTALLVIDMQRDFICSGGFGEIQGGNLEAVQASIAPTKQLLEACR |
| | SVSMLIVHTREGQVPSLADCPSSKLIRQAAAPGNKQHLKVIGDKGDMGLLLVRGEYGHDIVDELQPLPAE |
| | VVIDKPGKGSFWNTQILHKLKARGITHLLVSGVTTECCFSTSIREANDRGFECCGIVQSTAGYNPAFKTA |
| | SLDMIYWSQGLFGFVADLQPVLDVLSPWQTQSNGVSTPPQTPPAWNGKLGFDDLQASYKNGLSPLELVNA |
| | LYDRIEKYDKIDLAVWIRRESRDAVLEQARRLLELYPDKHSRPALFGVPFTVKDSIDVQGVETTTACPPL |
| | AFVATKSAMVYQKVITQGALYLGKVNLDQLATGLSGCRSPFGVTHSVFSDDHISGGSSSGSCVSVGADLA |
| | SFSLATDTAGSGRVPAGYNGVVGFKPTRGLVSFEGITPACLSLDCMAFATRTVDDARTLWQLCEEYDEND |
| | RYSRDTFPAERHVNSLGAQREAFRFGIPPPDVLEVCSPTFRKLFNEAVHQLQSMGGSLVPIDWTPFQKAG |
| | DLLYAGTFVSERLASLPDDLLERNRQHLHPVILELFEEVVARQSTAVQLFRDLQAKALYTRQATSQFAAA |
| | DRLGIDVLVVPTVPEHPTIKAMLADPIRLNAKMGTFTHFGNVLDMCAVAVPASTYRDGEAGPQLPFSVTL |
| | LGCRCSDSEVLGIASRFQAMTGQ |
| 377 | XP_007728308.1 hypothetical protein A1O1_09262 [Capronia coronata CBS |
| | 617.96]: |
| | MAPYMSSPTRPSSNEVSSTPDQGKISFEAQPYAFSFDPTKTALVIIDMQRDFLLEAGFGYIQAGEAGVAT |
| | VQATIQPTRAVLRAFRDSGLHVIHTREGHRPDLRDLPTTKLVRQARAPKSRHSMVIGDKGPMGRLLTRGE |
| | YGHDIIDELQPVSGEYVVDKPGKGSFFSTGLHQHLVDRGITYLIVAGVTVECCVTTTVREANDRGFDACI |
| | LSDCTDGFVSTFKKASLDMIHFSEGLFGFVSSSPPLLAALSAYSARQINQPAAWDGSTDMDALKMAYASG |
| | LSPVAVVERVMENIKSGKASQPSTWISLTPHDELLRRAKMLEESGDTSLPLYGVPFAVKDNIDVAGMPTT |
| | AACRSFAYTPSESATVVTRLEAAGAIVIGKTNLDQFATGLVGTRSPYGACHCVFDDSRISGGSSSGSAVA |
| | VALGQVSFSLGTDTAGSGRIPAAFNGIVGLKPTKGTVSTRGVVPACATLDCVSFFARSLEDARTAWLAAK |
| | AFDAEDPYARSSLPLTALTNRALLCEDATYTFAVPPDNILESLLSPEYRRAFAKTEALLARLVGAEQVDF |
| | DFASYLAASDLVYKGSFVVERAVTLSSFTSSAANKASMLPVTAAIIDAAASIPGSKTFEDIYQAQRLTRL |
| | IERQFDRCDVLVLPTAPRHPTLREVEEDPLGPNLELGTFVSAVNILDLAAIAVPMGMVEGLPFGISLVGP |
| | AFREGVLLEVASRVQRLLSA |
| 378 | XP_007745372.1 hypothetical protein A1O5_06589 [Cladophialophora |
| | psammophila CBS 110553]: |
| | MGMSKDKQGFLTIEAKPYPFSFPLKHTALLVIDMQRDFICAGGFGEIQGGNLEAVQASIAPTKQLLDACR |
| | DAGMHVFHTREGQVPSLADCPSSKLVRQAAAPGNTQHLKVIGDKGEMGRLLVRGEYGHDIVDELQPLPAE |
| | VVIDKPGKGSFWNTQILHKLKAYGITHLLVSGVTTECCFSTTIREANDRGFECCGIVQTTAGYNSDFKTA |
| | SLDMIYWSQGLFGFVADLQPVLDVLSPWQIQSKGVSTPPQTPPSWDGKLGIADLQASYKNGLSPLELVNA |
| | LYDRIEKYEHIDGAVWIRRESREAVLAQVRRLLELYPDKHARPALFGVPFTVKDSIDVQGVETTTACPPL |
| | AFVATKSAACYQKVIGQGALYLGKVNLDQLATGLSGCRSPFGITHSVFSDEHISGGSSSGSCVSVGADLA |
| | TFSLATDTAGSGRVPAGFNGVVGYKPTRGLVSFEGVTPACLSLDCIAFTARTVEDARTLWQICEGYDESD |
| | RYARDTFPAERHVNSLGAQREAFRFGIPPPELLEVCSPSFRKLFNEAIARLQAMGGTLMPIDWTPFQKAG |
| | DLLYEGTFVSERLASLPDDFLDKNRPHLHPVILELFEKVVARQSTAVQLFRELQAKALYSRQATSQFASA |
| | TQLGIDVVVVPTAPWHPTIKEMLADPIGLNAKMGTFTHFANVLDMCGIAVPSSTYQESEAGPRLPFSVTF |
| | LGSRCSDSEVLGIASRYQEATAR |
| 379 | XP_007799724.1 hypothetical protein EPUS_00753 [Endocarpon pusilium |
| | Z07020]: |
| | MATSRDVPTLTFIAKPYPFTFPIRTTALLVIDMQRDFICQGGFGEIQGGNLEAVQASIGPTKALLEACRA |
| | AGLSIFHTREGHVRDLSDCPSSKIIRQAAAPGNSQHLKVIGDKGELGRLLVRGEYGHNIVDELRPLPGEV |
| | VIDKPGKGAFWNTRIMHKLKARGITHLLVSGVTTECCFSTSIREANDRGFECCGIVQATAGYNSDFKAAS |
| | LDMIHWSQGLFGFVGELQPLQDALHSYRQPHISLGPTTPPQTPPYWDGSLDITSLHAAYRNGLSPISVAE |
| | ALYDRIEKYQRIDPGVWIYLRSKDAVLADAKKLAEKYPEKHALPSLYGIPFNVKDSIDVAGLHTTTACPP |
| | LAHIPPKSARAYDLVLEQGGLFMGKVNLDQLATGLSGCRSPYGIPHSVFNEKYISGGSSSGSCVSVGADL |
| | VTFSLATDTAGSGRVPSGYNGVVGYKPTRGLISIEGVTPACPSLDCVAIIAKNVEDARAVWQCCEAYDGN |
| | DRYARNSFPLERHVNSLGQLARSFKFGVPPPEVLEICSPVYRRMFNQAIQHLQIIGGTLVAVDWAPFQKA |
| | GDLLYQGTFVSERLASLPDDFFEKNRQDLHPVILKLFEDVIARQSTAVQAYRDLQAKSLLTRQASSQFAA |
| | AGTDGLSVIVVPTAPEHPLISSMLVDPIDLNAKLGTFTHFGNVLDLCAVAVPAGTYQASEIDSSGKGELP |
| | FSITFLGASCTDSEILGIAQRFFEAVGQGDRA |
| 380 | XP_007829518.1 hypothetical protein PFICI_02746 [Pestalotiopsis fici |
| | W106-1]: |
| | MSARPNHKDARAKIDDDNDDATGLVFTTAVPYAYKFPRRRTALVLIDIQRDFVDPDGFGAMQCGNADIFA |
| | SVRAVVDTSQRALAAARSLGLHIVHTREGHAPDLSDLSAAKARRQVDAPGGHHTLGIGETGPMGRLLVRG |
| | EYGHDIVDELRPRPGEVVVDKSGKGSFWATDLHRRLMARGITHLILCGVTTECCVTTTAREANDRGFQCC |
| | ILSDCTGGFDANYVKTSLDMISAFDGLFGFTSTSGELIDQAKRSNLPTPPTTPPTWDGKSLDLATLSSMY |
| | RSHTLTPTEMVESIYEQIKEYGKKDPSIWIHLRPKEAVLKDAANLEAEHAGVSKHELPVLYGIPFAVKDN |
| | FDVASIETTAACPAYAYTPNTTAVSVQLLLQAGALLIGKTNMDQLATGLNGCRSPYGTPASVHGHGKYIS |
| | GGSSSGSAVAVAAGLISFALGTDTAGSGRVPAALNGIVGYKPTKGTISATGIVPACKSLDTASIFALSIE |
| | DARRVWYVLDAYDPRDACAKAPSALPLALVDYRHLSKRGFNFAVPPTSALLTCSAAYRAAFEKAVARLQY |
| | IGGKKITLSEELYQPFRKATDLLYSGSLVAERIACIGPDFVTTKLDQLHPTTKALFSAVLERESKPWDVF |
| | ADQIAQAQATRQVAELFSKHGGRIDVLVTPTVPSHPMITEMEAEPISLNAKMGEFTHFGNVLDLCAVSVG |
| | AGFVEDDMPFAISLVCASGMDGNMFDLAEAFERT |
| 381 | XP_007923590.1 hypothetical protein MYCFIDRAFT_131788 |
| | [Pseudocercospora fijiensis CIRAD86]: |
| | MELPSARPYPYKFPQESTALIIIDMQRDFVDLSGFGMIQCGNDEIFKKVRNIVPRTRKALEAARALGLQV |
| | IHTREGHKPDLSDLPASKRLRQVSAPSGHHTMTIGDQGPMGRLLVRGEYGHGIIDELTPIPGESVIDKPG |
| | KGSMWDTSLHRTLLARGITHLLFAGVTTECCVSTTARECADRGFEVCVLSDCTDGFDSAFYTSTLDMLCS |
| | YDGLFGFVGTSNELLSYAPPQTQTPPTTPPGFTGDISLSALRKQYSSGQLRPTEVIKQISARIEDFKKKD |
| | PAVWTHVEEPEKLLRAAKAVEDQFASKPLPELYGVPFGVKDNIDVAGVKTTNGCEAYAFVPQQSARVVED |
| | LLEAGAIFVGKTNMDQLATGLSGCRSPYGTPRSVYGNNRISGGSSSGSAVAVAAGLVSFALGTDTAGSGR |
| | VPAAFNGLVGHKPTKGTLSARGLAPACQSLDTITIMASTVEDARKVWLAADTGIDENDPYAKSPLSLALW |
| | QSEFRGVKAAGFRFGVPPASALSNCSQTYQSQFIAAVERLKRAGGTPHEVEWEPFEGGSDLLYDASLVQE |
| | RIACIGPDFIASNLNKFQPATKKVFEAALNKDIKPWQVFRDQHLQAKYTREAAKIFKNIDVLLVPTTTCH |
| | PTVAEMEADPLALNAKLGYFTHFANVLDLCGIALPASIHQATNGERLPFGVTLLGAPGTDGRVYDIAREF |
| | ERTT |
| 382 | XP_008027613.1 hypothetical protein SETTUDRAFT_137572 [Exserohilum |
| | turcica Et28A]: |
| | MVSFDAKPYAFSFPLARTALVIIDMQRDFLLPQGFGEIQGGNLEAVQASIAPTKRLLEACRSAGMTIVHT |
| | REGHKPDLSDCPSSKLIRQEAAPGNTQHKLVIGDKGGLGRLLVRGEYGHDIIDELKPLPGEVVIDKPGKG |
| | SFWNTPFLHQLKARAITHLIVSGVTTECCFATTIREANDRGFECCGIEEATSGYNDACFKQSTLDMIHWS |
| | QGLFGFIGSLQPLLEALAPLSTKQNKADSTPPQTPPAFDGDLTISALQQAYQNGLSPLTVVEAVYDKIEA |
| | YKKIDPAVWIHIQPREVALDAARNLAIRFPDRSALPPLFGIPFSVKDSIDVAGLPTTTACPPIAHIPSTS |
| | APVYEKAIAQGALFIGKTNLDQLATGLVGCRSPYGIPHSVYHKDYISGGSSSGSAVSVAANLVSFSLATD |
| | TAGSGRVPAGLNGIVGYKPTRGTISFRGVTPACLSLDCIALSARNIPDTRTLWHVLEGYDALDPYAKPEL |
| | PFERHVNSIGLASRSFRFGIPPPEALALCSAPTRRMFNNTISKLQALGGVLTPINWTPFHEAGQLLYDGT |
| | FVSERLASLPDDFLAKHRAALHPVTAQLMDAVAARKSSAVDAYRDLQAQARHTRDAEAVFAYSSTGVHVV |
| | VVPTTPTHWRIDEVLADPIAKNSVLGAFTHCGNVLDLCGVAVPAGTYPVAELSGHEQDEGELPFSVTFLS |
| | GSRLDAEMLEVARRLEESVGV |
| 383 | XP_008078149.1 Amidase signature (AS) enzyme [Glarea lozoyensis ATCC |
| | 20868]: |
| | MASSLSLPTARPYTYTFPPSTTALLLIDMQRDFVDPSGFGSIQCGNPEIFSAVRKIVPTLQRVLEVSRSL |
| | GMQVIHTREGHRPDLSDLPQSKKVRQVNAPNGHHSMGIGDQGPMGRLLVRGEYGHEIIDELRQLPGEPVI |
| | DKPGKGSFWGTGLHRVLLARGITHILFAGVTTECCVTTTLRECNDRGFECCILSDCTGGFDQQMVTTSMD |
| | IICGQDGLFGYIGDSTDFLAAASKANTLTPPTTPPATEDILPSISELQKGYKSGLFDPETTVTSVFERIE |
| | KYKAIDPAVWISIQPKEQVLLAAKALSAKYAGKPLPPLYGIPFALKDNIDVSGIPTTATCPQFAYTPTST |
| | APAVQHLLDAGALYIGKLNMDQLATGLSGCRSPYGTPHSVYSTSHISGGSSSGSAVAVAAGLVSFTLGTD |
| | TAGSGRVPAALNGIVGFKPTKGTISARGVVPACKSLDTLSIMAPTLAEARSVWYILDVHDALDPYAKPPL |
| | SLNLWKSDYRGARNGGFTFAIPPPSELSACTEEYATLFAGTVEKLRSLGGRLVEIDYTPFAQASGLLYNA |
| | SLVHERLSSIGHSFLTTHLTSLHPTTQSLFASALTTDLKPWQVFHDQDLQRQYTMAAQRTFDTLEGGIDV |
| | LLVPSTPCHPTIAEMEAEPLSLNAKMGTFTHAGNVVDLCGVSVNAGWTGEKLPFGVTFLGGSGYDGRVLD |
| | IAAVFEEGISGESKA |
| 384 | WP_024364804.1 cysteine hydrolase [Lysinibacillus sphaericus]: |
| | MNKVYTIEAKPYSFEFELETTALIIIDMQRDFCAPGGFGEKLGNDITLTRSIIPTIKTVLEVAREKGMMV |
| | IYTREGHRLDLSDCPPSKLKRGSKQGAGIGDEGPMGRILIRGEYGHDIVDELKPVEGEVIIDKPGKGSFY |
| | QTDLEVILNNKGITHLLVAGVTTHVCVQTTIREANDRGFDCLLLEDCSAAFDPKDHEDSIHMINQQGGIF |
| | GWTAPSKNLLVALED |
| 385 | WP_024522161.1 cysteine hydrolase [Edwardsiella hoshinae]: |
| | MTQQVFQAQPFALPFAPQSTALLMIDMQRDFVEAGGFGEALGNDVSLVRSAIAPCQQVLAAARAHQLLVI |
| | HTREGHRADLSDCPAAKLTRGGKTFIGEPGPMGRILVRGEPGHDIIPELYPIAGEPVVDKPGKGAFYQTD |
| | LQLILQNHSIKTLIVCGVTTEVCVNTSVREANDRGYQCIIPADCVGSYFPEFQTAALAMIKAQGAIFGWV |
| | SDAKAIIAGLQG |
| 386 | WP_024529547.1 MULTISPECIES: cysteine hydrolase [Serratia]: |
| | MTQKTFHAEPFALPFDIASTALVMIDMQRDFVEPGGFGEALGNDVSLVRSAIEPCKKVLAAARSQGLLVI |
| | HTREGHRADLSDCPPAKLTRGGQTFIGTHGPMGRILVRGEAGHDIIPELYPQAGEPVIDKPGKGAFYQTD |
| | LHLILQNHGIKTLIVCGVTTEVCVTTTVREANDRGYECIIPQDCVGSYFPEFQKYALEMIKAQGAIFGWV |
| | SDASAIVGGLQD |
| 387 | WP_024580749.1 MULTISPECIES: cysteine hydrolase [Bradyrhizobium]: |
| | MANSSGTIAAEPAPITLDWSKTALVIIDMQRDFMEPGGFGETLGNDVSQLARAVQPIAAVLAAVRDAGLL |
| | VVHTREGHLPDLSDAPPAKVERGAPSLRIGDPGPMGRILIRGEAGHDIIPELYPQDSEIVIDKPGKGAFY |
| | ATEFADILQKYGIENLLVCGVTTEVCVNTTVREANDRGYRCVVIADGCASYFPEFHEMGLKMIKAQGGIF |
| | GWVTDSAAVLEALGG |
| 388 | WP_024649650.1 cysteine hydrolase [Pseudomonas syringae]: |
| | MIKVNARPDRFAFDTSRTAVVIIDMQRDFLEPGGFGAALGNDVAPLQAIVPSVQRLLTLARDEGMAVIHT |
| | RESHRPDLADCPQAKRDHGSPGLRIGDPGPMGRILIRGEPGNQIIDTLAPRAGEWVIDKPGKGMFFATDL |
| | QQRLTEAGITHLIFAGVTTEVCVQTSMREANDRGYRCLLIEDATESYFPAFKQATLEMITAQGGIVGRVA |
| | SLTDLEQALLTRSAL |
| 389 | WP_024662132.1 cysteine hydrolase [Pseudomonas syringae]: |
| | MIKVNARPDRFAFDTSRTAVVIIDMQHDFLEPGGFGAALGNDVAPLQAIVPSVQRLLALARDEGMAVIHT |
| | RESHRPDLADCPQAKRDHGSPGLRIGDPGPMGRILIRGEPGNQIIDTLAPLAGEWIIDKPGKGMFFATDL |
| | QQRLSEAGITHLIFAGVTTEVCVQTSMREANDRGYRCLLIEDATESYFPPFKQATLEMITAQGGIVGRVA |
| | SLTDLEQALLTRSTL |
| 390 | WP_024671286.1 MULTISPECIES: cysteine hydrolase [Pseudomonas syringae |
| | group]: |
| | MIRINARPDSFSCELSQTAVVIIDMQRDFLEPGGFGAALGNDVTLLQAIVPLVQRLLALAREQDLIVIHT |
| | RESHPADLSDCPQAKIDHGLPGLRIGDPGPMGRILIQGEPGNQIIEALTPVAGEWVIDKPGKGMFFATDL |
| | HLRLTEAGITHLIFAGVTTEVCVQTSMREACDRGYRCLLIEDATDSYFPAFKQATLDMITAQNAIVGRVA |
| | SLADVQQALPARSTQ |
| 391 | WP_024675392.1 MULTISPECIES: cysteine hydrolase [Pseudomonas syringae |
| | group]: |
| | MISLCARPDPFTFEPSCTAVVIIDMQRDFLEPGGFGAALGNDVAPLQAIVPAVQRLLALARDQGLAVIHT |
| | RESHHPDLSDCPQAKLDHGLPGLRIGDPGPMGRILIRGEPGNQIIDALTPSAGEWIIDKPGKGMFYATDL |
| | HSRLAEAGITHLIFAGVTTEVCVQSSMREANDRGYRCLLLEDATDSYFPAFKQATLDMITAQGAIVGRVA |
| | SLADLAQALHTRSTP |
| 392 | WP_024882090.1 MULTISPECIES: cysteine hydrolase [Methylocystaceae]: |
| | MAKIMAEPFAFEFEPSALALVIIDMQCDFIEPGGFGESLGNDVSRLRAIVPTVLRLLLSFRARSLPVIHT |
| | MECHRPDLSDCPPAKRDRGAPRLRIGDPGPMGRVLIAGEPGAAILPELAPLPSEIVIEKPGKGAFYATSL |
| | QEELTQLGARQLVFAGVTTEVCVQTTMREANDRGYECLLAEDATASYFPHFETATLEMIRAQGAIIGWTA |
| | KTEQILAGLRDG |
| 393 | WP_024904724.1 cysteine hydrolase [Robbsia andropogonis]: |
| | MTKLRIAAQPGPFDFDTATTALLIIDMQRDFIEPGGFGASLGNDVTQLKKIVPTVVTLLAWAREHQMLVV |
| | HTRESHAPDLADCPRAKRERGVPNSRIGDMGPMGRILVRGEFGNAIIPELAPANNEWVIDKPGKGAFYET |
| | RLAERLSARKITHLLFAGVTTEVCVQTSMREANDRGYDSLLVTDATASYFPVFWQATIEMVHSQGGIVGW |
| | TAPFAALNNPI |
| 394 | WP_024912337.1 cysteine hydrolase [Chania multitudinisentens]: |
| | MTQKTFHAEPFALPFEIGSTALVMIDMQKDFVEPGGFGEALGNDVSLVRSAIEPCKRVLDAARRQGLLVI |
| | HTREGHRADLSDCPPAKLTRGGQTFIGTHGPMGRILVRGEAGHDIIPELYPQAGEPVIDKPGKGAFYQTD |
| | LHLILQNHGIKTLIVCGVTTEVCVNTTVREANDRGYECIIPQDCVGSYFPEFQKYALEMIKAQGAIFGWV |
| | SDASAIVDGLQR |
| 395 | WP_024959554.1 cysteine hydrolase [Pseudomonas syringae]: |
| | MIKVNARPDRFAFDTSRTAVVIIDMQRDFLEPGGFGAALGNDVAPLQAIVPSVQRLLTLARNEGMAVIHT |
| | RESHRPDLADCPQAKRDHGSPGLRIGDPGPMGRILIRGEPGNQIIDTLAPRAGEWVIDKPGKGMFFATDL |
| | QQRLSEAGITHLIFAGVTTEVCVQTSMREANDRGYRCLLIEDATESYFPAFKQATLEMITAQGGIVGRVT |
| | SLTDLEQALLTRSTL |
| 396 | WP_025034737.1 cysteine hydrolase [Bradyrhizobium sp. DOA9]: |
| | MLNSAKPTKGVVSAEPEPITLDWPATALLIIDMQRDFMEPGGFGETLGNDVSQLARAVKPIGAVLTAARD |
| | TGMLVIHTREGHLPDLSDAPPAKVERGAPSLRIGDPGPMGRILIRGEAGHDIIPELYPLDSEIVIDKPGK |
| | GAFYATELGEVLEKYGIENLLVCGVTTEVCVNTTVREANDRGYRCVVIADGCASYFPEFHEMGLRMIKAQ |
| | GGIFGWVADSAAVLEAMKISTT |
| 397 | WP_025219576.1 MULTISPECIES: cysteine hydrolase [Lysinibacillus]: |
| | MNQVYTIEAKPYSFEFELETTALIIIDMQRDFCAPGGFGEKLGNDITLTRSIIPTIKTVLEVAREKGMMV |
| | IYTREGHRLDLSDCPPSKLKRGSKQGAGIGDEGPMGRILIRGEYGHDIVDELKPVEGEVIIDKPGKGSFY |
| | QTDLEVILNNKGITHLLVAGVTTHVCVQTTIREANDRGFDCLLLEDCSAAFDPKDHEDSIHMINQQGGIF |
| | GWTAPSKNLLVALED |
| 398 | WP_025390513.1 cysteine hydrolase [Pseudomonas syringae]: |
| | MISLSARPDPFTFEPSCTAVVIIDMQRDFLEPGGFGAALGNDVAPLQAIVPTVQRLLALARNQGLAVIHT |
| | RESHHPDLSDCPQAKLDHGLPGLRIGDPGPMGRILIRGEPGNQIIDALTPIAGEWIIDKPGKGMFYATDL |
| | HAQLAEAGITHLIFAGVTTEVCVQTSMREANDRGYRCLLLEDATESYFPAFKQATLDMITAQGAIVGRVA |
| | SLADLEQALHTRSTH |
| 399 | WP_025398325.1 cysteine hydrolase [Rhizobium leguminosarum]: |
| | MMEIKAEPFAFPVKHDELALIVIDMQRDFAEPGGFGASLGNDVSRITRIVPDVKRLIQGFRNAGLPVIHT |
| | MECHRPDLSDLPPAKRDRGNPALRIGDEGPMGRILISGEPGTAILAELAPVKGEVVIEKPGKGAFYATEL |
| | GTVLQQKGIKQLVFAGVTTEVCVQTTMREANDRGYECLLAEEATESYFPEFKAAAIAMIRAQGAIVGWTA |
| | HVDDILESIAHA |
| 400 | WP_025437049.1 cysteine hydrolase [Peptoclostridium acidaminophilum]: |
| | MTKYLVDAKPYGYEFDLERTALVIIDMQRDFCAPGGFGEKLGNDITPTRSIIEPLRKVLDAAREKGMFVI |
| | HTREGHRPDLSDCPPSKLNRGKRQGAGIGDMGPMGRILIRGEYGHDIVDELKPMEGEPIIDKPGKGAFYQ |
| | TDLEVILQNRGITHLIVTGVTTHVCVQTTIREANDRGFDCLMLEDCTAAFDPRDQEASIRMINQQGGIFG |
| | WTAMSKNLLEELVK |
| 401 | WP_025797958.1 MULTISPECIES: cysteine hydrolase [Hafniaceae]: |
| | MTTHQFHAEPFALPFNPATTALLMIDMQRDFVEPNGFGEALGNDVSLVRSAIEPCQRVLEAARAQGLFVI |
| | HTREGHRSDLSDCPPAKLTRGGKTFIGTAGPMGRILVRGEAGHDIIPELYPIDGEPVIDKPGKGAFYQTD |
| | LHLVLQNHGIKTLIVCGVTTEVCVTTTVREANDRGYECIIPQDCVGSYFPEFQKYALAMIKAQGAIFGWV |
| | SDANAIVDGLQR |
| 402 | WP_026456678.1 cysteine hydrolase [Aeromonas enteropelogenes]: |
| | MNKRISAQPFDFTFDPATTALIVIDMQRDFVEPNGFGHALGNDVSLVRRAIDPCRKVLDAARANGMLVIH |
| | TREGHRPDLTDCLPAKLVRGGKRFIGEQGAMGRILVQGEAGHDIIPELYPIAGEPVIDKPGKGAFYSTDL |
| | HLILQARGIRSLIICGVTTEVCVQTTAREANDRGYELLIPADCCASYFPEFHRVTLEMIQAQGAIVGWVS |
| | DSAHWTALKG |
| 403 | WP_026949424.1 MULTISPECIES: cysteine hydrolase [Alcanivorax]: |
| | MLNVSAKPNAFPLDPGHCALVVIDMQRDFIEPGGFGAALGNDVSRLAPVVPKVAALLALAREQRLTVVHT |
| | RESHLPDLSDCPPLKRNKLPAGRRIGDDGPMGRILVRGEPGNRILDAVAPEPGEWQVDKPGKGMFHATGL |
| | DQCLRDAGITQLIFAGVTTEVCVQTSMREACDRGYDCLVIEDATESYFPEFKAATLAMIVAQGGIVGRCA |
| | SLDALRRAFQQGANA |
| 404 | WP_027195194.1 cysteine hydrolase [Paraburkholderia sprentiae]: |
| | MPTVNLALPSPFAFEPSKTALVVIDMQRDFIEAGGFGAALGNDVSLLADIVPDVARLIAHARTHGWHVVH |
| | TRESHVPDLSDCPPAKRLRGQPSARIGDKGPMGRILVRGEPGNAIIDALAPIEGELVIDKPGKGAFYATR |
| | LGEELAMRGVTHLVFAGVTTEVCVQTSMREANDRGYDCLVIEGATASYIPAFKEATLAMIRSQGGIVGWT |
| | ATLEQLLEADA |
| 405 | WP_027509691.1 cysteine hydrolase [Rhizobium sullae]: |
| | MDQIRALPFAFPLQKDHLALIVIDMQRDFAEPGGFGASLGNDVSRITRIIPDVKRLIEGFRRAGRPVIHT |
| | MECHKPDLSDLPDAKRNRGSPKLRIGDEGPMGRILITGEPGTAILPELAPTKGEVVIEKPGKGAFYATDL |
| | GKVLKVKDIRQLVFAGVTTEVCVQTTMREANDRGYECLLAEEATESYFPEFKEAAIAMIRAQGAIVGWTA |
| | HVGDILEAIGA |
| 406 | WP_027546369.1 MULTISPECIES: cysteine hydrolase [Bradyrhizobium]: |
| | MLNSTKPTLGVISAEPEPIRLDWPATALLIIDMQRDFMEPGGFGETLGNDVSQLARAVKPIGAVLKAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKISTSA |
| 407 | WP_027683078.1 cysteine hydrolase [Rhizobium leguminosarum]: |
| | MAKIKAEPFAFPVKHDELALIVIDMQRDFAEPGGFGASLGNDVSRITRIVPDVKRLILGFRNAGLPVIHT |
| | MECHRPDLSDLPPAKRDRGNPSLKIGDEGPMGRILISGEPGTAILPELAPVKGEVVIEKPGKGAFYATEL |
| | GTVLQEKGIRQLVFAGVTTEVCVQTTMREANDRGYECLLAEEATESYFPEFKAAAIAMIRAQGAIVGWTA |
| | HVDDILESIAHA |
| 408 | WP_028143122.1 MULTISPECIES: cysteine hydrolase [Bradyrhizobium]: |
| | MLDSSKPTLGVINAEPEPIKLDWPSTALLIIDMQRDFMEPGGFGETLGNDVSQLARAVKPIGAVLTAARD |
| | TGMLVIHTREGHLPDLSDAPPAKVERGAPSLRIGDPGPMGRILIRGEAGHDIIPELYPLDSEIVIDKPGK |
| | GAFYATELGDVLEKYGIENLLVCGVTTEVCVNTTVREANDRGYRCVVMSDGCASYFPEFHEMGLKMIKAQ |
| | GGIFGWVATSAAVLEAMKVSTT |
| 409 | WP_028156964.1 cysteine hydrolase [Bradyrhizobium japonicum]: |
| | MLNSTKPTPGVISAEPEPIRLDWPATALLIIDMQRDFMEPGGFGETLGNDVSQLARAVKPIGAVLKAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKVSTT |
| 410 | WP_028194476.1 MULTISPECIES: cysteine hydrolase [Paraburkholderia]: |
| | MTQQTELTIDAQPGPFTLDSTKTALIVIDMQRDFIEPGGFGESLGNDVSLLAEIVPTVAALLAFARGHRW |
| | LVVHTRESHAPDLSDCPAAKRLRGAPNARIGDAGPMGRILIRGEPGNAIIEPLAPLAGELVIDKPGKGAF |
| | YATRLGEELAIRGITHLVFAGVTTEVCVQTSMREANDRGYDSLLIEDATASYFPAFKQATLDMISSQGGI |
| | VGWTAPFSSLTKLDEAIPAWR |
| 411 | WP_028481720.1 cysteine hydrolase [Nesiotobacter exalbescens]: |
| | MAKVHSEAEPFAFEFDTETTALVMIDMQADFVEPGGFGEALGNDVSLVRSAIEPCRKMLEAAREAGLFVI |
| | HTREGHRSDLTDCPPAKLTRGGKTFIGEQGPKGRILVRGEKGHDIIPELYPVDGEPIIDKPGKGAFYQTD |
| | LSLILESRGIKSLIICGVTTEVCVNTTAREANDRGYEVVIPSDCTASYFPEFYRSALDMIKAQGAIVGWV |
| | SNSESLVAAIKK |
| 412 | WP_028598382.1 cysteine hydrolase [Paenibacillus pasadenensis]: |
| | MSRELAGALPYPFRFEPERTALLVIDMQNDFCAPGGFGERLGNDIAPARAIIPAIARLLAAARGTGMPVV |
| | HTREGHLPDLSDCPPSKLQRSRRQGAGIGDEGPMGRILIRGELGHGIVPELAPLPVEIVIDKPGKGAFYA |
| | TSLEAELRRLGVRSLIVCGVTTHVCVHTTVREANDRGYECVVVADASAAFDPEDHRSALRMLVQQGAIFG |
| | WTAETDEVERVLRAGG |
| 413 | WP_028739229.1 MULTISPECIES: cysteine hydrolase [Rhizobium]: |
| | MADIVAQPFAFPLRRHAVALVVIDMQRDFAEAGGFGASLGNDVARVGKIVPDVKRLIEGFREAGLPVIHT |
| | MECHRPDLSDLPPAKRDRGNPSLRIGDEGPMGRILIAGEPGTAILAELAPIDGEIVIEKPGKGAFYATPL |
| | GEILKQRGISQLVFAGVTTEVCVQTTMREANDRGYECLLVEEATESYFPQFKAAAIEMIRAQGAIVGWTA |
| | HLDDLLEGIACA |
| 414 | WP_029094642.1 cysteine hydrolase [Budvicia aquatica]: |
| | MTQHTFRAEPFALPFDVKTTALVMIDMQRDFVEPGGFGEALGNDVSLVRTAIKPCGTVLDAARQSKMLVI |
| | HTREGHRADLSDCPPAKLTRGGQTFIGTDGPMGRILVRGEAGHDIIPELYPVAGEPVIDKPGKGAFYQTD |
| | LHLILQNHGIKTLIVCGVTTEVCVTTTVREANDRGYECIIPQDCVGSYFPEFQKYALEMIKAQGAIFGWV |
| | SDSAAIVDGLK |
| 415 | WP_029242026.1 cysteine hydrolase [Pseudomonas viridiflava]: |
| | MIDVSARPTRFAFEPASTALVIIDMQRDFLEPGGFGAALGNDVLPLQAIIPTVQQLLALARDQHMTVIHT |
| | RESHVEDLADCPPAKLEHGLPGLRIGDAGPMGRILVRGEPGNQIINALAPIAGEWVIDKPGKGMFFGTGL |
| | HGRLNTAGITHLIFAGVTTEVCVQSSMREANDRGYRCLLIEDATESYFPAFKQATLDMITAQGGIVGRVT |
| | SLSALEQALQTRSTH |
| 416 | WP_029571945.1 cysteine hydrolase [Pseudomonas syringae]: |
| | MIKVNARPDRFAFDTSRTAVVIIDMQRDFLEPGGFGAALGNDVAPLQAIVPSVQRLLTLARNEGMAVIHT |
| | RESHRPDLADCPQAKRDHGSPGLRIGDPGPMGRILIRGEPGNQIIDTLAPLAGEWVIDKPGKGMFFATDL |
| | QQRLTEAGITHLIFAGVTTEVCVQTSMREANDRGYRCLLIEDATESYFPAFKQATLEMITAQGGIVGRVA |
| | SLTDLEQALLTRSTL |
| 417 | WP_029709336.1 cysteine hydrolase [Rhodoferax saidenbachensis]: |
| | MTSPSSPVLSLPATPFAYDFAVAHTALVIIDMQRDFVEPGGFGETLGNDVSLLTAIVPACQTMLSAWRKA |
| | GGTVVHTREAHSADLSDCPPAKRNRGNPKLRIGDVGPMGRILVAGEPGNQIIPELAPMPGEIVIDKPGKG |
| | AFYATGLQEMLAERAITHLLFMGVTTEVCVQTSMREANDRGYDCLLLVDCTESYFPHFKAAAVEMIHAQG |
| | AIVGWTAPSTMVLTALAA |
| 418 | XP_008714432.1 allophanate hydrolase [Cyphellophora europaea CBS |
| | 101466]: |
| | MASSPLLSLPSARPYGFQFDPEHTALVIIDVQRDFVDPDGFGAIQCGNAEIFNSVRSIVPAIKDTLTASR |
| | RLGLHVIHTREGHRPDLSDLPASKRDRQVNAPSGHHTMGIGDQGPMGRLLVQGEYGHDIIDDLRPLPGES |
| | VIDKPGKGSFWETSLHRVLMARDITHLLFCGVTTECCVTTTAREANDRGFECCILTDCTAGFNATSVEVS |
| | LNMFCSYDGLFGYVASSNELVAHGSQLLRTPPDSPAAWKGDMDLEAISTHIRSRSLPLLDLVTRVFDRVE |
| | KADPHIWTYVRSRQEVLADANALEARYATSSSDQLPWLYGVPFAVKDNFDVAGMPTEAACPAYRYFPKET |
| | APVIKLLQSAGALLIGKTNMDQLATGLNGCRSPSGNPVSIFGRGKYISGGSSSGSGVAVAAGLVTFSLGT |
| | DTAGSGRVPAALNGIVGVKPTKGTLSARGIVPACRSLDTASIFAKTVEDARRVWYAVDQYDAEDVYAKDP |
| | SSLPLAMSDYRANPTFTFAVPPESVVKACDPSYQKAFANALARLQNMGGLMLTLSKDGYKPFQMASDLLY |
| | SGTLVNERIACIGPEFLTTNLDTLHPATQALFRGVIERPTKAWEVYRDQELQATATAEAARLFSRFAGKV |
| | DVLVTPTVPCHPTSEEMESDPIQLNAKLGLFTHFGNVLDLCAISVPAGVVVASEGSQLPFGISLVCARGL |
| | DGKMFSIARRFEKGSK |
| 419 | XP_008722539.1 allophanate hydrolase [Cladophialophora carrionii CBS |
| | 160.54]: |
| | MGVTKDKQGFLTIEARPYPFSFPLKHTALLVIDMQRDFICSGGFGEIQGGNLEAVQASIAPTKQLLEACR |
| | SASMLIVHTREGQVPSLADCPSSKLIRQAAAPGNKQHLKVIGDKGEMGRLLVRGEYGHDIVDELQPLPAE |
| | VVIDKPGKGSFWNTQILHKLKAHGITHLLVSGVTTECCFSTTVREANDRGFECCGIVQSTAGYNPAFKTA |
| | SLDMIYWSQGLFGFVADLQPVVDVLSPWQNQSKGVNTPPQTPPAWNGRLGIADLHASYKNGLSPLELANA |
| | LCDRIEKYEKIDPAVWIRRESRDAVLEQARRLLELYPDKHSRPALFGVPFTVKDSIDVQGVETTTACPPL |
| | AFVATRSAAVYQKVMAQGALYLGKVNLDQLATGLSGCRSPFGVTHSVFSDDHISGGSSSGSCVSVGADLA |
| | TFSLATDTAGSGRVPAGYNGVVGFKPTRGLVSFEGITPACLSLDCIAFTTRTVDDARTLWQLCEDYDEND |
| | RYSRDTFPAERHVNSLGAQREAFRFGIPPPEVLEVCSPTFRKLFNEAVHQLQSMGGSLVSIDWTPFQKAG |
| | DLLYAGTFVSERLASLPDDFLEKNRQHLHPVILELFEEVVARQSTAVQLFRDLQAKALYTRQATAQFAAA |
| | DRLGIDVLVVPTAPEHPTIKAMLADPIRLNAKMGTFTHFGNVLDMCAVAVPAATYREGEAGPQLPFSVTL |
| | LGCRCSDSEVLGIASRFQARTGQ |
| 420 | XP_009033335.1 hypothetical protein AURANDRAFT_20579 [Aureococcus |
| | anophagefferens]: |
| | MQHGVFGAVAAADAVVAALDALPRAAPRGGAPTWPPPAPRVLAAAPPGPGGGAVARAKPFAFAWPSARAL |
| | GVLMIDWQRDFLDPEGFGASLGNDVAPLRSAVPAAARVLEAARARGLFVAHTLEAHAADLGDCPPSKKRR |
| | CEAIGETLDASRGRVLVRGEPGNAVVPELAPAAGELVVHKPGKGAFYGTTLERDLRAAGVTHLVVTGVTT |
| | EVCVQSTLREANDRGFDCLLVEDATESYFPAFKRATLDMVVAQGGIVGWTATAGDVAAALAAAA |
| 421 | WP_032353458.1 MULTISPECIES: cysteine hydrolase [Enterobacteriaceae]: |
| | MTISIFQAQPFELPFDPCTTALIMIDMQRDFVEAGGFGEALGNDVSLVRTAIAPCKEVLVAARQKGIMVI |
| | HTREGHREDLSDCPSAKLTRGGKTFIGEPGPMGRILVRGEAGHDIIPELYPVTGEPVIDKPGKGAFYQTD |
| | LHLILQKRGIKTLIVCGVTTEVCVTTTVREANDRGYECIIPEDCVGSYFPEFQKYALEMIKAQGAIFGWV |
| | TDSKAIIAGLEG |
| 422 | XP_009650549.1 glutamyl-tRNA(Gln) amidotransferase subunit A |
| | [Verticillium dahliae VdLs.17]: |
| | MSSTSRPSLSLPNARPYPFDFPLATTALVIIDIQRDFVDPGGFGSVQCGNDEIFSKARSIVPAVQRVLEI |
| | FRSTRGHVIHTREGHQPDLADLPAAKKLRQINNPNGHHFMGIGDQGPMGRLLVRGEYGHDIIDELQPWPT |
| | EVVIDKPGKGSFWGTDIHRVLLARGITHLLFAGVTTECCVTTTLRECNDRGYQCCVLEDCTQGFDAQQVT |
| | TSLDTICAQDGLFGFVGNSADFVAATTDVSTAPVSQLVTSGPFPSIDDFQALYKDGRITPTDVVNATFDR |
| | IEAYQKDDPAVWTSLAKRTDVLVAAKALAEKYKEKPLPPLFGVPFGVKDSIDVAGVETTAACPSYAYVPK |
| | ATAICVQHILDAGGIYVGKTNLDQLATGLSGCRSPYGVPHSTFSKDLIAGGSSSGGCVAVAARLVPFTVA |
| | TDTAGSGRVPAAFNGVVGFKPTKGTISARGLVPACKTLDSIAIVATSVADARAVWRVIAKHDKADPYSKL |
| | PHTLPTWKTDFRGPKDGGFDFAVPPSAALEACTPEYRRLFAEAVKKLQSAGGRLRNTDWEAFERAGELLY |
| | EGALLHERITCIGREFLQSSIKGGSLHPVIEELFSQALDSAPDAYDVFRDQATQAELSRRAHMAFDTLCG |
| | GVDVLVVPTTVCHPTFEEIAADPIRLNARLGTFTHFANIVDLCGLSVPAGTYLDVKGTELPFGVTILAGS |
| | GFDAKALDVARVLEEVMKAK |
| 423 | WP_034164290.1 MULTISPECIES: cysteine hydrolase [Edwardsiella]: |
| | MTQQAFQAQPFALPFDPQSTALVMIDMQRDFVEAGGFGEALGNDVSLVRSAIAPCQKVLAAARAHQLLVI |
| | HTREGHRADLSDCPAAKLTRGGKTFIGEPGPMGRILVRGEPGHDIIPELYPIAGEPVVDKPGKGAFYQTD |
| | LQLILQNHGIKTLIVCGVTTEVCVNTTVREANDRGYQCIIPADCVGSYFPEFQKSALEMIKAQGAIFGWV |
| | SDADSIIAGLQG |
| 424 | WP_034461659.1 cysteine hydrolase [Buttiauxella noackiae]: |
| | MTHAFEAQPFALPFDRKTTALVMIDMQRDFVEAGGFGEALGNDVSLVRSAIEPCKHVLEVARNKDLLVIH |
| | TREGHRPDLTDCPPAKLTRGGKTFIGEPGPMGRTLVRGEAGHDIIPELYPVAGEPIIDKPGKGAFYQTDL |
| | HLILQNNGIKTLIVCGVTTEVCVNTTVREANDRGYECIIPEDCVGSYFPEFQKYALEMIKAQGAIFGWVS |
| | NADAIIKGLKG |
| 425 | WP_034517272.1 cysteine hydrolase [Agrobacterium rhizogenes]: |
| | MVEVPAQPFAFPLQRNGVALVVIDMQRDFAEPGGFGASLGNDVSRITRIVPDVKRLIEGFRKAGLPVIHT |
| | MECHKPDLSDLPPAKLNRGNPTLRIGDEGPMGRILIAGEPGTAILPELAPIDGEIVIEKPGKGAFYATKL |
| | GDILKDNGIRQLVFAGVTTEVCVQTTMREANDRGYECLLAEEATESYFPEFKAAAIAMIRAQGAIVGWTA |
| | HIDDILEAINHA |
| 426 | WP_035038197.1 cysteine hydrolase [Aquabacterium sp. NJ1]: |
| | MITTVHAQPFDFSFNIRHTALLIIDMQRDFVEPGGFGASLGNDVSLLQAIVPTCQRVLQAWRDMGGWVVH |
| | TREAHRPDLSDCPPAKLNRGSPMLRIGDAGPMGRILIRGEPGHAIIPELAPIEGELVIDKPGKGAFYATC |
| | LSEALTVREITHLIVMGVTTEVCVQTTMREANDRGYDCLLVEDGTESYFPAFKQATLEMIRAQGAIVGWT |
| | APSAALLAALDTSVSPPALARSA |
| 427 | WP_035077614.1 cysteine hydrolase [Devosia riboflavina]: |
| | MVDIPARPYPYPLDPGHTALVVIDMQRDFIEPGGFGDSLGNDVSRLEAIIPATAALIALFREEGWPIIHT |
| | REAHMPDLSDCPPAKISRGKPGLRIGDTGAMGRILIAGEPGNQIVDALAPIAGEIVIDKPGKGMFYATGI |
| | HERLQDMGISHLVFAGVTTEVCVQTSMREANDRGYECLLIEDATESYFPQFKASAIEMIAAQGGIVGWVT |
| | PLSELQKTLARDHAHV |
| 428 | WP_035252308.1 cysteine hydrolase [Actibacterium atlanticum]: |
| | MAVIKAQPFDFTFEPSTTGLVMIDFQRDFMEPGGFGETLGNDVSLLRAAIEPAQALLAAFRKAGLPVIHT |
| | RECHRPDLSDLPDAKRDRGAPSLRIGDAGPMGRILISGEPGADIIPELYPIKGETVIDKPGKGAFYSTEF |
| | GAVLADLGLKQLIFAGVTTEVCVQTTMREANDRGFDCLLATDASESYFPAFKAAAIDMITAQGGIVGWAS |
| | PVAQIVEVLDG |
| 429 | WP_035256303.1 cysteine hydrolase [Actibacterium mucosum]: |
| | MMDILAEPFPFPCERETLGLVVIDMQRDFVEPGGFGETLGNDVSRLGAIVPTVAQLINGFRAAGLAVIHT |
| | RECHKPDLSDLPDAKRDRGAPSIRIGDPGPMGRILVAGEPGAEIIPELAPLPDELVLDKPGKGAFCRTEF |
| | ETHLQKMGLKQLVFAGVTTEVCVQTTMREANDRGYDCLLATDATESYFAEFKAAAIQMIIAQGGIVGWAT |
| | PTGRILEALNA |
| 430 | WP_035530882.1 cysteine hydrolase [Hoeflea sp. BAL378]: |
| | MVEIQASPLPFRLDPDRAALVIIDMQRDFVEPGGFGETLGNDVSACRAIVPTVRKLLDACRKAGLTIVHT |
| | RECHRPDLSDCPLAKRERGNPGLRIGDEGPMGRILIAGEPGAAIVAELAPLPGEIVIDKPGKGAFYATDL |
| | GDQLGRRGVTQLIFAGVTTEVCVQTTMREANDRGYECLLITDATESYFPEFKLAAIAMIVAQGGIVGWAA |
| | ASEDLIGQLA |
| 431 | WP_035597944.1 cysteine hydrolase [Edwardsiella tarda]: |
| | MTQQTFQAQPFALPFDPQSTALVMIDMQRDFVEAGGFGEALGNDVSLVRSAIAPCQKVLAAARAHQLLVI |
| | HTREGHRADLSDCPAAKLTRGGKTFIGEPGPMGRILVRGEPGHDIIPELYPIAGEPVVDKPGKGAFYQTD |
| | LQLILQNHGIKTLIVCGVTTEVCVNTTVREANDRGYQCIIPADCVGSYFPEFQKSALEMIKAQGAIFGWV |
| | SDADSIIAGLQG |
| 432 | WP_035609961.1 cysteine hydrolase [Hylemonella gracilis]: |
| | MRIHAQPFPYECDPRATALVLIDMQRDFIEPGGFGETLGNDVALLAAIVPACRTVLAAWRRAGGLVVHTR |
| | EAHQPDLSDCPPAKRLRGNPSLRIGDVGPMGRILVAGEPGNQIIDALAPAPGELVIDKPGKGMFWATGLH |
| | EKLQARGVSHLIFMGVTTEVCVQTSMREANDRGYDNLLLEDCTESYFPAFKAATLEMVRAQGAIVGWTAR |
| | SEALLAALK |
| 433 | WP_035687887.1 cysteine hydrolase [Avibacterium paragallinarum]: |
| | MLKQFQAEPFPLSFNPQTTALLMIDMQRDFVEPGGFGEALGNDVNLVRSAIQPCKRMLSAARQAGIFVLH |
| | TREGHRADLSDCPPAKLTRGGKTFIGECGPKGRILIRGEEGHDIIPELYPIAGEPIIDKPGKGAFYQTDL |
| | HLILQNRGIKTLIVCGVTTEVCVNTTVREANDRGYECIIPEDCVGSYFPEFQEYALKMIKAQGAIFGWVS |
| | TSTEIINALMS |
| 434 | WP_035752748.1 MULTISPECIES: cysteine hydrolase [Gordonia]: |
| | MSETVTLEALPGPIELDLDHTALIIIDMQRDFLLPGGFGEALGNDVAQLQRVVEPLAALLDAARAAGMLV |
| | IHTREGHLPDLSDCPPAKLHRGAPSKRIGDPGAFGRILIRGEYGHDIVDELAPLDTEVVIDKPGKGAFYA |
| | TELSKVLADNQITQLLVTGVTTEVCVHTTTREANDRGFECVVVSDCVGSYFPGFQRVGLEMIAAQGGIFG |
| | WTAPGAAIIPLLKERAPAEPAV |
| 435 | WP_035935337.1 MULTISPECIES: cysteine hydrolase [Burkholderiaceae]: |
| | MLTIDAQPGPFTFEPSKTALVVIDMQRDFIEPGGFGESLGNDVSLLAAIVPTVASLLALARREGWLVVHT |
| | RESHAADLSDCPPAKRARGAPNARIGDPGRMGRILIRGEPGNAIVDELAPLGSELVIDKPGKGAFYATPL |
| | GGELAARGITHLVFAGVTTEVCVQTSMREANDRGYECLLVEDATASYIPAFRQATLEMVRSQGGIVGWTA |
| | PFASLAQSHGEKRGWN |
| 436 | WP_035963210.1 cysteine hydrolase [Caballeronia grimmiae]: |
| | MPVLTRARPSPFSFDASHTALIVIDMQRDFIEPGGFGEALGNDVSLLESIVPAVARLLDHARDRGWLVVH |
| | TRESHAPDLSDCPDAKRLRGAPQARIGDMGPMGRILVRGEPGNAIVDAVAPVGGEILIDKPGKGAFYATR |
| | LGEELAQRGITHLVFAGVTTEVCVQTSMREANDRGYECVLIEDATASYIPAFKTATIEMIRSQGGIVGWT |
| | ATFADLSEN |
| 437 | WP_036026000.1 cysteine hydrolase [Bradyrhizobium yuanmingense]: |
| | MLNSAKPTKGVVSAEPEPITLDWSATALLIIDMQRDFMEPGGFGETLGNDVSQLGRAVKPIGAVLTAARD |
| | SGMLVIHTREGHLPDLSDAPPAKVERGAPSLRIGDPGPMGRILIRGEAGHDIIPELYPLDSEIVIDKPGK |
| | GAFYATELGEVLEKYGIENLLVCGVTTEVCVNTTVREANDRGYRCVVIADGCASYFPEFHEMGLKMIKAQ |
| | GGIFGWVADSAAVLEAMTVSTT |
| 438 | WP_036050191.1 cysteine hydrolase [Burkholderia gladioli]: |
| | MHFEVPARPAPYRYDPAHTALIVIDMQRDFIEPGGFGAALGNDVAPLAAIVPSVAALLAFAREQRWSVVH |
| | TRESHAPDLSDCPPAKRLRGAPDLRIGDSGPMGRILVRGEPGNQIVEALAPLAGETVIDKPGKGAFHATA |
| | LDALLRERGITHLVFAGVTTEVCVQTSMREANDRGYDCLLVEDATASYFPAFKAACLEMISSQGGIVGWT |
| | APLRALLEAAPLPAAPSASPQP |
| 439 | WP_036347395.1 cysteine hydrolase [Mycolicibacterium aromaticivorans]: |
| | MATINAEPFPLDIDIASTALVIIDMQRDFVLPGGFGEALGNDTSLLLAAVEPIERVLAQARKIGMLVIHT |
| | REGHRPDLTDCPPAKLHRGGKTFIGEPGPMGRILVRGEQGHDIIDQLYPIDGEPVIDKPGKGSFHATDLG |
| | QILADRGIKTLVVCGVTTEVCVHTTVREANDRGFECLVLSDCVASYFPEFQRVALEMIKAQGAIFGWVAD |
| | ADEFIAATS |
| 440 | WP_037146239.1 cysteine hydrolase [Rhizobium phaseoli]: |
| | MAEIKAEPFAFPVKHDQLALIVIDMQRDFAEPGGFGASLGNDVSRIGRIVPDVKRLIQGFRYAGLPVIHT |
| | MECHRPDLSDLPPSKRDRGNPMLRIGDEGPMGRILIAGEPGTAILPELAPIDGEVVIEKPGKGAFYATGL |
| | AEALQRKGIRQLVFAGVTTEVCVQTTMREANDRGYECLLAEEATESYFPEFKAAAIAMIRAQGAIVGWTA |
| | RVDDILESIAHA |
| 441 | WP_037148297.1 cysteine hydrolase [Rhizobium sp. YS-1r]: |
| | MGDIKAEPFAFPAIPEALALIVIDMQRDFVEPGGFGASLGNDVSRIMKIVPDVKRLIEGFRSANLPVIHT |
| | MECHRPDLSDLPPAKRDRGNPSLRIGDEGPMGRILISGERGTEILSELAPIDGEVVIEKPGKGAFYATEL |
| | GEVLKAKGIRQLVFAGVTTEVCVQTTMREANDRGYECLLAEEATESYFPEFKAAAIAMIRAQGAIVGWTA |
| | HVDDILEGIVPRGMTNA |
| 442 | WP_037189234.1 MULTISPECIES: cysteine hydrolase [Rhodococcus]: |
| | MTSAPTPVTSIPSASPSEFTIDAETTALIVIDMQRDFLLPGGFGESLGNDVGLLRTVIEPLAGLIAVARE |
| | AGIPVIHTREGHLPDLSDCPPAKLRRGTPSQRIGDRGAFGRILVRGEYGHDIVDELAPLEGETVIDKPGK |
| | GAFYATELSEVLTSAGITTLLVTGVTTEVCVHTTVREANDRGYECLVVTDCVGSYFPEFQRVGLEMISAQ |
| | GGIFGWTAPSEDVVAALVAFVPSTASR |
| 443 | WP_037192957.1 MULTISPECIES: cysteine hydrolase [Rhizobium]: |
| | MREIPAQPFAFPLQRDAVALVVIDMQRDFAEPGGFGASLGNDVSRITRIVPDVKRLIEGFRKAGLPVIHT |
| | MECHRPDLSDLPAAKRNRGNPTLRIGDEGPMGRILIVGEPGTAILPELAPIDGETVIEKPGKGAFYATEL |
| | GDILGDRGIRQLVFAGVTTEVCVQTTMREANDRGYECLLAEEATESYFPEFKAATIAMIRAQGAIVGWTA |
| | HVDDILETINHA |
| 444 | WP_037209118.1 cysteine hydrolase [Rhodovulum sp. NI22]: |
| | MGLIRAEPFDFSFDPATLGLVVIDMQRDFVEPGGFGASLGNDVALLQAIIPTVQALIGGFRAAGLPVIHT |
| | RECHRPDLSDLPPAKRDRGAPALRIGDEGPMGRILIAGEPGADIVPELAPAPGEPVIDKPGKGAFYGTEF |
| | AQVLADRNLRQLVFAGVTTEVCVQTTMREANDRGFDCLLATDATESYFPDFKQAAIRMIIAQGGIVGWAA |
| | PTAHVLEAL |
| 445 | WP_037403680.1 MULTISPECIES: cysteine hydrolase [unclassified |
| | Serratia]: |
| | MTQKTFHAEPFALPFEMGSTALVMIDMQKDFVEPGGFGEALGNDVSFVRSAIEPCKRVLDAARRQGLLVI |
| | HTREGHRADLSDCPPAKLTRGGQTFIGTHGPMGRILVRGEAGHDIIPELYPQAGEPVIDKPGKGAFYQTD |
| | LHLILQNHSIKTLIVCGVTTEVCVNTTVREANDRGYECIIPQDCVGSYFPEFQKYALEMIKAQGAIFGWV |
| | SDASAIVAGLQG |
| 446 | WP_037484935.1 cysteine hydrolase [Sphaerotilus natans]: |
| | MPDSSILTVPARPGPFMLPLRHAALVVIDMQRDFVEPGGFGASLGNDVTRLQAIVPALQRLLAAWRAAGG |
| | AVVHTREGHRADLSDCPPAKRLRGSPGLRIGDTGPMGRLLVQGEPGHAIIAELAPTEGERVIDKPGKGAF |
| | FGTDLQAWLAARGISHLVFTGVTTEVCVQTSMREANDRGFDCVLIEDATESYFPEFKAATLAMVRAQGAI |
| | VGWTATSADLIAALAAMRTEGPCP |
| 447 | WP_037942159.1 MULTISPECIES: cysteine hydrolase [unclassified |
| | Sulfitobacter]: |
| | MTQIPARPFDFPLARDQVALIIIDMQRDFVEPGGFGASLGNDVRPLQAIVPTVARLLAGFRAAGLPIFHT |
| | REAHRPDLSDCPPAKRLRGAPALRIGDAGPMGRVLIAGAPGCEIIPALTPLPDEPVIDKPGKGAFYATDL |
| | GDQLAERGITQLVCAGVTTEVCVQTTMREANDRGFECLLATDATESYFPSFKAAAIEMIVAQGGIVGWAT |
| | DTDTILGAING |
| 448 | WP_038091623.1 cysteine hydrolase [Acidihalobacter prosperus]: |
| | MSFEIDARSFAYRCPADGTALLLIDLQRDFVEPGGFGASLGNDVSRLRPAIKACRRLLETFRALGLPVLH |
| | TREAHRPDLADCPPAKRLRGEPPLRIGDAGPMGHLLVAGETGTEIVPECRPLPGETVIDKPGKGAFYATD |
| | FGTHLERLGITHLVVGGVTTEVCVQSTLREANDRGYECLLVEEATESYFPEFKRATLEMVRAQGAIVGWT |
| | AALADVLRAFAPPFPKDRSLT |
| 449 | WP_038587761.1 cysteine hydrolase [Neorhizobium galegae]: |
| | MGQIKAEPFAFPAKPEALALIVIDMQRDFAEPGGFGASLGNDVGRITKIVPDVKRLIQGFRDAGLPVIHT |
| | MECHKPDLSDLPPAKRDRGNPTLRIGDVGPMGRVLISGEPGTAIISELAPIDGEVVIEKPGKGAFYATEL |
| | GEVLKAKGINQLVFAGVTTEVCVQTTMREANDRGYECLLAEEATESYFPEFKAAAIAMIRAQGAIVGWTA |
| | HVDDILEGIAPKGTTNA |
| 450 | WP_038691063.1 cysteine hydrolase [Rhizobium sp. IE4771]: |
| | MAAIKAEPFAFPVQYDQLALIVIDMQRDFAEPGGFGASLGNDVSRIGRIVPDVRRLIQGFRYAGLPVIHT |
| | MECHRPDLSDLPPAKRNRGNPVLRIGDEGPMGRILIRGEPGTAILPELAPINGEVIIEKPGKGAFYATGL |
| | GEILQQKGIKQLVFAGVTTEVCVQTTMREANDRGYECLLAEEATESYFPEFKAATIAMIRAQGAIVGWTA |
| | HVDDILESIAHA |
| 451 | WP_039080206.1 MULTISPECIES: cysteine hydrolase [Metakosakonia]: |
| | MRTIKAQPFDFQFDPATTALVVIDMQRDFVERGGFGEALGNDVSLVRRAIEPCAALLKSAREAGLLVIHT |
| | REGHRDDLSDCLPAKRTRGGKTFIGEPGPMGRILVRGQPGHDIIPELAPQPGEPVIDKPGKGAFYATDLH |
| | LILQSHRIASLIICGVTTEVCVQSTAREANDRGYELVIPEDCCASYFPEFHQAALAMIKAQGAIVGWVSH |
| | SAEVIAALRP |
| 452 | WP_039096643.1 MULTISPECIES: cysteine hydrolase [Pasteurellaceae]: |
| | MLKQFQAEPFPLSFNPQTTALLMIDMQRDFVEPGGFGEALGNDVNLVRSAIQPCKRMLSAARQAGIFILH |
| | TREGHRADLSDCPPAKLTRGGKTFIGECGPKGRILIRGEEGHDIIPELYPIAGEPIIDKPGKGAFYQTDL |
| | HLILQNRGIKTLIVCGVTTEVCVNTTVREANDRGYECIIPEDCVGSYFPEFQEYALKMIKAQGAIFGWVS |
| | TSTEIINALMS |
| 453 | WP_039133141.1 MULTISPECIES: cysteine hydrolase [Pasteurellaceae]: |
| | MLKQFQAEPFPLSFNPQTTALLMIDMQRDFVEPGGFGEALGNDVNLVRSAIQPCKRMLSAARQAGIFILH |
| | TREGHRADLSDCPPAKLTRGGKTFIGECGPKGRILIRGEEGHDIIPELYPIAGEPIIDKPGKGAFYQTDL |
| | HIILQNRGIKTLIVCGVTTEVCVNTTVREANDRGYECIIPEDCVGSYFPEFQEYALKMIKAQGAIFGWVS |
| | TSTEIINALMS |
| 454 | WP_039151605.1 cysteine hydrolase [Bradyrhizobium japonicum]: |
| | MLNATKPTPGVISAEPEPIRLDWPATALLIIDMQRDFMEPGGFGETLGNDVSQLARAVKPIGAVLKAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKVFDHLGVTS |
| 455 | WP_039621983.1 cysteine hydrolase [Rhizobium sophoriradicis]: |
| | MAAIKAEPFAFPVKYDQLALIVIDMQRDFAEPGGFGASLGNDVSRIGRIVPDVRRLIQGFRYAGLPVIHT |
| | MECHRPDLSDLPPAKRNRGNPVLRIGDEGPMGRILIRGEPGTAILPELAPINGEVIIEKPGKGAFYATGL |
| | GEILQQKGIKQLVFAGVTTEVCVQTTMREANDRGYECLLAEEATESYFPEFKAATIAMIRAQGAIVGWTA |
| | HVDDILESIAHA |
| 456 | XP_011121646.1 hypothetical protein AOL_s00078g81 [Arthrobotrys |
| | oligospora ATCC 24927]: |
| | MAISKSLPENISFSAKPYAFSFPSAATALLIIDMQRDFLLENGFGHIQGGNLTNVQAAIKPTARLLEVWR |
| | NLGLPVVHTREGHVPDLSDCPSSKLVRQAAAPGNKQHAQIIGDKGPMGRLLVRGEYGHDFVDELQPYESE |
| | IVVDKPGKGAFYNTKLMEILKNNGITHLIIGGVTTECCVTTTLREANDRGFECCALTEITDGYNPPYKTA |
| | SLDMIYWSQGLFGYVGSMDPLIEALKQFSSVPASTTVEKPSDIGYISSDGSEVKSPPQTPPAWDGSLLID |
| | DLQRSYATGVSPITVLEALYKKIEEYSQVDPAVFIYLVEKKTAFARAEELIKLFPDRRNLPPLWGVPFSV |
| | KDSIDVAGIPTTTACPPLAFVPTRSAAIYDKLIVQGAIHIGKTNLDQYATGLNGTRTPYGIPRSVFNKDY |
| | ISGGSSSGSAVSVGARLVSFSLATDTAGSGRVPALFNGVIGFKPTRGTVSFMGVTPACLSLDCCSFMTSN |
| | IKDARIVWSLVEGYDAADRYSKGTPPILRSVDAHFTKFKFGIPPPEALSVCSFTFRQMFNDTVKKLQDIG |
| | GQLVPVDWAPFDNAGKLLYDGTFVIERLASLPDDFLEKNRDALHPVIRELFEQVVARKSTAVDVFRDLHK |
| | QALYIRQMMEIFSPSGISVLVVPTAPLHPTVEQMLAEPISLNSTLGAFTHFGNVNDLCAVAVPAGTYPVL |
| | STDNSSESSNGILPFGVTFLGGSRTDSEVLDIASRFEAYMKQEST |
| 457 | XP_011111407.1 hypothetical protein H072_5538 [Dactylellina haptotyla |
| | CBS 200.50]: |
| | MAISKSLPGKISFSAKPYAFTFPSAATALLVIDMQRDFLLENGFGHIQGGNLTNVQAAIKPTARLLEVWR |
| | NLGLPVVHTREGHVPDLSDCPSSKLVRQAAAPGNKQHVQIIGDKGPMGRLLVRGEYGHDFVDELQPHESE |
| | IVVDKPGKGAFYNTRLMEILKKNGITHLIIGGSLSFVEIMVEYLLIYEKTTECCVTTTLREANDRGFECC |
| | ALTEITDGYNPPYKTASLDMVYWSQGLFGYVGSMDPLIEVLKSFSSTSMASASEKPKSDIGYVSSDGSEI |
| | KSPPQTPPAWDGSLLIDDLQRSYRSGVSPITVLETVYTKIEEYSKVDPAVFIHLVERKTAFARAEELIKA |
| | HPDRHNLPPLWGVPFSVKDSIDVAGVPTTTACPPLAFVPTRSAAVYDKVIAEGAIHIGKTNLDQYATGLN |
| | GTRTPYGIPRSVFNKDYISGGSSSGSAVSVGAKLVSFSLATDTAGSGRVPALFNGVVGFKPTRGTVSFMG |
| | VTPACLSLDCCSFMTSNTADARTVWSLVEGYDAADRYAKATPPILRSVDAHFTKFKFGIPPPEALGVCSF |
| | TFRQMFNDTVKKLQEIGGQLVPVDWAPFDNAGKLLYDGTFVIERLASLPDDFLEKNRDALHPVIRELFEQ |
| | VVARKSSATDVFRDLHKQALYIRQMMEIFSPAGISVLVVPTAPLHPTVEQMLAEPISLNSTLGTYTHFGN |
| | VNDLCAVAVPAGTYPLPSTDTTEGSPKEVLPFGITFLGGSRTESEVLNIASRFEAYMTQTAV |
| 458 | WP_040119807.1 MULTISPECIES: cysteine hydrolase [Enterobacteriaceae]: |
| | MIRVAATPDAFCFMAAHCALVIIDMQRDFIEPGGFGSALGNNVAPLREIIPAVERLLLLARRHAIQVIHT |
| | RESHLPDLSDCPPAKYEHGRPGLRIGDAGPMGRILIRGEPGNQIIDQLAPLTGEWTVDKPGKGMFFATGL |
| | DARLRHNGISHLLFAGVTTEVCVQTSMREACDRGYRCLLIEDATESYFPAFKQATLAMIVAQGGIVGRTA |
| | SLAALESALNTQ |
| 459 | WP_040973826.1 cysteine hydrolase [Mesorhizobium sp. ORS 3324]: |
| | MAEIAAQPFAFAFKPATTALIVIDMQRDFTEPGGFGASLGNDVSRVAAIVPTVKKLIEGFRAAGLPVIHT |
| | MECHRPDLSDLPPAKRDRGNPSIRIGDIGPMGRVLIAGEPGTAILDELAPLPEEIVIEKPGKGAFYATSL |
| | GDDLKRIGARQLVFAGVTTEVCVQTTMREANDRGYECLVAEDATESYFPEFKAAALAMIRAQGAIVGWTA |
| | TTDQVLEGIANA |
| 460 | WP_041013827.1 cysteine hydrolase [Pseudomonas xanthomarina]: |
| | MISLAAKPSAFSFDPAHTALVVIDMQRDFLEPGGFGAALGNDVSLLQAAIPAVASLLALARERHMLVIHT |
| | RESHQQDLSDCPAAKREGGAAGLRIGDPGPMGRILVRGEPGNQIIAPLAPMAGEWVIDKPGKGMFYATGL |
| | EDRLLAQGIEYLIFAGVTTEVCVQTSMREANDRGYRCLLIEEATESYFPAYKQATLKMIVAQGGIVGRTA |
| | TLAALHAAMNEEPR |
| 461 | WP_041014574.1 MULTISPECIES: cysteine hydrolase [Pseudomonas stutzeri |
| | group]: |
| | MISVPGKPGAFSFDPARTALVVIDMQRDFLEPGGFGAALGNDVSLLQAIVPAVESLLALAREKGMLVIHT |
| | RESHLPDLSDCPAAKREGGAEGLRIGDPGPMGRILVRGEPGNQIIPSLAPIAGEWVIDKPGKGMFYATGL |
| | GDRLAAQGIECLIFAGVTTEVCVQTSMREANDRGYRCLLIEDATDSYFPAFKQATLEMIVAQGGIVGHTA |
| | TLAALDAAMNEE |
| 462 | WP_042011368.1 MULTISPECIES: cysteine hydrolase [Aeromonas]: |
| | MNKRISAQPFDFTFDPATTALIVIDMQRDFVEPNGFGHALGNDVSLVRRAIEPCRKVLDAARAKGMLVIH |
| | TREGHRPDLTDCLPAKLVRGGKRFIGEQGAMGRILVQGEAGHDIIPELYPIAGEPVIDKPGKGAFYSTDL |
| | HLILQARGIRSLIICGVTTEVCVQTTAREANDRGYELVIPADCCASYFPEFHRVTLEMIQAQGAIVGWVS |
| | DAEQLVAALKD |
| 463 | WP_042417192.1 cysteine hydrolase [Geomicrobium sp. JCM 19038]: |
| | MSEIVVKKSEPHSIQFEFEKSALLIIDMQNEFLLPGGFGERLGNSLANIQSCIEPIQAILQSYRRLNGMV |
| | IHTKEGHSTDLSDCNKSKLERSRLQGAEIGGEGPLGRLLIAGEYGNEIIDKLKPIESEWVIQKPGKGAFY |
| | NTNLEETLRENNITHLIVTGVTTHVCVHSTVREANDRGFNCLIITDGTAAFDLQDHHSALHMITQQGGIF |
| | GWTTSANQLIQAMPS |
| 464 | WP_042580404.1 cysteine hydrolase [Variovorax paradoxus]: |
| | MRIEEANPFAYEFELKSTALVLIDMQRDFIEPGGFGETLGNDVSLLEAIVPATKAALAAWRQAGGLVVHT |
| | REAHKADLSDCPPAKRNRSNPTLRIGDEGPMGRILVAGEPGNQIIDALAPVEGELVIDKPGKGMFYATGL |
| | HELLQQRGITHLLFGGVTTEVCVQTSMREANDRGYDGLLLEDCTESYFPAFKAATLDMIRAQGAIVGWTA |
| | PSAALLAALNHTA |
| 465 | WP_042640635.1 MULTISPECIES: cysteine hydrolase [unclassified |
| | Mesorhizobium]: |
| | MAEIAAQPFAFAFRPETTALIVIDMQRDFAEPGGFGASLGNDVSRVTAIVPTVKRLIEGFRAAGLPVIHT |
| | MECHRPDLSDLPPAKRDRGNPSIRIGDVGPMGRVLIAGEPGTAILDELAPLPGEIVIEKPGKGAFYATGL |
| | GDDLKRLGARQLVFAGVTTEVCVQTTMREANDRGYECLVAEDATESYFPEFKAAALAMIRAQGAIVGWTA |
| | TTDQVLEGIANA |
| 466 | WP_043494107.1 cysteine hydrolase [Hafnia alvei]: |
| | MTTHQFHAEPFALPFNPATTALLMIDMQRDFVEPSGFGEALGNDVSLVRCAIEPCQRVLEAARAQGLFVI |
| | HTREGHRNDLSDCPPAKLTRGGKTFIGTAGPMGRILVRGEAGHDIIPELYPIDGEPVIDKPGKGAFYQTD |
| | LHLVLQNHGIKTLIVCGVTTEVCVTTTVREANDRGYECIIPQDCVGSYFPEFQKYALEMIKAQGAIFGWV |
| | SDANAIVDGLQR |
| 467 | WP_043748933.1 cysteine hydrolase [Pseudooceanicola atlanticus]: |
| | MIFDARPFGLSADPATTALIVIDMQRDFIEPGGFGASLGNDVSLLQAIIPATARLIAGCRAAGIPVIHTR |
| | ECHQPDLSDCPPAKRDRGNPDLRIGDPGPMGRILIAGEPGAQIIPELAPTPGEKVIDKPGKGAFYATDLG |
| | EYLAGLGTKTLIFAGVTTEVCVQTTMREANDRGFDCLLAEDATESYFPRFKQATLDMIRAQGAIVGWTAS |
| | VDEILSALAPVGA |
| 468 | WP_044310314.1 cysteine hydrolase [Pseudomonas syringae]: |
| | MNKVNARPDRFAFDTSRTAVVIIDMQRDFLEPGGFGAALGNDVAPLQAIVPSVQRLLTLARDEGMTVIHT |
| | RESHRPDLADCPQAKRDHGSPGLRIGDPGPMGRILIRGEPGNQIIDALVPLADEWVIDKPGKGMFFATDL |
| | QQRLSQAGITHLIFAGVTTEVCVQTSMREANDRGYRCLLIEDATESYFPAFKKATLEMITAQGGIVGRVA |
| | SLTDLEQALQTRSTH |
| 469 | WP_044311888.1 cysteine hydrolase [Pseudomonas syringae]: |
| | MIKVNARPDRFAFDTSRTAVVIIDMQRDFLEPGGFGAALGNDVAPLQAIVPSVQRLLALARDEGMAVIHT |
| | RESHRPDLADCPQAKRDHGSPGLRIGDPGPMGRILIRGEPGNQIIDTLAPLAGEWIIDKPGKGMFFATDL |
| | QQRLTDAGIIHLIFAGVTTEVCVQTSMREANDRGYRCLLIEDATESYFPAFKQATLEMITAQGGIVGRVA |
| | SLTDLEQALLTRSTL |
| 470 | WP_044321145.1 MULTISPECIES: cysteine hydrolase [Pseudomonas syringae |
| | group]: |
| | MISVNARPDCFTFAPSCAAVVIIDMQRDFLEPGGFGAALGNDVAPLQAIVPSVQQLLALARDQGIVVIHT |
| | RESHSADLADCPPAKLAHGSPGLRIGDSGPMGRILIRGEPGNQIIDSLTPLACEWIIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTP |
| 471 | WP_044391034.1 cysteine hydrolase [Pseudomonas syringae group |
| | genomosp. 3]: |
| | MISISARPDPFTFEPSRTAVVIIDMQRDFLEPGGFGAALGNDVAPLQAIVPTVQQLLALAREQGIAVIHT |
| | RESHRPDLSDCPQAKLDHGLPGLRIGDPGPMGRILVRGEPGNQIIDALTPLASEWVIDKPGKGMFFATDL |
| | HQRLTVAGITHLIFAGVTTEVCVQTSLREACDRGYRCLLIEDATESYFAAFKQATLDMITAQGAIVGRVA |
| | SLANLQHALHTRSTQ |
| 472 | WP_044421333.1 cysteine hydrolase [Pseudomonas syringae group |
| | genomosp. 3]: |
| | MISISARPDTFTFEPSRTAVVIIDMQRDFLEPGGFGAALGNDVAPLQAIVPTVQQLLALAREQGLVVIHT |
| | RESHLPDLSDCPQAKLDHGLPGLRIGDPGPMGRILVRGEPGNQIIDALTPLASEWVIDKPGKGMFFATDL |
| | HQRLTAAGITHLIFAGVTTEVCVQTSLREACDRGYRCLLIEDATESYFPAFKQATLDMITAQGAIVGRVT |
| | SLANLQHALHTRSTP |
| 473 | WP_044538375.1 cysteine hydrolase [Bradyrhizobium sp. LTSP885]: |
| | MTNSSGIIAAEPEPITLDWTKTALVIIDMQRDFMEPGGFGETLGNDVSQLARAVKPIASVLAAARASGML |
| | VVHTREGHLPDLSDAPPAKVERGAPSLRIGDPGPMGRILIRGEAGHDIIPALYPLDSEIVIDKPGKGAFY |
| | ATELGEILQNYGVENLLVCGVTTEVCVNTTVREGNDRGYRCVVIGDGCASYFPEFHEMGLKMIKAQGGIF |
| | GWVSDSAAILKAMET |
| 474 | WP_044587361.1 cysteine hydrolase [Bradyrhizobium sp. LTSPM299]: |
| | MTNSSGIIAAEPEPITLDWMKTALVIIDMQRDFMEPGGFGETLGNDVSQLARAVKPIASVLAAARASGML |
| | VVHTREGHLPDLSDAPPAKVERGAPSLRIGDPGPMGRILIRGEAGHDIIPALYPLDSEIVIDKPGKGAFY |
| | ATELGEILQNYGVENLLVCGVTTEVCVNTTVREGNDRGYRCVVIGDGCASYFPEFHEMGLKMIKAQGGIF |
| | GWVSDSAAILKAMET |
| 475 | WP_044883352.1 cysteine hydrolase [Frankia torreyi]: |
| | MSETATTPTAPLTVSARPYDFTFDPATTALVVIDMQRDFLEPGGFGESLGNDVSQLRSTIEPLQAVLAAV |
| | RAAGLTVIHTREGHLPDLSDLPPAKLHRGDAALRIGDLGPKGRILIRGEYGQDIIDELAPVDGEYVIDKP |
| | GKGAFYATAFGDVLAEKGITSLVVAGVTTEVCVHTTVREANDRGFECLVLSDCVGSYFPEFQRMALEMVA |
| | AQGGIFGWVAPSADFLAALASSAPAADSTVPAPAVTAS |
| 476 | WP_045002889.1 MULTISPECIES: cysteine hydrolase [unclassified |
| | Bradyrhizobium]: |
| | MLNSTKPTLGVISAEPEPIKLDWASTALVIIDMQRDFMEPGGFGETLGNDVSQLARAVKPIGALLTAARD |
| | TGMLVVHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKTSTT |
| 477 | WP_045195078.1 MULTISPECIES: cysteine hydrolase [unclassified |
| | Rhodococcus]: |
| | MTSAQTPETSIPSASPSEFTIDPTTTALLVIDMQRDFLLPGGFGESLGNDVGLLRSVIEPLAGLIAVARE |
| | AGIPVIHTREGHLPDLSDCPPAKLRRGTPSQRIGDPGAFGRILVRGEYGHDIVDELAPLEGETVIDKPGK |
| | GAFYATELSEILTSAGITTLLVTGVTTEVCVHTTVREANDRGYECLVVTDCVGSYFPEFQRVGLEMISAQ |
| | GGIFGWTAPSEDVEAALVALVPTSASR |
| 478 | WP_045231533.1 cysteine hydrolase [Agrobacterium rubi]: |
| | MVEIKALPFAFPARPQELALIVIDMQRDFAEPGGFGASLGNDVSGIARIVPDVKRLIEGFRAAGLPVIHT |
| | MECHRPDLSDLPAAKRDRGNPSMRIGDVGPLGRVLIAGEPGTAILPELAPIEGEVVIQKPGKGAFYATDL |
| | SGVLKDKGITQLVFAGVTTEVCVQTTMREANDRGFECLLVEDATESYFPEFKATTIAMIRAQGAIVGWTA |
| | MIDDILEGIAHG |
| 479 | WP_045367604.1 cysteine hydrolase [Methyloceanibacter caenitepidi]: |
| | MPFIDAKPFPFQFDFDHIALICIDMQRDFCQPGGFAESLGNNIANIQPCIPVIGKLQAAFRKAGLPIIHT |
| | KECHQPDLSDLPTAKRNRGNPKVKIGEFGPMGRILVDGEPGVEFVSENEPREYEHVISKPGKDSFYRTDL |
| | DEYLTRRKISGLVITGVTTEVCVQTTMRCANDRGYDCLLVEDGTDSYFPEFKEMTLKALVAQGGIVGWTC |
| | KSDVLLDMMAKEVPGQTSPHKAA |
| 480 | WP_045672421.1 cysteine hydrolase [ Paenibacillus beijingensis]: |
| | MNAYVPSMQVENALPYPFGFDPASTAVVVIDMQNDFCAPGGFGQRLGNDIAAVRAIIPTISRVLDAARSA |
| | GLLIIHTREGHLPDLSDCPPSKQERSRRQGAGIGDAGPMGRILIRGEPGHEIIPELTPIPGEPVVDKPGK |
| | GAFYQTNFHDILIEYGIESLILCGVTTHVCVHTTLREANDRGYRCLVLEDATAAFDPDDHAAAIHMVRQQ |
| | GGIFGWTSASISLIHTLRK |
| 481 | WP_045774122.1 cysteine hydrolase [Elstera litoralis]: |
| | MIDIPAEPGPFPLDPAAVALIVIDMQRDFVEPGGFGASLGNDVSRLTAIIPAVADLIGLFRQKGWPVIHT |
| | RESHLPDLSDCPPAKRLRGKPSLRIGDPGPMGRILVRGEPGNQIVDGCAPLPGEVVIDKPGKGAFYKTNL |
| | DALLMQTGIRQLVFAGVTTEVCVQTSMREANDRGFECLLVEEATESYFPEFKAATLAMIHAQGGIVGWTC |
| | TLPALQKAVAP |
| 482 | WP_046021799.1 cysteine hydrolase [Magnetospira sp. QH-2]: |
| | MPVIANALPFAFEFDPATTALVVIDMQRDFLEPGGFGEALGNDVSQLAPVVPATEKLLAACRAAGLEIVH |
| | TRESHLPDLSDCPPAKRNRGSCKLRIGDPGPMGRILVRGEPGNDIVPSLAPLPGETIIDKPGKGAFYKTG |
| | LTHRLADDGITHLIFAGVTTEVCVQTSMREANDRGFDCLLVADCTGSYFPEFKQATLEMVRAQGGIVGWT |
| | ADLYAVLEALDG |
| 483 | WP_046104329.1 cysteine hydrolase [Devosia chinhatensis]: |
| | MLISIPARPYPYSLDPQHTALVVIDMQRDFIEPGGFGDSLGNDVRRLEAIVPATAALIDLFRRQGWPIVH |
| | TREAHQPDLSDCPPAKRARGKPGLRIGDEGSMGRILIAGEPGNQIVDALAPREGEIVIDKPGKGMFHATG |
| | INERLRETGITHLVFAGVTTEVCVQTSMREANDRGYECLLVEDATESYFPAFKAATIEMIVAQGGIVGWV |
| | ATLSALVHAVAKEHADA |
| 484 | WP_046266756.1 cysteine hydrolase [Pseudomonas syringae]: |
| | MIKVNARPDRFAFDTSRTAVVIIDMQRDFLEPGGFGAALGNDVAPLQAIVPSVQRLLTLARNEGMAVIHT |
| | RESHRPDLADCPQAKRDHGSPGLRIGDPGPMGRILIRGEPGNQIIDTLAPRAGEWLIDKPGKGMFFATDL |
| | QQRLTDAGITHLIFAGVTTEVCVQTSMREANDRGYRCLLIEDATESYFPAFKQATLEMITAQGGIVGRVA |
| | SLTDLEQALLTRSTL |
| 485 | WP_046363294.1 cysteine hydrolase [Mycolicibacterium obuense]: |
| | MNPIPIAAEPSPFPLIAGKTALVVIDMQRDFLLPGGFGESLGNDVARLATVVPPLAALLAAARSAGLMVI |
| | HTREGHRPDLSDCPPAKLRRGAPTQRIGDPGAFGRILIRGEYGHDIVDELAPIDGEVVIDKPGKGAFYGT |
| | DLSEVLTDAGITQLLITGVTTEVCVHTTTREANDRGYECLVVSDCVGSYFDDFHRVGLQMIAAQGGIFGW |
| | VADTAAVIPALQQLTTTAA |
| 486 | WP_046580802.1 cysteine hydrolase [Burkholderia gladioli]: |
| | MHFEVPARPAPYRYDPAHTALIVIDMQRDFIEPGGFGAALGNDVAPLAAIVPSVAALLAFARARGWHVVH |
| | TRESHAPDLSDCPPAKRLRGAPNLRIGDSGPMGRILVRGEPGNQIVEALAPLAGETVIDKPGKGAFHATA |
| | LDALLRERGITHLVFAGVTTEVCVQTSMREANDRGYDCLLVEDATASYFPAFKAACLEMISSQGGIVGWT |
| | APLGALLEAAPLPAAPSASPQP |
| 487 | WP_046608667.1 cysteine hydrolase [Neorhizobium galegae]: |
| | MGQIKAEPFAFPAKPEALALIVIDMQRDFAEAGGFGASLGNDVGRITKIVPDVKRLIQGFRDAGLPVIHT |
| | MECHKPDLSDLPPAKRNRGNPSLRIGDLGPMGRVLISGEPGTAIISELAPIDGEVVIEKPGKGAFYATEL |
| | GEVLKAKGISQLVFAGVTTEVCVQTTMREANDRGYECLLAEEATESYFPEFKAAAIAMIRAQGAIVGWTA |
| | HVDDILEGIAPKGMTNA |
| 488 | WP_046625846.1 cysteine hydrolase [Neorhizobium galegae]: |
| | MVQIKAEPFAFPAKPEELALIVIDMQRDFAEPGGFGASLGNDVGRITRIVPDVKRLIQGFRDAGLPVIHT |
| | MECHKPDLSDLPPAKRDRGNPTLRIGDVGPMGRVLISGEPGTAIISELAPVDGEVVIEKPGKGAFYATEL |
| | GEVLKEKGISQLVFAGVTTEVCVQTTMREANDRGYECLLAEEATESYFPEFKAAAIAMIRAQGAIVGWTA |
| | HVDDILEGIAPKGMTNA |
| 489 | WP_046667526.1 cysteine hydrolase [Neorhizobium galegae]: |
| | MGQIKAQPFAFPAKPGALALIVIDMQRDFAEPGGFGASLGNDVGRITKIVPDVKRLIQGFRDAGLPVIHT |
| | MECHKPDLSDLPPAKRDRGNPTLRIGDVGPMGRVLISGEPGTAIISELAPIDGEVVIEKPGKGAFYATEL |
| | GDVLKAKGISQLVFAGVTTEVCVQTTMREANDRGYECLLAEEATESYFPEFKAAAIAMIRAQGAIVGWTA |
| | HVDDILDGIAQKGMTDA |
| 490 | WP_046793590.1 cysteine hydrolase [Rhizobium sp. LC145]: |
| | MAAIEAQPFPFPARPNELALIVIDMQRDFAEAGGFGESLGNDVSRIGKIVPDVKRLLEGFRAASLPVIHT |
| | MECHRPDLSDLPPAKRDRGNPTLRIGDQGPMGRVLIAGEAGTSIIAELAPVDGEIVIEKPGKGAFYATGL |
| | GQALAEKGITQLVFAGVTTEVCVQSTMREANDRGFECLLAEEATESYFPEFKVAALSMIRAQGAIVGWTA |
| | HVDDILKGISHA |
| 491 | WP_046977204.1 cysteine hydrolase [Rhizobium phaseoli]: |
| | MAEIKAEPFAFPVKHDQLALIVIDMQRDFAEPGGFGASLGNDVSRIGRIVPDVKRLIQGFRYAGLPVIHT |
| | MECHRPDLSDLPPSKRDRGNPMLRIGDEGPMGRILIAGEPGTAILPELAPIDGEVVIEKPGKGAFYATGL |
| | AEALQRKGIRQLVFAGVTTEVCVQTTMREANDRGYECLLAEEATESYFPEFKAAAIAMIRAQGAIVGWTA |
| | HIDDILESIAHA |
| 492 | WP_047331555.1 cysteine hydrolase [Mycobacterium sp. EPa45]: |
| | MVTINAEPFALDFDVSSAALVIIDMQRDFVLPGGFGEALGNDTSLLLAAVEPIERVLARAREIGMLVIHT |
| | REGHRPDLSDCPPAKLHRGGKTFIGEPGPMGRILVRGEQGHDIIDQLYPIDGEPVIDKPGKGSFHATDLG |
| | QILADRGIKTLVVCGVTTEVCVHTTVREANDRGYECLVLRDCVASYFPEFQRVALEMIKAQGAIFGWVSD |
| | ADEFIAATS |
| 493 | WP_047372681.1 MULTISPECIES: cysteine hydrolase [Enterobacterales]: |
| | MRSIKAQPFDFQFDPATTALVVIDMQRDFVERGGFGEALGNDVSLVRRAIEPCAALLKSAREAGLLIIHT |
| | REGHRDDLSDCLPAKRTRGGKTFIGEPGPMGRILVRGQPGHDIIPELAPRPGEPVIDKPGKGAFYATDLH |
| | LILQSQRISSLIICGVTTEVCVQSTAREANDRGYELVIPEDCCASYFPEFHQAALAMIKAQGAIVGWVSH |
| | SAEVIAALRP |
| 494 | WP_047784112.1 cysteine hydrolase [Variovorax paradoxus]: |
| | MRIETANPFPYDFELKNTALVLIDMQRDFIEPGGFGETLGNDVSLLEAIVPATKAALQAWREAGGLVVHT |
| | REAHKADLSDCPPAKRNRGNPSLRIGDEGPMGRILVAGEPGNQIIDALAPVDGEIVIDKPGKGAFYATGL |
| | HELLQRRGITHLLFGGVTTEVCVQTSMREANDRGYDSLLLEDCTESYFPAFKAATLDMVRAQGAIVGWTA |
| | PSAALMAALRQGQ |
| 495 | WP_047845980.1 cysteine hydrolase [Caballeronia mineralivorans]: |
| | MTITIPALPGPFTFEPSMTALVIIDMQRDFIEPGGFGESLGNDVSLLAQIVPTVASLLAFARRSGWFVVH |
| | TRESHAADLSDCPPAKRLRGAPNARIGDDGPMGRILIRGEPGNAIVDAVAPVEGELVIDKPGKGAFYATS |
| | LTPELEARHITHLVFAGVTTEVCVQTSMREANDRGYDCLLVEDATASYFPAFKQAALDMVRSQGGIVGWT |
| | APLSSFTAA |
| 496 | WP_048421216.1 cysteine hydrolase [Mycolicibacterium chubuense]: |
| | MNPIPVAAEPAPFPLVAGKTALIVIDMQRDFLLPGGFGESLGNDVERLRTVVPPLSALLAAARAAGIMVV |
| | HTREGHRPDLSDCPPAKLQRGAPSKRIGDPGTYGRILIRGEYGHDIIDELAPLEGEVVIDKPGKGAFYGT |
| | DLSDVLTGADITQLLITGVTTEVCVHTTTREANDRGYECLVVSDCVGSYFPDFHRVGLQMVTAQGGIFGW |
| | VADSAAVIPALHQLTTTAA |
| 497 | WP_048471100.1 cysteine hydrolase [Mycolicibacterium |
| | chlorophenolicum]: |
| | MNPIPVAAEPAPFPLVAGKTALIVIDMQRDFLLPGGFGESLGNDVERLRTVVPPLSALLGAARAAGITVI |
| | HTREGHRPDLSDCPPAKLQRGAPSKRIGDPGTYGRILIRGEYGHDIIDELAPLEGEVVIDKPGKGAFYGT |
| | DLSDVLTGAGITQLLITGVTTEVCVHTTTREANDRGYECLVVSDCVGSYFPEFHRVGLQMVTAQGGIFGW |
| | VADSAAVIPALHQLTTTAA |
| 498 | WP_048823376.1 cysteine hydrolase [Bacillus sp. B-jedd]: |
| | MTKQLTMKAKPFDFEFNPEHTALVIIDMQRDFCYPGGFGEKLGNDITLTRSIIPQLQRVLEKARESGLTV |
| | IHTREGHRQDLSDCPPSKLNRGKKQGAGIGDEGPMGRILVRGEYGHDIVDELKPVNGEIIIDKPGKGAFY |
| | RTDLDLILKNKEITHLLVGGVTTHVCVQTTIREANDRGYECLLLEDCAAAFDPQDHEDSIRMIHQQGGIF |
| | GWTAPSESLLKVL |
| 499 | WP_049637924.1 cysteine hydrolase [Methylophilus sp. TWE2]: |
| | MKILSVPALPEPFDVDLTHTALLIIDMQRDFIEEGGFGQSLGNDVSLLKAAIAPCQAVLAAARAQGILVI |
| | HTREGHRSDMTDAFPAKVERGSPKLRIGDPGPMGRILIRGEPGHDIIPALSPIAGEPVIDKPGKGAFYAT |
| | DLELLLRKRNIEALIVCGVTTEVCVHTSVREANDRGFRCLIPGDCCASYNPEFHAVSLRMFAAQGAIFGW |
| | VTDSQQLVNVLQK |
| 500 | WP_050453040.1 cysteine hydrolase [Candidatus Burkholderia |
| | verschuerenii]: |
| | MPTLAHAQPSPFSFEPRRTALVVIDMQRDFIEPGGFGEALGNDVSLLASIVPTVESLLAFARGNGWHVVH |
| | TRESHAPDLSDCPDAKRLRGAPHARIGDAGPMGRILVRGEPGNAIIDALTPVEGELVIDKPGKGAFYATR |
| | LGEELALRGVTHLVFAGVTTEVCVQTSMREANDRGYECVLIEDATASYIPAFKAATIEMIRSQGGIVDWT |
| | ATLADVLEA |
| 501 | XP_013281726.1 allophanate hydrolase [Fonsecaea pedrosoi CBS 271.37]: |
| | MGMSKDKQGFLTIEAKPYPFSFPLKHTALLVIDMQRDFICAGGFGEIQGGNLEAVQASIAPTKQLLDACR |
| | DAGLHIFHTREGQVPSLADCPSSKLVRQAAAPGNTQHLKVIGDKGDMGRLLVRGEYGHDIVDELQPLPSE |
| | VVIDKPGKGSFWNTPILHKLKAGGITHLLVSGVTTECCFSTTIREANDRGFECCGIVQSTAGYNAAFKTA |
| | SLDMIHWSQGLFGFVADLQPVLDVLSPWQSQNKGVSTPPQTPPSWDGKLGIADLQASYKRGLSPLELVNA |
| | LFDRIEKYEHIDGAVWIRRESRAGVLDQARRLLELYPDKNARPALFGVPFTVKDSIDVQGVETTTACPPL |
| | AFVATRSATCYQKVVGQGALYLGKVNLDQLATGLSGCRSPFGITHSVFSDEHISGGSSSGSCVSVGADLA |
| | TFSLATDTAGSGRVPAGFNGVVGYKPTRGLVSFEGVTPACLSLDCIAFTARTVEDARTLWQVCEGYDEND |
| | RYARDTFPAERHVNSIGAQRETFRFGIPPPELLEVCSPSFRKLFNEAISRLQGMGGTLVPMDWTPFQKAG |
| | DLLYEGTFVSERLASLADDFLEKNRQHLHPVILELFEKVVARQSTAVQLFRELQAKALYTRQATSQFRSA |
| | DRSGLDVVVVPTAPWHPTIKEMLADPIRLNAKMGTFTHFANVLDMCGIAVPSSTYQESEAGPRLPFSITL |
| | LGSRCSDSEVLDIASRYQEATAR |
| 502 | XP_013260639.1 hypothetical protein A109 05972 [Exophiala aquamarina |
| | CBS 119918]: |
| | MSMPSQTSGFLTIEAKPYPFAFPRQHTALLVIDMQRDFICAGGFGEIQGGNLEAVQASIAPTKSLLEACR |
| | NAGLQIFHTREGQVPSLADCPSSKLIRQAAAPENTQHLKVIGDKGEMGRLLVRGEYGHDIVDELQPRASE |
| | VVIDKPGKGSFWNTGIMHKLKARGITHLLVSGVTTECCFSTTIREANDRGFECCGIAQSTAGYNPAFKTA |
| | SLDMIYWSQGLFGFVADLQPVLDALAPWKRESNGETTPPQTPPMWDGEIGISELQQSYRTGLSPIELVNT |
| | LYDTIEKYDRIDPAVWIKRESRDSVLDSARKLLEQYPDKNSLPPLFGIPFTVKDSIDVQGIETTTACPPL |
| | AYVASKSAVVFQKVISQGALYLGKVNLDQLATGLSGCRSPYGVTHSVFSDKHISGGSSSGSCVSVGAGLA |
| | TFSLATDTAGSGRVPAGFNGVVGYKPTRGLISFEGVTPACLSLDCIAFTARTVADARTLWQACEAFDVND |
| | RYSRDTFPLERHVNSLGSQRCEFRFGIPPPEILEICSPTFRKLFNEAVQQLQQLGGILTPIDWTPFQQAG |
| | DLLYAGTFVSERLASLPDDFLDKNRQHLHPVILELFEQVVARQSTAVQLFRDLQTKALCTRNATSQFASA |
| | DKLGIDVLVVPTAPEHPTIEAMLADPIRLNSKMGTFTHFGNVLDLCGVAVPSGTYVPADEAAPQLPFSIT |
| | FLGARCTDSEVLEIASRFQRRR |
| 503 | XP_013273664.1 allophanate hydrolase [Rhinocladiella mackenziei CBS |
| | 650.93]: |
| | MGEANDKPGFLTIEAKPYPFTFPLKHTALLVIDMQRDFICSGGFGEIQGGNLEAVQASIGPTKSLLEACR |
| | HAGLPIFHTREGQVPSLADCPSSKLIRQAAAPGNTQHRKVIGDKGEMGRLLVRGEYGHDIVDELQPLASE |
| | VVIDKPGKGSFWNTPILHKLKARGITHLLVSGVTTECCFSTSIREANDRGFECCGITQSTAGYNPAFKTA |
| | SLDMISWSQGLFGFVADLQPVLDALSPWQKKSNGVSTPPQTPPTWDGKLGIPDLQRAYRKGLSPMEVVNA |
| | VFDRIEKYDDVDPAVWIKRESRDAVLESARHLLELYPNRSALPPLFGVPFTVKDSIDVQGIETTTACPPL |
| | AFVATKSAACYQKVISQGALYLGKVNLDQLATGLSGCRSPYGVPHSVFSKDHISGGSSSGSCVSVGAGLA |
| | TFSIATDTAGSGRVPAGFNGVVGFKPTRGLVSFEGVTPACLSLDCIAFTAKTVEDARTLWQVCEEYDEND |
| | RYARDTFPAERHVNALGTQHEAFRMGIPPPELLEVCSPTFRKLFNEAIKRLQSMGGILVPIDWTPFQKAG |
| | DLLYEGTFVSERLASLPDDFLERNRVHLHPVTLELFEKVVARQSTAVQLFRDLQTKALCTRQATSQFASA |
| | DKLGMDVLVVPTVPEHPTIEAMLADPIRLNAKMGTFTHFANVLDMCGVACPAAEYLSGEAGPRLPFSITF |
| | LGCRCLDSEVLEIASRFLEGMPREV |
| 504 | XP_013327636.1 hypothetical protein T310 4930, partial [Rasamsonia |
| | emersonii CBS 393.64]: |
| | VIHTREGHQPDLADLPAAKRLRQISAPDGHHTMGIGDRGPMGRLLIRGEYGHDIIDELTPRPGELVIDKP |
| | GKGSFWGTGFHRALLARGITHLLVTGVTTECCVTTTLRECNDRGYECCLLTDCTAGFDAQMVQTAMDTIC |
| | GQDGLFGYVGQSSDLLSFSDQANTPPATPPTTAESYLPSIQELRQRYQSGLEDPVRIVNLVFDRIEEYQK |
| | TDPAVWVSTRPREDCVAAAQALSAKYAGQALPPLFGIPFGVKDNIDVQGIRTTAACEKYAYVAQSHAFAV |
| | QLLLEAGALYIGKLNMDQLATGLSGCRSPYGAPRCVYSKDHIAGGSSSGSAVAVAAGLVSFALGTDTAGS |
| | GRIPAAFNGVVGLKPTKGTISARGVVPACKSLDTISITAPTLSDARTVWLILDQHDPHDPYAKVPSSLPT |
| | WHIDFRGPRTGGFKFAIPPPSVLETCSKPYQEQFARSVQLLRSCGGSLVKIDYTPVQAAGELLYNASLLY |
| | ERIASIGSEFLLANLDALHPTTRALFQAALYRKIEPWTVFRDQDLQRRYTRQVQRIFDPLAGGSIDVLLV |
| | PTAPCHPTMQEMERDPLGLNSTLGTFTHAANVLDLCGVSVNAGWIEETGLPFGVTFLGGMGYDGKILDIA |
| | AVFVEKIKGRKTDK |
| 505 | XP_013310898.1 allophanate hydrolase [Exophiala xenobiotica]: |
| | MGGSPKDVLAIEAKPYPFTFPLQSTALLVIDMQRDFICSGGFGEIQGGSLEAVQASIAPTKALLQACRHA |
| | GMHIFHTREGHVPSLADCPSSKLIRQAAAPGNSQHLKVIGDKGEMGRLLVRGEFGHDIVGELQPLPSEVV |
| | IDKPGKGSFWNTPLLHKLKSSGITHLLVSGVTTECCFSTTIREANDRGFECCGIRESTAGYNAAYKTASL |
| | DMIHWSQGLFGFVADLQPVLDALSPWQKSSPEVSTPPQTPPAWDGNLGISDLLASYKQGLSPVVMVNELE |
| | DRIEKYDAIDPAVWIKRQSREEVLNNVTHLLERFPDRNALPPLFGVPFTVKDSIDIQGIETTTACPPLAF |
| | VASKSAVCYQKVIDAGAIYLGKVNLDQLATGLSGCRSPYGITHAVASKDHVSGGSSSGSAVSVGADLATF |
| | SLATDTAGSGRVPAGFNNVVGFKPTRGLISFQGVTPACLSLDCIALIAKTVEDARIVGQVCEGFDPNDRY |
| | ARDTFPLPRHVNSIGPQRDAFHFGIPPPEVLEICSPTYRKLFNEAVQQLQGLGGVLTSVNWDPFKKAGDL |
| | LYEGTFVSERLASLPDDFLEKNAQYLHPVILELFEKVVARQSTAVQLFRELQRKAIVTRQSTNQFASADR |
| | FGVDVLVVPTAPEHPTIEAMLADPINLNAKLGTFTHFANVLDLCGVAVPSGSYFADDKAASPRKLPFSIT |
| | FLGCRCSDSEMLSVASRYQERHGA |
| 506 | XP_013338985.1 hypothetical protein AUEXF2481DRAFT_71274 |
| | [Aureobasidium subglaciale EXF-2481]: |
| | MELPSARPYAFRFRPESTAVVIIDMQRDFLDRGGFGELQCGNAEIFENVRQIVPQTKEVLKAARKLGLHV |
| | IHTREGHTPNLSDLPASKRLRQKAAPSGHHHIGIGDEGPMGRLLVQGEYGHDIIDDLKPVPGETVIDKPG |
| | KGSFWNTTLHRSLLARGITHLLIAGVTTECCVNTTFREASDRGFECCVLTDCTSGFEGSFVDSTLNMLCS |
| | YDGLFGYVCASNELLNYAHDSHPTPPRTPPGFQGDLSLASLQRQFKNREITVVEVAKDVSRRVSEYQKKD |
| | PAVWTYLQSGEKLLKAAQALEERYMHQPLPPLYGIPFAVKDNIDVEGIFTTGACQQASYMPKKSAKVVTA |
| | LIRAGALFIGKTNLDQLAAGLSGCRSPFGYPRSVFDHERVSGGSSSGSAVAVAAGLVTFALGTDTAGSGR |
| | VPAAFNGITGFKPTRSTLSAEGLVPACRSLDTISILALTVIEARVVWLVADEGPDMSDPFAKTQQSLPLW |
| | HVDFRGVREGGFVFGVPPASALAICTPTYRKHFDIAVERLERSGGVRKEVEWTPFEGGSQLLYNGALMNE |
| | RVQCADPEFLLNNQQHLHPTTRKLFEAAMGRDLKPWDVYRDQHLQATYTRQAALIFEEIDVLLVPTTTCH |
| | PTVAEMESDPITLNAKLGEFTHFANVLDLCGIAVPASCYEENAAEPLPFGVTLIGASGTDGKVFDIAKVF |
| | EETA |
| 507 | WP_052065069.1 MULTISPECIES: cysteine hydrolase [Rhodococcus]: |
| | MTSTHRPESTIATASPSEFTIDAARTALLVIDMQRDFLLPGGFGESLGNDVGLLRSVIEPLAGLISAARE |
| | SGIPVIHTREGHLPDLSDCPPAKLRRGTPSQRIGDPGAFGRILIRGEYGHDIVDELAPIDGETVIDKPGK |
| | GAFYATELAEILIAAGITTLLVTGVTTEVCVHTTVREANDRGYECLVVTDCVGSYFPEFQRVGLEMISAQ |
| | GGIFGWTASSEDWAALSAFVPTSASR |
| 508 | XP_013430626.1 Isochorismatase hydrolase [Aureobasidium namibiae CBS |
| | 147.97]: |
| | MELPNAIPYAFEFRPESTALVIIDMQRDFVEPGGFGSIQCGDDKVFNAVRRIVPVVQRALEASRKLGLYV |
| | MHTREGHLPDLSDLPASKRLRQTNAPNGHHTIGIGEPGPMGRLLIRGEYGHDIVDELRPLPGEVIIDKPG |
| | KGSFWKTGFHRALLNRGITHSLLAGVTTECCVNTTAREAADRGFECCILNDCTSGFDANLVTSANATICA |
| | YDGLFGYVALKSMSISDLSQAYRQNTLRPMDVIRAAAEKASEYLRQNPCVATILTNPETLIREAESLQQK |
| | FVGKPLPPLYGIPFTIGRYEDYAEIDALVDAGALLVGSLSEPSASVAGLGVSFALDSYPSSIARTRTSTG |
| | VTVFDPTSTGPTSIVAQSSEEAHKVWLVIDQGPSDEENTLIVPRSVVDSWVWHVDFCGTKTGGFVFGLLR |
| | DKSCCSDVSHHLRTQKAIQQLQAAGGRAQEIDYAVFEQAKKVNRDMFLLAVKETVSMQAVLELQVRQLEL |
| | SRAATKILETVDVLDDPMTTCLCHSACERAEQTRCLVESLGLCGISVDSGLIQTQQGYNGLTLMLVGGTG |
| | RDGRILDIARELEKTMSHRFA |
| 509 | WP_053199924.1 cysteine hydrolase [Herbaspirillum hiltneri]: |
| | MITVDAIPYPYQFDSRHTALVVIDMQRDFVEEGGFGSVLGNDVRPLTTIVPTVAKLLALARENGLLVVHT |
| | RESHLPDLSDCPPAKLKRGNPALGIGDEGPMGRILVRGEPGNQILPLLAPQDGELVIDKPGKGAFYATGL |
| | HTELQARGVTHLLFAGVTTEVCVQTSMREANDRGYECLIVEDACASYFPVFHQATLAMLTAQGGIVGWQA |
| | PLSTLQTAFKETSGETTS |
| 510 | WP_053310043.1 cysteine hydrolase [Vibrio alginolyticus]: |
| | MIKSFNADPFALEFDPTTTALVMIDMQRDFVEPGGFGEALGNDVSLVRTAIEPCVAVLEAARQAGLTVIH |
| | TREGHRADLTDCPAAKLTRGGKTFIGEMGPKGRILIRGEEGHDIIPELYPIAGEPIIDKPGKGAFYQTDL |
| | HLILQTRNIKTLIVCGVTTEVCVTTTVREANDRGYECIVPEDCVGSYFPEFQKYALEMIKAQGGIFGWVS |
| | HSKDIIEVIK |
| 511 | XP_013897805.1 isochorismatase hydrolase [Monoraphidium neglectum]: |
| | MRDANDMGYECLLLSDCTAATAAANHLAACDMVKKQGGVFGAVADSGALLAAIEKLPAPAAPPAPPPAAA |
| | SPIATVAARPYPYSLPLASTAVIMIDFQKDFMLKGGFGDTLKNDVGLLMECVPGAQRLLAVARAAKLPIV |
| | HTLEAHKPDLSDLHTSKLTRGNLPEELRIGATGAMGRILVAGEEGNWIIDELVPLPGEELVHKPGKGAFY |
| | ATGLEPYLKSKGITHLLFAGVTTEVCVQTTMREANDRGYECLLVTDATASYFPAFKDAAIEMIVAQGGIV |
| | GWAADSVALEEALKQADTA |
| 512 | XP_013945944.1 hypothetical protein TRIATDRAFT_316778 [Trichoderma |
| | atroviride IMI 206040]: |
| | MAPDGKLLLRNARPYAFSCPAATTALVIIDMQRDFLDPDGFGSVVCANPAAFSSARKIVPNVRKALEAAR |
| | SIGMHVIFTREGHLPNLSDLPAAKRLRQTSAPNGSKSLGIGDEGPMGKLLVRGEKGHDIIDELKPHPGEP |
| | IIDKPGKGSFWGTEFHRLLLARGITHLILAGVTTEEGNDRGYECCALSDCTAGFNENMVATSLDILCCQN |
| | GLFGYVGHGSEFAAEVEQFCQLIPSSADYNLNSPTLPSIDQLRSLYKDGRITPEAIIISVFDRIAKYENI |
| | NPAVWISRQSQEDVLAAARKLSATYAGKPLPPLFGIPFAIKDNIDVEGVVTTAACESYAYTATFTAPSIQ |
| | HLLDAGAIYIGKLNLEQLATGLVGRRSPYGDLHCFHSKDHVPGGSSSGSAVAVAAGLVSFAIGTDTAGSV |
| | RAPAAFNGVVGFKPTKGTISARGAVPACQSLDTIGVLAPSVADARQVWYVLDRHDSLDPYAKPPASLPTW |
| | AVDFRGPKEGGFTFGVPPDSLLHLCSKEYQEMFRKAVDTLQSIGGTLVEIDYTPFATAGDLIYGASLIHE |
| | RLASIGYEFLSEKIDTLHRTTKLVIQKVLSSDLKGWEVYRDQAIQMECTAKGRQVFNKFEDGIDVLVMPT |
| | VPWHPTIQEIEESPITPNSKIGIFTHPGNVIDLCGVSVNAGWAEDGGVRLPFGITFQGGSGYDGKVLDIA |
| | AAFEKDLAEKNILVQ |
| 513 | XP_013951523.1 hypothetical protein TRIVIDRAFT 205920 [Trichoderma |
| | virens Gv29-8]: |
| | MTSKMELSLPNARPYEFAFPLATTAFIVIDMQRDFLDPDGFGSIACGNPAIFSAVRKIVPNVQRALEAAR |
| | SMGLHVIYTREGHLSNLSDLPATKRFRQVNAPNGNQLIGIGDEGPMGKLLVRGERGHDIIDELKPYPGEP |
| | IIDKPGKGSFWGTGFHRLLLARGITHLILTGVTTECCVTSTLRECNDRGYECCVLSDCTEGFDPAMVATS |
| | LDIVCCQDGLFGYVGHSGEFISQTNEAHSLKPALTVDLDATALPSINELRGLYRNGLLNPEAVIQSVLER |
| | IAKCESINPSVWISKESPVDILAAVRTLSATYAGKELPPLFGIPFAVKDNIDIKGVVTTVACDSFAYTAT |
| | ATAPAIQHLLDAGAIYIGKLNLDQLATGLTGCRSPYGIPHSYYSKRHISGGSSSGSSIAVAAGLVSFAIG |
| | TDTAGSVRAPAAFSGVVGFKPTKGTISARGAVPACQSLDTLGILAPSLSDARQVWYVMDQHDHLDPYAKP |
| | PSSLPTWIVDYRGFREGGFTFGIPPDSLLQMCSAKYQELFKVAVGKLQSCGGTLIDIDYAPFAKAGDLIY |
| | NASLVHERLASIGYEFIVENIDTFHPTTKSIFQGVLSSNLKAWEVFRDQATQMQCIAEARRTFNKLEEGI |
| | DVLVVPSMPWHPTIQEILDDPLALNSKLGLFTHPANVVDLCGVSVNAGWIDEEGIRLPFGITFLGDSGYD |
| | GKVLDIAAIFENLIK |
| 514 | XP_014075737.1 hypothetical protein COCC4DRAFT_203337 [Bipolaris |
| | maydis ATCC 48331]: |
| | MAKTTAKPNMVSFDAKPYAFSFPLDHTALLIIDMQRDFLLPQGFGEIQGGNLEAVQASIAPTKRLLDACR |
| | SAGLTIVHTREGHKPDLSDCPSSKLTRQEAAPGNTQHKLVIGDKGELGRLLTRGEYGHDIIDELRPLPGE |
| | VVVDKPGKGSFWNTSILHQLKARAITHLIVSGVTTECCFATTIREANDRGFECCGIEEATSGYNDACFKK |
| | PTLDMIHWSQGLFGFIGSLQPLVEALEPFSTKQIQLASTPPQTPPEFDGDLTISSLQRAYRNGLSPLTVI |
| | EAVYSKIEAYKKIDPAVWIHLQPLESALEAARDLITKFPDRTALPPLFGIPFSVKDSIDIAGLPTTTACP |
| | PLAHIPSTSAVVYEKVISQGAIFIGKTNLDQLATGLVGCRSPYGTPHSVYHPSYISGGSSSGSAVSVAAN |
| | IVSFSLATDTAGSGRVPAGLNGIVGFKPTRGTISFRGITPACLSLDCVALATKTIPDARTLWQILESYDP |
| | LDPYSKPALAFERHINSIGPQSSTFKFGIPPQEALGVCSAPTRRLFNATVSKLQALGGVLTPINWSPFHK |
| | AGELLYEGTFVSERLASLPDDFLDKNRGGLHPVTAQLMDAVLQRKSSAVDAYRDLQAKALYTRQAEDVFA |
| | YSAHGIDVLVVPTTPTHWRIDEVLQDPIRKNSALGEFTHCGNVLDLCGVAVPAGEYPVRELSGREEDEGI |
| | LPFSVTFLSGSRLDAEMLEIARRFEESVRG |
| 515 | WP_053939146.1 cysteine hydrolase [Amantichitinum ursilacus]: |
| | MSQSAFIAEPFALPFDKKTTALVMIDMQRDFVEPAGFGEALGNDVSLVRVAIQPCKQVLEAARKAGLLVV |
| | HTREGHRPDLTDCPPAKLTRGGKTFIGSKGPMGRILVRGEAGHDLIPELYPIAGEPVIDKPGKGAFYQTD |
| | LHLILQNRGIKTLIVCGVTTEVCVTTTVREANDRGFECLVPADCVGSYFPEFQKASLEMIKAQGAIFGWV |
| | SNASNVIAALAA |
| 516 | WP_054019083.1 cysteine hydrolase [Ideonella sakaiensis]: |
| | MTPTLPLPATPFPYPFAPGRSALVVIDMQRDFVEPGGFGASLGNDVTRLHGAIGPIAALLAAWRARGWPV |
| | VHTRESHRPDLSDCPPAKRERGEPSLRIGDPGPMGRLLIRGEPGADIIPALAPAPGERVVDKPGKGMFWA |
| | TGLHEALQAEGITHLVFTGVTTEVCVQTSMREANDRGYVCLIVEDATESYFPEFKAAALAMLTAQGAIVG |
| | WSMPSAALLAGLPAP |
| 517 | WP_054069001.1 MULTISPECIES: cysteine hydrolase [Pseudomonas syringae |
| | group]: |
| | MISISARPDPFTFEPSRTAVVIIDMQRDFLEPGGFGAALGNDVAPLQAIVPTVQQLLALAREQGIAVIHT |
| | RESHRPDLSDCPQAKLDHGLPGLRIGDPGPMGRILVRGEPGNQIIDALTPLASEWVIDKPGKGMFFATDL |
| | QQRLTVAGITHLIFAGVTTEVCVQTSLREACDRGYRCLLIEDATESYFAAFKQATLDMITAQGAIVGRVA |
| | SLANLQHALHTRSTQ |
| 518 | WP_054071372.1 cysteine hydrolase [Pseudomonas amygdali]: |
| | MISVNARPDCFTFAPSCAAVVIIDMQRDFLEPGGFGAALGNDVVPLQAIVPSVQQLLALARDQGITVIHT |
| | RESHSADLADCPPAKLAHGSPGLRIGDSGPMGRILIRGEPGNQIIDSLTPLACEWIIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSTP |
| 519 | WP_054079126.1 cysteine hydrolase [Pseudomonas amygdali]: |
| | MISVNARPDCFTFAPSCAAVVIIDMQRDFLEPGGFGAALGNDVAPLQAIVPSVQQLLALARDQGIVVIHT |
| | RESHSADLADCPPAKLAHGSPGLRIGDPGPMGRILIRGEPGNQIIDSLTPLACEWVIDKPGKGMFFATDL |
| | HQRLTDAGITHLIFAGVTTEVCVQTSMREASDRGYRCLLIKDATESYFPAFKQATLDMITAQNAIVGRAA |
| | SLADLQQALQTRSTS |
| 520 | WP_054080702.1 MULTISPECIES: cysteine hydrolase [Pseudomonas]: |
| | MIKVNARPDSFAFDTSRTAVVIIDMQRDFLEPGGFGAALGNDVAPLQAIVPSVQRLLTLARDEGIAVIHT |
| | RESHRPDLADCPQAKRDHGSPGLRIGDPGPMGRILIRGEPGNQIIDTLAPLAGEWVIDKPGKGMFFATDL |
| | QPRLTDAGITHLIFAGVTTEVCVQTSMREANDRGYRCLLIEDATESYFPAFKRATLEMITAQGGIVGRVA |
| | SLTDLEQALQTRSTH |
| 521 | WP_054087477.1 MULTISPECIES: cysteine hydrolase [Pseudomonas syringae |
| | group]: |
| | MISLQARPSAFLFEPSRTAVVIIDMQRDFLEPGGFGAALGNDVAPLQAIVPAVRQLLALARDQGLAVIHT |
| | RESHHPDLADCPQAKLEHGSPGLRIGDPGPMGRILVRGEPGNQIIDALTPIAGEWIIDKPGKGMFFATDL |
| | HAKLAEAGITYLIFAGVTTEVCVQTSMREANDRGYRCLLLEDATESYFPAFKQATLDMITAQGAIVGRVA |
| | SLADLQLALHTRSTQ |
| 522 | WP_054090498.1 cysteine hydrolase [Pseudomonas syringae group |
| | genomosp. 3]: |
| | MISISARPDPFTFEPSRTAVVIIDMQRDFLEPGGFGAALGNDVAPLQAIVPTVQQLLALAREQGLVVIHT |
| | RESHLPDLSDCPQAKLDHGLPGLRIGDPGPMGRILVRGEPGNQIIDALTPLASEWVIDKPGKGMFFATDL |
| | QQRLTVAGITHLIFAGVTTEVCVQTSLREACDRGYRCLLIEDATESYFAAFKQATLDMITAQGAIVGRVA |
| | SLANLQHALHTRSTQ |
| 523 | WP_054154446.1 cysteine hydrolase [beta proteobacterium AAP51]: |
| | MSKQQGPAPEAGPVTVAARPSAFELQPGRAALLVIDMQRDFVEPGGFGASLGNDVTLLQAAIAPTRALLD |
| | AWRARGWPVLHTRESHAADLSDCPPAKRLRGQPALRIGDLGPMGRLLVRGEPGCAIVPELAALPGEVVID |
| | KPGKGAFHATPLQATLQALGVTQLVVAGVTTEVCVQSTMREANDRGYDCLLVEEATASYFPAFKAAAIEM |
| | IVAQGGIVGWAAPLSAVLSALPAEPAAAAAP |
| 524 | WP_054158501.1 cysteine hydrolase [Rhizobium sp. AAP43]: |
| | MAVIKARPFDITITSEKTALIVIDMQRDFIEPGGFGATLGNDVTLLQAIIPATARLIEGFRRAGLTVIHT |
| | RECHAPDLSDCPPAKRTRGKPALRIGDPGPMGRILIAGEDGADIVAALSPLPGETVIDKPGKGAFYSTPL |
| | SDILQEKGISQLVFAGVTTEVCVQTTMREANDRGYECLLATDATESYFPEFKKAAIDMMTAQGAIVGWAA |
| | TVDQIVEALDA |
| 525 | WP_054165574.1 cysteine hydrolase [Rhodopseudomonas sp. AAP120]: |
| | MAPPTSAATTMIAAEPAPIGLDWASTALLIIDMQRDFLEPGGFGETLGNDVSQLARAVPPIAAVLAAARR |
| | IGLPVIHTREGHLPDLSDAPPAKVARGAPSLRIGDPGPMGRILIRGEPGHDIVPELYPRADEIVIDKPGK |
| | GAFYATELSDVLQKYGIETLLVCGVTTEVCVNTTVREANDRGYRCIVIADGCASYFPEFHAAGLAMIKAQ |
| | GGIFGWVAESPAVLAAMAEQG |
| 526 | WP_054183557.1 cysteine hydrolase [Rhizobium acidisoli]: |
| | MVGIKAEPFAFPVRHDELALIVIDMQRDFAEPGGFGASLGNDVSRITRIVPDVKRLIQGFRNAGLPVIHT |
| | MECHRPDLSDLPPAKRDRGNPSLRIGDEGPMGRILISGEPGTAILAELAPVKGEIIIEKPGKGAFYATEL |
| | GAVLRQKGISQLVFSGVTTEVCVQTTMREANDRGYECLLAEEATESYFPEFKAAAIAMIRAQGAIVGWTA |
| | HVDDILESIANA |
| 527 | WP_054360928.1 cysteine hydrolase [Prosthecomicrobium hirschii]: |
| | MVTVPAKPFAYDLDPARVALVVIDMQRDFVEPGGFGETLGNDVSLLQAIVPTVRDLIGLFRAKGWTIVHT |
| | RESHSADLADCPPAKRDRGAPSLRIGDEGPMGRILVRGEPGNDIVPDLAPQPGEIVIDKPGKGAFYATAL |
| | GDILRLKGITHLVFAGVTTEVCVQTTMREANDRGYECLLVEDATESYFPEFKAAAIRMMTAQGGIVGWST |
| | DLATLKAAVG |
| 528 | WP_054538254.1 cysteine hydrolase [Confluentimicrobium sp. EMB200- |
| | NS6]: |
| | MGVIRAEPFDFSFDPATLGLVVIDMQRDFVEPGGFGASLGNDVALLQAIIPTVQALIGGFRAAGLPVIHT |
| | RECHRPDLSDLPPAKRDRGAPALRIGDEGPMGRILIAGEPGADIVPELAPAPGEPVIDKPGKGAFYGTEF |
| | AQVLADRNLRQLVFAGVTTEVCVQTTMREANDRGFDGLLATDATESYFPEFKQAAIRMIIAQGGIVGWAA |
| | PTAHVLEAL |
| 529 | WP_054985870.1 cysteine hydrolase [Pseudomonas syringae group |
| | genomosp. 7]: |
| | MISVNARPDSFTFDRSCAAVVIIDMQRDFLEPGGFGAALGNDVALLQAIVPSVQRLLALARDQGIAVIHT |
| | RESHSSDLADCPPAKLDHGSPGLRIGDPGPMGRILIRGEPGNQIIDSLTPLACEWVIDKPGKGMFFATDL |
| | HQRLSEAGITHLIFAGVTTEVCVQTSMREANDRGYRCLLIEDATESYFPAFKQATLEMITAQNAIVGRVA |
| | SLADLQRAVRTRSTP |
| 530 | WP_054990364.1 cysteine hydrolase [Pseudomonas coronafaciens]: |
| | MIRINARPDSFGCELSQTAVVIIDMQRDFLEPGGFGAALGNDVTLLQAIVPSVQRLLALAREQDLIVIHT |
| | RESHPADLSDCPQAKIDHGLPGLRIGDPGPMGRILIQGEPGNQIIEALTPVAGEWVIDKPGKGMFFATDL |
| | HLRLTEAGITHLIFAGVTTEVCVQTSMREACDRGYRCLLIEDATDSYFPAFKQATLEMITAQNAIVGRVA |
| | SLADVQQALPARSTQ |
| 531 | WP_054992163.1 cysteine hydrolase [Pseudomonas syringae pv. coryli]: |
| | MIKVNARPDRFAFDTSRTAVVIIDMQRDFLEPGGFGAALGNDVAPLQAIVPSVQRLLALARDEGMTVIHT |
| | RESHRPDLADCPQAKRDHGSPGLRIGDPGPMGRILIRGEPGNQIIDALAPRAGEWVIDKPGKGMFFATDL |
| | QQRLTDAGITHLIFAGVTTEVCVQTSMREANDRGYRCLLIEDATESYFPAFKQATLEMITAQGGIVGRVA |
| | SLTDLEQALQTRSTH |
| 532 | WP_055004059.1 cysteine hydrolase [Pseudomonas coronafaciens]: |
| | MIRINARPDSFSCELSQTAVVIIDMQRDFLEPGGFGAALGNDVTLLQAIVPSVQRLLALAREQDLIVIHT |
| | RESHPADLSDCPQAKIDHGLPGLRIGDPGPMGRILIQGEPGNQIIEALTPVAGEWVIDKPGKGMFFATDL |
| | HLRLTEAGITHLIFAGVTTEVCVQTSMREACDRGYRCLLIEDATDSYFPAFKQATLDMITAQNAIVGRVA |
| | SLADVQQALPARSTQ |
| 533 | WP_055010774.1 cysteine hydrolase [Pseudomonas caricapapayae]: |
| | MISVNARPDSFTFDRSCAAVVIIDMQRDFLEPGGFGAALGNDVALLQAIVPSVQRLLALARDQGIAVIHT |
| | RESHSPDLADCPPAKLDHGSPGLRIGDPGPMGRILIRGEPGNQIIDSLTPLACEWVIDKPGKGMFFATDL |
| | HQRLSEAGITHLIFAGVTTEVCVQTSMREANDRGYRCLLIEDATESYFPAFKQATLEMITAQNAIVGRAA |
| | SLADLQRALQTRSTP |
| 534 | WP_055406267.1 cysteine hydrolase [Frankia sp. ACNlag]: |
| | MSETATTPTAPLTVSARPYDFTFDPATTALVVIDMQRDFLEPGGFGESLGNDVSQLRSTIEPLQAVLAAV |
| | RAAGLTVIHTREGHLPDLSDLPPAKLHRGDAALRIGDLGPKGRILIRGEYGQDIIDELAPVDGEYVIDKP |
| | GKGAFYATAFGDVLAEKGITSLVVAGVTTEVCVHTTVREANDRGFECLVLSDCVGSYFPEFQRVALEMVA |
| | AQGGIFGWVAPSADFLAALASSAPAADSTVPAPAVTAS |
| 535 | WP_055675924.1 cysteine hydrolase [Labrenzia alba]: |
| | MITVDANPFEYCFDPASAALVVIDMQRDFVEPGGFGETLGNDVSHLQRAVDPTKRLLQLFRDRKMPVIHT |
| | RENHLSDLSDCPLAKRARGNPSLRIGDEGHMGRILIRGEPGAEIIPECAPIAGELVIDKPGKGAFYDTGL |
| | DDVLQKLGTRSLVFAGVTTEVCVQTTMREANDRGYECLLIEEATESYFPAFKEATIEMIRAQGGIVGWTA |
| | PLKALVEALSTTSE |
| 536 | WP_055800477.1 cysteine hydrolase [Variovorax sp. Root318Dl]: |
| | MRIEEAIPFPYEFEIRNTALVLIDMQRDFIEPGGFGETLGNDVSLLEAIVPATRTALQAWREAGGLVVHT |
| | REAHKPDLSDCPPAKRNRGNPALRIGDEGPMGRILVAGEPGNQIIDALAPIGGEIVVDKPGKGAFYATGL |
| | HELLQQRGITHLLFGGVTTEVCVQTSMREANDRGYDCLLLEDCTESYFPAFKAATLDMVRAQGGIVGWTA |
| | PSAALLAALLGGR |
| 537 | WP_055837564.1 cysteine hydrolase [Xylophilus sp. Leaf220]: |
| | MTDDTLTLDANPFAYRFAPARTALVVIDMQRDFLEPGGFGAALGNDVSRLQAIVPACAAVLRAWRAIGGM |
| | VVHTREAHRPDLSDCPPAKRLRGTPALRIGDAGPMGRILVAGEPGCEIVPALAPLESETVIDKPGKGAFH |
| | ATGLQDLLQRRGIDHLLFMGVTTEVCVQTSMREANDRGYDGLLLEDCTESYFPAFKQATLEMLCAQGAIV |
| | GWTAPSAALLAALPAGR |
| 538 | WP_055877683.1 cysteine hydrolase [Devosia sp. Root105]: |
| | MISVPSRPYPYALDPAHTALVVIDMQRDFIERGGFGDSLGNDVKRLEAIIPTTAALLALFRAQGWPVIHT |
| | REAHKPDLSDCPPSKIRRGNPSLHIGEMGAMGRLLVRGEPGNQIVDALAPLEGEMVIDKPGKGMFWATGL |
| | HEQLVELGITHLVFAGVTTEVCVQTSMREANDRGYECLLIEDATESYFAEFKATTLKMIAAQGGIVGWVT |
| | PLAALEGAVKA |
| 539 | WP_055958214.1 MULTISPECIES: cysteine hydrolase [unclassified |
| | Methylobacterium]: |
| | MPHILAAEPAPLTIDPATTALVVIDMQRDFLEPGGFGESLGNDVSLLQAAVPPIRAVLTAARGAGLLVVH |
| | TREGHKPDLSDAPPAKLERGEPSARIGAPGPMGRILIRGEPGHGIVPALAPMRGEVVIDKPGKGAFYATD |
| | LGAVLAARRIATLLVCGVTTEVCVHTTIREGNDRGYRCVAVGDGCASYFPEFHRVGLAMIAAQGGIFGWV |
| | ASSAAVIAALSGTS |
| 540 | WP_055985869.1 MULTISPECIES: cysteine hydrolase [unclassified |
| | Pseudomonas]: |
| | MIRLPARPATFSFEPTRTALVVIDMQRDFLEPGGFGAALGNDVTLLQTIVPAVASLLALAREQGMLVIHT |
| | RESHLADLSDCPAAKREGGAVGLRIGDAGPMGRILVRGEPGNQIIPPLAPIAGEWVIDKPGKGMFYATGL |
| | GDRLAAQGIDYLIFAGVTTEVCVQTSMREANDRGYRCLLIEEATESYFPAFKQATLEMIVAQGGIVGHTA |
| | NLAALSAAMNEEQA |
| 541 | WP_056004099.1 cysteine hydrolase [Devosia sp. Root413D1]: |
| | MISVPARPYPYALDPAHTALVVIDMQRDFIERGGFGDSLGNDVKRLEAIIPTTAALIGLFRAQGWPVIHT |
| | REAHKPDLSDCPPAKIRRGNPSLHIGEVGAMGRLLVRGEPGNQIVDALAPLEGEMVIDKPGKGMFWATGL |
| | HEQLVELGITHLVFAGVTTEVCVQTSMREANDRGYECLLIEDATESYFAEFKAATLKMIAAQGGIVGWVT |
| | PLAALQGAVKA |
| 542 | WP_056111546.1 MULTISPECIES: cysteine hydrolase [Methylorubrum]: |
| | MPAPQPLLDAEPAPLPFDPARTALVVIDMQRDFLEPGGFGESLGNDVSLLAAAVPPARALLAAARAAGLL |
| | VVHTREGHAPDLSDAPPAKRERGAPTARIGEPGPMGRILIRGEPGHDIIPELAPLDGEPVIDKPGKGAFY |
| | ATGLAALLEARGIETLIVCGVTTEVCVHTTVREANDRGYRCVVVADACGSYIPAFHEAGLAMIKAQGGIF |
| | GWVSRSAAVIAALGQA |
| 543 | WP_056143502.1 cysteine hydrolase [Methylobacterium sp. Leaf85]: |
| | MPNLLDAQPSPLPFDASSTALLIIDMQRDFLEPGGFGESLGNDASLLASAVPPTRALLDVARASGLLVVH |
| | TREGHRPDLSDAPPAKLERGEPTARIGQPGPMGRILIRGEPGHDIIPALAPQDGEPVIDKPGKGAFYATE |
| | LADVLAARGIATLLVCGVTTEVCVHTTVREANDRGYRCVVVADACASYIPEFHAAGLAMIKAQGGIFGWV |
| | SDSDSVIAALGRHG |
| 544 | WP_056167903.1 MULTISPECIES: cysteine hydrolase [unclassified |
| | Methylobacterium]: |
| | MPSLLDAEPSPLPFEASRTALVIIDMQRDFLEPGGFGESLGNDVSLLAAAVPPCRAVLDAARAAGLLVVH |
| | TREGHRPDLSDAPPAKLERGEPTARIGQPGPMGRILIRGEPGHGIVPALAPQDGEPVIDKPGKGAFYATE |
| | LADVLATRAIATLLVCGVTTEVCVHTTVREANDRGYRCVVIADACASYIPEFHEAGLAMITAQGGIFGWV |
| | SDAHRVVAALEGRASETLD |
| 545 | WP_056187621.1 cysteine hydrolase [Pseudorhodoferax sp. Leaf267]: |
| | MNSGEVRDARTLVVEAQPFDFPFEVASTALVIIDMQRDFIEPGGFGASLGNDVSLLAAIVPACRTVLQAW |
| | RAQGGLVLHTREAHRPDLRDCPPAKRLRGNPSLRIGDAGPMGRVLVSGEPGVQIIPALAPLPGEIVVDKP |
| | GKGMFHATPVDLLLQQAGIRTLLFMGVTTEVCVQTSMREANDRGYECLVLEDCTESYFPAFKAAALAMIR |
| | AQGGIVGWTAGSAELLAALHSG |
| 546 | WP_056190582.1 cysteine hydrolase [Methylobacterium sp. Leaf113]: |
| | MRPVIAAEPAPASFDPATTALVIIDMQRDFLEPGGFGETLGNDVSLLQTAVPPIRSVLAAARNAGLLIVH |
| | TREGHKPDLSDAPPAKLERGTPTARIGAPGPMGRILIRGEPGHGIVPELAPIRGEVVIDKPGKGAFYATD |
| | LGAVLSARRIATLLVCGVTTEVCVHTTIREGNDRGYRCIAIGDGCASYCPEFHRVGLAMIAAQGGIFGWV |
| | TSDAVVEALAGAR |
| 547 | WP_056198540.1 cysteine hydrolase [Methylobacterium sp. Leaf123]: |
| | MSVPRPLLDAEPAPLPFDPGSTALLVIDMQRDFLEPGGFGESLGNDVSSLAAAVPPARALLAAARGAGLL |
| | VVHTREGHAPDLSDAPPAKLERGAPTARIGEPGPMGRILIRGEPGHDIVPELAPLAGEPVIDKPGKGAFY |
| | ATGLAALLEARGIETLIVCGVTTEVCVHTTVREANDRGYRCVVVADACGSYIPAFHEAGLAMIKAQGGIF |
| | GWVAQSAAVITALGQA |
| 548 | WP_056205655.1 cysteine hydrolase [Pelomonas sp. Root1237]: |
| | MNTLLTLATAQPFPYTFNPAHTALVVIDMQRDFIEPGGFGASLGNDVTRLQAIVPAVRRMLDAWRAIEAV |
| | VLHTREAHRPDLSDCPPAKRLRGQPSLRIGDVGPMGRVLIAGEPGAEIIPELAPLPGELVVDKPGKGMFY |
| | ATPVDALLKERGITHLLFMGVTTEVCVQTSMREANDRGYECLLIEDGSASYFPEFKAAALAMLTAQGAIV |
| | GWAAPSSAVIEAIT |
| 549 | WP_056239520.1 cysteine hydrolase [Methylobacterium sp. Leaf456]: |
| | MPVLEAEPSPLPIDLATAALIVIDMQRDFLEPGGFGESLGNDVSLLAAAVPPTRALLAAARAAGLLVVHT |
| | REGHAPDLSDAPPAKRERGAPSLRIGEPGPMGRILIRGEPGHDIVAELAPQPGEPVIDKPGKGAFYATGL |
| | GALLEERAIATLIVCGVTTEVCVHTTVREANDRGYRCVVVSDACASYIPAFHEAGLAMIKAQGGIFGWVA |
| | ESAAVTAALR |
| 550 | WP_056248958.1 cysteine hydrolase [Methylobacterium sp. Leaf93]: |
| | MPNLLDAQPSPLPFDASSTALLIIDMQRDFLEPGGFGESLGNDVSLLASAVPPTRALLDAARASGLLVVH |
| | TREGHRPDLSDAPPAKLERGEPTARIGQPGPMGRILIRGEPGHDIIPALAPQDGEPVIDKPGKGAFYATD |
| | LADVLAARGIATLLVCGVTTEVCVHTTVREANDRGYRCVVVADACASYIPEFHAAGLAMIKAQGGIFGWV |
| | SDSDSVIAALGRHG |
| 551 | WP_056326008.1 cysteine hydrolase [Methylibium sp. Root1272]: |
| | MSVPATPFDYRLAPGTTALVVIDMQRDFIEPGGFGASLGNDVSLLVPAIAPIAALLAAWRARGWPVVHTR |
| | EAHKADLSDCPPAKRLRGEPTLRIGDPGPMGRLLISGEPGTEIIAALAPQAGEIVLDKPGKGMFWATGLH |
| | ERLQSLGVSHLVFTGVTTEVCVQTSMREANDRGYDCLLVEDGTESYFPAYKAAVLEMIAAQGAIVGWHAP |
| | SAAVLAALPEP |
| 552 | WP_056364586.1 cysteine hydrolase [Burkholderia sp. Leaf177]: |
| | MPQKQFQAEPFPLPFNAESTALVMIDMQRDFVEPGGFGEALGNDVSFVRSAIEPCRKLLKAARDARLLII |
| | HTREGHRADLADCPPAKLTRGGKRFIGEDGPMGRILVRGEAGHDIIPELYPALGEPIIDKPGKGAFYQTD |
| | LQLILQNHGIKTLIVCGVTTEVCVTTTVREANDRGYECIIPADCVGSYFPEFQKSALEMIKAQGGIFGWV |
| | SNAAEVIDGLRS |
| 553 | WP_056421401.1 MULTISPECIES: cysteine hydrolase [Acidovorax]: |
| | MSLTTIHAHPFDYRFSLPHTALVIIDMQRDFIEPGGFGETLGNDVSLLEAIVPACQAVLLAWRAAGGLVV |
| | HTRESHRPDLSDCPPAKRLRGNPQLRIGDAGPMGRILVAGEPGNQIIPALAPVVGEIVVDKPGKGMFYAT |
| | GLHETLQARGITHLVFMGVTTEVCVQTSMREANDRGYDGLLLEDCTESYFPHFKAAAIEMLRAQGAIVGW |
| | TAPSAALLPRLADALLAP |
| 554 | WP_056425063.1 cysteine hydrolase [Methylobacterium sp. Leaf91]: |
| | MSVTLSAEPADLGFDPATTALVIIDMQRDFLEPGGFGETLGNDVSLLLAAVAPCRSVLAAARRTGMLVVY |
| | TREGHLPDLSDAPPAKLERGEPTARIGAPGPMGRILIRGEPGHDIVPDLAPSAGEIVIDKPGKGAFYATE |
| | LGAVLAERGIATLLVCGVTTEVCVHTTIREGNDRGYRCVALADCCASYFPEFHRIGLEMIKAQGGIFGWV |
| | SSSEAVLTALATPG |
| 555 | WP_056453050.1 cysteine hydrolase [Methylobacterium sp. Leaf86]: |
| | MSVTLSAEPADLGFDPATTALVIIDMQRDFLEPGGFGETLGNDVSLLLAAVAPCRSVLAAARRTGMLVVH |
| | TREGHLPDLSDAPPAKLERGEPTARIGAPGPMGRILIRGEPGHDIVPDLAPSAGEIVIDKPGKGAFYATE |
| | LGAVLAERGIATLLVCGVTTEVCVHTTIREGNDRGYRCVALADCCASYFPEFHRIGLEMIKAQGGIFGWV |
| | SSSEAVLTALATPG |
| 556 | WP_056471207.1 cysteine hydrolase [Methylobacterium sp. Leaf104]: |
| | MSQILAAEPAPLPIDPATTALVVIDMQRDFLEPGGFGETLGNDVSLLQAAVPPIRAVLAAARRAGLLIVH |
| | TREGHKPDLSDAPPAKLERGEPSARIGAPGPMGRILIRGEPGHGIVPELAPMRGEVVIDKPGKGAFYATD |
| | LGAVLAARGIGTLLVCGVTTEVCVHTTIREGNDRGYRCVAIGDGCASYFPEFHRVGLAMIAAQGGIFGWV |
| | AASSAVIAVLGAAR |
| 557 | WP_056472753.1 cysteine hydrolase [Rhizobacter sp. Root404]: |
| | MPIAATPFPYPFAPGGRTALVVIDMQRDFVEPGGFGASLGNDVSLLHTAIEPIAALLAAWRARGWPVVHT |
| | REAHLPDLSDCPPAKRLRGAPSLRIGETGTMGRLLVRGEPGTSIIPALAPQRGELAIDKPGKGMFWATGL |
| | HEMLQALGVTHLVFTGVTTEVCVQTSMREANDRGYDCLLVEDATESYFPEFKAAALAMIAAQGAIVGWHT |
| | PSAALLAALPASAVKSPGA |
| 558 | WP_056492193.1 cysteine hydrolase [Methylobacterium sp. Leaf111]: |
| | MLPVIAADPAPLTFDPATTALVIIDMQRDFLEPGGFGETLGNDVTLLQTAVPPIRAVLAAARSAGLLIVH |
| | TREGHKPDLSDAPPAKLERGTPTARIGAPGPMGRILIRGEPGHAIVPELAPIRGEVVIDKPGKGAFYATD |
| | LGAVLSARRIATLLVCGVTTEVCVHTTIREANDRGYRCVAIGDGCASYRPEFHRVGLAMIAAQGGIFGWV |
| | SSSAAEVEALGGAR |
| 559 | WP_056502177.1 cysteine hydrolase [Aureimonas sp. Leaf454]: |
| | MAEIPAAPFPFPLDRGTVGLIVIDMQRDFLEHGGFGESLGNDVTRLQAIVPATARLIQGFRAAGRPVIHT |
| | RECHRPDLSDCPPAKLARGRPGLRIGDEGAMRRILVKGEPGAEIVPELFPEPGETVIDKPGKGAFYATGL |
| | GDVLSAGGITQLVFAGVTTEVCVQTTMREANDRGFECLLAEDATESYFPEFKRAAIEMITAQGAIVGWVA |
| | PVDAVLAGLGA |
| 560 | WP_056515509.1 cysteine hydrolase [Variovorax sp. Root411]: |
| | MRIEEANPFPYEFDVESTALVLIDMQRDFIEPGGFGETLGNDVSLLEAIVPATKAALAAWRKAGGLVVHT |
| | REAHKADLSDCPPAKRNRGNPTLRIGDEGPMGRILVAGEPGNQIIDALAPIDGELVIDKPGKGAFHATGL |
| | HELLQQRGITHLLFGGVTTEVCVQTSMREANDRGYDSLLLEDCTESYFPAFKAATLDMVRAQGAIVGWTA |
| | PSAALLAALNSSPS |
| 561 | WP_056539150.1 cysteine hydrolase [Rhizobium sp. Root1220]: |
| | MMEINAQPFAFPTRRHELALIVIDMQRDFAEPGGFGASLGNDVDRVTRIIPDVKRLLQGFRDAGLPVIHT |
| | MECHRPDLSDLPPAKRNRGNPSLRIGDDGPMGRILIAGEPGTAILRELAPIDGEVVIEKPGKGAFYATEL |
| | GDVLKQSGVSQLVFAGVTTEVCVQTTMREANDRGYECLLAEEATESYFPEFKEAAIAMIRAQGAIVGWTA |
| | HVDDILEVIGHA |
| 562 | WP_056546772.1 cysteine hydrolase [Mycobacterium sp. Root135]: |
| | MTVSAEVPAEPFAFPLVAGKTALIVIDMQRDFILPGGFGESLGNDVDQLLKVVPPLAALIAAAREAGIMV |
| | IHTREGHVPDLSDCPAAKLNRGAPSKRIGDPGKYGRILIRGEYGHDILDELAPVDGEVVIDKPGKGAFYA |
| | TELSSILTDAGITQLLITGVTTEVCVHTTTREANDRGYECLVVSDCVGSYFPEFQRVGLEMIKAQGGIFG |
| | WVADTSAVIPAIQSLAIATPSQA |
| 563 | WP_056571532.1 MULTISPECIES: cysteine hydrolase [Mesorhizobium]: |
| | MAEIDAQPFAFAFKPVTMALVVIDMQRDFAEPGGFGASLGNDVSRITAIVPTVKRLIEGFRAAGLPVIHT |
| | MECHRADLSDLPPAKRDRGNPSIRIGDVGPMGRVLIVGEPGTAILDELAPLPGEIIIEKPGKGAFYATRL |
| | SEELKHLGAQQLVFAGVTTEVCVQTTMREANDRGYECLLAEDATESYFPEFKAAAIAMIRAQGAIVGWTA |
| | TTDQVLQGLANG |
| 564 | WP_056588049.1 cysteine hydrolase [Variovorax sp. Root434]: |
| | MRIEEANPFSYEFDVASTALVLIDMQRDFIEPGGFGETLGNDVSLLEAIVPATNAALAAWRKAGGLVVHT |
| | REAHKADLSDCPPAKRNRGKPTLRIGDEGPMGRILVAGEPGNQIIDALAPIDGELVIDKPGKGAFYATGL |
| | HEVLQQRGITHLLFGGVTTEVCVQTSMREANDRGYDSLLLEDCTESYFPAFKAATLDMVRAQGAIVGWTA |
| | PSAALLAALNGNPS |
| 565 | WP_056671862.1 cysteine hydrolase [Pseudorhodoferax sp. Leaf265]: |
| | MAVQARPFDFPFDLATTALVIIDMQRDFIEPGGFGASLGNDVSLLEAIVPACRRTLQAWRAAGGLVLHTR |
| | EAHRPDLRDCPPAKRLRGNPSLRIGDVGPMGRVLVSGEPGVEIIPALAPVPGEIVVDKPGKGMFHGTPVQ |
| | NLLQQAGIRSLVFMGVTTEVCVQTSMREANDRGYECLVLEDCTESYFPQFKAAALEMVRAQGGIVGWTAT |
| | SAQLLAALHSE |
| 566 | WP_056713331.1 cysteine hydrolase [Bosea sp. Leaf344]: |
| | MQCQVPAQPEPLAVDFRRSALLIIDMQRDFLEPHGFGAALGNDVSLLGRAVAPCKAMLEGARAAGILVLH |
| | TREGHRPDLSDAPKTKIERGAPERRIGVAGPMGRILVRGEAGHGIIADLQPLPSEPVIDKPGKGAFYQTD |
| | LELLLRNRGIDTLLIAGVTTEVCVHSTVREANDRGFRCLVLGDACASYHPEFHEVGLRMIAAQGAIFGWV |
| | TTTEAVLAALSDSQAARPAAPVETVAEVESA |
| 567 | WP_056775776.1 cysteine hydrolase [Serratia sp. Leaf51]: |
| | MTTHQFQAEPFPLAFDPQTTALVMIDMQRDFVEPGGFGEALGNDVSKVRTAIAPCKKVLDAARAQGMLVI |
| | HTREGHRADLSDCPPAKLTRGGQTFIGTDGPMGRILVRGEAGHDIIPELYPVAGEPVIDKPGKGAFYQTD |
| | LHLVLQNHGIRTLIVCGVTTEVCVTTTVREANDRGFECIIPQDCVGSYFPEFQKSALEMLKAQGAIFGWV |
| | SDANAIINGLKA |
| 568 | WP_056817413.1 MULTISPECIES: cysteine hydrolase [unclassified |
| | Rhizobacter]: |
| | MIQVDALPGPFEFEPAHTALVMIDMQRDFIEPGGFGAALGNDVSLLAPVVPAAAELVALCRAMGVLVVHT |
| | QECHRPDLSDCPPAKRLRGKPSLRIGDPGPMGRILIDGEPGAGFVPELMPEPGDVVIAKPGKGAFYGTRL |
| | AELLQDQQITRLIFGGVTTEVCVQTTMREANDRGYECLLVEEATGSYFPQFKAATLAMIRAQGGIVGWTA |
| | SLRAVQAAFQRQAAN |
| 569 | WP_056819633.1 MULTISPECIES: cysteine hydrolase [Nocardia]: |
| | MTEIPADPTPLPFDPATTALVIIDMQRDFLLPGGFGESLGNDVALLRTVIEPLAELLASARAAGITVIHT |
| | REGHLPDLSDCPPAKLRRGNPSQRIGDPGRFGRILIRGEYGHDIIDELAPLDGETVIDKPGKGAFYATEL |
| | AAVLQQNSITTLLVAGVTTEVCVHTTVREANDRGYECLVVADCVGSYFPEFQRVGLAMIAAQGAIFGWVA |
| | DSADVIAALTTTAPVTA |
| 570 | WP_056898202.1 cysteine hydrolase [Pseudorhodoferax sp. Leaf274]: |
| | MAVQAQPFDFPFDLATTALVIIDMQRDFIEPGGFGASLGNDVSLLEAIVPACRRTLQAWRAAGALVLHTR |
| | EAHRPDLRDCPPAKRLRGNPSLRIGDAGPMGRVLVSGEPGVEIIPALAPLPGEIVVDKPGKGMFHGTPVQ |
| | NLLQQAGIRSLVFMGVTTEVCVQTSMREANDRGYECLVLEDCTESYFPQFKAAALEMVRAQGGIVGWTAT |
| | SAQLLAALHSE |
| 571 | WP_057015261.1 cysteine hydrolase [Bradyrhizobium pachyrhizi]: |
| | MANSSGTIAAEPAPITLDWSRTALVIIDMQRDFMERGGFGETLGNDVSRLARAVKPIAAVLAAVRDAGLL |
| | VVHTREGHLPDLSDAPPAKVERGAPSLRIGDPGPMGRILIRGEAGHDIIPELYPLDSEIVIDKPGKGAFY |
| | ATEFGDILQKYGIENLLVCGVTTEVCVNTTVREANDRGYRCWISDGCASYFPEFHEMGLKMIKAQGGIF |
| | GWVTDSAAVLEALGD |
| 572 | WP_057026813.1 cysteine hydrolase [Bradyrhizobium yuanmingense]: |
| | MLNSAKPTKGVVSAEPEPIALDWPATALLIIDMQRDFMEPGGFGETLGNDVSQLARAVKPIGAVLTAARD |
| | TGMLVIHTREGHLPDLSDAPPAKVERGAPSLRIGDPGPMGRILIRGEAGHDIIPELYPLDSEIVIDKPGK |
| | GAFYATELGEVLEKYGIENLLVCGVTTEVCVNTTVREANDRGYRCVVIADGCASYFPEFHEMGLKMIKAQ |
| | GGIFGWVADSAAVLEAMKVSTT |
| 573 | WP_057147092.1 cysteine hydrolase [Mycobacterium sp. Soil538]: |
| | MNPIPVAAEPAPFQLVAGKTALIVIDMQRDFLLPGGFGESLGNDVERLRTVVPPLAALLGAARAAGIMVI |
| | HTREGHRPDLSDCPPAKLRRGAPSKRIGDPGTYGRILIRGEYGHDIIDELAPVEGEVVIDKPGKGAFYGT |
| | DLSDVLTGAGITQLLITGVTTEVCVHTTTREANDRGYECLVLSDCVGSYFPEFHRVGLQMVTAQGGIFGW |
| | VADSAAVIPALHQLTTTAA |
| 574 | WP_057165500.1 cysteine hydrolase [Mycobacterium sp. Root265]: |
| | MSTTSVDVPAEPSPFPLISGRTALIIIDMQRDFLLPGGFGESLGNDVDQLLKVVPPLAALVAAAREAGIL |
| | VIHTREGHVPDLSDCPPAKLSRGAPSKRIGDPGKYGRILIRGEYGHDIVDELSPVDGEVVIDKPGKGAFY |
| | ATELQDVLGRAGITQLLVTGVTTEVCVHTTTREANDRGYECLVVSDCVGSYFPEFQRVGLEMIKAQGGIF |
| | GWVADSSAVIPALHNLALSAA |
| 575 | WP_057195967.1 cysteine hydrolase [Bradyrhizobium sp. Leaf396]: |
| | MLNSPEPTRGVISAEPEPIELDWSKSALLIIDMQRDFLEPGGFGETLGNDVSQLSRAVKPIGAVLTAARD |
| | AGMLVIHTREGHLPDLSDAPRAKIERGAPSLRIGDAGPMGRILIRGEAGHDIIPELYPLDSEIVIDKPGK |
| | GAFHATELGEVLERYGIENLLVCGVTTEVCVNTTVREANDRGYRCVVISDGCASYFPEFHAMGLKMIKAQ |
| | GGIFGWVASSAAVLEAMTSSTTLGATS |
| 576 | WP_057203102.1 cysteine hydrolase [Acidovorax sp. Root217]: |
| | MSLTTIHANPFAFRFALAHTALVIIDMQRDFIEPGGFGETLGNDVSLLEAIVPACQAVLSAWRTAGGLVV |
| | HTRESHRPDLSDCPPAKRLRGNPQLRIGDAGPMGRILVAGEPGNQIIPALAPVDGEIVVDKPGKGMFYAT |
| | GLHETLQARGITHLVFMGVTTEVCVQTSMREANDRGYDGLLLEDCTESYFPHFKAAAIEMLRAQGAIVGW |
| | TAPSAALLSALADAVLAP |
| 577 | WP_057267682.1 cysteine hydrolase [Acidovorax sp. Root219]: |
| | MSLTTIHANPFAFRFALAHTALVIIDMQRDFIEPGGFGETLGNDVSLLEAIVPACQAVLQAWRTAGGLVV |
| | HTRESHRPDLSDCPPAKRLRGNPQLRIGDAGPMGRILVAGEPGNQIIPALAPVDGEIVVDKPGKGMFYAT |
| | GLHETLQARGITHLVFMGVTTEVCVQTSMREANDRGYDGLLLEDCTESYFPHFKAAAIEMLRAQGAIVGW |
| | TAPSAALLSALADAVLAP |
| 578 | WP_057298550.1 cysteine hydrolase [Pelomonas sp. Root1217]: |
| | MNTLLTLATAQPFPYTFNPAHTALVVIDMQRDFIEPGGFGASLGNDVTRLQAIVPTVRRMLDAWRALNDG |
| | KGGVVLHTREAHRPDLSDCPPAKRLRGQPSLRIGDVGPMGRVLIAGEPGAEIIPELAPLPGELVVDKPGK |
| | GMFYATPVDALLKERGITHLLFMGVTTEVCVQTSMREANDRGYECLLIEDGTASYFPEFKAAALAMLTAQ |
| | GAIVGWAAPSSAVIEAIT |
| 579 | WP_057411262.1 cysteine hydrolase [Pseudomonas amygdali]: |
| | MISVNARPDCFTFAPSCAAVVIIDMQRDFLEPGGFGAALGNDVAPLQAIVPSVQQLLALARDQGIVVIHT |
| | RESHSADLADCPPAKLAHGSPGLRIGDPGPMGRILIRGEPGNQIIDSLTPLACEWIIDKPGKGMFFATDL |
| | HQRLTDAGITHLIFAGVTTEVCVQTSMREASDRGYRCLLIEDATESYFPTFKQATLDMITAQNAIVGRAA |
| | SLADLQQALQTRSIP |
| 580 | WP_057418534.1 cysteine hydrolase [Pseudomonas syringae group |
| | genomosp. 3]: |
| | MISISARPDPFTFEPSRTAVVIIDMQRDFLEPGGFGAALGNDVAPLQAIVPTVQQLLALAREQGLVVIHT |
| | RESHLPDLSDCPQAKLDHGLPGLRIGDPGPMGRILVRGEPGNQIIDALTPLASEWVIDKPGKGMFFATDL |
| | HQRLTVAGITHLIFAGVTTEVCVQTSLREACDRGYRCLLIEDATESYFAAFKQATLDMITAQGAIAGRVA |
| | SLANLQHALHTRSTQ |
| 581 | WP_057436414.1 cysteine hydrolase [Pseudomonas syringae group |
| | genomosp. 3]: |
| | MISISARPDTFTFEPSRTAVVIIDMQRDFLEPGGFGAALGNDVAPLQAIVPTVQQLLALAREQGLVVIHT |
| | RESHRPDLSDCPQAKLDHGLPGLRIGDPGPMGRILVRGEPGNQIIDALTPLASEWVIDKPGKGMFFATDL |
| | HHRLTAAGITHLIFAGVTTEVCVQTSLREACDRGYRCLLIEDATESYFPAFKQATLDMITAQGAIVGRVT |
| | SLANLQHGLHTRSTP |
| 582 | WP_057453393.1 cysteine hydrolase [Pseudomonas savastanoi]: |
| | MISVNARPDCFTFSPSCAAVVIIDMQRDFLEPGGFGAALGNDVAPLQAIVPSVQQLLALARDQGIVVIHT |
| | RESHSADLADCSPAKLAHGSPGLRIGDSGPMGRILIRGEPGNQIIDSLTPLACEWVIDKPGKGMFFATDL |
| | HQRLTDAGITHLIFAGVTTEVCVQTSMREASDRGYRCLLIEDATESYFPAFKQATLDMITAQNAIVGRAA |
| | SLADLQQALQTRSTS |
| 583 | WP_057479411.1 cysteine hydrolase [Rhodococcus sp. Leaf278]: |
| | MTSEYPPESVVPSASPSEFTIGTATTALLVIDMQRDFLLPGGFGESLGNDVGLLRSVIEPLARLISVARE |
| | TGIPVIHTREGHLPDLSDCPPAKLRRGTPSQRIGDPGAFGRILIRGEYGHDIVDELAPIAGETVIDKPGK |
| | GAFYATELAEILIAAGITTLLVTGVTTEVCVHTTVREANDRGYECLVVTDCVGSYFPEFQRVGLEMISAQ |
| | GGIFGWTAPSEDWAALSAFVPTSASR |
| 584 | WP_057592794.1 cysteine hydrolase [Variovorax paradoxus]: |
| | MQIAQALPFPYDFDPKTTALVLIDMQRDFIEPGGFGETLGNDVSLLEAIVPATQRVLAAWRAAGGLVVHT |
| | REAHRPDLSDCPPAKRNRGNPTLRIGDEGPMGRILVAGEPGNQIIEALAPVAGEIVIDKPGKGAFYATEL |
| | HELLRARGITHLLFGGVTTEVCVQTSMREANDRGYDGLLLEDCTESYFPAFKTATVEMVRAQGGIVGWTA |
| | TGGQLIEALGGA |
| 585 | WP_057673702.1 cysteine hydrolase [Curvibacter sp. PAE-UM]: |
| | MKTIAAQPFAYAFEPAHTALVIIDMQRDFIEPGGFGETLGNDVSLLEAIVPACQSVLLAWRKTGGLVVHT |
| | REAHKPDLSDCPPAKRNRGNPTLRIGDAGPMGRILVMGEPGNQIIPALAPIAGEIVIDKPGKGAFYATGL |
| | HEMLQARGITHLLFMGVTTEVCVQTSMREANDRGYDSLLLEDCTESYFPHFKAAAVEMIHAQGAIVGWTA |
| | ASAQLLAALR |
| 586 | WP_057753445.1 cysteine hydrolase [Bradyrhizobium manausense]: |
| | MLNSAKPTLGVISAEPEPIRLDWSSTALVIIDMQRDFMEPGGFGETLGNDVSQLARAVKPIGAVLKAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVASSAAVLEAMTISTT |
| 587 | WP_057833466.1 cysteine hydrolase [Bradyrhizobium jicamae]: |
| | MANSAKLVAEPEPIEIDWSVTALLIIDMQRDFMEPGGFGETLGNDVSQLARAVKPIAAVLDAARGAGMLV |
| | IHTREGHLPDLSDAPSAKVERGAPSLRIGDPGPMGRILIRGEPGHDIIPELYPLDTEIVIDKPGKGAFYA |
| | TELGEVLQRYGIENILVCGVTTEVCVNTTVREANDRGYRCVVLADGCASYFPEFHEMGLKMIKAQGGIFG |
| | WVSDSAAVLEALSPEIPTTAVAGASR |
| 588 | WP_057843254.1 cysteine hydrolase [Bradyrhizobium retamae]: |
| | MANSRKLAAEPYPIELDWAAAALLIIDMQRDFMEPGGFGETLGNDVSQLARAVKPIAAVLEAARAAGMLV |
| | IHTREGHLPDLSDAPPAKVERGAPSLRIGDPGPMGRILVRGEPGHDIIPELYPLDSEIVIDKPGKGAFYA |
| | TELGDVLQRYGIENLLVCGVTTEVCVNTTVREANDRGYRCVVLADGCASYFPEFHEMGLKMIKAQGGIFG |
| | WVSDSAAVLDALSPEIPTTAVAGASR |
| 589 | WP_057851703.1 cysteine hydrolase [Bradyrhizobium valentinum]: |
| | MANSAKLAAEPGPIELDWAATALLIIDMQRDFMEPGGFGETLGNDVSQLARAVKPISAVLDAARAAGMLV |
| | IHTREGHLPDLSDAPPAKVERGAPSLRIGDPGPMGRILIRGEAGHDIISELYPLDSEIVIDKPGKGAFYA |
| | TELGDVLQRYGIDNLLVCGVTTEVCVNTTVREANDRGYRCVVLADGCASYFPEFHEMGLKMIKAQGGIFG |
| | WVSDSAAVLDALLPEIPTTAVAGASR |
| 590 | WP_057859253.1 cysteine hydrolase [Bradyrhizobium lablabi]: |
| | MANSVKLAAEPEPIELDWAATALLIIDMQRDFMEPGGFGETLGNDVSQLARAVKPIASLLDAARGAGMLV |
| | IHTREGHLPDLSDAPPAKVERGAPSLRIGDPGPMGRILIRGEAGHDIIPELYPLDSEIVIDKPGKGAFYA |
| | TELGEVLQRYGIENLLVCGVTTEVCVNTTVREANDRGYRCVVLADGCASYFPEFHEMGLKMIKAQGGIFG |
| | WVSDSAAVLGALSPEIPTTAVAGASR |
| 591 | XP_014555164.1 hypothetical protein COCVIDRAFT_103080 [Bipolaris |
| | victoriae FI3]: |
| | MAKTNTKPNMISFEAKPYAFSFPLDHTALLIIDMQRDFLLPQGFGEIQGGNLEAVQVSIEPTKRLLDACR |
| | SAGMAVFHTREGHKPDLSDCPSSKLVRQEAAPGNTQHKLVIGDKGELGRLLTRGEYGHDIIDELKPLPGE |
| | VIIDKPGKGSFWNTPILHQLKARAITHLIVSGVTTECCFATTIREANDRGFECCGIEEATSGYNDACFKK |
| | PTLDMIHWSQGLFGFIGSLQPLVEALEPFSTKQIQLGSTPPQTPPEFDGDLTISSLQRAYRNGLSPLTVV |
| | EAVYRKIEAYKKIDPAVWIHIQPLESALEAARDLITKFPDRTALPPLFGIPFSVKDSIDIAGLPTTTACP |
| | PLAHIPSTSAVVYEKVISQGALFIGKTNLDQLATGLVGCRSPYGIPHSIYHPSYISGGSSSGSAVSVAAN |
| | LVSFSLATDTAGSGRVPAGLNGIVGFKPTRGTISFRGITPACLSLDCIALATKTIPDARTLWQVLESYDP |
| | LDPYSKPALLAFERHINSTGPQSSTFKFGIPPQEALAVCSAPTRRLFNATVSKLQSLGGILTPIPWSPFQ |
| | KAGNLLYEGTFVSERLASLPNDFLEKNRERLHPVTAQLMDAVTQRKSTAVDAYRDLQAKTLYTRQAEDVF |
| | AYAAHGIDVLVVPTTPTHWRIDEVLEDPIAKNSVLGEFTHCGNVLDLCGVAVPAGTYPVGELSGKEEDEG |
| | VLPFSVTFLSGSRLDAEMLEIARRFEESMCG |
| 592 | WP_058401721.1 cysteine hydrolase [Pseudomonas savastanoi]: |
| | MISVNARPDCFTFDPSCAAVVIIDMQRDFLEPGGFGAALGNDVAPLQAIVPSVQQLLALARDQGIVVIHT |
| | RESHSADLADCPPAKLAQGSPGLRIGDLGPMGRILIRGEPGNQIIDSLTPLACEWVIDKPGKGMFFATDL |
| | HQRLTDAGITHLIFAGVTTEVCVQTSMREASDRGYRCLLIEDATESYFPAFKQATLDMITAQNAIVGRAA |
| | SLADLQHALQTRSTS |
| 593 | WP_058408964.1 MULTISPECIES: cysteine hydrolase [Pseudomonas]: |
| | MISVNARPDCFTFDPSCAAVVIIDMQRDFLEPGGFGAALGNDVAPLQAIVPSVQQLLALARDQGIVVIHT |
| | RESHSADLADCPPAKLAQGSPGLRIGDLGPMGRILIRGEPGNQIIDSLTPLACEWVIDKPGKGMFFATDL |
| | HQRLTDAGITHLIFAGVTTEVCVQTSMREASDRGYRCLLIEDATESYFPAFKQATLDMITAQNAIVGRAA |
| | SLADLQHALQTRSTP |
| 594 | WP_058416410.1 MULTISPECIES: cysteine hydrolase [Pseudomonas]: |
| | MIKVNARPDSFTFDTSRTAVVIIDMQRDFLEPGGFGAALGNDVAPLQAIVPSVQRLLTLARDEGIAVIHT |
| | RESHRPDLADCPQAKRDHGSPGLRIGDPGPMGRILIRGEPGNQIIDTLAPLAGEWVIDKPGKGMFFATDL |
| | QPRLTDAGITHLIFAGVTTEVCVQTSMREANDRGYRCLLIEDATESYFPVFKRATLEMITAQGGIVGRVA |
| | SLTDLEQALQTRSTH |
| 595 | WP_058642667.1 cysteine hydrolase [Pseudacidovorax intermedius]: |
| | MRIDARPFAYDFDLASTALVLIDMQRDFIEPGGFGETLGNDVSLLAAIVPATQAVLAAWRQAGGLVVHTR |
| | EAHRPDLSDCPPAKRLRGAPSLRIGDEGPMGRILVAGEPGNQIIDALAPIEGEWVIDKPGKGAFHATGLH |
| | ELLQARGITHLVFGGVTTEVCVQTSMREANDRGYDCLLLEDCTESYFPQFKAAAVEMIRAQGAIVGWTAT |
| | GPQLMAALASAPPQG |
| 596 | WP_058824180.1 cysteine hydrolase [Pseudomonas syringae]: |
| | MISLCARPDPFTFEPSCTAVVIIDMQRDFLEPGGFGAALGNDVAPLQAIVPAVQRLLALARDQGLAVIHT |
| | RESHHPDLSDCPQAKLDHGLPGMRIGDPGPMGRILIRGEPGNQIIDALTPSAGEWIIDKPGKGMFYATDL |
| | HSRLAEAGITHLIFAGVTTEVCVQSSLREANDRGYRCLLIEDATESYFPAFKQATLDMITAQGAIVGRVA |
| | SLADLAQALHTRSTP |
| 597 | WP_058894686.1 cysteine hydrolase [Herbaspirillum rubrisubalbicans]: |
| | MIHIDALPYPYQFHPRSTALWIDMQRDFVEEGGFGSALGNDVRPLGAIVPTVAALLTLARAHQMLWHT |
| | RESHLPDLSDCPRAKRLRGNPTLGIGDVGPMGRILVRGEPGNQILPQLAPIEGEIVIDKPGKGAFYATDL |
| | HAQLLERGITHLLIAGVTTEVCVQTSMREANDRGYECLVVQDACASYFPEFHRATLDMLTAQSGIVGWRA |
| | PLAQLQSAMAAYAGDHP |
| 598 | WP_058998539.1 cysteine hydrolase [Leptolyngbya sp. NIES-2104]: |
| | MPSIAAQPYEYELPIESEIALIVIDMQRDFLEPGGFGEALGNDVELATAIVPTVKRLLEGCRAMNLSIFH |
| | TQEGHRSDLSDCPQSKLKRGRGNLAIGDPGKFGRILVLGEPGNEIIPELAPIPGEVLIPKPGKGAFYSTD |
| | LEVQLIARNVTHLLIAGVTTEVCVQTTMREANDRGYECLLVEDATASYFPEFKQATLEMVRAQSGIVGWT |
| | ATTDQVLEGLRSWKEN |
| 599 | WP_059096191.1 cysteine hydrolase [Mycobacterium sp. IS-1742]: |
| | MSQTVDVPAEPTPFAVTADSTALIVIDMQRDFLLPGGFGESLGNDVEQLLKVVPPLAALIAAARTAGVTV |
| | IHTREGHRRDLSDCPPAKLNRGAPTKRIGDPGRYGRILVRGEYGHDIVDELAPLPGEVVIDKPGKGAFYA |
| | TELQDILTAAGITRLLVTGVTTEVCVHTTTREANDRGYECLVVSDCVGSYFPEFQRIGLEMIKAQGGIFG |
| | WVADSAAVIPAMQALTTTAV |
| 600 | WP_059186024.1 cysteine hydrolase [Mesorhizobium loti]: |
| | MAEIEAQPFAFAFKPETTALIVIDMQRDFAEPGGFGASLGNDVSRVTAIVPTVKRLIEGFRAAGLPVIHT |
| | MECHRPDLSDLPPAKRNRGNPSIRIGDAGPMGRVLIAGELGTAILEELAPLPGEIVIEKPGKGAFYATGL |
| | GDDLKRIGARQLVFAGVTTEVCVQTTVREANDRGYECLVAEDATESYFPEFKAAALAMIRAQGAIVGWTA |
| | TTDQVLEGIANA |
| 601 | WP_059193389.1 cysteine hydrolase [Streptomyces antibioticus]: |
| | MAVAESLRVEAAPYAFTFDLAETALVLIDMQRDFLEPGGFGESLGNDVEQLRKTIAPLRAVLDACRAAGM |
| | AVMHTREGHLPDLSDCPPSKLLRGNPSMRIGDPGPKGRILVRGEEGHDIIEELYPVAGEPVIDKPGKGAF |
| | YATEFGELLTARGIRRLVVTGVTTEVCVHTTVREANDRGYECLVLSDCVGSYFPQFQQAGLEMVAAQGGI |
| | FGWTAESAAFLAALATASPVGPDTPEAAREPAPQPR |
| 602 | WP_060403895.1 cysteine hydrolase [Pseudomonas amygdali]: |
| | MISVNARPDCFTFAPSCAAVVIIDMQRDFLEPGGFGAALGNDVAPLQAIVPSVQQLLALARDQGIVVIHT |
| | RESHSADLADCPPAKLAHGSPGLRIGDSGPMGRILIRGEPGNQIIDSLTPLACEWIIDKPGKGMFFATDL |
| | HQRLTDAGITHLIFAGVTTEVCVQTSMREASDRGYRCLLIEDATESYIPTFKQATLDMITAQNAIVGRAA |
| | SLADLQQALQTRSTP |
| 603 | WP_060414461.1 MULTISPECIES: cysteine hydrolase [Pseudomonas syringae |
| | group]: |
| | MISISARPDPFTFEPSRTAVVIIDMQRDFLEPGGFGAALGNDVAPLQAIVPTVQQLLALAREQGIAVIHT |
| | RESHRPDLSDCPQAKLDHGLPGLRIGDLGPMGRILVRGEPGDQIIDALTPLASEWVIDKPGKGMFFATDL |
| | HQRLTVAGITHLIFAGVTTEVCVQTSLREACDRGYRCLLIEDATESYFAAFKQATLDMITAQGAIVGRVA |
| | SLANLQHALHTRSTQ |
| 604 | WP_060602513.1 cysteine hydrolase [Aureimonas altamirensis]: |
| | MGTIAASPSPFRYEDGRVALIVIDMQRDFLEPGGFGESLGNDVSRLRAIVPATRRLIELFRARGAPIVHT |
| | RECHRPDLSDCPPAKWRRGPEGRRIGDIGPMGRILVAGAPGAEIVPELFPLPGETVIDKPGKGAFHATDL |
| | SEVLEGFGVTALVLAGVTTEVCVQTTMREANDRGYDCLLAEDATESYFAHFKEATLDMVRAQGGIVGWTA |
| | TVDAIAEGLTT |
| 605 | WP_060710524.1 cysteine hydrolase [Pseudonocardia sp. HH130629-09]: |
| | MISVDADPGAFTFDPATTALLIIDMQRDFCEPGGFGETLGNDVSLLRSVIPPLQEVLRVVRALGMTVIHT |
| | REGHVPDLSDCPPAKLNRGEPSLRIGDPGPKGRILVRGEYGHDIIDELRPEPGELVIDKPGKGSFHGTTF |
| | GAELRSRGITSLVVAGVTTEVCVQTTVREANDRGHECLVLSDCTGSYFPEFHRVALEMVAAQGGIFGWVA |
| | PSSALLTALTRDAVA |
| 606 | WP_060717601.1 cysteine hydrolase [Agrobacterium vitis]: |
| | MVDIKAQPFAFPLKLDQAALVIIDMQRDFTEPGGFGETLGNDVSLVSAIVPDVKRLLEAARTHGLTVIHT |
| | MECHRPDLSDLPDAKRNRGNPTLRIGDEGPMGRILIAGEYGTGILPELAPVDGELVIEKPGKGAFYATAL |
| | GEELTSRGITQLIFAGVTTEVCVQTTMREANDRGYDCLLIEEATASYFPAFKQATLEMIRAQGGIVGWTA |
| | HLDPFLEALAHG |
| 607 | WP_060737625.1 cysteine hydrolase [Bradyrhizobium sp. CCGE-LA001]: |
| | MLNSTNPAPAVINAEPEPIKLDWLATALLIIDMQRDFMEPGGFGETLGNDVSQLARAVKPIGAVLKAARD |
| | TGMLVIHTREGHLPDLSDAPPAKIERGAPSLRIGDPGPMGRILIRGEAGHDIIPELYPLDSEVVIDKPGK |
| | GAFYATELGEVLESYGIENLLVCGVTTEVCVNTTVREANDRGYRCIVISDGCASYFPEFHEMGLQMIKAQ |
| | GGIFGWVADSAAVLEAMNTSTG |
| 608 | WP_060769349.1 cysteine hydrolase [Methylobacterium sp. AMS5]: |
| | MPAPRPLLDAEPAPLPFDPARTALVVIDMQRDFLEPGGFGESLGNDVSLLAAAVPPARAVLTAARAAGLL |
| | VIHTREGHAPDLSDAPPAKLERGAPTARIGEPGPMGRILIRGEPGHDIVPELAPLGGEPVIDKPGKGAFY |
| | ATGLAALLEERGIETLIVCGVTTEVCVHTTVREANDRGYRCVVVADACGSYIPAFHEAGLAMIKAQGGIF |
| | GWVSRSTAVTAALGQA |
| 609 | WP_060817635.1 cysteine hydrolase [Caballeronia sordidicola]: |
| | MPQKLFQAEPFPLPFNAETTALVMIDMQRDFVEPGGFGEALGNDVSLVRSAIEPCRKLLKAARDAKLLVV |
| | HTREGHRADLADCPPAKLTRGGKRFIGTDGPMGRILVRGEAGHDIIPELYPALGEPIIDKPGKGAFYETD |
| | LQLILQNHGIKTLIVCGVTTEVCVTTTVREANDRGYECIIPADCVGSYFPEFQKSALEMIKAQGAIFGWV |
| | SNATAVIDGLRG |
| 610 | WP_060848264.1 MULTISPECIES: cysteine hydrolase [Methylobacterium]: |
| | MPRPVTIPAEPAPLTIDLDASALVVIDMQRDFLEPGGFGESLGNDVSLLAAAVPACRALLAAARAAGLLV |
| | VHTREGHAPDLSDAPPAKVARGAPRARIGEPGPMGRILVRGEAGHAIVPDLAPASGEIVIDKPGKGAFYA |
| | TGLGTLLEERGIANLIVCGVTTEVCVHTTVREANDRGYRCLVVSDACASYIPAFHEAGLAMIVAQGGIFG |
| | WVAPSPAVVPLVAGGGGIGGGSRTA |
| 611 | WP_061006523.1 cysteine hydrolase [Mycolicibacterium mucogenicum]: |
| | MTSVEVPAAPTPFSLVPGQTALIVIDMQRDFLLPGGFGESLGNDVDQLLKVVPPLAALIAAARAAGIMVI |
| | HTREGHEPDLSDCPPAKLNRGAPSKRIGDPGKYGRILIRGEYGHDIVDELAPIDGELVIDKPGKGAFYAT |
| | GLQDALTGAGITQLLVTGVTTEVCVHTTTREANDRGYECLVVSDCVGSYFPEFQRVGLEMIAAQGGIFGW |
| | VADTAAVIPALQQLAAPSASAV |
| 612 | WP_061075748.1 cysteine hydrolase [Citrobacter amalonaticus]: |
| | MTQQIFQAQPFALPFNPQTTALVMIDMQRDFVEAGGFGEALGNDVSFVRSAIEPCKKVLAAARSKGLMVI |
| | HTREGHRADLSDCPPAKLTRGGKTFIGEAGPMGRILVRGEAGHDIIPELYPVAGEPIIDKPGKGAFYQTD |
| | LHLILQNRGIKTLIVCGVTTEVCVNTTVREANDRGYECIIPEDCVGSYFPEFQKYALEMIKAQGAIFGWV |
| | SDANAIIAGLKG |
| 613 | WP_061133984.1 cysteine hydrolase [Caballeronia fortuita]: |
| | MPTLTHARPSPFTFDAAHTALIVIDMQRDFVEPGGFGEALGNDVSLLASIVPTVAQLLGHARERGWLVVH |
| | TRESHAPDLSDCPDAKRLRGAPSARIGDMGPMGRILVRGEPGNAIVDAVAPVAGEIVIDKPGKGAFYATR |
| | LGEELARFGITHLVFAGVTTEVCVQTSMREANDRGYECVLIEDATASYIPSFKTATIEMIRSQGGIVGWT |
| | ATLADLVESRWN |
| 614 | WP_061164088.1 cysteine hydrolase [Caballeronia temeraria]: |
| | MKQKHFRAEPFDLPFEPQRTALVMIDMQRDFVEPGGFGEALGNDVSFVRTAIEPCKRLLAAAREAGMLVI |
| | HTREGHRADLTDCPPAKLTRGGKTFIGTDGPMGRILVRGEKGHDLIPELYPVAGEPVIDKPGKGAFYETD |
| | LHLILKNSDIRTLIVCGVTTEVCVTTTVREANDRGFECIVPQDCVGSYFPAFQKSALEMIKAQGAIFGWV |
| | SDSGAVIEALRG |
| 615 | WP_061171675.1 cysteine hydrolase [Caballeronia hypogeia]: |
| | MAQKQFRAEPFNLVFDPLSTALVMIDMQRDFVEPGGFGEALGNDVSLVRTAIEPCRRVLEAARRTGMLVI |
| | HTREGHRPDLTDCPPAKLTRGGKTFIGTDGPMGRILVRGENGHDLIPELYPVAGEPVIDKPGKGAFYETD |
| | LHLILKNHGIKTLIVCGVTTEVCVTTTVREANDRGFECIVPQDCVGSYFPAFQKSALEMIKAQGAIFGWV |
| | SDSAAVIEGIAA |
| 616 | WP_061612643.1 cysteine hydrolase [Sorangium cellulosum]: |
| | MTPRAPEIAARPYPFRLAGPESVALLVIDMQRDFLEPGGFGAALGNDVRRLRRIVPTVRRLLDAFRERDL |
| | PILHTKEGHRPDLSDCPPAKRYRGAPGMRIGDAGPMGRILVLGEPGNDFVPELAPAPGEIVVPKPGKGAF |
| | YRTGLDARLASLGISQLLLAGVTTEVCVQSTMREANDRGYECLLIEDATESYFPEFKAATLEMIRAQGAI |
| | VGWTAPATTVLAAL |
| 617 | WP_061849110.1 cysteine hydrolase [Bradyrhizobium sp. DOA1]: |
| | MLNSTNPAPGVINAEPEPIKLDWLATALLIIDMQRDFMEPGGFGETLGNDVSQLARAVKPIGAVLKAARD |
| | TGMLVIHTREGHLPDLSDAPPAKIERGAPSLRIGDPGPMGRILIRGEAGHDIIPELYPLDSEVVIDKPGK |
| | GAFYATELGEVLESYGIENLLVCGVTTEVCVNTTVREANDRGYRCILISDGCASYFPEFHEMGLKMIKAQ |
| | GGIFGWVADSAAVLEAMKTSTG |
| 618 | WP_061878300.1 cysteine hydrolase [Bradyrhizobium liaoningense]: |
| | MLNSAKPTKGVISAEPEPITLDWPATALLIIDMQRDFMEPGGFGETLGNDVSQLARAVKPIGTVLTAARD |
| | TGMLVIHTREGHLPDLSDAPPAKVERGAPSLRIGDPGPMGRILIRGEAGHDIIPELYPLDSEIVIDKPGK |
| | GAFYATELGDVLGKYGIENLLVCGVTTEVCVNTTVREANDRGYRCVVISDGCASYFPEFHEMGLKMIKAQ |
| | GGIFGWVADSAAVLEAMKVSTT |
| 619 | WP_062137730.1 cysteine hydrolase [Paraburkholderia monticola]: |
| | MTTVARAKPAPFTFDARHTALIVIDMQRDFIEPGGFGEALGNDVSLLSGIVPTVARLIAHARSAGWLLVH |
| | TRESHAPDLGDCPPAKRLRGRPNARIGDHGPMGRILIRGEPGNAIIDTLKPLDGELVIDKPGKGAFYATR |
| | LGEELSMRGITHLVFAGVTTEVCVQTSMREANDRGYECLLIEDATASYIPAFRDATIAMIHSQGGIVGWT |
| | APLDALLEAA |
| 620 | WP_062168385.1 MULTISPECIES: cysteine hydrolase [Burkholderiaceae]: |
| | MPQKSFQAEPFALPFNPATTALVIIDMQRDFVEPGGFGEALGNDVSLVRTAIEPCRKLLKAARDAQLLII |
| | HTREGHRADLADCPPAKLTRGGKQFIGTDGPMGRILVRGEAGHDIIPELYPAIGEPIIDKPGKGAFYETD |
| | LQLILQNHGIKTLIVCGVTTEVCVTTTVREANDRGYECIIPADCVGSYFPEFQKSALEMIKAQGAIFGWV |
| | SDATAVIDGLRG |
| 621 | WP_062243908.1 cysteine hydrolase [Streptomyces griseorubiginosus]: |
| | MAVAESLSVEAAPYAFTFDPAETALVLIDMQRDFLEPGGFGESLGNDVEQLRRTVAPLRAVLDACRAAGM |
| | AVLHTREGHLPDLSDCPPSKLLRGNPSLRIGDPGPKGRILIRGEEGHDIIEELYPLAGEPVIDKPGKGAF |
| | YATEFGELLSARGIRRLVVTGVTTEVCVHTTVREANDRGYECLVLSDCVGSYFPQFQQAGLEMVAAQGGI |
| | FGWTAESAAFLTTLATAAPVGPDAPETAREPAPQPH |
| 622 | WP_062257858.1 cysteine hydrolase [Caballeronia megalochromosomata]: |
| | MKQKHFRAEPFDLAFEPQRTALVMIDMQRDFVEPGGFGEALGNDVSLVRTAIEPCRRVLAAAREAGMLVI |
| | HTREGHRADLTDCPPAKLTRGGKTFIGTDGPMGRILVRGEKGHDLIPELYPAEGEPVIDKPGKGAFYETD |
| | LHLILKNRDIRTLIVCGVTTEVCVTTTVREANDRGFECIVPQDCVGSYFPAFQKSALEMIKAQGAIFGWV |
| | SDAGAVIEALRG |
| 623 | WP_062277538.1 MULTISPECIES: cysteine hydrolase [unclassified |
| | Rhizobium]: |
| | MADIKALPFSFPLRREAVALIVIDMQRDFAEPGGFGESLGNDVSHVTAIVSDVKRLIEGFRAAGLPVIHT |
| | QECHRPDLSDLPLAKRNRGTPTLRIGDVGPMGRILISGEPGTAILPELAPIEGEIVIEKPGKGAFYATPL |
| | GETLKANGIEQLVFAGVTTEVCVQTTMREANDRGYECLLVEEATASYFPQFKRAALDMIRAQGGIVGWTA |
| | HLDDVLEAIDA |
| 624 | WP_062322404.1 MULTISPECIES: cysteine hydrolase [Halolactibacillus]: |
| | MTNKLAVEAKPYEFTFDPDKTALVIIDMQRDFLYPGGFGEQLGNDVSKTNVIIPTVQKMLEKAREKDMFI |
| | IHTREGHRPDLSDVPPSKQRRGGNIGEVGPMGRILVRGEYGHDIVDELQPKSGEVVLDKPGKGAFYQTDL |
| | DSILKNKGIESLIVAGVTTHVCVQTTIREANDRGFECLMLEDACAAFDPKDHEDSIRMINQQGGIFGWTA |
| | PTENVLRVLG |
| 625 | WP_062371641.1 cysteine hydrolase [Rhizobium altiplani]: |
| | MADIKAQPFAFPARPDQLALIVIDMQRDFAEPGGFGASLGNDVSRITRIIPDVKRLIEGFREAGLPVIHT |
| | MECHKPDLSDLPPAKRNRGAPTLRIGDDGPMGRILIAGEAGTAILAELAPVEGEIVIEKPGKGAFYATEL |
| | GDILKARGISQLVFAGVTTEVCVQTTMREANDRGYECLLAEEATESYFPEFKDAAIAMIRAQGGIVGWTS |
| | HVDDILEAIGHA |
| 626 | WP_062460032.1 cysteine hydrolase [Rhizobium sp. Leaf306]: |
| | MSMIKAEPFDFPSRPAEMALVVIDMQRDFAEAGGFGASLGNDVSRITRIVPDVKRLIEGFRASGIPVIHT |
| | MECHRPDLSDLPPAKRDRGNPALRIGDEGPMGRVLIAGEPGTAILPELAPIAGEVVIEKPGKGAFYATAL |
| | GEILKQKGITQLVFAGVTTEVCVQTTMREANDRGYECLLCEEATESYFPEFKAAAIAMIRAQGAIVGWTA |
| | HIDDILKGLPHA |
| 627 | WP_062557086.1 cysteine hydrolase [Rhizobium sp. Root149]: |
| | MPTIKALPFDFRFEPASTALIVIDMQRDFIEPGGFGETLGNDVSRVSVIVPDVKRLIEGCRREGLTVIHT |
| | MECHRPDLSDLPDAKRNRGNPTLRIGDAGPMGRILIAGEAGTEIVTELAPLPGEIVIEKPGKGAFYATGL |
| | SQVLAERGITHLIFAGVTTEVCVQTTMREANDRGFNCLLVEEATASYFPEFKQAALEMIRAQGGIVGWTA |
| | HLADFLEGVNHG |
| 628 | WP_062584720.1 MULTISPECIES: cysteine hydrolase [unclassified |
| | Rhizobium]: |
| | MPEKLTATIKAEPFPFPLKRDAIALVVIDMQRDFAEPGGFGASLGNDVSRITKIVPDVKKLIEGFRSAGL |
| | PVIHTMECHRPDLSDLPPAKRNRGNPTLKIGDEGPMGRVLIVGEPGTAILPELAPVDDEIVIEKPGKGAF |
| | YATPLGDILKSKGIEQLVFAGVTTEVCVQTTMREANDRGYECLLAEEATESYFPEFKAAAIAMMKAQGAI |
| | VGWVGSVDDIIEGIE |
| 629 | WP_062593886.1 cysteine hydrolase [Rhizobium sp. Leaf371]: |
| | MAEITAEPFAFPARSGEMALVVIDMQRDFAEPGGFGASLGNDVSRIGRIVPDVKRLIEGFRTAGLPVIHT |
| | MECHRPDLSDLPPAKRDRGSPALRIGDEGPMGRVLIVGEPGTAILPELAPIDGEIVIEKPGKGAFYATPL |
| | GEILKQKGITQLVFAGVTTEVCVQTTMREANDRGYECLLCEEATESYFPDFKAATLAMIRAQGAIVGWTA |
| | HLDDILKGLPHA |
| 630 | WP_062656996.1 cysteine hydrolase [Mycolicibacterium canariasense]: |
| | MSASVPAEPSAFTLEPGRTALIVIDMQRDFLLPGGFGESLGNDVHQLLKVVGPLADLIAAARAAGLLVIH |
| | TREGHQPDLSDCPPAKLNRGAPSQRIGDPGKYGRILIRGEYGHDIVDELAPIAGEVVIDKPGKGAFYATD |
| | LQDVLTGAGITQLLITGVTTEVCVHTTTREANDRGYECLVVSDCVGSYFPEFQRVGLEMITAQGGIFGWV |
| | ADTAAVIPALQQLTPHAA |
| 631 | WP_062763343.1 cysteine hydrolase [Tistrella mobilis]: |
| | MPITIDAAPYAFTAPRDRLALIVIDMQRDFLEPGGFGASLGNDVTRVRPSIAPTRRLLEGFRTAGLPVFH |
| | TRECHLPDLSDCPPAKHGRGPGPLRIGDPGPMGRILIRGEPGADIIPELAPLPGEVVIDKPGKGAFHATP |
| | LGDELARRGISHLVFAGVTTEVCVQTTMREANDRGFDCLLATDATDSYFPEFKAATIAMITAQAGIVGWA |
| | APVDAVLTALRADT |
| 632 | WP_062944041.1 cysteine hydrolase [Rhizobium leguminosarum]: |
| | MMEIKAEPFAFPVKHDELALIVIDMQRDFAEPGGFGASLGNDVSRITRIVPDVKRLIQGFRNAGLPVIHT |
| | MECHRPDLSDLPPAKRDRGNPSLRIGDEGPMGRILISGEPGTAILPELAPVKGEVVIEKPGKGAFYATDL |
| | GTVLQQKGIKQLVFAGVTTEVCVQTTMREANDRGYECLLAEEATESYFPEFKAAAIAMIRAQGAIVGWTA |
| | HVDDILESIAHA |
| 633 | WP_063142283.1 cysteine hydrolase [Alcanivorax sp. KX64203]: |
| | MLNVSAKPNAFPLDPGHCALVVIDMQRDFIEPGGFGAALGNDVSRLAPVVPKVAALLALAREQRLTVVHT |
| | RESHLPDLSDCPPLKRNKLPAGRRIGDDGPMGRILVRGEPGNRILDAVAPEPGEWQVDKPGKGMFHATGL |
| | DQRLRDAGITQLIFAGVTTEVCVQTSMREACDRGYDCLVIEGATESYFPEFKAATLAMIVAQGGIVGRCA |
| | SLDALRRAFQQGANA |
| 634 | WP_063196387.1 cysteine hydrolase [Bradyrhizobium sp. AT1]: |
| | MLSSSKSTLGVISAEPEPIKLDWATTALLIIDMQRDFMEPGGFGETLGNDVSQLARAVKPIGALLNAARD |
| | TGMLVIHTREGHLPDLSDAPAAKVERGAPSLRIGDPGPMGRILIRGEAGHDIIPELYPLDSEIVIDKPGK |
| | GAFYATELADVLEKYGIENLLVCGVTTEVCVNTTVREANDRGYRCVVMSDGCASYFPEFHEMGLKMIKAQ |
| | GGIFGWVASSAAVLEAMKVSIT |
| 635 | WP_063391010.1 cysteine hydrolase [Ralstonia mannitolilytica]: |
| | MTCTVAAQPFDYHFEPAHTALLIIDMQRDFVEPGGFGASLGNDVTPLQAIIPTVQRVLATWRALGGLVVH |
| | TREAHLPDLSDCPPAKRERGQPSLRIGDVGPMGRVLIRGEPGHAIVPALAPVDGEIVIDKPGKGAFYATG |
| | LDETLRERGITHLIVMGVTTEVCVQTSMREANDRGYECLLVEDGTDSYFPEFKAATLAMIRAQGAIVGWT |
| | APSALLLQALPAPKP |
| 636 | XP_016220556.1 allophanate hydrolase [Exophiala mesophila]: |
| | MGVDSKRGFLTIEAKPYPFTFPLATTALLVIDMQRDFILAGGFGEIQGGNLEAVQASIAPTKELLQASRD |
| | AGLAIFHTREGQVPSLADCPSSKLVRQAAAPGNSQHLKVIGDKGEMGRLLVRGEYGHDIVDELQPRTAEV |
| | VIDKPGKGSFWNTDILHKLKARGITHLLVSGVTTECCFATTIREANDRGFECCGITQSTAGYNAEFKTAS |
| | LDMIHWSQGLFGFVADLQPVLEAFSPWKKTYGGEGTPPQTPPTWDGNLGISDLQASYSSGVSPLEVINAV |
| | FDRIEKYDAVDPAVWIKRESRTAVMEAVRQLLARFPDRNSLPPLFGVPFTVKDSIDVKGIQTTTACPPLA |
| | FVATKSAVVYDKVIAQGAIYLGKVNLDQLATGLNGCRSPYGVTHSVFSDKHISGGSSSGSCVSVGADLAT |
| | FSVATDTAGSGRVPAGFNGIVGYKPTRGLVSFEGVTPACLSLDCIAFSARTVQDARTLWQVAEEFDPNDR |
| | YARDVFPMERHVNSIGAQRNSFRFGIPPPEVLEVCSHKYRQLFNEAVNRLQKMGGVLTSVDWTPFQKAGD |
| | LLYAGTFVSERLASLPDDFLDKNRAHLHPVILELFEQVVSRQSSAVQLFRELQAQALYKRQATSQFASAN |
| | TLGIDVLVVPTAPEHPTIEAMQADPIRLNAKLGTFTHFGNVLDMCAVAVPAGMYASETGENGEQLPFSIT |
| | LLGARCTDAEVLTIAGHFQEAMTHVGS |
| 637 | XP_016218308.1 allophanate hydrolase [Verruconis gallopava]: |
| | MTTKLPESITFDAKPYGFTFNPRHTALVIIDMQRDFLLKDGFGEIQGGNLEMVQASIAPTKRLLDLCRKA |
| | GLSIFHTREGHKPDLSDLPSSKLHRQAAMPGNTQHRFVIGEKGPLGRLLTRGEYGHDIVDELAPLPGEVV |
| | IDKPGKGSFWNTDILHKLKARAITHLIVSGVTTECCFATTIREANDRGFECCGIEEATAGYNADFKCASL |
| | DMIHWSQGLFGFVSSLQSFVDALEPYLPHAESTLSPPLTPSVWDGDVRISSLRAAYRAGLSPVTVVESLY |
| | KTIEAYENENSGVWIHLQPKDQLINEAKALVAKYPDKSALPPLYGIPFNLKDSLDVAGLPTTTACPPLTH |
| | IASTSTPTYEKCVAQGALFLGKVNLDQLATGLNGTRSPYGIPHSVYHEDYISGGSSSGSSVSVGASLVSF |
| | SIATDTAGSGRVPAAFNGVVGYKPTRGILSTVGLVPACLSLDCIAIIARTVEDARTVWQICEGYDPRDRY |
| | SKTPIGFDRHVNSIGPEATRFKFGIPPPESLSVCSPVYRKMFHESVKQLQNIGGVLTPIDWTPFEKGGRL |
| | LYDGTFVSERLANLPDDFLEKNSKHLHPVILELFQQVQARNSSAVQAYRDLQAKALYTRQAEEVLGYSGT |
| | GVDIVVVPTTVTHWKTKELLADPIKKNSQLGEFTHCGNVLDLCAISIPAGEYEISELSGNKDDRGKLPFG |
| | VMFLASSRMDAEILELARRFEARMSSKS |
| 638 | XP_016235744.1 allophanate hydrolase [Exophiala spinifera]: |
| | MGSLLEDVLTIEAKPYPFTFPLSRTALLVIDMQRDFLLPSGFGEIQGGNLSAVQASIAPTKALLEACREA |
| | GMKIFHTREGHVPSLADCPSSKLIRQAASPSSQHLKVIGDKGDMGRLLVRGEYGHDIVDELKPLPSEVVI |
| | DKPGKGSFWNTAILHKLKSYGITHLLISGVTTECCFATTLREANDRGFECVGIRESTAGYNPEFKTASLD |
| | MISWSEGLFGFVANLQPVLDVLSPWKKVNSGENTPPQTPPPWDGKLEISDLLASYKNGLSPAVMVNELFD |
| | RIEKYDTVDSTVWIRREPREDVLRRVAELVAQFPDRNALPPLFGIPFTVKDSIDVQGIETTTACPPLAFV |
| | ASKSAVCYQKVIDAGAIYLGKVNLDQLATGLSGCRSPYGITHSVFSDEYISGGSSSGSAVSVGADLATFS |
| | LATDTAGSGRVPSGFNGVVGFKPTRGLISFDGVTPACLSLDCVALIAKNVEDARTVWQVCEGFDPNDRYA |
| | RDTFPAPRHVNATGPQKDSFRFGIPPPEALEICTPTYHRLFTEAVRRLQAMGGTLVPIDWVPFQRAADLL |
| | YEGTFVSERLASLPDNFLDKNARHLHPVILKLFRQVVERQSTAVQLFRDLHNKALYTRQATAQFTSADQL |
| | GIDVLVVPTAPEHPTIEAMLADPIKLNAKLGTFTHFGNVLDLCAVAVPSGSFPASTTELPFSITFLGCRC |
| | SDSETLTVASRYQRHVTRQMS |
| 639 | XP_016255615.1 allophanate hydrolase [Cladophialophora immunda]: |
| | MGMSKDKQGFLTIEAKPYPFSFPLQHTALLVIDMQRDFISAGGFGEIQGGNLEAVQASIAPTKQLLDACR |
| | DAGMHIFHTREGQVPSLADCPSSKLVRQAAAPGNTQHLKVIGDKGEMGRLLVRGEYGHDIVDELQPLPSE |
| | VVIDKPGKGSFWNTPILHKLKAYGITHLLVSGVTTECCFSTTIREANDRGFECCGIVQSTAGYNSAFKTA |
| | SLDMIHWSQGLFGFVADLQPVLDVLSPWQSQSKGVSTPPQTPPSWDGKLGIADLQASYRSGLSPLELVNA |
| | LFDRIEKYEHIDGAVWIRRESREAVLDQARRLLELYPDKNARPALFGVPFTVKDSIDVQGVETTTACPPL |
| | AFVATKSAMCYQKVVGQGALYLGKVNLDQLATGLSGCRSPFGITHSVFSDEHISGGSSSGSCVSVGADLA |
| | TFSLATDTAGSGRVPAGFNGVVGYKPTRGLVSFEGVTPACLSLDCIAFTARTVEDARTLWQVCEGYDEND |
| | RYARDTFPAERHVNSIGTQREAFRFGIPPPELLEVCSPSFRKLFNEAVARLQGMGGTLVAMDWTPFQKAG |
| | DLLYEGTFVSERLASLPDDFLEKNRQHLHPVILELFEKVVARQSTAVQLFRELQAKALYSRQATSQFRSA |
| | DRLGLDVVVVPTAPWHPTIQEMLADPIRLNAKMGTFTHFANVLDMCGIAVPSSTYQESEAGPRLPFSVTF |
| | LGSRCSDSEVLGIAGRYQEATGR |
| 640 | XP_016589526.1 allophanate hydrolase [Sporothrix schenckii 1099-18]: |
| | MDELTFATARPYAFSFVRAHTALVLIDLQRDFVDPGGFGAIQCGSSEIFAGVRKVVPATLPVLAAARKLG |
| | LHIVHTREGHRPDLADLSAAKRNRQLDAPGTQHTAGIGDKGPMGRLLVRGEHGHDFVDELRPRPDEIVVD |
| | KPGKGAFWATSMHRQLMARGVTHLLFCGVTTECCVASTAREASDRGFQCCILEDCTGGFEASFATASVDM |
| | YVSFGGLFGFAAPSTELVVYAKAETGKTESLSTPQVPWDGKSLDLTTLQAYYKRSALSPIEIVNAVYDHI |
| | EAYEKENPHVWILLRPRGDVIEDAQALQDKYAAIGRDSLPPLYGVPFAVKDSFDIKGMNTTAACPDFAYL |
| | ATETAPTVTSILDAGALLIGKTNMDQLATGLSGCRSPYGVSSSVFSPADMKYCSGGSSSGSAVAVGAHLV |
| | TFSLATDTAGSGRVPASFNGIVGYKPTKGTLSYRGIVPCCRSIDTATILAQSVADARRIWHIVDQYDDQD |
| | IFAKDPQSLPLTLADYRGIQKAGFTCAVPPNSALAVASCSPAYQAAFTAALQVVRRIGGRLRTLSDTAYQ |
| | PFMTATDLLYNGTLVNERIACMGVDFVRDHITNFHPTTKTLFEQVLERDSKPWDVYGDQLVQATATQQAG |
| | RLLSGGQGSLGGVGAGSGGESSVVDVLVVPTAPFHPTIAEMQADPIALNAKLGVFTHFGNVLDLCAMSVN |
| | AGFVEDGMPFGVCFVCARGMDGRLFDIASEFERAVAATK |
| 641 | XP_016631078.1 hypothetical protein Z520_07069 [Fonsecaea |
| | multimorphosa CBS 102226]: |
| | MGMAKDGKQGQGVLTIEAKPYPFSFPLKHTALLVIDMQRDFICAGGFGEIQGGNLEAVQASIAPTKSLLD |
| | ACRDAGMHIFHTREGQVPSLADCPSSKLVRQAAAPGNTQHMKVIGDKGELGRLLVRGEYGHDIVDELQPL |
| | PSEVVIDKPGKGSFWNTPILHKLKAYGITHLLVSGVTTECCFSTTIREANDRGFECCGIVQSTAGYNSAF |
| | KTASLDMIHWSQGLFGFVADLQPVLDVLSPWQTQNKGVSTPPQTPPAWDGKLGIADLQASYKHGLSPLEL |
| | VNALFDRIQKYEHIDPAVWIRRESREAVLDQARRLLELYPDKNARPALFGVPFTVKDSIDVQGIETTTAC |
| | PPLAFVATKSAMCYQKVVGQGAIYLGKVNLDQLATGLSGCRSPFGITHSVFSDEHISGGSSSGSCVSVGA |
| | DLATFSLATDTAGSGRVPAGFNGVVGYKPTRGLVSFEGVTPACLSLDCIAFTARTVEDARTLWQACEGYD |
| | ENDRYARDTFPAERHVNSIGAQKGTFRFGIPPPELLEVCSPSFRKLFNEAISRLQAMGGTLVPMDWTPFQ |
| | KAGDLLYEGTFVSERLASLPDDFLEKNRQHLHPVILELFEKVVARQSTAVQLFRELQAKALYTRQATSQF |
| | SSADRLGLDVVVVPTAPWHPTIKEMLADPIRLNAKMGTFTHFANVLDMCGIAVPSTTYQESEAGPRLPFS |
| | VTFLGSRCSDSEVLGIASRYQEATGR |
| 642 | XP_016617098.1 allophanate hydrolase [Cladophialophora bantiana CBS |
| | 173.52]: |
| | MGMSKDKQGFLTIEAKPYPFSFPLKHTALLVIDMQRDFICAGGFGEIQGGNLEAVQASIAPTKQLLDACR |
| | DAGMHIFHTREGQVPSLADCPSSKLVRQAAAPGNTQHLKVIGDKGEMGRLLVRGEYGHDIVDELQPLPAE |
| | VVIDKPGKGSFWNTQILHKLKAYGITHLLVSGVTTECCFSTTIREANDRGFECCGIVQSTAGYNSDFKTA |
| | SLDMIYWSQGLFGFVADLQPVLDVLSPWQIQSKGVSTPPQTPPSWDGKLGIADLQASYKNGLSPLELVNA |
| | LFDRIEKYEHIDGAVWIRRESREAVLEQARRLLELYPDKNARPALFGVPFTVKDSIDVQGVETTTACPPL |
| | AFVATKSAACYQKVVEQGALYLGKVNLDQLATGLSGCRSPFGITHSVFSDEHISGGSSSGSCVSVGADLA |
| | TFSLATDTAGSGRVPAGFNGVVGYKPTRGLVSFEGVTPACLSLDCIAFTARTVEDARTLWQICEGYDESD |
| | RYARDTFPAERHVNSLGAQREAFRFGIPPPELLEVCSPSFRKLFNEAIARLQAMGGTLMPIDWTPFQKAG |
| | DLLYEGTFVSERLASLPDDFLDKNRQHLHPVILELFEKVVARQSTAVQLFRELQAKALYTRQATSQFASA |
| | TQLGIDVVVVPTAPWHPTIKEMLADPIRLNAKMGTFTHFANVLDMCGIAVPSSTYQESEAGPRLPFSVTF |
| | LGSRCSDSEVLGIASRYQEATGR |
| 643 | XP_016641923.1 Uncharacterized protein SAPIO_CDS6367 [Scedosporium |
| | apiospermum]: |
| | MASLDYTTTTLSIEAKPYAYTFRPSCTALLLIDMQHDFLHPVGFGESCGADLKMVQACIEPARKLLGACR |
| | ASGLTIFHTREGHRQDMSDCPSSKITQQAEAFGMERKPRIGEKGPMRKTPIKGEYGHDFVDELQPVPGEI |
| | VIDKPGKGAFWDTELMHKFKAHGITHLLVAGITTKGSVSTTFREASDRGFHCCVITEATAGYDSSFTAAS |
| | LDILCSTNGGFGFVAHLQPILSELSHIPRPLPSYTETSQETSPEWDGKLDIVSLQTAYQAGFSPLTVVED |
| | IFTRIEAYENVNPGSWIYRVPKSVVLEATRDLLNRFPDRSKRPPLFCVPFSIKDSIDVAGIPTTTACPPL |
| | SHIPSVNAPLHIALIEQGGLFIGKSNLDQLATGLTGQRSPYGAPSSAINSSYVPGGSSSGSSVLASTVRD |
| | ARTVWRILETFDPRDPYTKPEELRKCPHVVHSTGQTETTFRFGIPPHDVLGICSEPYRRLFAETVTQLON |
| | IGGRLQHINWKPFDKAGRLLYDGTFVLERVASIPDLPGSGGIDGPTWFEKHKADLHPVISELFTAVINRK |
| | VTAVDVFRDLQAQRRYTALVHNEVFSQGASGVDVVVLPTAPTHWTVDEVKEDPIVKNSALGVFTHCANVL |
| | DLCAISCPAGEFAAKELGGQGVLPFGVMFMGRRGGDSEVLDLATRFEDSFKEDVDASRG |
| 644 | WP_063678358.1 cysteine hydrolase [Bradyrhizobium neotropicale]: |
| | MLNSTKPTSGVISAEPEPIKLDWSTTALLIIDMQRDFLEPGGFGETLGNDVSQLARAVKPVAAVLQAARE |
| | SGMLVIHTREGHLPDLSDAPPAKIERGAPSLRIGDPGPMGRILIRGEAGHDIIPELYPLDSEVVIDKPGK |
| | GAFYATELSDVLEKYGIETLLVCGVTTEVCVNTTVREANDRGYRCLVISDGCASYFPEFHEMGLKMIKAQ |
| | GGIFGWVTDSAAVLQALKASGVSS |
| 645 | WP_063705729.1 cysteine hydrolase [Bradyrhizobium centrolobii]: |
| | MLNSTKPTLGVISAEPEAIKLDWASTALLIIDMQRDFMEPGGFGETLGNDVSQLARAVKPIGAVLAAARD |
| | TGMLVIHTREGHLPDLSDAPPAKVERGAPSLRIGDPGPMGRILIRGEAGHDIIPELYPLDSEVVIDKPGK |
| | GAFYATELSDVLEKYGIENLLVCGVTTEVCVNTTVREANDRGYRCVVISDGCASYFPEFHEMGLKMIKAQ |
| | GGIFGWVADSAAVLEALKTSSI |
| 646 | XP_016763590.1 glutamyl-tRNA amidotransferase subunit A [Sphaerulina |
| | musiva SO2202]: |
| | MATVMELPSARPYPYKFPPESTALIIIDMQRDFVDYHGFGQIQCGNDEVFKKVRNIVPRTRQALEAARSL |
| | GLHVVHTREGHTPDLSDLPPSKRLRQISAPSGHHTMGIGDAGPMGRLLVRGEYGHDIIDELRPIPGEPVI |
| | DKPGKGSMWDTNLHRVLLARGITHLLFAGVTTECCVNTTARECADRGFETCILADCTDGFDEGFYSSTLD |
| | MLCSYDGLFGFVGTSAELLKYAPPQAPTPPTTPPGFSGDISIANLRKQYTAGQLRPTDTVKEVHARAASY |
| | RLKDSAVWTHLRDVEEILKDAQALEDRFSGKPLPELYGIPFAVKDNIDVANVKTTAACEAYAYLPGRSAK |
| | VVETLLEAGAIFIGKTNLDQLATGLSGCRSPYGTPHSVFSTHHISGGSSSGSAVAVGAGLVSFALGTDTA |
| | GSGRVPAAYNGIVGHKPTKGTFSASGLVPACKSLDTITVLAPSVNDARKVWLVADIGGDETDPYSKSPSS |
| | LALWHADFRGVKVGGFTFGIPPATALEKCDGKYQELFAASVDRLTRAGGTPKEVDWVAFEGGSNLLYEAS |
| | LVQERIASIGPEFIEKNINTLHSTTNKLFSEAAHKDVKPWQVFRDQHLQAQYTRDAASIFQSIDVLLVPT |
| | TPCHPTISEMESDPLALNAKLGYFTHFANVLDLCGVAVPAATYQDATGTTLPFGVTLLGASGRDGRVYDI |
| | AREFERTV |
| 647 | WP_064243178.1 cysteine hydrolase [Ensifer glycinis]: |
| | MAEIKAEPFPLRLDRDAVALIVIDMQRDFTEEGGFGASLGNDVARVAKIVPDVKRLIEGFRAAGLPVIHT |
| | MECHRPDLSDLPRAKRERGSPRLRIGDEGPMGRVLIAGEPGTAILPELAPLKGETVIEKPGKGAFYATPL |
| | DYILKERRIVQLVFAGVTTEVCVQTTMREANDRGYECLLVEEATESYFPEFKAATMAMIRAQGAIVGWTA |
| | HLADVLKGIAHA |
| 648 | WP_064246125.1 cysteine hydrolase [Rhizobium leguminosarum]: |
| | MAEIKAEPFAFPVKHDELALIVIDMQRDFAEPGGFGASLGNDVSRITRIVPDVKRLIQGFRNAGLPVIHT |
| | MECHRPDLSDLPPAKRDRGNPALRIGDEGPMGRILIAGEPGTAILPELAPVKGEIVIEKPGKGAFYATQL |
| | GTVLLQKGIKQLVFAGVTTEVCVQTTMREANDRGYECLLAEEATESYFPEFKAAAIAMIRAQGAIVGWTA |
| | HVDDILESIAHA |
| 649 | WP_064283501.1 cysteine hydrolase [Mycolicibacterium iranicum]: |
| | MNHIVEVPAEPSSFRLVRDSTALVVIDMQRDFLLPGGFGESLGNDVDQLLKVVPPLAALIAAARAAGIMV |
| | VHTREGHQPDLSDCPPAKLNRGAPSKRIGDPGKYGRILIRGEYGHDIVDELAPIDGEVVIDKPGKGAFYA |
| | TELSDLLTEAGITQLLVTGVTTEVCVHTTTREANDRGYECLVVSDCVGSYFPEFQRVGLEMIKAQGGIFG |
| | WVADTSAVIPALSKLTTTAA |
| 650 | WP_064654454.1 cysteine hydrolase [Rhizobium sp. WYCCWR10014]: |
| | MAKIKAEPFAFPVKHDELALIVIDMQRDFAEPGGFGASLGNDVSRITRIVPDVKRLIQGFRNAGLPVIHT |
| | MECHRPDLSDLPPAKRDRGNPSLKIGDEGPMGRILISGEPGTAILPELAPVKGEVVIEKPGKGAFYATEL |
| | GTVLQEKGIRQLVFAGVTTEVCVQTTMREANDRGYECLLAEEATESYFPEFKAAAIAMIRAQGAIVGWTA |
| | HVDDILESIAHA |
| 651 | WP_064868049.1 MULTISPECIES: cysteine hydrolase [Gordonia]: |
| | MSESVTVMALPEPIDLDLDRTALVIIDMQRDFLLPGGFGETLGNDVGQLQKVVEPLQALLAAARSAGMLV |
| | VHTREGHLPDLSDCPPAKLSRGAPSKRIGDPGAFGRILIRGEYGHDIVDELAPLADEVVIDKPGKGAFYA |
| | TDFGKVLESNGITQLLVTGVTTEVCVHTTTREANDRGFECVVVSDCVGSYFPEFQRVGLEMIAAQGGIFG |
| | WVAASADLLPAMLGRRAEASSDWS |
| 652 | WP_064978905.1 cysteine hydrolase [Mycolicibacterium mucogenicum]: |
| | MTSVAVPAAPTPFTLTAGQTALIVIDMQRDFLLPGGFGESLGNDVDQLLKVVPPLAALLAAARAAGVMVI |
| | HTREGHEADLSDCPPAKLNRGAPSKRIGDPGKYGRILIRGEYGHDIVDELAPIDGELVIDKPGKGAFYAT |
| | GLQDALTAAGITQLLVTGVTTEVCVHTTTREANDRGYECLVVSDCVGSYFPEFQRVGLEMIAAQGGIFGW |
| | VADTAAVIPALQKLAAPSPSAV |
| 653 | WP_065007119.1 cysteine hydrolase [Mesorhizobium sp. AA22]: |
| | MAEIDALPFPFGCKPEAMALVVIDMQRDFAEPGGFGASLGNDVSRVVAIVPMVKRLIEGFRAAGLPVIHT |
| | MECHRPDLSDLPPAKRSRGNPSIRIGDVGPMGRVLIVGEPGTAILDELAPLPGEIVIEKPGKGAFYATSF |
| | GENLKRLGVQQLVFAGVTTEVCVQTTMREANDRGYECLLAEDATESYFPEFKAAVLAMIRAQGAIVGWTA |
| | TTDQVLKGIADA |
| 654 | WP_065058370.1 MULTISPECIES: cysteine hydrolase [Paraburkholderia]: |
| | MPTLAGALPSPFVFEAPKTALVVIDMQRDFIEPGGFGAALGNDVSLLGGIVPDVARLLHHARERGWFVVH |
| | TRESHAADLSDCPPAKRLRGQPSARIGDAGPMGRILVRGEPGNAIVDALAPVGGELVIDKPGKGAFHATR |
| | LGEELAQRGITHLVFAGVTTEVCVQTSMREANDRGYDCLLIEDATASYIPAFKAATLAMIHSQGGIVGWT |
| | ASLAQLLEADA |
| 655 | WP_065227979.1 MULTISPECIES: cysteine hydrolase [Escherichia]: |
| | MTQSIFQAQPFELPFDPRTTALVMIDMQRDFVEAGGFGEALGNDVSLVRTAIAPCTEVLAAARQKGIMVI |
| | HTREGHRADLSDCPPAKLTRGGKTFIGEAGPMGRILVRGEAGHDIIPELYPVAGEPVIDKPGKGAFYQTD |
| | LHLILQNHGIKTLIVCGVTTEVCVTTTVREANDRGYECIIPEDCVGSYFPEFQKYALEMIKAQGAIFGWV |
| | TDSKAIIAGLEG |
| 656 | WP_065277314.1 cysteine hydrolase [Rhizobium leguminosarum]: |
| | MAKIKAEPFAFPVKHDELALIVIDMQRDFAEHGGFGASLGNDVSRITRIVPDVKRLILGFRNAGLPVIHT |
| | MECHRPDLSDLPPAKRDRGNPSLRIGDEGPMGRILISGEPGTAILPELAPVKGEVVIEKPGKGAFYATEL |
| | GTVLQEKGIRQLVFAGVTTEVCVQTTMREANDRGYECLLAEEATESYFPEFKAAAIAMIRAQGAIVGWTA |
| | HVDDILESIAHA |
| 657 | WP_065546087.1 cysteine hydrolase [Vibrio scophthalmi]: |
| | MIKSFNAEPFALEFDPTTTALVMIDMQRDFVEPGGFGEALGNDVSLVRTAIEPCVAVLEAARQAGLTVIH |
| | TREGHRADLTDCPAAKLTRGGKTFIGEMGPKGRILIRGEEGHDIIPELYPIAGEPIIDKPGKGAFYQTDL |
| | HLILQTRNIKTLIVCGVTTEVCVTTTVREANDRGYECIVPEDCVGSYFPEFQKYALEMIKAQGGIFGWVS |
| | HSKDIIEAIK |
| 658 | WP_065691257.1 cysteine hydrolase [Rhizobium sp. AC44/96]: |
| | MTEIKAQPFAFPAKPGELALVVIDMQRDFAEPGGFGASLGNDVGRITKIVPDVKRLIQGFRDAGLPVIHT |
| | MECHKPDLSDLPPAKRNRGKPSLRIGDDGPMGRILIAGEPGTAILPELAPIDGETVIEKPGKGAFYATEL |
| | GDVLKEKGITQLVFAGVTTEVCVQTTMREANDRGYECLLAEEATESYFPEFKDAAIAMIRAQGAIVGWTA |
| | HVDDILEVIGHA |
| 659 | WP_065730879.1 cysteine hydrolase [Bradyrhizobium icense]: |
| | MANSRKLAAEPEPIELDWAATALLIIDMQRDFMEPGGFGETLGNDVSQLARAVKPIAAVLDAARAAGMLV |
| | IHTREGHLPDLSDAPPAKVERGEPSLRIGDPGPMGRILIRGEPGHDIIPELYPLDSEIVIDKPGKGAFYA |
| | TELGDVLQRYGIENLLVCGVTTEVCVNTTVREANDRGYRCVVLADCCASYFPEFHEMGLKMIKAQGGIFG |
| | WVSDSAAVLRAISPEIPTTAVAGGLR |
| 660 | WP_065748817.1 cysteine hydrolase [Bradyrhizobium sp. LMTR 3]: |
| | MANSRKLAAEPEPIELDWAATALLIIDMQRDFMEPGGFGETLGNDVSQLARAVKPIAAVLEAARATGMLV |
| | IHTREGHLPDLSDAPPAKVERGAPSLRIGDPGPMGRILIRGEPGHDIIPELYPLDSEIVIDKPGKGAFYA |
| | TELGDVLQRYGIENILVCGVTTEVCVNTTVREANDRGYRCVVLADGCASYFPEFHEMGLKMIKAQGGIFG |
| | WVSDSAAVLNALSPEIPTTAVAGASR |
| 661 | WP_065751207.1 cysteine hydrolase [Bradyrhizobium paxllaeri]: |
| | MANSAKLAAEPEPIELDWAATALLIIDMQRDFMEPGGFGETLGNDVSQLARAVKPIASVLDAARGAGMLV |
| | IHTREGHLPDLSDAPPAKVERGAPSLRIGDPGPMGRILIRGEAGHDIIPELYPLDSEIVIDKPGKGAFYA |
| | TELGEVLQRYGIENLLVCGVTTEVCVNTTVREANDRGYRCVVLADGCASYFPEFHEMGLKMIKAQGGIFG |
| | WVSDSAAVLGTLSPEIPTTAVAGASR |
| 662 | WP_066067912.1 cysteine hydrolase [Frankia sp. EI5c]: |
| | MTPTAPLTVRARPYDYTFDPASTALVLIDMQRDFLEPGGFGESLGNDVSLLRSTIEPLRVVLAAARAAGL |
| | TVIHTREGHLPDLSDLPPSKLARGNATARIGDLGPNGRILIRGEYGQDIIDELAPAAGEPVIDKPGKGAF |
| | HATEFGDVLQARGITHLVVTGVTTEVCVHTTVREANDRGYECLVLADCVGSYFPEFQRVALEMVAAQGGI |
| | FGWVAPSESFVAALAELGPAAGSGPATGSGSAVAPGSASSAVATVASAAAAS |
| 663 | WP_066510632.1 cysteine hydrolase [Bradyrhizobium macuxiense]: |
| | MTNLNGTIAAEPEPITLDWSRTALVIIDMQRDFMEPGGFGETLGNDVGQLVRAVKPIATVLQAARAVGLL |
| | VVHTREGHLPDLSDAPPAKIERGAPRLRIGDPGPMGRILIRGEAGHDIIPELYPLDSEVVIDKPGKGAFY |
| | ATELGDILQKYGIENLLVCGVTTEVCVNTTVREANDRGYRCVVISDGCASYFPEFHEMGLKMIKAQGGIF |
| | GWVTDSAAVLEALAA |
| 664 | WP_066620311.1 cysteine hydrolase [Bosea sp. PAMC 26642]: |
| | MTGAPPVSRSEIAAKPFPLSVDFARTALVIIDMQRDFLEPGGFGAALGNDVSLLMAAVGPCQAILGGARE |
| | AGMLVIHTREGHRPDLSDAPPAKVNRGDPAKRIGAAGPMGRILIRGEAGHDIVPSLSPLPDEPVIDKPGK |
| | GAFYQTDLDLMLRNRGIETLLVAGVTTEVCVHTTVREANDRGYRCVVLGDACASYFPEFHEVGLRMIAAQ |
| | GGIFGWVSTTGDVLAALGKARQAA |
| 665 | WP_066811962.1 cysteine hydrolase [Defluviimonas alba]: |
| | MACIPGALPFAFDFDPASTALIVIDMQRDFVEPGGFGASLGNDVTRLQAIIPTVAALIQAARNAGLPVIH |
| | TRECHKPDLSDLPPAKRDRGSPSLRIGDPGPMGRILIAGEPGADIIPELAPLPAEIVLDKPGKGAFYATP |
| | LADHLARLGVRSLIFAGVTTEVCVQTTMREANDRGFACLLAEDATESYFPAFKAAAVKMIRAQGGIVGWT |
| | AFTAEIATALKGGAE |
| 666 | WP_067705403.1 cysteine hydrolase [Actinoplanes awajinensis]: |
| | MPTVEASPTPFTLEKTSTALLVIDMQRDFLLPGGFGESLGNDVAQLRRTIEPLAALLAAWRAAGWKVIHT |
| | REGHLPDLSDCPPAKLRRGPMIGQAGRFGRVLIRGEYGHDIIDELAPVEGEDVVDKPGKGAFYATDLAKI |
| | LENDGITSLVVTGVTTEVCVHTTVREANDRGYECLVLADCVGSYFPEFQRVGLQMIAAQGGIFGWVAESS |
| | ALIEEITR |
| 667 | WP_067955197.1 cysteine hydrolase [Mycobacterium sp. NAZ190054]: |
| | MNHTVDVPAEPSPFPLVAGKTALLVIDMQRDFLLPGGFGESLGNDVGRLREVVPPLAALLTAARSAGVLV |
| | VHTREGHEPDLSDCPPAKLNRGAPSKRIGDPGRYGRILIRGEYGHDIIDELAPIEGEVVIDKPGKGAFYA |
| | TGLSDVLGRAGITQLVITGVTTEVCVHTTTREANDRGYECLVVSDCVGSYFPEFQRVGLDMIKAQGGIFG |
| | WVAGTSAVIPALHQLTVTAA |
| 668 | WP_067996881.1 cysteine hydrolase [Mycobacterium sp. YC-RL4]: |
| | MSPITVSAEPSAFPLIPGQTALIVIDMQRDFLLPGGFGESLGNDVGQLLKVVPPLAALIAAARDAGILVI |
| | HTREGHLPDLSDCPPAKLNRGAPSRRIGDPGKYGRILIRGEYGHDIVDELSPVEGEVVIDKPGKGAFYAT |
| | ELQDVLTAAGVTQLLVTGVTTEVCVHTTTREANDRGYECLVVSDCVGSYFPEFQRVGLDMIKAQGGIFGW |
| | VADSTAVIPALHTLALSAA |
| 669 | WP_068015336.1 cysteine hydrolase [Rhodoplanes sp. Z2-YC6860]: |
| | MAVKRIEAEPSTVVVELDHAALVIIDMQRDFLESGGFGETLGNNVALLKAAVAPLQTMLAAARKSGMLII |
| | HTREGHRPDLSDAPKHKVERGEPSLRIGQPGPMGRILVRGEPGHDIIPELYPAPGEPVIDKPGKGAFYQT |
| | DLELMLKNREIDTLLVCGVTTEVCVNTTVREANDRGFRCVVLSDCCASYFPEFHEAGLAMIKAQGGIFGW |
| | VTPSTKVLAALDSH |
| 670 | WP_068047446.1 MULTISPECIES: cysteine hydrolase [unclassified |
| | Rhodococcus]: |
| | MTSTHTPVVSIPSASPSEFTLDASTTALLVIDMQRDFLLPGGFGESLGNDVGQLRTVIEPLVGLIAVARE |
| | AGIPVIHTREGHLPDLSDCPPAKLRRGAPSRRIGDRGAFGRILVRGEYGHDIVDELAPLEGETVIDKPGK |
| | GAFYATELSEVLTSAGITTLLVTGVTTEVCVHTTVREANDRGYECLVVTDCVGSYFPEFQRVGLEMISAQ |
| | GGIFGWTAPSEDVVAALVAFVPTSASR |
| 671 | WP_068375023.1 cysteine hydrolase [Rhodococcus sp. EPR-157]: |
| | MSESYVSGASPTPFTVPAGKTALLVIDMQRDFLLPGGFGESLGNDVNMLRSVIEPLAALIAAARESGVPV |
| | IHTREGHLPDLSDCPPAKLNRGMPSQRIGDPGEFGRILIRGEYGHDIVDELAPIDGETVIDKPGKGAFYA |
| | TELTEVLEDAGITTLLVTGVTTEVCVHTTVREANDRGYECLVVSDCVGSYFPEFQRVGLDMIAAQGGIFG |
| | WTSPSDDVIDALRELTPSPALHTNRI |
| 672 | WP_068388092.1 cysteine hydrolase [Leptolyngbya sp. NIES-3755]: |
| | MPFIAAQPYEYELPTESEIALIVIDMQRDFLEAGGFGEALGNDVNRANAIVPTVKRLLEGCRAMNLPIFH |
| | TQEGHRSDLSDCPQSKLKRGRGNLSIGDPGKLGRILILGEPGNEIIPELAPLPGEVLIPKPGKGAFYNTD |
| | LEVQLIARNITHSLIAGVTTEVCVQTTMREANDRGYECLLVEDATESYFPEFKQATLEMVRAQGGIVGWT |
| | ATTDQVLEGLRSRKAA |
| 673 | WP_068413428.1 cysteine hydrolase [Labrenzia sp. OB1]: |
| | MITVEAHPFAFAFDPASVALIVIDMQRDFIEPGGFGETLGNDVSHLQRVIQPTADLLALFRREGWPVIHT |
| | REDHLPDLSDCPPAKRERGSPSLRIGDPGPMGRILVRGEPGADIVPACAPVDGEIVIDKPGKGAFHATDL |
| | GGVLAKLGVRSLVFAGVTTEVCVQTTMREANDRGFECLLIEEATESYFPEFKAATLEMIRAQGAIVGWTA |
| | PLAALERAVEREQVNG |
| 674 | WP_068509608.1 cysteine hydrolase [Leptolyngbya sp. O-77]: |
| | MGLTITALPYEYTLPDDLSKLALVIIDMQRDFMEPGGFGDALGNDVTRLQAIVPALKELLAAFRALGLPV |
| | IHTIECHQPDLSDCPPAKLNRGKGSLKIGDEGPMGRILVLGEPGNGIIPDLAPLPGEVVITKPGKGAFYA |
| | TPLGAILAERGITHLLVTGVTTEVCVQTTMREANDRGYECLMVEDCTESYFPEFKQATLDMVRAQGGIVG |
| | WTAPSANVLDTFAAFRASEASPV |
| 675 | WP_068630663.1 cysteine hydrolase [Variovorax sp. PAMC 28711]: |
| | MTTTLHIDANPFAYDFALAKTALVLIDMQRDFIEPGGFGETLGNDVALLEAIVPATKAVLEAWRAAGGLV |
| | VHTREAHKADLSDCPPAKLNRGNPTLRIGDAGPMGRILVRGEPGNQIIDALAPMDGELVIDKPGKGMFYA |
| | TGLHETLQARGITHLLFGGVTTEVCVQTSMREANDRGYDGLLLEDCTESYFPAFKAAAIDMIRAQGAIVG |
| | WTAPSRLLLAALPRGIND |
| 676 | WP_068667272.1 cysteine hydrolase [Paenibacillus oryzisoli]: |
| | MSENVNLPAKPFSFQCDKRTTALVVIDMQNDFCSPGGFGELLGNDISQTRAIIPKLQQVLAACRQHGVLV |
| | VHTREGHQPDLSDCPPTKLRRSQLQGAGIGDTGPMGRILVRGERGHEIVSELAPAEGELVIDKSGKGAFY |
| | RTELDALLQARGIASLVLTGVTTHVCVHTTLREANDRGYECLVLEDGTAAFDPADQEAAIRMVHQQGGIF |
| | GWVGWADDWIVALESK |
| 677 | WP_068675940.1 MULTISPECIES: cysteine hydrolase [unclassified |
| | Variovorax]: |
| | MQIDAA.PFPYEFDFPRTALVIIDMQRDFIEPGGFGESLGNDVSLLQAIIPATQSMLHAWRSKGGLWHTR |
| | EAHRPDLADCPPAKRNRGKPALRIGDPGPMGRILIAGEPGNQIIDALAPVEGEIVIDKPGKGAFYATGLQ |
| | SLLQQRGIRSLVFMGVTTEVCVQTSMREANDRGYDSLLLEDCTESYFPAFKAAALEMIRAQGAIVGWTAP |
| | SARLLAALGD |
| 678 | WP_068737622.1 cysteine hydrolase [Tardiphaga robiniae]: |
| | MADSPHSATVVAEPGPIAVDWAATALVIIDMQRDFMEPGGFGETLGNDVSQLASAVAPIAAVLKAARETG |
| | MMVVHTREGHLPDLSDAPPAKIERGAPSLRIGDPGPMGRILIRGEAGHDIIPALYPVEGEIVIDKPGKGA |
| | FYATTLGADLKARDIDTLLVCGVTTEVCVNTTVREANDRGYRCIVISDGCASYFPEFHEMGLKMIKAQGG |
| | IFGWVASSAAILEAMTLSENAPETSNKLAAGASR |
| 679 | WP_068803411.1 cysteine hydrolase [Immundisolibacter cernigliae]: |
| | MIEITAKPYPLRLDPASSALIVIDMQRDFLEPGGFGAMLGNDVSLLRSAIVPCQRLLEVARKAGMLVIHT |
| | REGHRPDLSDAPPAKLARGKGPTRIGDLGPMGRILIRGEPGHDFIPELYPLPGEVVLDKPGKGSFCQTDL |
| | ALILANRGIRSLLVAGVTTEVCVHTTVREANDRGFECVVVSDAVASYFPEFHRVALDMISAQGGIFGWVA |
| | DSTAVCSALTRIAA |
| 680 | WP_068916996.1 cysteine hydrolase [Mycobacterium sp. djl-10]: |
| | MSTAVDIPAEPSPFPLIAGKTALIVIDMQRDFLLPGGFGESLGNDVGRLAAVVAPLAALIDVARRAGIMV |
| | IHTREGHKPDLSDCPPAKLSRGAPSKRIGDPGKYGRILIQGEYGHDIVDELAPAPGELVIDKPGKGAFYA |
| | TELQDVLSANGITQLLMTGVTTEVCVHTTTREANDRGYECLVVSDCVGSYFPEFQEVGLKMIAAQGGIFG |
| | WVADTAAVIPALTSLTPTPA |
| 681 | WP_069044323.1 cysteine hydrolase [Agrobacterium sp. RAC06]: |
| | MAVIKARPFDITITPEQTALVVIDMQRDFIEPGGFGATLGNDVTLLQAIIPATARLIEGFRRAGLPVIHT |
| | RECHASDLSDCPPAKRARGKPSLRIGDPGPMGRILIAGEDGADIVAALAPLPGEPVIDKPGKGAFYATHL |
| | GEILQEKGISQLVFAGVTTEVCVQTTMREANDRGYECLLATDATESYFPEFKKAAIDMMTAQGAIVGWAA |
| | TVDQIVEALDA |
| 682 | WP_069047694.1 cysteine hydrolase [Hydrogenophaga sp. RAC07]: |
| | MSTVTARPFNFEFDPAHAALVIIDMQRDFVEPGGFGESLGNTVEPLQAIVPAIANVLAAWRAMGGLVVHT |
| | RESHAPDLSDCPPAKRLRGSPSLRIGDVGPMGRVLVRGEPGNQIVPELAPVAGELVIDKPGKGAFYATDL |
| | QQQLQLRGITQLVVAGVTTEVCVQSTLREANDRGYDCLVLEDGTASYFPEFHAAALAMITAQGAIVGWSA |
| | TSKELLAGV |
| 683 | WP_069277737.1 cysteine hydrolase [Bradyrhizobium elkanii]: |
| | MANSSGTIAAEPAPITLDWSKTALVIIDMQRDFMERGGFGETLGNDVSQLARAVKPIAAVLAAARDAGLL |
| | VVHTREGHLPDLSDAPPAKLERGAPRLRIGDPGPMGRILIRGEAGHDIIPELYPQGSEIVIDKPGKGAFY |
| | ATEFGDVLQKYGIENLLVCGVTTEVCVNTTVREANDRGYRCVVISDGCASYFPEFHEMGLKMIKAQGGIF |
| | GWVTDAAAVLEALAA |
| 684 | WP_069307251.1 cysteine hydrolase [Methylobrevis pamukkalensis]: |
| | MITVDAAPFPYAFPPDRVALIVIDMQRDFVEPGGFGETLGNDVARLQAILPGVAGLLKVFRDNGWPVIHT |
| | REGHRPDLSDCPPAKRLRGEPTLRIGDAGPMGRVLIHGEPGHGIVEECAPVDGEHVIDKPGKGAFYATEL |
| | GDILARHGITHLVFAGVTTEVCVQTTMREANDRGFENLLIEDATESYFAEFKAAAMAMIRAQGAIVGWTA |
| | PLANLQAAVTRELA |
| 685 | WP_069441524.1 cysteine hydrolase [Methyloceanibacter superfactus]: |
| | MPIIDAKPFHYDFSFDHVALVCIDMQRDFCQPGGFAESLGDNIKNLQPCIPVIGKLQEAFRKAELPIIHT |
| | KECHKPDLSDLPTAKRNRGNPKLKIGDKGPMGRILIDGEPGSDFIPECAPAQWELVISKPGKDTFYNTEF |
| | DDYMKLRKITHLVITGVTTEVCVQTTMRCANDRGYDCVLVEDGTDSYFPEFKAMTLKALVAQGGIVGWTC |
| | KSDALLELLAKEQPGQVSSATSFAA |
| 686 | WP_069444897.1 cysteine hydrolase [Methyloceanibacter stevinii]: |
| | MPTIPDAKPFHYEFGIDHVALVCIDMQRDFCQAGGFAESLGDDLSKLQPCIPVIAKLQEAFRKAELPIIH |
| | TKECHKPDFSDLPTAKRNRGNPKLKIGDVGPMGRILIDGEPGADFIPECYPQEWELVISKPGKDTFYNTE |
| | FDDYLKMRKITHLIITGVTTEVCVQTTMRCANDRGYDCVLVEDGTDSFFPEFKEMTLKALVAQGGIVGWT |
| | ATSDQVLKSLEPILAAR |
| 687 | XP_018001122.1 Allophanate hydrolase [Phialophora attae]: |
| | MEGQQLILSTARPYAFKYEPEHTALVIIDVQRDFVDPNGFGAIQCGNDEIFSSVRSIVPAIKSALGAARK |
| | LGLHVIHTREGHRPDLSDLAATKRDRQMKAPSGHHTIGIGDEGPMGRLLVQGEYGHDIIDELRPLPDEPV |
| | IDKPGKGSFWNTTLHRTLMARGITHLLICGVTTECCVTTTAREANDRGFECCILTDCTSGFNAVSVDVSL |
| | NMFCSYDGLFGFVAKSGEYANTLGRSLDMASLAPHIRSRKMPLGELYRKVMIAAAASDSRIWTFLRSRDD |
| | VLAEVTALERKYIDNELLPPLYGIPFAVKDNFDFKGLPTEAACVEYRYMPTENAKTIQLLIDAGAILIGK |
| | TSMDQLATGLNGCRCPSGDPVSVYGNGRYISGGSSSGSGVAVASNLVTFALGTDTAGSGRVPAAFNGIVG |
| | YKPTKGTLSARGIVPACASLDTASIFAHDISDARKVWYAADQYDCDDAYAKPQSTLPLVPSNYRPNPHFT |
| | FAIPPSSIIADSCTPEYRTAFASTVTKLRSMGGTLVQLTADQYAPFQTASDLLYSGTLVHERIASIGPDF |
| | IAQNLYKLHPATRALFSAVLERPTKPYEIYRDQHLQAQLTRQAASLFSPHEGKIDVLVTPTTTCHPTREE |
| | MDADPIGLNAKLGNFTHFGNVLDLCAVSLNAGWVEGSSGAGQMPFGASLVCASGLDGKMFDLARRLERCI |
| | AADSKA |
| 688 | XP_018031755.1 glutamyl-tRNA amidotransferase subunit A |
| | [Paraphaeosphaeria sporulosa]: |
| | MATTDQYFRLPSARPYAYEFPFATTALIIIDIQRDFVDPGGFGSVQCGNDTVFSKARSIVPAVQKVLDIF |
| | RSVNGHVIHTREGHQADLADLPASKKLRQISAPSGHHTLGIGDNGPMGRLLVRGEYGHDIIDELTPFPGE |
| | SVIDKPGKGSFWGTGLHRELLARGITHLLFAGVTTECCVATTVRECNDRGFQCCVLQDCTAGFDAQQVTT |
| | ALDTICGQDGLFGFVGSSTDFLAATSGLVPACDIGGVPLLAGDRLPSIGALLHHYRKGTLKPAEVIHAIY |
| | DRIDRCNSSSNKAVWITLKSRAQTLAAAEELSAKYASKPLPPLFCIPFSVKDNIDVACLPTTSALPSLSV |
| | IPCSSAPAVQHVLDAGALVIGKVNLDQLATGLAGTRSPYGLVHSVFSNEHLSGGSSSGSAVSVAAGLVAF |
| | SLGTDTAGSGRVPAALNHVVGLKPTLGTVSARGVVPAVRSLDTISVMANSIDDARMVWRTIARYDPDDAF |
| | AKPPSSLAVWHSDFRGIHAAGFTFAVPPVSALQVCSPTYRAQFDAAVSALEARGGRRVSDTDFDYTPFER |
| | AGALLYNGALLYERVDSIGVDVLSKHAAALHPTTQKVLLPTLNNPPSAFTIFADQLLRRTLTHAAQRTFD |
| | KLRGGIDVLVVPSVPKHPRIADMEAAPLALNAEMGTFTHFGNVLDLCGVSVPFSMYEEDGVKLPFGITLL |
| | GGSGMDARVLGIAEAVEGGLGSRQP |
| 689 | WP_069624472.1 cysteine hydrolase [Methyloceanibacter marginalis]: |
| | MPIIDAKPFHYDFSFDHVALVCIDMQKDFCQPGGFAESLGDNIKNLQPCIPVIAKLQEAFRKAELPIIHT |
| | KECHKPDLSDLPTAKRNRGNPKLKIGDKGPMGRILIDGEPGSDFIPECYPAEWELVISKPGKDTFYNTEF |
| | DDYMKLRKITHLVITGVTTEVCVQTTMRCANDRGYDCILIEDGTDSYFPEFKEVTLRALVAQGGIVGWTG |
| | KSDALLDLLAKEQPGQVRSATSFAA |
| 690 | XP_018068583.1 amidase signature enzyme [Phialocephala scopiformis]: |
| | MAPSIKMSLPNARPYTYNFPVERTALVIIDMQRDFVDPNGFGSIQCGNPEIFSVVRTIVPTIQKVLEVCR |
| | STGIQVIHTREGHRPDLSDLPSSKKMRQVGNPNGHHTMGIGDQGPMGRLLVRGEWGHDIIDELRQLPGEP |
| | VIDKPGKGSYWGTGLHRTLLARGITHLLFSGVTTECCVTTTVRECHDRGFECCILSDCTGGFDAQQVTTS |
| | LDTICGQDGLFGFVGHSSDFFAAVSKSREMTPPSTPPATEDALLPIPQLQQRYKSGLLNPEEVVKSVFNR |
| | IERYEKIDPAVWISKQSREEVLTAAKSLTERFSEKPMPPLYGVPFALKDNIDIEGVVTTATCETFAYSAK |
| | STAPAVQLLLDAGALYIGKLNMDQLATGLSGCRSPYGTPHSVYSSEYISGGSSSGSAVAVAAGLVSFTLG |
| | TDTAGSGRVPAAFNGIVGYKPTKGTISARGVVPACKSLDTLSIMAPTLAEARKVWFVINHYDDLDPFAKK |
| | PLGLSLWKQEFRGYKEGGFTFGVPPQSVLETCSKEYQELYETSVQKLRSCGGRLVEIDYTPFEKAADLLY |
| | DASLVHERIASIGHDFLMSHLDSLHPTTKALFEAALSSPLRPWNVYHDQALQAEYTRQAQRTFDTLEGGV |
| | DVLLVPSTPCHPTIKEMEEEPLKLNAKVGTFTHAGNVVDLCGVSVNAGFFEKGGVKLPFGVTFLSGSGYD |
| | GKILDIAAVFEEAVKGERKS |
| 691 | XP_018182596.1 glutamyl-tRNA(Gln) amidotransferase subunit A |
| | [Purpureocillium lilacinum]: |
| | MATTKISLPNARPYSFQFPIATTAFIIIDMQRDFLDPNGFGYIQCGDPEIFSSVRKIVPTVRRALDAARA |
| | IGMHVIHTREGHRPDLSDLPAAKKLRQVSAPTGHHTMGIGDQGPMGRLLVRGEWGHDIIGELTPHPGETI |
| | IDKPGKGSFWGTGLHRALLARGITHLLFSGVTTECCVTTTLRECNDRGYECCILSDCTGGFDQQMVTTSL |
| | DIICGQDGLFGYIGHSSDFLSQVEQIPDLNTPSGILAPDAELPSISELQRLYKHGLVDLTTVVNSVFDKI |
| | EKYETVDPAVWISKRPREDVLRAAEALSAQYAGKPLPPLFGVPFAVKDSIDVEGVVTTVACESFAYEASS |
| | TAPSVQHLLDAGALYIGKTNLDQLATGLSGCRSPYGIPHSVYSKDHISGGSSSGSGVAVAAGLVSFAVGT |
| | DTAGSTRVPAAFNGIVGFKPTKGTISARGLVPACKSLDATTVLAPSIADARQVWYIIDRHDPLDPYAKPP |
| | KSLSTWKVDFRGPKEGGFTFGIPPPSLLENCSEAYRDLFKAAIQKLQSCGGRMVDIDYTPFAKAGDLLYD |
| | ATLVHERLASIGHDFFVKNINTLHPTTKSIYESALSTRLKPWQVFADQAAQTQYTMQARNIFDTLEGGID |
| | VLVVPSVPCHPTIKEMSEDPIALNSKLGLFTHAANVVDLCGVSVNAGLVDNGQGVKLPFGVTILGGSGYD |
| | GKVLDIAGVFETSLKERDSIST |
| 692 | WP_069692570.1 cysteine hydrolase [Bosea vaviloviae]: |
| | MSRSEIPAQPFPLSVDFARTALVIIDMQRDFLEPGGFGAALGNDVSLLVAAVAPCQAILAGAREARMLVI |
| | HTREGHRPDLSDAPPAKVLRGDPKKRIGAAGPMGRILIRGEAGHDIVPALAPLAEEPVIDKPGKGAFYQT |
| | DLDLMLRNRGIETLLVTGVTTEVCVHTTVREANDRGYRCLVLGDACASYFPEFHEVGLRMIAAQGGIFGW |
| | VSTTGEVLAALSAARRAA |
| 693 | XP_018380527.1 amidase signature enzyme [Alternaria alternata]: |
| | MPLNTTMLSFDAKPYAFSFPLEHTALLIIDMQRDFLLTKGFGEIQGGNLEAVQASIAPTKKLLEACRSAG |
| | LTVLHTREGHKPDLSDCPSSKLIRQEAAPGNTQHKLVIGDKGELGRLLTRGEYGHDIIEELKPLPGEVII |
| | DKPGKGSFWNTTILHQLKARAITHLIVSGVTTECCFATTIREANDRGFECCGIEEATSGYNDACFKKSTL |
| | DMIHWSQGLFGYIGSLDPLLDALAPVSSKSVEADSTPPQTPPIFDGDLTIPALQRAYKNGLSPLTVVDAV |
| | YDKIEAYKKIDPAVWIHLPPRVVTLDAARQLISTFPDRNALPPLFGVPFSVKDSIDIAGLPTTTACPPLA |
| | HVPSSSAPVYEKVIAAGALFVGKANLDQLATGLVGCRSPYGITHSVYHEDYISGGSSSGSTVSVGANLVS |
| | FSLATDTAGSGRVPAGFNGIVGYKPTRGTISFRGITPACLSLDCIALSTKTISDARTLWQILEGHDPLDP |
| | YAKPEITFERHVNSIGPQSRKFKFGIPPPEALAICSTPARRMFNETVSKLQAIGGVLTPIDWSPFQKAGQ |
| | LLYDGTFVSERLASLPDDFLKKNRSALHPVTAQLMDAVVARKSSAVDVYRDLQAQALYTRQAEKVFAYSA |
| | SGVDVVVVPTTPTHWKIDEVLADPIKKNSILGEFTHCGNVLDLCGVAVPAGTYPVKELSGKEEDGGVLPF |
| | SVTFLSGSRLDAEMLEIARRFEESMSV |
| 694 | WP_069907671.1 cysteine hydrolase [Devosia insulae]: |
| | MISVPSRPYPYTLDPAHTALVVIDMQRDFIERGGFGDSLGNDVKRLEAIIPTTAALLGLFRAQGWPVIHT |
| | REAHKPDLSDCPPAKIRRGNPSLHIGETGAMGRLLVRGEPGNQIVDALAPLEGEMVIDKPGKGMFWATGL |
| | HEQLVELGITHLVFAGVTTEVCVQTSMREANDRGYECLLIEDATESYFAEFKAATLKMIAAQGGIVGWVT |
| | SLAALEEAVKA |
| 695 | WP_069967975.1 cysteine hydrolase [Desertifilum sp. IPPAS B-1220]: |
| | MTTIAAQPYEYELPEDLQCCALVIIDMQRDFLELGGFGDALGNDVTRLQAIVPTVKQLLEAFRQFNLPII |
| | HTLECHKPDLSDCPPAKLNRGKSSLKIGDAGPMGRILIDGEPGNQIIPELTPLPGEIVLTKPGKGAFCRT |
| | DLELQLHRKGITHLLFTGVTTEVCVQTTMREANDRGFECLLIEDATDSYFPEFKTATIEMLRAQGGIIGW |
| | TTTADEVISVLSPVLSTKV |
| 696 | WP_070071702.1 cysteine hydrolase [Acidihalobacter prosperus]: |
| | MSFEIDARPFAYRCRADSTALLLIDLQRDFVEPGGFGASLGNDVSRLRPAIEACRRLLETFRALGLPVLH |
| | TREAHRPDLADCPPAKRLRGEPPLRIGDAGPMGRLLVAGETGTEIVPECRPLPGETVIDKPGKGAFYATD |
| | FGAHLERLGITHLVVGGVTTEVCVQSTLREANDRGYECLLVEEATESYFPEFKRATLEMVRAQGAIVGWT |
| | AALADVLRAFASHPVSPVIRSSP |
| 697 | WP_070147969.1 cysteine hydrolase [Agrobacterium vitis]: |
| | MVDIKAQPFAFPLKLDKAALIIIDMQRDFTEPGGFGETLGNDVSLVSAIVPDVKRLLEAARAHGLTVIHT |
| | MECHRPDLSDLPDAKRNRGNPTLRIGDEGPMGRILIAGEYGTGILPELAPVDAELVIEKPGKGAFYATNL |
| | GEELSKRSITQLIFAGVTTEVCVQTTMREANDRGYDCLLIEEATASYFSAFKQATLEMIRAQGGIVGWTA |
| | HLDPFLEALAHG |
| 698 | WP_070165726.1 cysteine hydrolase [Agrobacterium vitis]: |
| | MVDIKAQPFAFPLKLDQAALVIIDMQRDFTEPGGFGETLGNDVSLVSAIVPDVKHLLEAARAHGLTVIHT |
| | MECHRPDLSDLPDAKRNRGNPTLRIGDEGPMGRILIAGEYGTGILPELAPVDGELVIEKPGKGAFYATSL |
| | GEELTARGITQLIFAGVTTEVCVQTTMREANDRGYDCLLIEEATASYFPAFKQSTLEMIRAQGGIVGWTA |
| | HLDPFLEALAHG |
| 699 | XP_018659930.1 glutamyl-tRNA(Gln) amidotransferase subunit A |
| | [Trichoderma gamsii]: |
| | MASDRKLLLRNARPYAFSCPAATTALVIIDMQRDFLDPNGFGSVICANPAAFSSVRKIVPNVQKALEAAR |
| | SIGMHVIFTREGHLPNLSDLPAAKRLRQASAPNGSKSLTIGDEGPMGKLLVRGEKGHDIIDELKPYPGEP |
| | IIDKPGKGSFWGTEFHRVLLARGITHLVLAGVTTECCVTSTLREGNDRGYECCILSDCTAGFDESMAATS |
| | LDIVCCQNGLFGYVGHSSEFTTEAEQFRQLIPSSTNHGLNSPMLPSIDQLKSLYRDGRTTPEAVINSVFD |
| | RIAKYGDINPAVWISRQSQEDVLAAASKLSAAYAGKPLPPLFGIPFAIKDNIDVEGLVTTAACESYAYTA |
| | TSTAPSIQHLLDAGALYIGKLNLEQLATGLVGCRSPYGALHCFHSKDHVPGGSSSGSAVAVAAGLVSFAI |
| | GTDTAGSVRAPAALNGVVGFKPTKGTISARGAVPACQSLDTIGVLAPSVADARQVWYVLDRHDSLDPYAK |
| | PPASLPTWAVDFRGPKEGGFTFGIPPDSLLQLCSKEYQELFKKAVDVLQSIGGTLVDIDYTPLATAGDLI |
| | YGASLIHERLASIGYEFLSEKIDTLHPTTKLVIQKVLSSDLKGWEVYRDQAIQMECIAKGRQIFNKFEDG |
| | IDVLVVPTVPWHPTIQEIEDSPLIANSKLGIFTHPGNVIDLCGVSVNAGWAEDGGVRLPFGITFQGGSGY |
| | DGKVLDIAAVFENYSAK |
| 700 | XP_018692836.1 allophanate hydrolase [Fonsecaea erecta]: |
| | MGVSKDKQGLLTIEAKPYPFSFPLQHTALLVIDMQRDFICAGGFGEIQGGNLDAVQASIAPTKQLLDACR |
| | DAGMHIFHTREGQVPSLADCPSSKLVRQAAAPGNTQHLKVIGDKGELGRLLVRGEYGHDIVDELQPLPSE |
| | VVIDKPGKGSFWNTPILHKLKAYGITHLLVSGVTTECCFSTTIREANDRGFECCGIVQSTAGYNSAFKTA |
| | SLDMIHWSQGLFGFVADLQPVLDVLSPWHTQSKGVSTPPQTPPSWDGKLGIADLQASYKNGLSPLELVNA |
| | LFDRIERYEHIDGAVWIRRESREAVLDQARRLLELYPDKNARPALFGVPFTVKDSIDVQGIETTTACPPL |
| | AFVATKSAMCYQKVVGQGALYLGKVNLDQLATGLSGCRSPFGVTHSVFSDEHISGGSSSGSCVSVGADLA |
| | TFSLATDTAGSGRVPAGFNGVVGYKPTRGLVSFEGITPACLSLDCIAFTARTVEDARTLWQVCEGYDEND |
| | RYARDTFPAERHVNSIGAQKDAFRFGIPPPELLEVCSPSFRKLFNEAISRLQAMGGTLVPMDWTPFQKAG |
| | DLLYEGTFVSERLASLPDDFLEKNRQHLHPVILELFEKVVARQSTAVQLFRELQAKALYTRQATSQFRSA |
| | DRLGLDVVLVPTAPWHPTIKEMLADPIRLNAKMGTFTHFANVLDMCGIAVPSSTYQESEAGPRLPFSVTF |
| | LGSRCSDSEVLGIASRYQEATGR |
| 701 | WP_070664906.1 cysteine hydrolase [Actinomyces sp. HMSC065F11]: |
| | MIITKANPFNVEWDPASTALICIDFQRDFMEPGGFGETLGNNVSPLRETIEPTKRVLDRAREMGLLIIHT |
| | REGHRPDLKDLFPAKRDRGNPSLRIGDQGPMGRILVRGEKGHDIIPELYPADGEVILDKPGKDSFYGTDL |
| | DVMLRAQGIKTLIITGVTTEVCVQSTARAANDRGYECIILSDCTSSYFPEFKKSALEQFSAQGAIIGWVC |
| | DSTTLIESIDKAK |
| 702 | WP_071054189.1 cysteine hydrolase [Frankia sp. BMG5.36]: |
| | MTSAQLTVPARPYDFTFDPATTALVVIDMQRDFMEPGGFGESLGNDVSQLRSTIEPLTAVFAAARAAGLT |
| | VIHTREGHQPDLSDLPPAKLNRGNATLKIGDVGPKGRILIRGEYGQDIIDELAPIEGEIVIDKPGKGAFY |
| | ATSFGDILAEKGIRSLVVTGVTTEVCVHTTVREANDRGYECLVLSDCVGSYFPEFQRVALEMIAAQGGIF |
| | GWVAPSVAFIDALAPLSAASAAQ |
| 703 | WP_071059106.1 MULTISPECIES: cysteine hydrolase [unclassified |
| | Frankia]: |
| | MTPTAPLTVSARPYEYTFDPATTALVLIDMQRDFLEPGGFGESLGNDVSQLRSTIEPLAAVLAAARAVGL |
| | TVIHTREGHLPDLSDLPPAKLNRGGATLKIGDGGPKGRILIRGEYGQDIIDELAPAEGEPVIDKPGKGAF |
| | YATEFGDVLKARGITSLVVTGVTTEVCVHTTVREANDRGYECLVLSDCVGSYFPEFQRVALEMIAAQGGI |
| | FGWVASSEQFLDALAVLGASAVASSAVAAS |
| 704 | WP_071092137.1 MULTISPECIES: cysteine hydrolase [unclassified |
| | Rhizobium]: |
| | MVGIRAEPFAFPVRLDELALIVIDMQRDFAEPGGFGASLGNDVSRITRIVPDVKRLIQGFRNAGLPVIHT |
| | MECHRPDLSDLPPTKRDRGNPSLRIGDEGPMGRILISGEPGTAILPELAPLKGEIIIEKPGKGAFYATEL |
| | GAILQQKGISQLVIAGVTTEVCVQTTMREANDRGYECLLAEEATESYFPEFKAAAIAMIRAQGAIVGWTA |
| | HVDDILESVANA |
| 705 | WP_071830993.1 cysteine hydrolase [Pararhizobium antarcticum]: |
| | MPEKHTAEIKAEPFAFPLKRDAIALVVIDMQRDFAEPGGFGASLGNDVGRITKIIPDVKRLIEGFRMAGL |
| | PVIHTMECHRPDLSDLPPAKRNRGNPALKIGDAGPMGRVLIAGEAGTAILSELAPIDGEIVIEKPGKGAF |
| | YATPLGDILKARGIEQIVFAGVTTEVCVQTTMREANDRGYECLLAEEATESYFPEFKTAAIAMITAQGAI |
| | VGWVGSVDDIIKGIA |
| 706 | WP_071915999.1 cysteine hydrolase [Bradyrhizobium japonicum]: |
| | MLNSTKPTLGVIRAEPEPIRLDWPATALLIIDMQRDFMEPGGFGETLGNDVSQLARAVKPIGAVLKAARD |
| | TGMLVIHTREGHLPDLSDAPPAKVERGAPSLRIGDPGPMGRILIRGEAGHDIIPELYPLDSEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKTSTIQG |
| 707 | WP_071942875.1 MULTISPECIES: cysteine hydrolase [unclassified |
| | Mycobacterium]: |
| | MATINAEPFALDFDVSSTALVIIDMQRDFVLPGGFGEALGNDTSLLLAAVEPIERVLARAREIGMLVIHT |
| | REGHRPDLSDCPPAKLHRGGKTFIGEAGPMGRILVRGEQGHDIIPQLYPIDGEPVIDKPGKGSFHATDLS |
| | QILADRGIKTLWCGVTTEVCVHTTVREANDRGYECLVLSDCVASYFPEFQRVALEMIKAQGAIFGWVSD |
| | ADEFIAATS |
| 708 | WP_072607455.1 cysteine hydrolase [Mesorhizobium oceanicum]: |
| | MAISVPARPYDFSLDPRSVALVVIDMQRDFIEPGGFGAVLGNDVSRLLPAIPAVARLLELFRARGWPVIH |
| | TREAHRPDLSDCPPAKRLRGAPGLRIGDDGAMGRILIAGEPGNQIVPELAPVKGEIEIDKPGKGMFWATG |
| | LHERLQEMGITQLVFAGVTTEVCVQTSMREANDRGYECLLIEEATESYFPEFKAAAIEMIVAQGGIVGWA |
| | AGIAGLEQALSEEVAHA |
| 709 | WP_072642259.1 cysteine hydrolase [Rhizobium leguminosarum]: |
| | MMEIKAEPFAFPVKHAELALIVIDMQRDFAEPGGFGASLGNDVSRITRIVPDVKRLIQGFRNAGLPVIHT |
| | MECHRPDLSDLPPAKRDRGNPSFRIGDEGPMGRILISGEPGTAILPELAPVKGEVVIEKPGKGAFYATDL |
| | GTVLQQKGIKQLVFAGVTTEVCVQTTMREANDRGYECLLAEEATESYFPEFKAAALAMIRAQGAIVGWTA |
| | HVDDILESIAHA |
| 710 | WP_072692418.1 cysteine hydrolase, partial [Escherichia coli]: |
| | MTQSIFQAQPFELPFDPRTTALVMIDMQRDFVEAGGFGEALGNDVSRVRTAIAPCTEVLAAARQKGIMVI |
| | HTREGHRADLSDCPPAKLTRGGKTFIGEPGPMGRILVRGEAGHDIIPELYPVAGEPVIDKPGKGAFYQTD |
| | LHLILQNHGIKTLIVCGVTTEVCVTTTVREANDRGY |
| 711 | WP_072692420.1 cysteine hydrolase, partial [Escherichia coli]: |
| | MTISIFQAQPFELPFDPCTTALIMIDMQRDFVEAGGFGEALGNDVSLVRTAIAPCKEVLAAARQKGIMVI |
| | HTREGHREDLSDCPSAKLTRGGKTFIGEPGPMGRILVRGEAGHDIIPELYPVTGEPVIDKPGKGAFYQTD |
| | LHLILQKRGIKTLIVCGVTTEVCVTTTVREANDRGY |
| 712 | WP_073055715.1 cysteine hydrolase [Kaistia soli]: |
| | MITVDAKPFPYAFPPDKTALIVIDMQRDFVEPGGFGESLGNDVSLLRAIIPTVAALIGLFRTNGWPVIHT |
| | REGHAADLSDCPPSKICRGAPSMRIGDAGPMGRIMVRGEPGNAIIPELQPVDGEIVIDKPGKGAFYATPL |
| | GEDLARIGITHLIFAGVTTEVCVQTTMREANDRGFDCLLIEDATESYFPEYKAATVSMIAAQGAIVGCVA |
| | PFAALEAASA |
| 713 | WP_073069468.1 cysteine hydrolase [Phormidesmis priestleyi]: |
| | MTIISAQPYDYELPEDGNIALIVIDMQRDFMELGGFGDILGNDVSLLQAIVPTLKTLLAGCRSRNLPIFH |
| | TTEGHQPDLSDCPDSKRKRGKGKLTIGDAGSLGRILILGEPGNGIIPELAPLPGEIVIPKPGKGAFYNTD |
| | LEPLLKERNVTHLLITGVTTEVCVQTTMREANDRGYECLLVEDATESYFPEFKQATLEMVRAQGGIVGWT |
| | ATTDQVLQGLQTWKSASAIA |
| 714 | WP_073173309.1 cysteine hydrolase [Pseudomonas asturiensis]: |
| | MIDVNAHPARFAFDPASTALVIIDMQRDFLEPGGFGAALGNDVLPLQAIVPNVQRLLALARAHGIHTIHT |
| | RESHDSELADCPPSKLEHGLEGLRIGDVGPMGRILVRGEPGNQIIDALAPMAGEWVVDKPGKGMFFSTGL |
| | NGRLSAAGITHLIFAGVTTEVCVQTSMREANDRGYRCLLIEDATESYFPAFKQATLDMITAQGGIVGRVA |
| | PLSALEQALQTRNTH |
| 715 | WP_073467404.1 cysteine hydrolase [Rhizobacter sp. OV335]: |
| | MIQVDALPGPFEFEPAHTALVMIDMQRDFIEPGGFGAALGNDVSLLAPVVPAAAELVALCRAIGVLVVHT |
| | QECHRPDLSDCPPAKRLRGKPSLRIGDPGPMGRILIEGEPGAGFVPELMPQPGDVVIAKPGKGAFYGTRL |
| | AEVLQDQQITRLIFGGVTTEVCVQTTMREANDRGYECLLVEEATGSYFPQFKAATLAMIRAQGGIVGWTA |
| | SLRAVRAALDFARSGESLSFALPNESNQSKGA |
| 716 | WP_073548316.1 cysteine hydrolase [Chroogloeocystis sierophila]: |
| | MVLIAAQPYDYELPSDLQKVALLIIDMQRDFLEPGGFGEALGNDVSHLSATIPIIKSLLEIFRRRQLPVF |
| | HTVEGHQPDLSDCPPSKLRRGNGQLKIGDPGPMGRILILGESGNAIISELQPIPGEIVISKPGKGAFYQT |
| | SLESYLHKQGITHLMITGVTTEVCVQTTMREANDRGFECLLVEDATASYFPEFKESTLEMIRAQGGIVGW |
| | TATAANVMQAFGNYS |
| 717 | WP_073594010.1 cysteine hydrolase [Phormidium ambiguum]: |
| | MIFIPAQPYNYEVTNLSNVALVIIDMQRDFLEPGGFGAALGNDVSRLRSIVPVLQQLLITFRQLHLPIIH |
| | TIEGHQSDLSDCPPAKINRGNCKLKIGDIGPLGRILVLGEPGNNIINELTPLSGEIIINKPGKGAFYNTN |
| | LHDILIEQGITHLIFTGVTTEVCVQTTMREANDRGFECLLIEDATESYFPEFKQATIEMIRSQGGIVGWT |
| | TKAENLLQALQNISLSIK |
| 718 | WP_073609283.1 cysteine hydrolase [Phormidium tenue]: |
| | MPLPALPYPYPLPATGLALVIIDMQRDFIEPGGFGDALGNDVSLLRSIIPNVKALQEAFRRYDLPIFQTV |
| | EGHRPDLSDCPPSKRDRGHGSLKIGDRGPMGRILVLGEPGNAIIPELAPLPHEVIIPKPGKGAFYATELE |
| | AHLKALGITHLFITGVTTEVCVQTTMREANDRGYECLLVEDATESYFPKFKQSTLEMVRAQDGIIGWTGH |
| | TETLLNALAAQYAAQIVA |
| 719 | WP_073628722.1 cysteine hydrolase [Pseudoxanthobacter soli]: |
| | MTAPITVDAQPFAYSFDPARTALIVIDMQRDFIEPGGFGETLGNDVGLLQAIVPTVADLIGLFRSRGWLV |
| | IHTREGHKPDLSDCPPAKRERGAPSLRIGDPGPMGRILIHGEPGHGLVEACAALPSEPVIDKPGKGSFYG |
| | THLGVLLADHGITHLVIAGVTTEVCVQTTMREANDRGYECLLVEDATESYFPAFKAATLDMIRAQGGIVG |
| | WTAPLAALKEAVARRMPDALSA |
| 720 | WP_073694639.1 cysteine hydrolase [Mycobacterium sp. ST-F2]: |
| | MTSVEVPAEPTPFTLTAGQTALIVIDMQRDFLLPGGFGESLGNDVDQLLKVVPPLAALIAAARAAGITVI |
| | HTREGHEPDLSDCPPAKLNRGAPSKRIGDPGKYGRILIRGEYGHDIVDELAPIDGELVIDKPGKGAFYAT |
| | GLQDALTAAGITQLLVTGVTTEVCVHTTTREANDRGYECLVVSDCVGSYFPEFQRVGLEMIAAQGGIFGW |
| | VADTAAVIPALQQLAAPSPSAV |
| 721 | WP_073831675.1 cysteine hydrolase [Micromonospora sp. TSRI0369]: |
| | MLTITTARPGPYAFDIATTALLVIDMQRDFLEPGGFGESLGNDVGQLRCTIAPLAALLADARAIGLNIIH |
| | TREGHLPDLSDCPPAKLRRGAPSRRIGDPGPNGRILIRGEYGHDIVDELRPLPGEPVIDKPGKGAFYATD |
| | LDALLAERGIRSLLVAGVTTEVCVHTTVREANDRGYECLVLADCVGSYFPEFQRVGLDMIAAQGGIFGWV |
| | ADSAQVRAALPATPALQPSS |
| 722 | WP_073989736.1 cysteine hydrolase [Mesorhizobium plurifarium]: |
| | MAEIAAQPFAFAFKPETTALIVIDMQRDFAEPGGFGASLGNDVSRVTAIVPTVKRLIEGFRAASLPVIHT |
| | MECHRPDLSDLPPAKRDRGNPSIRIGDVGPMGRVLIAGEPGTAILDELAPLPGEIVIEKPGKGAFYATGL |
| | GDDLKRLGARQLVFAGVTTEVCVQTTMREANDRGYECLLADDATESYFPEFKAAALAMIRAQGAIVGWTA |
| | TTDQVLEGIANA |
| 723 | WP_074059704.1 MULTISPECIES: cysteine hydrolase [Rhizobium]: |
| | MVAIKAEPFPFAVKPGALALIVIDMQRDFAEPGGFGACLGNDVSRITKIVPDVKRLLEGFRAARLPVIHT |
| | MECHRPDLSDLPPAKRDRGNPSLRIGDVGPMGRILICGEPGTSILPQVAPIDGEVVIEKPGKGAFYATEL |
| | GDVLKEGGIKQLVFAGVTTEVCVQTTMREANDRGYECLLAEEATESYFPEFKAAAMAMIRAQGAIVGWTA |
| | HVDDILESIAHA |
| 724 | WP_074072736.1 cysteine hydrolase [Rhizobium gallicum]: |
| | MVAIKAEPFTFAVKPGALALIVIDMQRDFAEPGGFGACLGNDVSRITKIVPDVKRLIEGFRAAGLPVIHT |
| | MECHRPDLSDLPPAKRDRGSPSLKIGDEGPMGRILISGEPGTAILPEVAPIDGEVVIEKPGKGAFYATEL |
| | GDLLKEGGIKQLVFAGVTTEVCVQTTMREANDRGYECLLAEEATESYFPEFKAAAMAMIRAQGAIVGWTA |
| | HVDDILESITHA |
| 725 | WP_074121739.1 cysteine hydrolase [Bradyrhizobium sp. AS23.2]: |
| | MLNSTKPTLGVISAEPEPIKLDWSSTALVIIDMQRDFMEPGGFGETLGNDVSQLARAVKPIGAVLKAARD |
| | TCMLVIHTREGHLPDLSDAPPAKVERGAPSLRIGDPGPMGRILIRGEAGHDIIPELYPLDNEVVIDKPGK |
| | GAFYATELTDVLEKYGIENLLVCGVTTEVCVNTTVREANDRGYRCVVISDGCASYFPEFHEMGLKMIKAQ |
| | GGIFGWVADSAAVLEAMKISTTQG |
| 726 | WP_074131006.1 cysteine hydrolase [Bradyrhizobium sp. NAS96.2]: |
| | MANSGGTIAAEPAPITLDWSRTALVIIDMQRDFMEPGGFGETLGNDVSQLARAVQPIAAVLAAVRDAGLL |
| | VVHTREGHLPDLSDAPPAKVERGAPSLRIGDPGPMGRILIRGEAGHDIIPELYPLENEIVIDKPGKGAFY |
| | ATEFGDILRKFGIENLLVCGVTTEVCVNTTVREANDRGYRCVVISDGCASYFPEFHEMGLKMIKAQGGIF |
| | GWVADSTAVLEALAA |
| 727 | WP_074268395.1 cysteine hydrolase [Paraburkholderia phenazinium]: |
| | MSSEVQALPSPFVFEPRHTALVIIDMQRDFIEPGGFGESLGNDVSLLAEIVPTVAELLALARKAGLLVVH |
| | TRESHAPDLSDCPPAKRLRGAPQMRIGDPGPMGRILVRGEPGNAIVDALAPLADELVIDKPGKGAFYATP |
| | LSGELNARSITHLLFAGVTTEVCVQTSMREANDRGYECLLIEDATASYFPAFKQASLEMIRSQGGIVGWT |
| | APLAALKEGL |
| 728 | WP_074279967.1 cysteine hydrolase [Bradyrhizobium erythrophlei]: |
| | MTNSSATFGKVAAEPEPIELDWTKTALLIIDMQRDFMEPGGFGETLGNDVSQLARAVKPIAAVLNAARDT |
| | GMLVVHTREGHLPDLSDAPPAKVERGAPSLRIGDPGPMGRILIRGEAGHDIIPELYPLDSEVVIDKPGKG |
| | AFYATELSDVLQKYGIENLLVCGVTTEVCVNTTVREANDRGYRCVVLSDGCASYFPEFHEMGLKMIKAQG |
| | GIFGWVTDSAAVLEALLPETSKIAV |
| 729 | WP_074287497.1 cysteine hydrolase [Burkholderia sp. GAS332]: |
| | MTQETELTIDAQPGPFTLDPTKTALIVIDMQRDFIEPGGFGESLGNDVSLLAEIVPTVAALLAFARRHQW |
| | LVVHTRESHAADLSDCPAAKRLRGAPNARIGDAGPMGRILIRGEPGNAIIEPLAPLAGELVIDKPGKGAF |
| | YATRLGEELAMRGITHLVFAGVTTEVCVQTSMREANDRGYDSLLIEDATASYFPAFKQATLDMVSSQGGI |
| | VGWTAPFSSLTKLDETIPAWR |
| 730 | WP_074295526.1 cysteine hydrolase [Paraburkholderia phenazinium]: |
| | MMSIEVKALPSPFVFEPQHTALVIIDMQRDFIEPGGFGESLGNDVSLLAEIVPSVAELLALARRTGLLVV |
| | HTRESHAPDLSDCPPAKRLRGAPQMRIGEPGPMGRILVRGEPGNAIIDALAPVEGELVIDKPGKGAFYAT |
| | RLSEALSVRGITHLLFAGVTTEVCVQTSMREANDRGYECLLIEDATASYFPAFKQASLEMIRSQGGIVGW |
| | TAPLAALKKGL |
| 731 | WP_074637484.1 cysteine hydrolase [Sulfitobacter pontiacus]: |
| | MTQIPARPFDFPLARDRVALVIIDMQRDFVEPGGFGASLGNDVRPLQAIVPTVARLLAGFRTAGLPIFHT |
| | REAHRPDLSDCPPAKRLRGAPALRIGDAGPMGRVLIAGAPGCEIIPALTPLPDEPVIDKPGKGAFYATDL |
| | GDQLAARGITQLVCAGVTTEVCVQTTMREANDRGFECLLATDATESYFPSFKAAAIEMIVAQGGIVGWAT |
| | DTDTILGAING |
| 732 | WP_074768591.1 cysteine hydrolase [Paraburkholderia fungorum]: |
| | MSDNTSNSTISSTTHSIDAQPGPFTFDSKKTALVVIDMQRDFIEPGGFGESLGNDVSLLAEIVPTVAALL |
| | ALARRQNWLVVHTRESHAADLSDCPPAKRLRGAPNARIGDAGPMGRILIRGEPGNAIIEPLAPVAGEIVI |
| | DKPGKGAFYATRLGEELAMRGITHLVFAGVTTEVCVQTSMREANDRGYDSLLVDDATASYFPAFKQATLD |
| | MVRSQGGIVGWTAPLSSLTRIEGKN |
| 733 | WP_074805674.1 cysteine hydrolase [Pseudomonas syringae]: |
| | MIKVNARPDSFAFDTSRTAVVIIDMQRDFLEPGGFGAALGNDVAPLQAIVPSVQRLLTLARDEGIAVIHT |
| | RESHRPDLADCPQAKRDHGSPGLRIGDPGPMGRILIRGEPGNQIIDTLAPLAGEWVIDKPGKGMFFATDL |
| | QQRLTDAGITHLIFAGVTTEVCVQTSMREANDRGYRCLLIEDATESYFPAFKRATLEMITAQGGIVGRVA |
| | SLTDLEQALQTRSTH |
| 734 | WP_074810762.1 cysteine hydrolase [Pseudomonas syringae]: |
| | MISLQARPSPFLFEPSRTAVVIIDMQRDFLEPGGFGAALGNDVTPLQAIVPVVHRLLTLARDRGITVIHT |
| | RESHRTDLSDCPQAKLEHGSPGLRIGDPGPMGRILVRGEPGNQIIDALTPIAGEWVIDKPGKGMFFATDL |
| | HAQLAEAGITHLIFAGVTTEVCVQTSMREANDRGYRCLLLEDASESYFPAFKQATLDMITAQGAIVGRVA |
| | ALADLEQALPTRSTH |
| 735 | WP_074825894.1 MULTISPECIES: cysteine hydrolase [Bradyrhizobium]: |
| | MANSRGPLSGTVAAEPEPIALDFAATALLIIDMQRDFMEPGGFGETLGNDVSQLARAVKPIAAVLEAARD |
| | IGMLVVHTREGHLPDLSDAPPAKIERGAPSLRIGDPGPMGRILIRGEAGHDIIPELYPLDNEVVIDKPGK |
| | GAFYATELGDVLQQYGIENLLVCGVTTEVCVNTTVREANDRGYRCVVLADGCASYFPEFHEMGLKMIKAQ |
| | GGIFGWVSDSVAVLEALSPETSKTAAAGASR |
| 736 | WP_074830082.1 MULTISPECIES: cysteine hydrolase [Pseudomonas]: |
| | MIWVTANPNDFSFEPANTALVVIDMQRDFIEQGGFGAALGNDVTPLKAIVPAVRRLLELARQQGMLAIHT |
| | RESHLPDLSDCPDAKHAHGLPGLRIGDPGPMGRILVRGEPGNQIIADVAPAEGEWVIDKPGKGMFYATGL |
| | HERLQARGISHLLFAGVTTEVCVQTSMREANDRGYRCLLIEEATESYFPAFKRSTLEMIVAQGGIVGRTA |
| | YLTALEAALQEDRP |
| 737 | WP_074842463.1 MULTISPECIES: cysteine hydrolase [Pseudomonas syringae |
| | group]: |
| | MISISARPDTFTFEPSRTAVVIIDMQRDFLEPGGFGAALGNDVAPLQAIVPTVQQLLALAREQGIAVIHT |
| | RESHRPDLSDCPQAKLDHGLPGLRIGDPGPMGRILVRGEPGNQIIDALTPLASEWVIDKPGKGMFFATDL |
| | QQRLTAAGITHLIFAGVTTEVCVQTSMREACDRGYRCLLIEDATESYFAAFKQATLDMITAQGAIVGRVA |
| | SLANLQHALHTRSTQ |
| 738 | WP_074849504.1 cysteine hydrolase [Gordonia westfalica]: |
| | MSETVTLEALPGPIELDLDRTALIIIDMQRGFLLPGGFGETLGNDVSQLQRVVEPLAALLDAARASGMLV |
| | IHPRKGHLPDLSDCPPAKLNRGEPSKRIGDPGAFGRILIRGEYGHDIIDELAPLDTEVVIDKPGKGAFYA |
| | TGLSKVLADNEITQLLVAGVTTEVCVHTTTREANDRGFECVVVSDCVGSYFPEFQRVGLEMVAAQGGIFG |
| | WTAPGTAIIPLLKESAPAEPAV |
| 739 | WP_074907473.1 cysteine hydrolase [Pseudomonas syringae]: |
| | MIKVNARPDRFAFDTSRTAVVIIDMQRDFLEPGGFGAALGNDVAPLQAIVPSVQRLLALARDEGMAVIHT |
| | RESHRPDLADCPQAKRDHGSPGLRIGDPGPMGRILIRGEPGNQIIDTLAPLAGEWVIDKPGKGMFFATDL |
| | QQRLSEAGITHLIFAGVTTEVCVQTSMREANDRGYRCLLIEDATESYFPAFKQATLEMITAQGGIVGRVA |
| | SLTDLEQALHTRSTL |
| 740 | WP_074987391.1 cysteine hydrolase [Paraburkholderia tropica]: |
| | MPTLAGALPSPFVFEAPRTALVVIDMQRDFIEPGGFGAALGNDVSLLGGIVPDVARLLHHARERGWFVVH |
| | TRESHAADLSDCPPAKRLRGQPSARIGDAGPMGRILVRGEPGNAIVDALAPVGGELVIDKPGKGAFHATR |
| | LGEELAQRGITHLVFAGVTTEVCVQTSMREANDRGYDCLLIEDATASYIPAFKAATLAMIHSQGGIVGWT |
| | ASLAQLLEADA |
| 741 | WP_075159958.1 cysteine hydrolase [Paraburkholderia sp. SOS3]: |
| | MQTVSGAQPFPFHFDPHRTALVVIDMQRDFIEPGGFGEALGNDVSLLASIVPTVAALLAHARAQGWLVVH |
| | TRESHATDLSDCPPAKRARGAPLARIGDQGPMGRILVRGEPGNAIVDALAPAGGEIVIDKPGKGAFYATR |
| | LAEELALRAITHLIFAGVTTEVCVQTTMREANDRGYECLLIEDATASYIPAFKEATLAMMRSQGAIVGWT |
| | ATLANLMEA |
| 742 | WP_075310114.1 MULTISPECIES: cysteine hydrolase [unclassified |
| | Pseudonocardia]: |
| | MISVDADPGTFAFDPATTALLIIDMQRDFCEPGGFGETLGNDVSLLRSVIPPLQEVLRTVRALGMTVIHT |
| | REGHVPDLSDCPPAKLNRGEPSLRIGDPGPKGRILVRGEYGHDIIDELRPESGELVIDKPGKGSFHGTTF |
| | GAELRSRGITSLIVAGVTTEVCVHTTVREANDRGYECLVLSDCTGSYFPEFHRVALEMVAAQGGIFGWVA |
| | PSSALLTALTREAVA |
| 743 | WP_075596730.1 cysteine hydrolase [Leptolyngbya sp. ‘hensonii’]: |
| | MIPIAAQPYDYELPTDSKVALIMIDMQRDFLEHGGFGDALGNEVTRLQAIVPTVKQLLDAFRAANLLIIH |
| | TVEGHKPDLSDCPPSKLNRGKGSLKIGDPGPMGRILVLGEAGNGIVPELAPLPGEILLEKPGKGAFCRTN |
| | LETILQERGITHLVIGGVTTEVCVQTTMREANDRGYECLLVEDATESYFPEFKQATLEMVRAQGGIIGWT |
| | APASAVIEALQKLPALSSVS |
| 744 | WP_075614533.1 cysteine hydrolase [Rhizobium taibaishanense]: |
| | MVDIKAQPFAFPLNVEKAALIVIDMQRDFTEPGGFGETLGNDVSLVSAIVPDVKRLLEAARATGLTVIHT |
| | MECHRPDLSDLPEAKRNRGNPSLRIGDQGPMGRILISGEYGTDILPALAPTPGELVIEKPGKGAFYATPL |
| | GDELKSRGITQLIFAGVTTEVCVQTTMREANDRGYDCLLVEEATASYFPAFKQAALDMIRAQGGIVGWTA |
| | HIDDVLEALDHG |
| 745 | WP_075633395.1 cysteine hydrolase [Rhizobium rhizosphaerae]: |
| | MATIKARPFDFTLAPENAALIVIDMQRDFIEPGGFGATLGNDVTRLQAIVPATARLIAGFRKAGLPVIHT |
| | RECHAPDLSDCPPAKRTRGNPSLRIGDPGTMGRILIAGEAGADIIQALYPIPGETVIDKPGKGAFYATPL |
| | GEMLKEKGVRQLVFAGVTTEVCVQTTMREANDRGYECLLATDATESYFPEFKKAAIDMMTAQGAIVGWAA |
| | TVDQIVEAIDD |
| 746 | WP_075727937.1 cysteine hydrolase [Tissierella creatinophila]: |
| | MTKYTVDAKPYEFEFNLEETALIIIDMQRDFCAPGGFGEKLGNDITPTRKVIEPIKNVLEVAREAGMLVI |
| | HTREGHRPDLSDCPPNKLRRSKRQGAGIGDMGPMGRILIRGEYGHDIVDELTPIEGEPIIDKPGKGAFYQ |
| | TDLDIILKNKGITNLIVTGVTTHVCVQTTIREANDRGFNCLMLEDGTAAFDPKDQEGSIRMINQQGGIFG |
| | WTTESKYILETLKK |
| 747 | WP_075838558.1 cysteine hydrolase [Rhodococcus sp. CUA-806]: |
| | MNESYVSGASPTPFTIPAGKTALLVIDMQRDFLLPGGFGESLGNDVDMLRNVIEPLAALIAAAREHGVPV |
| | IHTREGHLPDLSDCPPAKLNRGMPSQRIGDPGAFGRILVRGEYGHDIVDELAPIDGETVIDKPGKGAFYA |
| | TDLAEILEMAGITTLLVTGVTTEVCVHTTVREANDRGYECLVVSDCVGSYFPEFQRVGLDMIAAQGGIFG |
| | WTSPSDEVIAAIGELAPSPALHTNRI |
| 748 | WP_075854495.1 cysteine hydrolase [Rhizobium hainanense]: |
| | MVDIKAQPFAFPAKPDQIALIVIDMQRDFAEPGGFGESLGNDVSRITKIVPDVKRLIEGFRKAGLPVIHT |
| | MECHRPDLSDLPPAKRDRGNPSLRIGDEGPMGRILIAGEPGTAILPDLAPINGEIVIEKPGKGAFYATDL |
| | GDILKQRCITQLVFAGVTTEVCVQTTMREANDRGYECLLAEEATESYFPEFKTAAIAMIRAQGAIVGWTA |
| | HVDDILEAIHA |
| 749 | WP_075944378.1 cysteine hydrolase [Pseudonocardia sp. CNS-139]: |
| | MITIPADPYPFTLDPATTALVVIDMQRDFVEPGGFGETLGNDVALLQSVVPPLRKVLDAFRAAGLTVIHT |
| | REGHVPDLSDCPPAKLNRGEPTLRIGDEGPKGRILVRGEYGHDIVDELAPLPGELVVDKPGKGSFHATGL |
| | QDELVARGITRLVVTGVTTEVCVHTTVREANDRGYECLVLSDCTGSYFPEFHRVGLAMIAAQGGIFGWVA |
| | PSEALITALVPQEVAS |
| 750 | WP_076199564.1 cysteine hydrolase [Rhodoferax koreense]: |
| | MQIDATPFPYRFDVAHTALVLIDMQRDFIEPGGFGETLGNDVSLLEAIVPAARAMLEAWRAAGGLVVHTR |
| | EAHRPDLSDCPPAKRDRGNPTLRIGDAGPMGRILVMGEPGNQIIEALAPVDGELVIDKPGKGAFYATGLH |
| | ETLQARGITHLLFGGVTTEVCVQTSMREANDRGYDCLLLEDCTESYFPHFKAAALEMIRAQGAIVGWTAP |
| | SSAVLAALHSG |
| 751 | WP_076397335.1 cysteine hydrolase [Rhizobium sp. RU33A]: |
| | MAVIKARPFDITITPQKTALVVIDMQRDFIEPGGFGATLGNDVTLLQAIIPATARLIDGFRRAGLPVIHT |
| | RECHAPDLSDCPPAKRARGKPSLRIGDPGPMGRILIAGEDGADIVAALAPLLGETVIDKPGKGAFYATPL |
| | DEILQEKGISQLVFAGVTTEVCVQTTMREANDRGYECLLATDATESYFPEFKKAAIAMMTAQGAIVGWAA |
| | TVDQIVEALDA |
| 752 | WP_076505569.1 cysteine hydrolase [Pseudacidovorax sp. RU35E]: |
| | MRLNDARPFPYDFDVARTALVLIDMQRDFIEPGGFGETLGNDVSLLAAIVPATQAVLAAWRQAGGLVVHT |
| | REAHLPDLSDCPPAKRLRGHPTLRIGDEGPMGRILVTGEPGNQIIDALAPIEGEWVIDKPGKGAFHATGL |
| | HELLQARGITHLVFGGVTTEVCVQTSMREANDRGYDCVLLEDCTESYFPQFKAAAVEMIRAQGAIVGWTA |
| | TSAQLIAALASAPPQT |
| 753 | WP_076584181.1 cysteine hydrolase [Haloterrigena daqingensis]: |
| | MVEFDSGRTAFLSIDMQQDFCGEDGYVDAMGYDLSQTQRAVQPIWNVLETVRQTDIDVIHTREGHKQDLS |
| | DAPFNKLLRSKMAGDGDGIGETPAGGIGPLLTRGHENWDIIDKLAPEPSEPVIDKPTKGAFANTNIGLVL |
| | ERLGTTHLVISGITTDVCVHTIMREANDRGYWCLLLKDATGATDNGNREAAIKQIKMQGGVFGWVSDSER |
| | FIKAVEEGVA |
| 754 | WP_076644116.1 cysteine hydrolase [Methylorubrum extorquens]: |
| | MPAPQPLLDAEPAPLPFDPARAALVVIDMQRDFLEPGGFGESLGNDVSLLAAAVPPARALLAAARAAGLL |
| | VVHTREGHAPDLSDAPPAKRERGAPTARIGEPGPMGRILIRGEPGHDIIPELAPLDGEPVIDKPGKGAFY |
| | ATGLAALLEARGIETLIVCGVTTEVCVHTTVREANDRGYRCVVVADACGSYIPAFHEAGLAMIKAQGGIF |
| | GWVSRSAAVIAALGQA |
| 755 | WP_076819008.1 cysteine hydrolase [Frankia asymbiotica]: |
| | MTSAPFTVPARPYDFTFDPATTALVVIDMQRDFMEPGGFGESLGNDVSQLRSTIEPLTAVFAAARAAGLT |
| | VIHTREGHQPDLSDLPEAKLNRGNATLKIGDVGPKGRILIRGEYGQDIIDELAPIEGEIVIDKPGKGAFY |
| | ATAFGDILAEKGIRCLVVTGVTTEVCVHTTVREANDRGYECLVLSDCVGSYFPEFQRVALEMIAAQGGIF |
| | GWVAPSAAFIDALAPLLAASAAQ |
| 756 | WP_076824447.1 MULTISPECIES: cysteine hydrolase [unclassified |
| | Bradyrhizobium]: |
| | MANSSGTIAAEPAPITLDWSRTALVIIDMQRDFMERGGFGETLGNDVSRLARAVQPIAAVLAAVRDAGLL |
| | VVHTREGHLPDLSDAPPAKVERGAPSLRIGDPGPMGRILIRGEAGHDIIPELYPLDSEIVIDKPGKGAFY |
| | ATEFGDILQKYGIENLLVCGVTTEVCVNTTVREANDRGYRCVVISDGCASYFPEFHEMGLKMIKAQGGIF |
| | GWVTDSAAVLEALGD |
| 757 | WP_076943533.1 cysteine hydrolase [Serratia oryzae]: |
| | MTQKTFHAEPFDLPFEIGSTALVMIDMQRDFVEPGGFGEALGNDVSFVRSAIEPCKRVLDAARRQGVLVI |
| | HTREGHRADLSDCPPAKLTRGGQTFIGTHGPMGRILVRGEAGHDIIPELYPQAGEPVIDKPGKGAFYQTD |
| | LHLILQNHGIKTLIVCGVTTEVCVNTTVREANDRGYECIIPQDCVGSYFPEFQKYALEMIKAQGAIFGWV |
| | SDSSAIVAGLQG |
| 758 | WP_077237805.1 cysteine hydrolase [Herbaspirillum sp. VT-16-41]: |
| | MIRIDATPYPYQFHPRSTALVVIDMQRDFIEEGGFGSALGNDVRPLAAIVPTVAALLQLAREAGMLVVHT |
| | RESHLPDLSDCPRSKRLRGNPTLGIGDVGPMGRILVQGEPGNQILPQLAPVEGELVIDKPGKGAFYATDL |
| | HAQLQERRITHLLVAGVTTEVCVQTSVREANDRGYECLVIEDACASYFPDFHRTTLEMLTAQGGIVGWRA |
| | PLAQLQAGVAAYTGDNP |
| 759 | XP_020122801.1 hypothetical protein UA08 01347 [Talaromyces |
| | atroroseus]: |
| | MVAAQSTKRLANGDRGDGVLTFSAQPYAFQFNPEKTALVIIDMQRDFLLKDGFGYIQAGEAGVEKVQATI |
| | KPTLAVLRAFRECGLHVIHTREGHRPDLRDLPTPKLLRQARAPETRHLMVIGDVGPMGRLLTRGEYGHDI |
| | VDELQPIAGEFVVDKPGKGGFFSTPLHEHLVDRGITHLIVVGVTVECCVTTTVREANDRGFEACILRDCT |
| | DGFVPAFKSAALDMIHFSEGLFGFVSESAPLLATLSGLPIYPKNGAPSWNGSVTFEALRNAYACGLSPTT |
| | VVKYILEKIEADSSTHPSVWISLKPMADLVHRAENLERLGDRNLPLFGIPFAVKDNIDVSGLPTTAACPS |
| | FQYVPDVSARVVERLEAAGAIVVGKTNLDQFATGLVGTRSPYGAVHCVDDSTRVSGGSSSGSALAVALGQ |
| | VSFSLGTDTAGSGRIPAAFNNIVGLKPTKGTVSTRGVIAACRTLDCVSFFASTLSDARSAWLAAKEYDPK |
| | DPYSKRSPSLLSLSNRSLLHEESTYSVSFPHAGILESLLSPAYLEHFGKIVALVRSMAYSEEVNFDWSSY |
| | FSASDLVYKSAFVAERSASLRELLSGKEEEITLHPVTAKVINQAKAMSATDAFRDMYHAQGLLKCAEAEF |
| | DKCDILIVPTAPNHPTIEEVEQDPIGPNLKLGIFASAVNVLDLAAIAIPAGHCQGLPFGISVIGPAFKEG |
| | FILEVAQRIQAHLNHFALD |
| 760 | >WP_091943349.1 \| Blastococcus endophyticus \| TrtA |
| | MDSLPTTPESPPRTVPAEPGPFPLPEGRVALLVIDMQRDFLLPGGFGESLGNDVTQLQRVVPPLTELLAG |
| | ARAAGLLVVHTREGHLPDLSDCPPAKLRRGAPSTRIGDPGPYGRILVRGEFGHDIVDELAPLPGEPVIDK |
| | PGKGAFYATGLGDLLRAAGVTHLLVTGVTTEVCVHTTVREANDRGYDCLWADCVGSYFPEFHRVGLQMV |
| | SAQGGIFGWVADSAAVRAALSPVPAPTSA |
| | Embodiments of Biuret Hydrolase Nucleic Acid Sequences |
| 761 | >Herbaspirillum sp BH-1 BiuH \| (codon optimized): |
| | ATGTCCATGGGTCCAGAATTGTTCATCAAAGCTGAGCCGTATGCCTGGCCGTATGATGGGGCCCTGACAC |
| | CAGCGAATACCGCCCTTATAGTCATAGACATGCAAACTGACTTCTGCGGGATAGGGGGGTATGTGGACAA |
| | GATGGGGTATGATCTGTCTCTGACTCGGGCTCCGATTGAACCGATCAAAAGAGTGCTGGCAGCGATGCGG |
| | GCCGGTGGCTATACCATCATCCATACCAGAGAGGGTCACCGCCCGGACCTTTCAGACTTACCCGCGAACA |
| | AGAGATGGCGGTCGAGACAGATCGGTACAAATGGAGTGGGGATTGGGGATGCAGGACCCTGTGGAAGAAT |
| | ATTGGTCCGGGGAGAGCCCGGCTGGGAGATCATACCTGAGTTGGCCCCTATAGCGGGCGAGATCATAATA |
| | GACAAACCTGGAAAAGGTTCGTTTTGCGCTACTGACTTAGAAATGATCTTACATACAAGAGGGATAAGAA |
| | ATATTGTCTTAACGGGAATTACCACCGATGTCTGCGTTCACACAACCATGAGAGAAGCTAATGATCGCGG |
| | TTTTGAATGTGTTATGCTGTCCGATTGCTGCGGCGCCACAGACCATAACAACCACTTAGCGGCGCTGAGT |
| | ATGATAAAGATGCAGGGAGGTGTGTTCGGGGCGGTGTCAGATTCTGCGGCATTAATCGATGTGATTGGTG |
| | CTAAGCTTGCGGCCGCACTCGAGGACTATAAAGACGATGATGATAAGTGA |
| 762 | >PKOI01000001.1 \| Herbaspirillum sp. BH-1 ctg1, whole genome shotgun |
| | sequence BiuH \| (native sequence): |
| | ATGCCCGAACTCTTCATCAAGGCCGAACCCTACGCCTGGCCCTACGATGGCGCCCTGACCCCGGCCAATA |
| | CCGCACTCATCGTCATCGACATGCAGACCGATTTCTGCGGCATCGGCGGTTACGTCGACAAGATGGGTTA |
| | CGACCTCTCGCTCACGCGCGCGCCGATCGAGCCGATCAAGCGCGTGCTGGCCGCCATGCGCGCCGGTGGC |
| | TACACCATCATCCACACCCGCGAAGGCCACCGCCCCGACCTCTCCGACCTGCCCGCCAACAAGCGCTGGC |
| | GCTCACGCCAGATCGGCACCAACGGCGTGGGCATCGGCGACGCCGGTCCCTGCGGCCGCATCCTGGTGCG |
| | CGGCGAGCCGGGCTGGGAAATCATCCCGGAACTGGCGCCCATCGCCGGCGAGATCATCATCGACAAACCC |
| | GGCAAAGGCTCCTTCTGCGCCACCGACCTGGAAATGATCCTGCACACCCGCGGCATCCGCAACATCGTGC |
| | TGACCGGCATCACCACCGATGTCTGCGTCCACACCACCATGCGCGAGGCCAATGACCGTGGCTTCGAATG |
| | CGTGATGCTCTCCGACTGCTGTGGCGCCACCGACCACAACAACCACCTGGCAGCGCTGTCGATGATCAAG |
| | ATGCAGGGTGGCGTCTTCGGTGCGGTCTCGGATTCGGCTGCGCTGATCGATGTGATCGGGGCCTGA |
| 763 | >AEX65081.1 \| Rhodococcus sp Mel BiuH \| (codon optimized): |
| | ATGGGTATTTATAGCACCGTGAACGCGAACCCGTATGCGTGGCCGTATGATGGTAGCATTGACCCGGCGC |
| | ACACCGCGCTGATTCTGATTGACTGGCAGATTGATTTCTGCGGTCCGGGTGGCTACGTGGACAGCATGGG |
| | TTATGATCTGAGCCTGACCCGTAGCGGCCTGGAGCCGACCGCGCGTGTTCTGGCGGCGGCGCGTGACACC |
| | GGTATGACCGTTATCCACACCCGTGAAGGTCACCGTCCGGACCTGGCGGATCTGCCGCCGAACAAACGTT |
| | GGCGTAGCGCGAGCGCGGGTGCGGAAATTGGTAGCGTGGGCCCGTGCGGTCGTATCCTGGTTCGTGGCGA |
| | GCCGGGTTGGGAAATTGTTCCGGAAGTGGCGCCGCGTGAGGGTGAACCGATCATTGATAAGCCGGGTAAA |
| | GGCGCGTTCTACGCGACCGACCTGGATCTGCTGCTGCGTACCCGTGGCATCACCCACCTGATTCTGACCG |
| | GTATCACCACCGACGTGTGCGTTCACACCACCATGCGTGAAGCGAACGATCGTGGTTATGAGTGCCTGAT |
| | TCTGAGCGACTGCACCGGTGCGACCGATCGTAAACACCACGAGGCGGCGCTGAGCATGGTGACCATGCAA |
| | GGTGGCGTTTTTGGTGCGACCGCGCACAGCGACGATCTGCTGGCGGCGCTGGGCACCACCGTTCCGGCGG |
| | CGGCGGGTCCGCGTGCGCGTACCGAA |
| 764 | >JN241637.1 \| Rhodococcus sp. Mel plasmid pMel2 BiuH \| (native |
| | sequence): |
| | ATGATCTACTCGACCGTGAACGCGAACCCCTACGCGTGGCCCTACGACGGCAGCATCGACCCCGCCCACA |
| | CTGCTCTAATCCTTATCGACTGGCAGATCGACTTTTGCGGCCCTGGCGGCTACGTCGACAGCATGGGTTA |
| | CGACCTCAGCCTCACGCGCTCAGGGCTGGAGCCGACCGCGCGCGTGCTGGCTGCGGCAAGAGACACCGGC |
| | ATGACCGTCATCCACACCAGGGAGGGCCACCGACCCGACCTCGCCGACCTCCCGCCTAACAAGCGCTGGC |
| | GATCCGCCAGCGCCGGGGCGGAGATCGGCAGCGTCGGACCATGCGGACGAATCCTGGTGCGAGGTGAGCC |
| | GGGATGGGAGATCGTTCCGGAAGTGGCGCCTCGCGAGGGTGAGCCGATCATTGACAAGCCGGGCAAAGGC |
| | GCGTTCTACGCCACGGACCTCGACCTGTTGCTGCGGACGCGGGGGATTACTCATCTGATCCTCACGGGTA |
| | TCACCACCGACGTCTGCGTGCACACCACGATGCGTGAGGCGAACGACCGCGGATACGAATGCCTGATCCT |
| | TTCGGACTGCACGGGCGCCACCGATCGGAAACACCACGAGGCCGCGCTCAGCATGGTCACCATGCAGGGA |
| | GGGGTCTTCGGCGCCACCGCCCACTCGGACGACCTCCTCGCCGCTTTGGGCACGACAGTGCCCGCGGCGG |
| | CCGGGCCTCGAGCGCGCACCGAATGA |
| 765 | >AM236084.1 \| Rhizobium leguminosarum bv. viciae plasmid pRL10 BiuH \| |
| | (native sequence): |
| | ATGGACGCGATGGTCGAAACCAACCGGCATTTTATCGACGCCGATCCGTATCCGTGGCCCTATAACGGAG |
| | CTCTGAGGCCTGACAATACCGCCCTCATCATCATCGACATGCAGACGGATTTCTGCGGCAAGGGCGGTTA |
| | TGTCGACCACATGGGCTACGACCTGTCGCTGGTGCAGGCGCCGATCGAACCCATCAAACGCGTGCTTGCC |
| | GCCATGCGGGCCAAGGGTTATCACATCATCCACACCCGCGAGGGCCACCGCCCCGACCTCGCCGATCTGC |
| | CAGCAAACAAACGCTGGCGCTCGCAACGGATCGGGGCCGGCATCGGTGATCCCGGCCCCTGCGGCCGAAT |
| | CCTGACGCGTGGCGAACCCGGCTGGGACATCATCCCCGAACTCTACCCGATCGAAGGCGAGACGATCATC |
| | GACAAGCCCGGCAAGGGTTCGTTCTGCGCCACCGACCTCGAACTCGTCCTCAACCAGAAACGCATCGAGA |
| | ACATTATCCTCACCGGGATCACCACCGATGTCTGCGTCTCGACGACGATGCGCGAGGCGAACGACCGCGG |
| | CTACGAATGCCTGCTGCTGGAGGACTGCTGTGGTGCGACCGACTACGGAAACCACCTCGCCGCCATCAAG |
| | ATGGTGAAGATGCAGGGCGGCGTCTTCGGCTCGGTCTCCAATTCCGCGGCTCTAGTCGAGGCGCTGCCCT |
| | GA |
| | Embodiments of Triuret Hydrolase Nucleic Acid Sequences |
| 766 | >PKOI01000001.1 \| Herbaspirillum sp. BH-1 ctg1, whole genome shotgun |
| | sequence \| TrtA (native sequence) |
| | ATGATCCGTATCGACGCCACGCCCTACCCTTACCAGTTCCACCCGCGCAGCACGGCGCTGGTGGTGATCG |
| | ACATGCAGCGCGACTTCATCGAGGAAGGCGGCTTCGGCAGCGCCCTCGGCAATGACGTGCGGCCGCTGGC |
| | GGCCATCGTGCCGACCGTGGCGGCGCTGCTGCAGCTGGCACGCGAGGCCGGCATGCTGGTGGTGCATACC |
| | CGCGAATCGCACCTGCCGGACTTGTCGGATTGCCCGCGCTCCAAGCGCCTGCGCGGCAATCCGACGCTGG |
| | GCATCGGCGATGTCGGGCCGATGGGCCGCATCCTGGTGCAAGGCGAGCCGGGCAACCAGATCCTGCCGCA |
| | ACTGGCCCCGGTGGAGGGCGAACTGGTCATCGACAAACCCGGCAAGGGCGCCTTCTACGCCACCGACCTG |
| | CATGCACAACTGCAGGAGCGTCGCATCACCCACCTGCTGGTGGCCGGCGTGACCACCGAAGTCTGCGTGC |
| | AGACCTCGATGCGCGAGGCCAATGACCGTGGCTATGAATGCCTGGTGATCGAGGATGCCTGCGCCAGCTA |
| | CTTCCCCGACTTCCATCGCATCACGCTGGAGATGCTGACGGCGCAGGGCGGCATCGTCGGCTGGCGTACC |
| | CCGCTGGCGCAACTGCAGGCCGGCGTGGCTGCCTACACAGGAGAAAATCCATGA |
| 767 | >AM236084.1 \| Rhizobium leguminosarum bv. viciae plasmid pRL10 |
| | complete genome, strain 3841 \| TrtA (native sequence) |
| | ATGGCGAAGATCAAGGCAGAACCCTTCGCCTTTCCGGTGAAGCACGATGAGCTCGCGCTCATCGTCATCG |
| | ACATGCAGCGCGATTTCGCCGAGCCGGGCGGCTTCGGTGCAAGCCTCGGCAATGATGTCAGCCGCATCAC |
| | CAGGATCGTGCCCGATGTCAAACGCCTGATCCAGGGCTTCCGCAATGCAGGCCTGCCTGTGATCCATACG |
| | ATGGAGTGCCACCGGCCTGATCTCTCCGACCTGCCGCCGGCCAAACGCGACCGCGGCAATCCTGCGCTCC |
| | GGATCGGCGACGAAGGCCCGATGGGCCGCATCCTGATTTCGGGGGAACCCGGCACGGCAATTCTTCCGGA |
| | ACTCGCTCCTGTGAAGGGCGAAGTCGTCATCGAAAAGCCCGGCAAGGGCGCCTTCTACGCGACCGACCTC |
| | GGCACCGTGCTGCAGCAGAAGGGCATCAAGCAGCTCGTCTTTGCCGGCGTCACCACCGAAGTCTGCGTGC |
| | AGACGACGATGCGCGAAGCAAACGACCGCGGTTATGAATGCCTTCTCGCCGAGGAGGCGACGGAAAGCTA |
| | TTTCCCCGAATTCAAAGCCGCCGCCATCGCCATGATCCGCGCCCAGGGCGCGATCGTCGGCTGGACCGCG |
| | CATGTCGACGACATTCTGGAAAGTATCGCCCATGCCTGA |
| 768 | >FOEE01000006.1 \| Blastococcus endophyticus strain DSM 45413 genome |
| | assembly, whole genome shotgun sequence \| TrtA (native sequence) |
| | ATGGATTCCCTCCCCACCACCCCCGAGAGCCCACCGCGCACCGTCCCCGCCGAGCCCGGCCCGTTCCCGC |
| | TCCCCGAGGGGAGGGTGGCCCTGCTGGTCATCGACATGCAGCGGGACTTCCTCCTGCCCGGCGGCTTCGG |
| | CGAGAGCCTGGGCAACGACGTCACGCAGCTGCAGCGCGTGGTCCCGCCGCTGACCGAGCTGCTCGCCGGC |
| | GCCCGCGCCGCAGGGCTGCTCGTCGTGCACACCCGCGAGGGCCACCTCCCCGACCTGTCGGACTGCCCGC |
| | CCGCGAAGCTGCGCCGCGGCGCCCCGAGCACGCGGATCGGCGACCCGGGCCCCTACGGCCGGATCCTGGT |
| | CCGCGGCGAGTTCGGCCACGACATCGTCGACGAGCTCGCTCCGCTCCCCGGGGAGCCGGTGATCGACAAG |
| | CCCGGCAAGGGCGCCTTCTACGCCACCGGGCTGGGCGACCTGCTCCGCGCCGCCGGCGTCACGCACCTGC |
| | TGGTCACCGGCGTCACGACCGAGGTGTGCGTGCACACCACCGTCCGCGAGGCCAACGACCGGGGCTACGA |
| | CTGCCTCGTCGTCGCCGACTGCGTCGGCTCCTACTTCCCCGAGTTCCACCGCGTCGGCCTGCAGATGGTC |
| | AGCGCGCAGGGCGGGATCTTCGGCTGGGTCGCCGACTCCGCCGCCGTCCGCGCCGCGCTCTCCCCCGTTC |
| | CCGCCCCCACCTCCGCCTGA |
| | Embodiment of a biuret Hydrolase Protein Sequence |
| 769 | >RKE06538.1 nicotinamidase-related amidase [Catellatospora citrea] |
| | MQTLVPQPDPPTGVRIGPVQADPYAWPYDGSVPVARTALLCIDWQTDFCGPGGYVDAMGYDIALTRAGLP |
| | ATAKLLDHVRSLGMLVVHTREGHDPDLSDLPANKRWRSARIGAEIGGPGPCGRILIKGEPGWEIVPEVAP |
| | APGEVVVDKPGKGAFYATNLDLVLRTRGITHLILTGITTDVCVHTTMREANDRGYECLILSDCTGATDPG |
| | NHAAALHMVTMQGGVFGCVATSDDVIAATTS |
| 771 | WP_037209122.1 cysteine hydrolase [Rhodovulum sp. NI22] |
| | MSYIDADPYNWPYNGDLRPDNTALIIIDMQTDFCGKGGYVDAMGYDLSLTQAPIGPIKALLGAMRDKGYL |
| | IIHTREGHRPDLADLPPNKRWRSQQIGAGIGDAGPCGKILIRGEPGWDIIPDLYPIAGEPIIDKPGKGSF |
| | CATDLELLLRTKGIDNIILTGITTDVCVHTTMREANDRGFECLLVEDCCGATDRGNHDAAIKMVKMQGGV |
| | FGAVSDSAKLIAALP |
| | Embodiment of a biuret Hydrolase Nucleic Acid Sequence |
| 770 | >RAPR01000001.1: 1618422-1619147 Catellatospora citrea strain DSM 44097 |
| | Ga0197484_11, whole genome shotgun sequence |
| | ATGCAAACCCTGGTGCCCCAACCTGATCCGCCGACCGGCGTACGCATCGGGCCGGTGCAGGCCGACCCGT |
| | ACGCGTGGCCGTACGACGGCTCGGTGCCCGTCGCACGGACGGCACTGCTGTGCATCGACTGGCAGACCGA |
| | CTTCTGCGGGCCGGGCGGCTACGTCGACGCGATGGGCTACGACATCGCGCTGACCCGGGCCGGGCTGCCC |
| | GCCACCGCCAAGCTGCTCGACCACGTGCGCTCGCTGGGCATGCTGGTCGTGCACACCCGCGAAGGCCACG |
| | ACCCGGACCTGTCCGACCTGCCCGCCAACAAGCGCTGGCGCTCGGCGCGGATCGGCGCGGAGATCGGCGG |
| | GCCCGGCCCGTGCGGGCGCATCCTGATCAAGGGCGAGCCGGGCTGGGAGATCGTGCCCGAGGTCGCCCCG |
| | GCGCCCGGCGAGGTCGTCGTCGACAAGCCCGGCAAGGGCGCGTTCTACGCCACCAACCTGGACCTGGTGC |
| | TGCGCACCCGCGGCATCACGCACCTGATCCTGACCGGCATCACCACCGACGTCTGCGTGCACACCACCAT |
| | GCGCGAGGCCAACGACCGCGGCTACGAGTGCCTGATCCTGTCCGACTGCACCGGCGCCACCGACCCCGGC |
| | AACCACGCCGCGGCCCTGCACATGGTCACCATGCAGGGCGGCGTCTTCGGCTGCGTGGCGACGTCCGACG |
| | ACGTCATCGCGGCCACCACCTCCTGA |
| 778 | >2646751319 Nicotinamidase-related amidase [Rhodovulum sp. NI22: |
| | Ga0070828_1034] (-)strand |
| | ATGAGCTATATCGACGCCGACCCCTATAACTGGCCCTATAATGGCGACCTGCGCCCCGACAATACCGCGC |
| | TGATCATCATCGACATGCAGACCGATTTCTGCGGCAAGGGCGGCTATGTCGATGCGATGGGCTATGACCT |
| | GTCGCTGACGCAGGCGCCGATCGGCCCGATCAAGGCGCTGCTGGGCGCGATGCGCGACAAGGGCTATCTG |
| | ATCATCCACACCCGCGAAGGGCATCGGCCCGATCTGGCCGACCTGCCGCCGAACAAGCGCTGGCGCAGCC |
| | AGCAGATCGGCGCGGGCATCGGCGATGCCGGCCCCTGCGGCAAGATCCTGATCCGGGGCGAGCCGGGCTG |
| | GGACATCATCCCCGACCTCTACCCGATCGCGGGCGAACCGATCATCGACAAGCCCGGCAAGGGCAGCTTC |
| | TGCGCCACCGATCTGGAGCTGTTGCTGCGTACCAAGGGCATCGACAACATCATCCTGACCGGCATCACCA |
| | CCGATGTCTGCGTTCACACCACCATGCGCGAGGCCAATGACAGGGGATTCGAATGCCTGCTGGTCGAGGA |
| | TTGCTGCGGCGCCACCGACAGGGGCAACCATGACGCGGCCATCAAGATGGTGAAGATGCAGGGCGGCGTG |
| | TTCGGCGCGGTATCGGACAGCGCCAAGCTGATCGCGGCGCTGCCATGA |
| | Embodiments of Cyanuric Acid amidohydrolases Protein Sequence |
| 772 | >AAC61577.1 cyanuric acid amidohydrolase [Acidovorax citrulli] |
| | MQAQVFRVPMSNPADVSGVAKLIDEGVIRAEEVVCVLGKTEGNGCVNDFTRGYTTLAFKVYFSEKLGVSR |
| | QEVGERIAFIMSGGTEGVMAPHCTIFTVQKTDNKQKTAAEGKRLAVQQIFTREFLPEEIGRMPQVTETAD |
| | AVRRAMREAGIADASDVHFVQVKCPLLTAGRMHDAVERGHTVATEDTYESMGYSRGASALGIALALGEVE |
| | KANLSDEVITADYSLYSSVASTSAGIELMNNEIIVMGNSRAWGGDLVIGHAEMKDAIDGAAVRQALRDVG |
| | CCENDLPTVDELGRVVNVFAKAEASPDGEVRNRRHTMLDDSDINSTRHARAVVNAVIASIVGDPMVYVSG |
| | GSEHQGPAGGGPVAVIARTA |
| 773 | >WP_011117191.1 MULTISPECIES: cyanuric acid amidohydrolase |
| | [Pseudomonas sp. ADP] |
| | MYHIDVFRIPCHSPGDTSGLEDLIETGRVAPADIVAVMGKTEGNGCVNDYTREYATAMLAACLGRHLQLP |
| | PHEVEKRVAFVMSGGTEGVLSPHHTVFARRPAIDAHRPAGKRLTLGIAFTRDFLPEEIGRHAQITETAGA |
| | VKRAMRDAGIASIDDLHFVQVKCPLLTPAKIASARSRGCAPVTTDTYESMGYSRGASALGIALATEEVPS |
| | SMLVDESVLNDWSLSSSLASASAGIELEHNVVIAIGMSEQATSELVIAHGVMSDAIDAASVRRTIESLGI |
| | RSDDEMDRIVNVFAKAEASPDGVVRGMRHTMLSDSDINSTRHARAVTGAAIASVVGHGMVYVSGGAEHQG |
| | PAGGGPFAVIARA |
| 774 | >WP_012172412.1 ring-opening amidohydrolase [Azorhizobium caulinodans] |
| | MPIAKVHRIATASPDDVSGLAAAIATGAIAPAGILAIFGKTEGNGCVNDFSRGFAVQSLQMLLRGHMGAA |
| | ADEVCLVMSGGTEGGMSPHFLVFERAEGNAPEAAPALAIGRAHTPDLPFEALGRMGQVRMVAQAVRRAMA |
| | AAGITDPEDVHFVQVKCPLLTAMRVKEAEARGATTATSDTLKSMGLSRGASALGIALALGEVAEDALSDA |
| | VICADYGLWSARASCSSGIELLGHEIVVLGMSEGWSGPLAIAHGVMADAIDVTPVKAALSALGAEAGEAT |
| | IVLAKAEPSRSGRIRGKRHTMLDDSDISPTRHARAFVAGALAGVVGHTEIYVSGGGEHQGPDGGGPVAVI |
| | AARTMG |
| | Embodiments of ammelide hydrolase Protein Sequence |
| 775 | >WP_011117177.1 MULTISPECIES: N-isopropylammelide isopropyl |
| | amidohydrolase [Pseudomonas sp. ADP] |
| | MSKDFDLIIRNAYLSEKDSVYDIGIVGDRIIKIEAKIEGTVKDEIDAKGNLVSPGFVDAHTHMDKSFTST |
| | GERLPKFWSRPYTRDAAIEDGLKYYKNATHEEIKRHVIEHAHMQVLHGTLYTRTHVDVDSVAKTKAVEAV |
| | LEAKEELKDLIDIQWAFAQSGFFVDLESESLIRKSLDMGCDLVGGVDPATRENNVEGSLDLCFKLAKEY |
| | DVDIDYHIHDIGTVGVYSINRLAQKTIENGYKGRVTTSHAWCFADAPSEWLDEAIPLYKDSGMKFVTCFS |
| | STPPTMPVIKLLEAGINLGCASDNIRDFWVPFGNGDMVQGALIETQRLELKTNRDLGLIWKMITSEGARV |
| | LGIEKNYGIEVGKKADLVVLNSLSPQWAIIDQAKRLCVIKNGRIIVKDEVIVA |
| 776 | >AAK00493.1 ammelide aminohydrolase [Acidovorax citrulli] |
| | MSMETHSYVDVAIRNARLADTEGIVDILIHDGRIASIVKSTKTKGSVEIDAHEGLVTSGLVEPHIHLDKA |
| | LTADRVPAGSIGDLRTRRGLEMAIRATRDIKRTFTVEDVRERAIRAALMASRAGTTALRTHVDVDPIVGL |
| | AGIRGVLEAREVCAGLIDIQIVAFPQEGLFCSAGAVDLMREAIKLGADAVGGAPALDDRPQDHVRAVFDL |
| | AAEFGLPVDMHVDESDRREDFTLPFVIEAARERRVPNVTVAHISSLSVQTDDVARSTIAALADADVNVVV |
| | NPIIVKITRLSELLDAGVSVMFGSDNLRDPFYPLGAANPLGSAIFACQIAALGTPQDLRRVFDAVTINAA |
| | RMLGFPSLLGVVEGAVADLAVFPSATPEEVVLDQQSPLFVLKGGRVVAMRLAAGSTSFRDYS |
| | Embodiments of AtzC Nucleic Acid Sequence |
| 777 | >gb\|LKAX01000023.1 \|: 22881-24209 Pseudomonas sp. ADP plasmid pADP1, |
| | whole genome shotgun sequence |
| | ATGAGTAAAGATTTTGATTTAATCATTAGAAACGCCTATCTAAGTGAAAAAGACAGTGTATATGATATTG |
| | GGATTGTTGGTGACAGAATAATCAAAATAGAAGCTAAAATTGAAGGAACCGTAAAAGACGAAATTGATGC |
| | AAAGGGTAACCTTGTGTCTCCCGGATTTGTCGATGCACATACCCATATGGATAAGTCATTTACGAGCACA |
| | GGTGAAAGATTACCGAAGTTTTGGAGCAGACCTTATACAAGGGATGCTGCCATCGAGGATGGCTTGAAAT |
| | ATTATAAAAATGCTACCCACGAAGAAATAAAAAGACATGTGATAGAACATGCTCACATGCAGGTACTCCA |
| | TGGGACTTTATACACCCGGACCCATGTAGATGTAGATTCAGTTGCTAAAACAAAAGCAGTGGAAGCAGTT |
| | TTAGAAGCCAAGGAAGAGTTAAAGGATCTTATCGATATACAAGTCGTAGCCTTTGCACAGAGTGGATTTT |
| | TCGTTGATTTGGAATCTGAATCATTGATTAGAAAATCCTTGGATATGGGCTGTGATTTAGTTGGGGGAGT |
| | TGATCCTGCTACGCGGGAAAATAATGTTGAGGGTTCTTTAGACCTATGCTTTAAATTAGCAAAGGAATAC |
| | GATGTTGATATCGACTATCACATACATGATATTGGAACTGTTGGAGTATATTCGATAAATCGTCTTGCCC |
| | AAAAGACAATTGAAAATGGGTATAAGGGTAGAGTAACTACGAGTCATGCCTGGTGTTTTGCAGATGCTCC |
| | GTCCGAATGGCTCGATGAGGCAATCCCATTGTACAAGGATTCGuGTATGAAATTTGTTACCTGTTTTAGT |
| | AGTACACCGCCTACTATGCCGGTGATAAAGCTGCTTGAAGCTGGCATCAATCTTGGCTGTGCTTCGGACA |
| | ATATCAGAGATTTTTGGGTTCCCTTTGGCAACGGTGATATGGTACAAGGGGCTCTGATCGAAACTCAGAG |
| | ATTAGAGTTAAAGACAAACAGAGATTTGGGACTAATTTGGAAAATGATAACGTCAGAGGGTGCTAGAGTT |
| | TTAGGAATTGAAAAGAACTATGGGATAGAAGTTGGTAAAAAGGCCGATCTTGTTGTATTAAATTCGTTGT |
| | CACCACAATGGGCAATAATCGACCAAGCAAAAAGACTATGCGTAATTAAAAATGGACGTATCATTGTGAA |
| | GGATGAGGTTATAGTTGCCTAA |

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of reducing biuret in a urea composition, the method comprising contacting the urea composition with an isolated or purified biuret hydrolase enzyme under conditions suitable to reduce the concentration of biuret in the urea composition.

2. The method of claim 1, wherein the biuret hydrolase enzyme comprises a R[E/D]AN motif.

3. The method of claim 1, wherein the biuret hydrolase enzyme comprises a R[E/D]ANDRG[F/Y][E/D]C motif.

4. The method of any one of claims 1-3, wherein the biuret hydrolase is derived from a bacterium of Catellatospora Citrea, Rhodovulum sp. NI22, Herbaspirillum, Rhizobium or Rhodococcus.

5. The method of any one of claims 1-3, wherein the biuret hydrolase is derived from a bacterium of Catellatospora Citrea, Rhodovulum sp. NI22, Herbaspirillum sp. BH-1, Rhizobium leguminosarum or Rhodococcus sp. Mel.

6. The method of any one of claims 1-5, wherein the biuret hydrolase comprises an amino acid sequence having at least about 80% sequence identity to any one of SEQ ID NOs:1-164, 769 and 771.

7. The method of any one of claims 1-5, wherein the biuret hydrolase comprises an amino acid sequence having at least about 95% sequence identity to any one of SEQ ID NOs:1-164, 769 and 771.

8. The method of any one of claims 1-5, wherein the biuret hydrolase comprises an amino acid sequence having at least about 95% sequence identity to SEQ ID NO:1, 2, 95, 769, or 771.

9. The method of claim 8, wherein the biuret hydrolase comprises SEQ ID NO:1, 2, 95, 769, or 771.

10. The method of any one of claims 1-9, wherein the biuret hydrolase enzyme is conjugated to a carrier or a solid support.

11. The method of any one of claims 1-9, wherein the biuret hydrolase enzyme is present in a composition comprising a matrix (e.g., a matrix comprising silica).

12. The method of any one of claims 1-9, wherein the biuret hydrolase enzyme is present in a device (e.g., a filter).

13. The method of any one of claims 1-12, further comprising contacting the urea composition with an isolated or purified cyanuric acid hydrolase (CAH) enzyme, an isolated or purified triuret hydrolase enzyme, and/or an isolated or purified ammelide hydrolase enzyme.

14. The method of claim 13, wherein the CAH enzyme, the triuret hydrolase enzyme, and/or ammelide hydrolase enzyme is conjugated to a carrier or a solid support.

15. The method of claim 13, wherein the CAH enzyme, the triuret hydrolase enzyme, and/or an ammelide hydrolase enzyme is present in a composition comprising the biuret hydrolase enzyme and a matrix.

16. The method of claim 13, wherein the CAH enzyme, the triuret hydrolase enzyme, and/or an ammelide hydrolase enzyme is present in a device comprising the biuret hydrolase enzyme.

17. The method of any one of claim 1-16, wherein the urea composition has a urea concentration between about 0.1M and 6.0M.

18. The method of any one of claims 1-17, wherein the urea composition is a liquid.

19. The method of any one of claims 1-17, wherein the method further comprises mixing a solid urea composition and the biuret hydrolase enzyme with water.

20. The method of any one of claims 1-19, wherein the urea composition is a fertilizer or a diesel exhaust fluid (DEF) composition.

21. The method of any one of claims 1-20, wherein the concentration of biuret in the urea composition is reduced to less than about 0.1%.

22. The method of any one of claims 1-20, wherein the concentration of biuret in the urea composition is reduced to less than about 0.01%.

23. The method of any one of claims 1-20, wherein the concentration of biuret in the urea composition is reduced to less than about 0.001%.

24. The method of any one of claims 1-20, wherein the concentration of biuret in the urea composition is reduced to an undetectable level.

25. The method of any one of claims 1-24, wherein the biuret hydrolase enzyme is comprised within a cell or cell lysate (e.g., a cross-linked and/or encapsulated cell).

26. The method of any one of claims 13-25, wherein the CAH enzyme, the triuret hydrolase enzyme, and/or an ammelide hydrolase enzyme is comprised within a cell or cell lysate (e.g., a cross-linked and/or encapsulated cell).

27. A composition comprising an isolated or purified biuret hydrolase enzyme and a matrix (e.g., a matrix comprising silica).

28. The composition of claim 27, wherein the biuret hydrolase enzyme comprises an amino acid sequence having at least about 80% sequence identity to any one of SEQ ID NOs:1-164, 769 and 771.

29. The composition of claim 27 or 28, further comprising a CAH enzyme, a triuret hydrolase enzyme, and/or an ammelide hydrolase enzyme.

30. The composition of any one of claims 27-29, wherein the biuret hydrolase enzyme, the CAH enzyme, the triuret hydrolase enzyme, and/or an ammelide hydrolase enzyme is comprised within a cell(s) or cell lysate (e.g., a cross-linked and/or encapsulated cell).

31. A device comprising an isolated or purified biuret hydrolase enzyme and a matrix.

32. The device of claim 31, wherein the biuret hydrolase enzyme comprises an amino acid sequence having at least about 80% sequence identity to any one of SEQ ID NOs:1-164, 769 and 771.

33. The device of claim 31 or 32, further comprising a CAH enzyme, a triuret hydrolase enzyme, and/or an ammelide hydrolase enzyme.

34. The device of any one of claims 31-33, wherein the biuret hydrolase enzyme, the CAH enzyme, the triuret hydrolase enzyme, and/or an ammelide hydrolase enzyme is comprised within a cell(s) or cell lysate (e.g., a cross-linked and/or encapsulated cell).

35. The device of any one of claims 31-34, further comprising a casing or housing for the matrix, wherein liquid flows through the at least one casing and contacts at least one enzyme or cell.

36. The device of claim 35, further comprising a permeable layer.

37. A kit comprising an isolated or purified biuret hydrolase enzyme and instructions for contacting a urea composition comprising biuret with the biuret hydrolase enzyme for reducing the concentration of biuret in the composition.

38. The kit of claim 37, wherein the biuret hydrolase enzyme comprises an amino acid sequence having at least about 80% sequence identity to any one of SEQ ID NOs:1-164, 769 and 771.

39. The kit of claim 37 or 38, further comprising an isolated or purified CAH enzyme, an isolated or purified triuret hydrolase enzyme, and/or an isolated or purified ammelide hydrolase enzyme.

40. The kit of any one of claims 37-39, wherein the biuret hydrolase enzyme, the CAH enzyme, the triuret hydrolase enzyme, and/or an ammelide hydrolase enzyme are present in a composition or device.

41. An isolated or purified triuret hydrolase enzyme comprising an amino acid sequence having an F at position 35, an L at position 39, an N at position 41, an E at position 160, a Y at position 187 and/or and I at position 205, wherein each position is relative to a triuret hydrolase amino acid sequence derived from Herbaspirillum sp. BH-1.

42. An isolated or purified triuret hydrolase enzyme comprising an amino acid sequence having at least 80% sequence identity to any one of SEQ ID NOs:169-760.

43. The isolated or purified triuret hydrolase enzyme of claim 41 or 42, wherein the amino acid sequence has at least 90% sequence identity to any one of SEQ ID NOs:169-760.

44. The isolated or purified triuret hydrolase enzyme of claim 42 or 43, wherein the amino acid sequence comprises any one of SEQ ID NOs:169-760.

45. The isolated or purified triuret hydrolase enzyme of claim 42 or 43, wherein the amino acid sequence comprises any one of SEQ ID NOs:169-171.

46. The isolated or purified triuret hydrolase enzyme of claim 42 or 43, consisting of SEQ ID NO:169, SEQ ID NO:170 or SEQ ID NO:171.

47. The isolated or purified triuret hydrolase enzyme of any one of claims 41-46, wherein the enzyme is derived from Herbaspirillum (e.g., Herbaspirillum sp. BH-1), Rhzobium or Actinoplanes.

48. An isolated or purified nucleic acid encoding a triuret hydrolase enzyme of any one of claims 41-47.

49. An expression cassette comprising the nucleic acid of claim 48.

50. A vector comprising the expression cassette of claim 49.

51. A cell comprising the expression cassette of claim 49 or the vector of claim 50.

52. A composition comprising the isolated or purified triuret hydrolase enzyme as described in any one of claims 41-47 and a matrix (e.g., a matrix comprising silica).

53. A device comprising a triuret hydrolase enzyme as described in any one of claims 41-47 or a composition as described in claim 52 and a matrix.

54. The device of claim 53, wherein the device further comprises a casing or housing for the matrix, wherein liquid flows through the at least one casing and contacts at least one enzyme.

55. The device of claim 54, further comprising a permeable layer.

56. A method of reducing triuret in a composition, the method comprising contacting the composition with an isolated or purified triuret hydrolase enzyme as described in any one of claims 41-47, under conditions suitable to reduce the concentration of triuret in the composition.

57. The method of claim 56, wherein the composition comprises water.

58. The method of claim 56 or 57, wherein the composition comprises urea.

59. A kit comprising a triuret hydrolase enzyme of any one of claims 41-47, a cell of claim 51, a composition of claim 52 or a device of any one of claims 53-55 and instructions for contacting a first composition comprising triuret with the triuret hydrolase enzyme, cell, composition or device, for reducing the concentration of triuret in the first composition.
